(19) United States
(12) Patent Application Publication
(10) Pub. No.: US 2024/0309466 A1
(43) Pub. Date: Sep. 19, 2024

(54) RAPID LAMP METHODS FOR DETECTING BACTERIAL AND VIRAL PATHOGENS

(71) Applicant: The Johns Hopkins University, Baltimore, MD (US)

(72) Inventor: Subhra Chakraborty, Lutherville Timonium, MD (US)

(21) Appl. No.: 18/572,850

(22) PCT Filed: Jun. 21, 2022
(86) PCT No.: PCT/US2022/034399

§ 371 Date: Dec. 21, 2023

Related U.S. Application Data
(60) Provisional application No. 63/212,876, filed on Jun. 21, 2021.

(51) Int. Cl. C12Q 1/689; C12Q 1/6844

(52) U.S. Cl. CPC C12Q 1/689; C12Q 1/6844

(57) ABSTRACT

Disclosed herein are methods for detecting a pathogenic microorganism in a sample. The methods can include the steps of contacting the sample with a lysis solution to form a mixture; filtering the mixture through a filter to form a filtered mixture, wherein the filtered mixture comprises DNA or RNA of the target microorganism; contacting the filtered mixture with loop mediated isothermal amplification (LAMP) reagents and one or more primer sets specific to the DNA or RNA of the target microorganism, amplifying the DNA or RNA of the target microorganism, thereby producing one or more amplicons; and detecting the presence or absence of the one or more amplicons, wherein the presence of the one or more of the amplicons indicates the presence of the pathogenic microorganism. The LAMP reagents and the one or more primer sets can be lyophilized.

### CROSS REFERENCE TO RELATED APPLICATIONS

This application claims the benefit of U.S. Provisional Application No. 63/212,876, filed on Jun. 21, 2021. The content of this earlier filed application is hereby incorporated by reference herein in its entirety.

### STATEMENT REGARDING FEDERALLY FUNDED RESEARCH

This invention was made with government support under grant numbers AI149760, A1145435, EB020539, A1153349, and A1137804 awarded by the National Institutes of Health. The government has certain rights in the invention.

### INCORPORATION OF THE SEQUENCE LISTING

The present application contains a sequence listing that is submitted via EFS-Web concurrent with the filing of this application, containing the file name “36406_0024P1_SL.txt” which is 274,432 bytes in size, created on Jun. 6, 2022, and is herein incorporated by reference in its entirety.

### BACKGROUND

Enterotoxigenic E. coli (ETEC) and Shigella spp (Shigella) are the primary causes of moderate-to-severe diarrhea in the children <5 years of age living in impoverished areas of the world (Qadri F, et al. Clin. Microbiol. 2005 Rev. 18(3), 465-483; and Kotloff K L, et al. Lancet. 2013; 382(9888):209-22). These pathogens are also the most frequent bacterial causes of diarrhea among travelers to Africa, Asia, and Latin America, including military personnel deployed to these areas (Jiang Z D, et al. J. Infect. Dis. 2002; 185(4), 497-502; Sack D A, et al. Vaccine 2007; 25(22), 4392-4400; Steffen R, and Connor B A. J. Travel Med. 2005; 12(1), 26-35; Hameed J M, et al. PLoS One. 2016; 11(5):e0154830; and Rivera F P, et al. J Clin Microbiol. 2013 51(2):633-5).

ETEC and Shigella are complex pathogens and the diagnostic assays used to detect these pathogens are either elaborate or complex. ETEC are characterized on a molecular basis by the presence of genes that encode the heat-stable (ST) and/or heat-labile (LT) enterotoxins (Qadri F, et al. Clin. Microbiol. 2005 Rev. 18(3), 465-483; and Chakraborty S, et al. J Clin Microbiol. 2001; 39(9):3241-6). In the absence of a selective media, the most frequently used diagnostic assay for ETEC is culturing the stool samples on MacConkey agar and isolating 3 to 5 E. coli colonies followed by PCR targeting the toxin genes (Qadri F, et al. Clin. Microbiol. 2005 Rev. 18(3), 465-483; Kotloff K L, et al. Lancet. 2013; 382(9888):209-22; Chakraborty S, et al. J Clin Microbiol. 2001; 39(9):3241-6; Kahali S, et al. Eur J Epidemiol. 2004; 19(5):473-9; and Lindsay B R, et al. FEMS Microbiol Lett. 2014; 352(1):25-31). The other diagnostic methods used are, GM1 ganglioside ELISA and DNA probe hybridization assays which also target the toxins using cultured E. coli colonies (Qadri F, et al. Clin. Microbiol. 2005 Rev. 18(3), 465-483; and Youmans B P, et al. Am J Trop Med Hyg. 2014 90(1):124-32). On the other hand, conventional bacterial culture is the gold standard for detection of Shigella (Kotloff K L, et al. Lancet. 2013; 382(9888):209-22; Eileen M. Barry, et al. Nat Rev Gastroenterol Hepatol. 2013; 10(4):245-55; and Livio S, et al. Clin Infect Dis. 2014 October; 59(7):933-41). For culture of Shigella, stool specimens are inoculated onto Xylose Lysine Deoxycholate (XLD), Hektoen Enteric agar (HEA), and/or Salmonella Shigella Agar (SSA). The Shigella like colonies are then selected for further biochemical analysis and confirmed serologically by slide agglutination using commercially available antisera (Kotloff K L, et al. Lancet. 2013; 382(9888):209-22; Eileen M. Barry, et al. Nat Rev Gastroenterol Hepatol. 2013; 10(4):245-55; and Livio S, et al. Clin Infect Dis. 2014 October; 59(7):933-41). Recent studies have shown that the current diagnostic methods for both ETEC and Shigella are not sufficiently sensitive to reflect the true burden of these pathogens. The sensitivity of these assays depends on the number of E. coli colonies or suspected Shigella colonies screened (Lindsay B R, et al. FEMS Microbiol Lett. 2014; 352(1):25-31; Youmans B P, et al. Am J Trop Med Hyg. 2014 90(1):124-32; and Lindsay B, et al. J Clin Microbiol. 2013 51(6):1740-6).

In addition, the current World Health Organization (WHO) guidelines for treatment of shigellosis (in the absence of a rapid, sensitive, simple and inexpensive diagnostic test) recommends treatment with antibiotics when presence of visible blood in stool (dysentery). The sensitivity of dysentery for identifying Shigella appears to have declined over time. A systemic review showed that between 1977 and 2016, dysentery identified 1.9-85.9% of confirmed Shigella infections, with sensitivity decreasing over time (p=0.04) (Tickell K D et al. Lancet Glob Health. 2017; 5(12):e1235-e1248. A simple and rapid test that is applicable to the health settings for diagnosis and treatment could reduce mortality and long-term growth potential among children infected with Shigella.

### BRIEF DESCRIPTION OF THE DRAWINGS

FIG. 1 shows lyophilized rapid LAMP-based diagnostic test (RLDT) strip tubes.

FIG. 2 shows comparison of time to reacting between wet and dry formulations of RLDT for detection of ETEC (e.g., LT, STh, and STp) and Shigella (e.g., ipaH) targets.

FIG. 3 shows the stability of the lyophilized RLDT assay strips.

FIG. 4 shows the lowest detection limits of ETEC (e.g., LT, STh, STp) and Shigella (e.g., ipaH) target genes in RLDT.

FIG. 5 shows the linearity of the TTR values for LT, STh, STp and ipaH.

FIG. 6 shows a comparison of RLDT to qPCR tests for each gene. Note: *Statistical significance (p<0.05).

FIG. 7 shows the results of a stability test of the lyophilized RLDT assay strips and reagents every 3 months for one year. The RLDT strips with lyophilized LAMP reagents and lyophilized lysis buffer B were placed in room temperature (24° C.), at 37° C. and at 42° C. 1 year and tested every 3 months with stool samples that were spiked with either Shigella or Vibrio cholerae at either 10⁷ CFU/gm of stool or 10⁵ CFU/gm of stool. RT: Room temperature; TTR: Time to Result (become positive); M: month; Baseline: Before placing the strips and reagents for stability.

### SUMMARY

Disclosed herein are method of detecting a target microorganism in a sample, the methods comprising: a) obtaining or having obtained a sample from a subject, wherein the sample comprises or is suspected of comprising the target microorganism; b) contacting the sample with a lysis solution to form a mixture; c) filtering the mixture of b) through a filter to form a filtered mixture, wherein the filtered mixture comprises DNA or RNA of the target microorganism; d) contacting the filtered mixture of c) with loop mediated isothermal amplification (LAMP) reagents and one or more primer sets specific to the DNA or RNA of the target microorganism, wherein the LAMP reagents and the one or more primer sets are lyophilized; e) amplifying the DNA or RNA of the target microorganism, thereby producing one or more amplicons; and f) detecting the presence or absence of the one or more amplicons; wherein the presence of the one or more of the amplicons indicates the presence of the target microorganism.

Disclosed herein are methods detecting pathogenic E. coli in a sample, the methods comprising: a) obtaining or having obtained a sample from a subject, wherein the sample comprises or is suspected of comprising the pathogenic E. coli; b) contacting the sample with a lysis solution to form a mixture; c) filtering the mixture of b) through a filter to form a filtered mixture, wherein the filtered mixture comprises DNA or RNA of the pathogenic E. coli; and d) contacting the filtered mixture of c) with loop mediated isothermal amplification (LAMP) reagents and one or more primer sets specific to the DNA or RNA of the pathogenic E. coli, wherein the LAMP reagents and the one or more primer sets are lyophilized; e) amplifying DNA or RNA of the pathogenic E. coli, thereby producing one or more amplicons; and f) detecting the presence or absence of the one or more amplicons; wherein the presence of the one or more amplicons indicates the presence of the pathogenic E. coli.

Disclosed herein are methods of detecting Shigella spp in a sample, the methods comprising: a) obtaining or having obtained a sample from a subject, wherein the sample comprises or is suspected of comprising the Shigella spp; b) contacting the sample with a lysis solution to form a mixture; c) filtering the mixture of b) through a filter to form a filtered mixture, wherein the filtered mixture comprises DNA or RNA of the Shigella spp; and d) contacting the filtered mixture of c) with loop mediated isothermal amplification (LAMP) reagents and one or more primer sets specific to the DNA or RNA of the Shigella spp, wherein the LAMP reagents and the one or more primer sets are lyophilized; e) amplifying DNA or RNA of the Shigella spp, thereby producing one or more amplicons; and f) detecting the presence or absence of the one or more amplicons; wherein the presence of the one or more amplicons indicates the presence of the Shigella spp.

Disclosed herein are methods of detecting a Salmonella spp. in a sample, the methods comprising: a) obtaining or having obtained a sample from a subject, wherein the sample comprises or is suspected of comprising the Salmonella spp.; b) contacting the sample with a lysis solution to form a mixture; c) filtering the mixture of b) through a filter to form a filtered mixture, wherein the filtered mixture comprises DNA or RNA of the Salmonella spp.; and d) contacting the filtered mixture of c) with loop mediated isothermal amplification (LAMP) reagents and one or more primer sets specific to the DNA or RNA of the Salmonella spp., wherein the LAMP reagents and the one or more primer sets are lyophilized; e) amplifying DNA or RNA of the Salmonella spp., thereby producing one or more amplicons; and f) detecting the presence or absence of the one or more amplicons; wherein the presence of the one or more amplicons indicates the presence of the Salmonella spp.

Disclosed herein are kits for detecting a target microorganism in a sample, the kits comprising: a lysis buffer; a filter; a lyophilized buffer; loop mediated isothermal amplification (LAMP) reagents; and one or more primer sets specific to the DNA or RNA of the target microorganism, wherein the LAMP reagents and the one or more primer sets are lyophilized.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile, sputum, pap smear, oropharyngeal, and nasopharyngeal) that contains cells or cell components. In some aspects, a sample can be an environmental sample (e.g., water, sewage, fruits, or vegetables).

As used herein, the term “subject” refers to the target of administration or the source of the sample, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In another aspect, a subject is a human. The term does not denote a particular age or sex. Thus, adult, child, adolescent and newborn subjects, as well as fetuses, whether male or female, are intended to be covered.

As used herein, the term “comprising” can include the aspects “consisting of” and “consisting essentially of.”

The term “contacting” as used herein refers to bringing a compound or solution and a sample, a cell, target receptor, or other biological entity together in such a manner that the compound or solution can affect the activity of the sample, either directly; i.e., by interacting with the sample itself or indirectly.

As used herein, the term “level” refers to the amount of a target molecule in a sample, e.g., a sample from a subject. The amount of the molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compares to a control (i.e., a non-affected or healthy subject or a sample from a non-affected or healthy subject) or a sham or an untreated sample).

The phrase “at least” preceding a series of elements is to be understood to refer to every element in the series. For example, “at least one” includes one, two, three, four or more.

The term “incubating” is used synonymously with “contacting” and “exposing” and does not imply any specific time or temperature requirements unless otherwise indicated.

As used herein, the term “patient” refers to a subject afflicted with a disease, disorder or infection. The term “patient” includes human and veterinary subjects. In some aspects of the disclosed methods the “patient” has been diagnosed or identified with a need for treatment, for having an infection (e.g., pathogenic E. coli), such as, for example, prior to the detecting or administering step.

By “specifically hybridizes” is meant that a probe, primer, or oligonucleotide recognizes and physically interacts (that is, base-pairs) with a substantially complementary nucleic acid under high stringency conditions, and does not substantially base pair with other nucleic acids.

The term “primer” refers to an oligonucleotide (synthetic or occurring naturally) that is capable of acting as a point of initiation of nucleic acid synthesis or replication along a complementary strand when placed under conditions in which synthesis of a complementary strand is catalyzed by a polymerase.

By “probe,” “primer,” or oligonucleotide is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for a microbe-specific antibody and have at least 80%-90% sequence complementarity, preferably at least 91%-95% sequence complementarity, more preferably at least 96%-99% sequence complementarity, and most preferably 100% sequence complementarity to the region of the microbe-specific antibody to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: loop mediated isothermal amplification (LAMP), nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

The term “hybridization” refers to a process of establishing a non-covalent, sequence-specific interaction between two or more complementary strands of nucleic acids into a single complex, which in the case of two strands is referred to as a double-stranded DNA or duplex.

By “high stringency conditions” is meant conditions that allow hybridization comparable with that resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, NY, 1998). The term “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

The term “amplicon” is used herein to refer to an elongation product. An amplicon is a piece of DNA or RNA that is the source and/or product of an amplification event. It can be formed, for example, using loop mediated isothermal amplification (LAMP) reactions.

“Label” refers to a molecule attached to an oligonucleotide (covalently or non-covalently) and capable of providing information about the oligonucleotide it is attached to, including, but not limited to, radioactive isotopes, fluorophores, chemiluminescent reagents, dyes, enzymes, enzyme substrates, or semiconductor nanocrystals, such as quantum dots. Labels can provide a detectable (and optionally quantifiable) signal.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

Enterotoxigenic E. coli and Shigella are the leading causes of moderate to severe diarrhea in the low and middle income countries. A constraint to control ETEC and Shigella diarrhea is the complex diagnostic methods currently required for detecting these infections. These methods are neither sufficiently sensitive nor standardized and are not feasible in the resource poor settings where these infections occur most commonly. To address this gap, and as described herein a rapid and simple diagnostic assay—“ETEC and Shigella Rapid LAMP based Diagnostic Test (RLDT)” was developed. Using RLDT, ETEC and Shigella could be detected directly from the stool and the assay could be performed in less than 1 hour with minimal hands on time. A battery operated; hand-held reader can be used to read the RLDT results in a user-friendly manner. Being rapid, simple and inexpensive, RLDT can be scaled up and is appropriate to apply in the resource poor endemic settings.

### Methods

Disclosed herein are methods of detecting a target microorganism in a sample. In some aspects, the method can comprise: a) obtaining or having obtained a sample from a subject, wherein the sample comprises or is suspected of comprising the target microorganism; b) contacting the sample with a lysis solution to form a mixture; c) filtering the mixture of b) through a filter to form a filtered mixture, wherein the filtered mixture comprises DNA or RNA of the target microorganism; d) contacting the filtered mixture of c) with loop mediated isothermal amplification (LAMP) reagents and one or more primer sets specific to the DNA or RNA of the target microorganism, wherein the LAMP reagents and the one or more primer sets are lyophilized; e) amplifying the DNA or RNA of the target microorganism, thereby producing one or more amplicons; and f) detecting the presence or absence of the one or more amplicons. In some aspects, the presence of the one or more of the amplicons indicates the presence of the target microorganism. In some aspects, the filter can comprise a LAMP inhibitor control DNA and wherein the filtered mixture of step c) comprises DNA or RNA of the target microorganism and the LAMP inhibitor control DNA. In some aspects, the sample can be a blood, stool, sputum, oropharyngeal, nasopharyngeal, pap smear, vaginal swab or saliva sample.

Disclosed herein are methods of detecting a target microorganism in a sample. In some aspects, the method can comprise: a) obtaining or having obtained a sample from a subject, wherein the sample comprises or is suspected of comprising the target microorganism; b) contacting the sample with a lysis solution to form a mixture; c) filtering the mixture of b) through a filter to form a filtered mixture, wherein the filtered mixture comprises DNA or RNA of the target microorganism; d) contacting the filtered mixture of c) with nucleic acid amplification reagents and one or more primer sets specific to the DNA or RNA of the target microorganism, wherein the nucleic acid amplification reagents and the one or more primer sets are lyophilized; e) amplifying the DNA or RNA of the target microorganism, thereby producing one or more amplicons; and f) detecting the presence or absence of the one or more amplicons. In some aspects, the presence of the one or more of the amplicons indicates the presence of the target microorganism. In some aspects, the filter can comprise a nucleic acid amplification inhibitor control DNA and wherein the filtered mixture of step c) comprises DNA or RNA of the target microorganism and the nucleic acid amplification inhibitor control DNA. In some aspects, the sample can be a blood, stool, sputum, oropharyngeal, nasopharyngeal, pap smear, vaginal swab, or saliva sample.

Also disclosed herein are methods of detecting pathogenic E. coli in a sample. In some aspects, the methods can comprise: a) obtaining or having obtained a sample from a subject, wherein the sample comprises or is suspected of comprising the pathogenic E. coli; b) contacting the sample with a lysis solution to form a mixture; c) filtering the mixture of b) through a filter to form a filtered mixture, wherein the filtered mixture comprises DNA or RNA of the pathogenic E. coli; and d) contacting the filtered mixture of c) with loop mediated isothermal amplification (LAMP) reagents and one or more primer sets specific to the DNA or RNA of the pathogenic E. coli, wherein the LAMP reagents and the one or more primer sets are lyophilized; e) amplifying DNA or RNA of the pathogenic E. coli, thereby producing one or more amplicons; and f) detecting the presence or absence of the one or more amplicons. In some aspects, the presence of the one or more amplicons indicates the presence of the pathogenic E. coli. In some aspects, the filter can comprise a LAMP inhibitor control DNA and wherein the filtered mixture of step c) comprises DNA or RNA of the pathogenic E. coli and the LAMP inhibitor control DNA. In some aspects, the sample can be a stool sample.

Also disclosed herein are methods of detecting pathogenic E. coli in a sample. In some aspects, the methods can comprise: a) obtaining or having obtained a sample from a subject, wherein the sample comprises or is suspected of comprising the pathogenic E. coli; b) contacting the sample with a lysis solution to form a mixture; c) filtering the mixture of b) through a filter to form a filtered mixture, wherein the filtered mixture comprises DNA or RNA of the pathogenic E. coli; and d) contacting the filtered mixture of c) with nucleic acid amplification reagents and one or more primer sets specific to the DNA or RNA of the pathogenic E. coli, wherein the nucleic acid amplification reagents and the one or more primer sets are lyophilized; e) amplifying DNA or RNA of the pathogenic E. coli, thereby producing one or more amplicons; and f) detecting the presence or absence of the one or more amplicons. In some aspects, the presence of the one or more amplicons indicates the presence of the pathogenic E. coli. In some aspects, the filter can comprise a nucleic acid amplification inhibitor control DNA and wherein the filtered mixture of step c) comprises DNA or RNA of the pathogenic E. coli and the nucleic acid amplification inhibitor control DNA. In some aspects, the sample can be a stool sample.

Also disclosed herein are methods of detecting Shigella spp in a sample. In some aspects, the methods can comprise: a) obtaining or having obtained a sample from a subject, wherein the sample comprises or is suspected of comprising the Shigella spp; b) contacting the sample with a lysis solution to form a mixture; c) filtering the mixture of b) through a filter to form a filtered mixture, wherein the filtered mixture comprises DNA or RNA of the Shigella spp; and d) contacting the filtered mixture of c) with loop mediated isothermal amplification (LAMP) reagents and one or more primer sets specific to the DNA or RNA of the Shigella spp, wherein the LAMP reagents and the one or more primer sets are lyophilized; e) amplifying DNA or RNA of the Shigella spp, thereby producing one or more amplicons; and f) detecting the presence or absence of the one or more amplicons. In some aspects, the presence of the one or more amplicons indicates the presence of the Shigella spp. In some aspects, the filter can comprise a LAMP inhibitor control DNA and wherein the filtered mixture of step c) comprises DNA or RNA of the Shigella spp and the LAMP inhibitor control DNA. In some aspects, the sample can be a stool sample.

Also disclosed herein are methods of detecting Shigella spp in a sample. In some aspects, the methods can comprise: a) obtaining or having obtained a sample from a subject, wherein the sample comprises or is suspected of comprising the Shigella spp; b) contacting the sample with a lysis solution to form a mixture; c) filtering the mixture of b) through a filter to form a filtered mixture, wherein the filtered mixture comprises DNA or RNA of the Shigella spp; and d) contacting the filtered mixture of c) with nucleic acid amplification reagents and one or more primer sets specific to the DNA or RNA of the Shigella spp, wherein the nucleic acid amplification reagents and the one or more primer sets are lyophilized; e) amplifying DNA or RNA of the Shigella spp, thereby producing one or more amplicons; and f) detecting the presence or absence of the one or more amplicons. In some aspects, the presence of the one or more amplicons indicates the presence of the Shigella spp. In some aspects, the filter can comprise a nucleic acid amplification inhibitor control DNA and wherein the filtered mixture of step c) comprises DNA or RNA of the Shigella spp and the nucleic acid amplification inhibitor control DNA. In some aspects, the sample can be a stool sample.

Also disclosed herein are methods of detecting Salmonella typhi in a sample. In some aspects, the methods can comprise: a) obtaining or having obtained a sample from a subject, wherein the sample comprises or is suspected of comprising the Salmonella typhi; b) contacting the sample with a lysis solution to form a mixture; c) filtering the mixture of b) through a filter to form a filtered mixture, wherein the filtered mixture comprises DNA or RNA of the Salmonella typhi; and d) contacting the filtered mixture of c) with loop mediated isothermal amplification (LAMP) reagents and one or more primer sets specific to the DNA or RNA of the Salmonella typhi, wherein the LAMP reagents and the one or more primer sets are lyophilized; e) amplifying DNA or RNA of the Salmonella typhi, thereby producing one or more amplicons; and f) detecting the presence or absence of the one or more amplicons. In some aspects, the presence of the one or more amplicons indicates the presence of the Salmonella typhi. In some aspects, the filter can comprise a LAMP inhibitor control DNA and wherein the filtered mixture of step c) comprises DNA or RNA of the Salmonella typhi and the LAMP inhibitor control DNA. In some aspects, the sample can be a blood sample or a stool sample.

Also disclosed herein are methods of detecting Salmonella typhi in a sample. In some aspects, the methods can comprise: a) obtaining or having obtained a sample from a subject, wherein the sample comprises or is suspected of comprising the Salmonella typhi; b) contacting the sample with a lysis solution to form a mixture; c) filtering the mixture of b) through a filter to form a filtered mixture, wherein the filtered mixture comprises DNA or RNA of the Salmonella typhi; and d) contacting the filtered mixture of c) with nucleic acid amplification reagents and one or more primer sets specific to the DNA or RNA of the Salmonella typhi, wherein the nucleic acid amplification reagents and the one or more primer sets are lyophilized; e) amplifying DNA or RNA of the Salmonella typhi, thereby producing one or more amplicons; and f) detecting the presence or absence of the one or more amplicons. In some aspects, the presence of the one or more amplicons indicates the presence of the Salmonella typhi. In some aspects, the filter can comprise a nucleic acid amplification inhibitor control DNA and wherein the filtered mixture of step c) comprises DNA or RNA of the Salmonella typhi and the nucleic acid amplification inhibitor control DNA. In some aspects, the sample can be a blood sample or a stool sample.

In some aspects, in any of the methods disclosed herein, during the step of contacting the sample (e.g., step b)), the mixture can be heated in a lysis reagent to assist in lysing the cells. In some aspects, the mixture can be heated after step of contacting the sample or before the step of filtering the mixture. In some aspects, the temperature can be dependent on the sample. In some aspects, the mixture can be heated in a lysis reagent at a temperature of about 80° C. to about about 120° C.

Genes. In some aspects, the one or more primer sets can be specific for one or more genes specific to the target microorganism. In some aspects, the one or more primer sets can be specific for one or more of heat labile toxin (LT) gene, heat stable toxin (STh, and STp) gene, eae gene, bfpA gene, aaiC gene, aatA gene, CVD432 gene, F1845 gene, stx1 gene, stx2 gene, rfbO157 gene, invasion plasmid gene (ipaH), cholera toxin A (ctxA) gene, O1 lipopolysaccharide (O1rfb) gene, O139 gene, 16S gene, IS 6110 gene, MPB 64 gene, 16 S RNA gene, rpoB gene, FliC flagellar gene, invA gene, norovirus G1 gene, norovirus G2 gene, RdRp gene, capsid gene, NSP3 gene, hexon gene, ORF-1 gene, E gene, M gene, N gene, S gene, L1 gene, E6 gene, or E7 gene.

Microorganisms. In some aspects, the target microorganism can be a virus, a bacteriophage, a parasite, or a bacteria. In some aspects, the target microorganism can be E. coli, Shigella spp, Vibrio cholerae, non-cholera Vibrio spp, Campylobacter spp, Mycobacterium spp, Salmonella spp, an enteric virus, Dengue virus, a coronavirus, or human papillomavirus. In some aspects, the parasite can be Cryptosporidium, Entamoeba histolytica, Giardia lamblia, and Plasmodium.

In some aspects, the E. coli can be pathogenic. Pathogenic E. coli can cause serious food poisoning, septic shock, meningitis, or urinary tract infections in humans. Unlike normal flora E. coli, pathogenic E. coli can produce toxins and other virulence factors that enable them to reside in parts of the body normally not inhabited by E. coli, and to damage host cells. These pathogenic traits are encoded by virulence genes carried only by the pathogens.

In some aspects, pathogenic E. coli can be classified as enterotoxigenic E. coli (ETEC), enteropathogenic E. coli (EPEC), enteroaggregative E. coli (EAEC), enteroinvasive E. coli (EIEC), enterohemorrhagic E. coli (EHEC), a shiga toxin-producing E. coli, a verocytotoxin-producing E. coli or a diffusely adherent E. coli.

In some aspects, the E. coli can be an enterotoxigenic E. coli, an enteropathogenic E. coli, an enteroaggregative E. coli, an enteroinvasive E. coli, an enterohemorrhagic E. coli, a shiga toxin-producing E. coli, a verocytotoxin-producing E. coli or a diffusely adherent E. coli.

In some aspects, the one or more primer sets can be specific for one or more genes specific to the pathogenic E. coli. In some aspects, the one or more primer sets can be specific for one or more of heat labile toxin (LT) gene, heat stable toxin (STh, and STp) gene, eae gene, bfpA gene, aaiC gene, aatA gene, CVD432 gene, F1845 gene, stx1 gene, stx2 gene, rfbO157 gene, or invasion plasmid gene (ipaH). In some aspects, the pathogenic E. coli can be ETEC H10407, ETEC B7A, ETEC E24377A, ETEC 335093, ETEC 335140, or ETEC 335152. In some aspects, the one or more primer sets can be specific for one or more of heat labile toxin (LT) gene or heat stable toxin (STh, and STp) gene. In some aspects, the one or more primer sets of step d) can be specific to heat labile toxin (LT) gene or heat stable toxin (STh and STp) gene.

In some aspects, the enterotoxigenic E. coli strain can be any ETEC strain. IN some aspects, the enterotoxigenic E. coli can be ETEC H10407, ETEC B7A, ETEC E24377A, ETEC 335093, ETEC 335140, or ETEC 335152. In some aspects, the one or more primer sets can be specific to the heat labile toxin (LT) gene or the heat stable toxin (STh and STp) gene. In some aspects, the one or more primer sets of step d) can be specific to the heat labile toxin (LT) gene or the heat stable toxin (STh and STp) gene.

In some aspects, the enteropathogenic E. coli can be any EPEC strain. In some aspects, the one or more primer sets can be specific to the eae gene or the bfpA gene. In some aspects, the one or more primer sets of step d) can be specific to the eae gene or the bfpA gene.

In some aspects, the enteroaggregative E. coli can be any EAEC strain. In some aspects, the one or more primer sets can be specific to the aaiC gene, the aatA gene or the CVD432 gene. In some aspects, the one or more primer sets of step d) can be specific to the aaiC gene, the aatA gene or the CVD432 gene.

In some aspects, the enteroinvasive E. coli can be any EAIC strain. In some aspects, the one or more primer sets can be specific to the ipaH gene. In some aspects, the one or more primer sets of step d) can be specific to the ipaH gene.

In some aspects, the enterohemorrhagic E. coli can be any EIEC strain. In some aspects, the enterohemorrhagic E. coli can be 0157-H7. In some aspects, the one or more primer sets can be specific to the stx1 gene, the stx2 gene or the rfbO157 gene. In some aspects, the one or more primer sets of step d) can be specific to the stx1 gene, the stx2 gene or the rfbO157 gene.

In some aspects, the shiga toxin-producing E. coli can be any shiga toxin-producing E. coli strain. In some aspects, the one or more primer sets can be specific to the stx1 gene, the stx2 gene or the rfb0157 gene. In some aspects, the one or more primer sets of step d) can be specific to the stx1 gene, the stx2 gene or the rfbO157 gene.

In some aspects, the verocytotoxin-producing E. coli can be any verocytotoxin-producing E. coli strain. In some aspects, the one or more primer sets can be specific to the stx1 gene, the stx2 gene or the rfbO157 gene. In some aspects, the one or more primer sets of step d) can be specific to the stx1 gene, the stx2 gene or the rfbO157 gene.

In some aspects, the diffusely adherent E. coli can be any diffusely adherent E. coli strain. In some aspects, the one or more primer sets can be specific to the F1845 gene. In some aspects, the one or more primer sets of step d) can be specific to the F1845 gene.

In some aspects, the Mycobacterium spp can be M. tuberculosis. In some aspects, the Mycobacterium spp can be M. leprae. In some aspects, the one or more primer sets can be specific to the IS 6110 gene, the MPB 64 gene, the 16 S rRNA gene, or the rpoB gene. In some aspects, the one or more primer sets of step d) can be specific to the IS 6110 gene, the MPB 64 gene, the 16 S rRNA gene, or the rpoB gene.

In some aspects, the Salmonella spp can be S. typhi or S. paratyphi. In some aspects, the Salmonella spp can be Salmonella enterica. For example, Salmonella spp can include typhoidal serotypes (Salmonella enterica var Typhi [S Typhi] and Salmonella enterica var Paratyphi [S Paratyphi]. Most non-typhoidal Salmonella infections are caused by S. enterica subspecies enterica serotype Enteritidis, S. Typhimurium, S. Newport, S. Heidelberg, and S. Javiana. In some aspects, the one or more primer sets can be specific for one or more genes specific to the Salmonella spp. In some aspects, the one or more primer sets can be specific for one or more genes specific to the S. typhi. In some aspects, the one or more primer sets can be specific to the invA gene or the Flagellar gene. In some aspects, the one or more primer sets of step d) can be specific to the invA gene or the Flagellar gene.

In some aspects, the Shigella spp can be S. flexneri, S. sonnei, S. dysenteriae, or S. boydii. In some aspects, the one or more primer sets can be specific for one or more genes specific to the Shigella spp. In some aspects, the one or more primer sets can be specific to the invasion plasmid gene (ipaH). In some aspects, the one or more primer sets of step d) can be specific to the invasion plasmid gene (ipaH).

In some aspects, the enteric virus can be norovirus, sapovirus, astrovirus, rotavirus, or adenovirus. In some aspects, the enteric virus can be norovirus and the one or more primer sets of step d) can be specific to the G1 gene or the G2 gene. In some aspects, the one or more primers sets can be specific for any of the genogroups of norovirus. In some aspects, the one or more primers can be specific for any of GI-X (e.g., GI, 27 GII, 3 GIII, 2 GIV, 2 GV, 2 GVI, GVII, GVIII, GIX, or GXGE). In some aspects, the enteric virus can be norovirus and the one or more primer sets can be specific to the G1 gene or the G2 gene. In some aspects, the enteric virus can be sapovirus and the one or more primer sets of step d) can be specific to the RdRp gene. In some aspects, the enteric virus can be sapovirus and the one or more primer sets can be specific to the RdRp gene. In some aspects, the enteric virus can be astrovirus and the one or more primer sets of step d) can be specific to the Capsid gene. In some aspects, the enteric virus can be astrovirus and the one or more primer sets can be specific to the Capsid gene. In some aspects, the enteric virus can be rotavirus and the one or more primer sets of step d) can be specific to the NSP3 gene. In some aspects, the enteric virus can be rotavirus and the one or more primer sets can be specific to the NSP3 gene. In some aspects, the enteric virus can be adenovirus and the one or more primer sets of step d) can be specific to the Hexon gene. In some aspects, the enteric virus can be adenovirus and the one or more primer sets can be specific to the Hexon gene.

In some aspects, the target microorganism can be a coronavirus. In some aspects, the coronavirus can be SARS-CoV-2. In some aspects, the one or more primer sets of step d) can be specific to the ORF-1 gene, the E gene, the M gene, the N gene and the S gene. In some aspects, the one or more primer sets can be specific to the ORF-1 gene, the E gene, the M gene, the N gene and the S gene.

In some aspects, the target microorganism can be a human papillomavirus. In some aspects, the one or more primer sets of step d) can be specific to the L1 gene, E6 gene or E7 gene. In some aspects, the one or more primer sets can be specific to the L1 gene, E6 gene or E7 gene.

In some aspects, the target microorganism can be Campylobacter spp. In some aspects, the Campylobacter spp can be C. jejuni and C. coli. In some aspects, the one or more primer sets can be specific for one or more genes specific to the Campylobacter spp. In some aspects, the one or more primer sets can be specific to C. jejuni or C. coli. In some aspects, the one or more primer sets can be specific to the 16S gene. In some aspects, the one or more primer sets of step d) can be specific to the 16S gene.

In some aspects, the target microorganism can be Vibrio cholera. In some aspects, the target microorganism can be a non-cholera Vibrio spp. In some aspects, the one or more primer sets can be specific for one or more genes specific to the Vibrio cholera. In some aspects, the one or more primer sets can be specific to the ctxA gene, the O1rfb gene or the O139 gene. In some aspects, the one or more primer sets of step d) can be specific to the ctxA gene, the O1rfb gene or the O139 gene.

In some aspects, the target microorganism can be Dengue virus. In some aspects, the Dengue virus can be a DENV1, DENV2 or DENV3. In some aspects, the one or more primer sets can be specific to the non structural 5 (NS5) gene or the capsid (C) gene.

In some aspects, the target microorganism can be Cryptosporidium spp, Entamoeba histolytica, Giardia lamblia, and Plasmodium sp. In some aspects, the one or more primer sets can be specific for one or more genes specific to the Cryptosporidium spp, Entamoeba histolytica, Giardia lamblia, and Plasmodium spp. In some aspects, the one or more primer sets can be specific to the 18SrRNA gene. In some aspects, the one or more primer sets of step d) can be specific to the 18SrRNA gene.

Detecting. In some aspects, the detecting step can be performed without the need for additional equipment directly from the sample. In some aspects, the detecting step can be performed by the naked eye to visually detect the presence or absence of the amplicon. In some aspects, the detecting step can be performed using a UV illuminator to visually detect the presence or absence of the amplicon. In some aspects, the UV illuminator or fluorometer (e.g., an isothermal fluorometer) can be a commercial reader (e.g., AmpliFire).

Amplifying. Disclosed herein are methods that include preparing or designing a primer or a probe that is capable of detecting, amplifying or otherwise measures the presence or absence of one or more genes disclosed herein. Amplifying or amplification refers to the production of one or more copies of a genetic fragment or target sequence, for example, an amplicon. Amplification of the DNA or RNA of the target microorganism can be carried out using gene-specific primers and loop mediated isothermal amplification (LAMP) or polymerase chain reaction (PCR) to generate an amplicon.

Primers and primer sets. Primers and/or primer sets can be prepared and designed according to the microorganism to be the target of the detection. Primers and primer sets can be prepared and designed to specifically hybridize to one or more of any of the following genes: heat labile toxin (LT) gene, heat stable toxin (STh, and STp) gene, eae gene, bfpA gene, aaiC gene, aatA gene, CVD432 gene, F1845 gene, stx1 gene, stx2 gene, rfbO157 gene, invasion plasmid gene (ipaH), cholera toxin A (ctxA) gene, O1 lipopolysaccharide (O1rfb) gene, O139 gene, 16S gene, IS 6110 gene, MPB 64 gene, 16 S RNA gene, rpoB gene, FliC flagellar gene, invA gene, norovirus G1 gene, norovirus G2 gene, RdRp gene, capsid gene, NSP3 gene, hexon gene, ORF-1 gene, NS5 gene, C gene, E gene, M gene, N gene, S gene, L1 gene, E6 gene, or E7 gene. Tables 1-7 provide examples of primers and primer sets that can be used in the methods disclosed herein. In some aspects, the set of primers comprises 1 or more primer pairs. For example, in some aspects, the methods utilize 6 or more primer sets.

TABLE 1
Primers and primer sets for
detecting ETEC.

| | | SEQ |
|---|---|---|
| Primer | | ID |
| name | Sequence | NO: |
| ETEC LT | GenBank: FN649414.1 | |
| F3 | ATCGTGTTAATTTTGGTGTGATTG | 1 |
| B3 | CTGGGTCTCCTCATTACAAGT | 2 |
| FIP | AACCATCCTCTGCCGGAGCTATAT | 3 |
| | TGAACGATTACATCGTAACAGGGAAT | |
| BIP | TTCCCACCGGATCACCAAGCTGTTCT | 4 |
| | TGATGAATCTCCACAACCTT | |
| LF | GATTTCTGTAATACCGGTCTCTAT | 5 |
| LB | AGAAGAACCCTGGATTCATCATGC | 6 |
| ETEC STp | GenBank: FN649414.1 | |
| F3 | GCAAAATCCGTTTAACTAATCTCAA | 7 |
| B3 | ACAGCAGTAAAATGTGTTGTTCAT | 8 |
| FIP | AAGAGGGGAAAGATAATACAGAAA | 9 |
| | TTTTTTAAACAACATGACGGGAGGT | |
| BIP | TAGTCAGTCAACTGAATCACTTGAT | 10 |
| | TTTTCTGTTGTTTTTTACAACATCACACT | |
| LF | GCCAACATTAGCTTTTTCATG | 11 |
| LB | CAAAAGAGAAAATTACATTAGAGAC | 12 |
| ETEC STh | GenBank: FN649414.1 | |
| F3 | CTCAGGATGCTAAACCAGT | 13 |
| B3 | CAGAACAAATATAAAGGGAACTGTT | 14 |
| FIP | TCATGCTTTCAGGACCACTTTTATT | 15 |
| | GAGTCTTCAAAAGAAAAAATCACACT | |
| BIP | AGTAGCAATTACTGCTGTGAATTGTC | 16 |
| | CCTTTATATTATTAATAGCACCCG | |
| LB | GTTGTAATCCTGCTTGT | 17 |

TABLE 2
Primers and primer sets for detecting
Shigella (GenBank: M76444.1).

| ipaH | | |
|---|---|---|
| Primer name | Sequence | SEQ ID NO: |
| F3 | AATTCTGGAGGACATTGCC | 18 |
| B3 | CGTACGCTTCAGTACAGC | 19 |
| FIP (F1c + F2) | CTTCACGGCAGTGGAGAGCT | 20 |
| | GAGATAGAAGTCTACCTGGC | |
| BIP (B1c + B2) | TATGGCGTGTCGGGAGTGAT | 21 |
| | TCATTCTCTTCACGGCTTC | |
| LoopF | CTCTGCGAGCATGGTCTG | 22 |
| LoopB | CACTGCCGAAGCTATGGT | 23 |
| F2 | TGAGATAGAAGTCTACCT | 24 |
| | GGC | |
| F1c | CTTCACGGCAGTGGAGAGC | 25 |
| B2 | TTCATTCTCTTCACGGCTTC | 26 |
| B1c | TATGGCGTGTCGGGAGTGA | 27 |

TABLE 3
Primers and primer sets for
detecting Cholera.

| Primer | | SEQ ID |
|---|---|---|
| name | Sequence | NO: |
| ctxA | GenBank: AF452584 | |
| F3 | GTTATATCGGGCAGATTCTAG | 28 |
| B3 | GTTTGACCCACTAAGTGG | 29 |
| FIP(F1c + F2) | TTTGAGTACCTCGGTCAAAGTA | 30 |
| | CACCTCCTGATGAAATAAAGC | |
| BIP(B1c + B2) | TGATCATGCAAGAGGAACTCAG | 31 |
| | ATTGAGGTGGAAACATATCC | |
| LoopF | TCTGTCCTCTTGGCATAAGACCA | 32 |
| LoopB | CGGGATTTGTTAGGCACGATGA | 33 |
| F2 | ACCTCCTGATGAAATAAAGC | 34 |
| F1c | TTTGAGTACCTCGGTCAAAGTAC | 35 |
| B2 | ATTGAGGTGGAAACATATCC | 36 |
| B1c | TGATCATGCAAGAGGAACTCAG | 37 |
| O1rfB | GenBank: X59554 | |
| F3 | TCTTCTGCTACCAGTGGCGTAC | 38 |
| B3 | TTCAAGTGGAGCACTTGGGCTA | 39 |
| FIP(F1c + F2) | TGCAAAACGGGCGACGTTTAGG | 40 |
| | CTCGTCGACAGCATCGAGCA | |
| BIP(B1c + B2) | TGATCCGACAAGCCCAAATGCCAC | 41 |
| | TCGATGTTGAGGCGAAGTTTAGGT | |
| LoopF | AACACCTCCTGCATAACTCTTGC | 42 |
| LoopB | GCTCGTATTGCGGCGGTAA | 43 |
| F2 | TCGTCGACAGCATCGAGCA | 44 |
| F1c | TGCAAAACGGGCGACGTTTAGGC | 45 |
| B2 | TCGATGTTGAGGCGAAGTTTAGGT | 46 |
| B1c | TGATCCGACAAGCCCAAATGCCAC | 47 |

TABLE 4
Primers and primer sets for
detecting Salmonella typhi.

| | SEQ |
|---|---|
| | ID |
| Sequence | NO: |
| CCAAGGCAGCATCAATT | 48 |
| TCTATGCCGCTACATATGA | 49 |
| TCTGAAGTTGTTACTGCTACC | 50 |
| GGCCTGTTCTGAAGTTATGT | |
| GCCACCAAATTTCACAGCTCC | 51 |
| AGGTGCAATTACTGCTAA | |
| CTTAGCAAGCGACCTTGA | 52 |
| TTGAGCAACGCCAGTAC | 53 |
| GCCTGTTCTGAAGTTATGT | 54 |
| TCTGAAGTTGTTACTGCTACCG | 55 |
| CAGGTGCAATTACTGCTAA | 56 |
| GCCACCAAATTTCACAGCTC | 57 |

TABLE 5
Primers and primer sets for
detecting Norovirus.

| | | SEQ |
|---|---|---|
| Primer | | ID |
| name | Sequence | NO: |
| G1 (Norwalk) | | |
| F3 | TGTGGACAGGAGATCGC | 58 |
| B3 | ACTTGTCCAGCAGTCGC | 59 |
| FIP | TAGCGCCATCCACGCTTG | 60 |
| | ATTTTCTTCTGCCCGAATTCGTAA | |
| BIP | CTGGTCAGTTGGTACCGGAGTTTT | 61 |
| | CTGTCGAAGAACCTGCTAC | |
| LoopF | CGTCCTTAGACGCCATCATCA | 62 |
| LoopB | GCTTCTGACCCTCTTGCAATG | 63 |
| F2 | CTTCTGCCCGAATTCGTAA | 64 |
| F1c | TAGCGCCATCCACGCTTGA | 65 |
| B2 | CTGTCGAAGAACCTGCTAC | 66 |
| B1c | CTGGTCAGTTGGTACCGGAG | 67 |
| G2 | | |
| (Lordsdale) | GenBank: X86557 | |
| F3 | CAGACAAGAGCCAATGTTCA | 68 |
| B3 | CAATAGCGGCACCAACAA | 69 |
| FIP(F1c + F2) | CGTCATTCGACGCCATCTTCA | 70 |
| | GATTCTCAGATCTGAGCACG | |
| BIP(B1c + B2) | CATCTGATGGGTCCGCAGCTC | 71 |
| | CAGAGCCATAACCTCATTA | |
| LoopF | CTGGGAGCCAGATTGCGATC | 72 |
| LoopB | AACCTCGTCCCAGAGGTCAA | 73 |
| F2 | GATTCTCAGATCTGAGCACG | 74 |
| F1c | CGTCATTCGACGCCATCTTCA | 75 |
| B2 | TCCAGAGCCATAACCTCATTA | 76 |
| B1c | CATCTGATGGGTCCGCAGC | 77 |

TABLE 6
Primers and primer sets for detecting
Campylobacter spp.

| | | SEQ |
|---|---|---|
| | | ID |
| Primer name | Sequence | NO: |
| Campy 16S | | |
| 16s Campy | CTGCTTAACACAAGTTGAGTAGG | 78 |
| F3 | | |
| 16s Campy | TTCCTTAGGTACCGTCAGAA | 79 |
| B3 | | |
| 16s Campy | GGACCGTGTCTCAGTTCCAGTGTGAC | 80 |
| FIP | GGATGAGACTATATAGTATCAGCTAG | |
| 16s Campy | CGGGAGGCAGCAGTAGGGAATATT | 81 |
| BIP | GCTAAGAAAAGGAGTTTACGCTCCG | |
| 16s Campy | GTTAAGCGTCATAGCCTTGGTAA | 82 |
| LF | | |
| 16s Campy | GCGTGGAGGATGACACTT | 83 |
| LB | | |
| Campy jejuni | | |
| CJ-FIP | ACAGCACCGCCACCTATAGT | 84 |
| | AGAAGCTTTTTTAAACTAGGGC | |
| CJ-BIP | AGGCAGCAGAACTTACGCATTGA | 85 |
| | GTTTGAAAAAACATTCTACCTCT | |
| CJ-F3 | GCAAGACAATATTATTGATCGC | 86 |
| CJ-B3 | CTTTCACAGGCTGCACTT | 87 |
| CJ-LF | CTAGCTGCTACTACAGAACCAC | 88 |
| CJ-LB | CATCAAGCTTCACAAGGAAA | 89 |
| Campy coli | | |
| CC-FIP | AAGAGATAAACACCATGATCCCAG | 90 |
| | TCATGAATGAGCTTACTTTAGC | |
| CC-BIP | GCGGCAAAGACTTATGATAAA | 91 |
| | GCTACCGCCATTCCTAAAACAAG | |
| CC-F3 | TGGGAGCGTTTTTGATCT | 92 |
| CC-B3 | AATCAAACTCACCGCCAT | 93 |
| CC-LF | CCACTACAGCAAAGGTGATG | 94 |
| CC-LB | CCACGATAGCCTTTATGGA | 95 |

TABLE 7
Alternative primer sequences.

| | | | SEQ |
|---|---|---|---|
| | Primer | | ID |
| | name | Sequence | NO: |
| 1 | ETEC LT | Accession no. NC_017722 | |
| | F3 | ACTGATTGCCGCAATTGAATTG | 96 |
| | B3 | GACTATCAGTCAGAGGTTGACA | 97 |
| | FIP | AGCGGCGCAACATTTCAGGTTT | 98 |
| | (F1c + F2) | GGTCTCGGTCAGATATGTGA | |
| | BIP | TTGCCTGCCATCGATTCCGTTCT | 99 |
| | (B1c + B2) | CTATGTGCATACGGAGCT | |
| | LoopF | CCGGGCAGTCAACATATAGACT | 100 |
| | LoopB | TGTGTTGCGATATTCCGAACAT | 101 |
| | F2 | TTGGTCTCGGTCAGATATGTGA | 102 |
| | F1c | AGCGGCGCAACATTTCAGGT | 103 |
| | B2 | TCTCTATGTGCATACGGAGCT | 104 |
| | B1c | TTGCCTGCCATCGATTCCGT | 105 |
| 2 | ETEC LT | | |
| | F3 | CCTCATTACAAGTATCACCTGT | 106 |
| | B3 | GCTCACTTAGCAGGACAGTCTA | 107 |
| | FIP | AGCTCCGGCAGAGGATGGTTGTG | 108 |
| | (F1c + F2) | GTGCATGATGAATCCAG | |
| | BIP | CCACCTAACGCAGAAACCTCCTAT | 109 |
| | (B1c + B2) | GTTATAGCGACAGCACCAA | |
| | LoopF | CCACCGGATCACCAAGCT | 110 |
| | LoopB | GGGTGAGGGCTGTATACGC | 111 |
| | F2 | TGTGGTGCATGATGAATCCAG | 112 |
| | F1c | AGCTCCGGCAGAGGATGGT | 113 |
| | B2 | ATGTTATAGCGACAGCACCAA | 114 |
| | B1c | CCACCTAACGCAGAAACCTCCT | 115 |
| 3 | ETEC LT | | |
| | F3 | AACCTTGTGGTGCATGATGAA | 116 |
| | B3 | GCTCACTTAGCAGGACAGTCTA | 117 |
| | FIP | AGCTCCGGCAGAGGATGGTTCC | 118 |
| | (F1c + F2) | AGGGTTCTTCTCTCCAAG | |
| | BIP | CCACCTAACGCAGAAACCTCCTAT | 119 |
| | (B1c + B2) | GTTATAGCGACAGCACCAA | |
| | LoopF | ACAGATTAGCAGGTTTCCCACC | 120 |
| | LoopB | GGGTGAGGGCTGTATACGC | 121 |
| | F2 | TCCAGGGTTCTTCTCTCCAAG | 122 |
| | F1c | AGCTCCGGCAGAGGATGGT | 123 |
| | B2 | ATGTTATAGCGACAGCACCAA | 124 |
| | B1c | CCACCTAACGCAGAAACCTCCT | 125 |
| 4 | ETEC LT | | |
| | F3 | AACCTTGTGGTGCATGATGAAT | 126 |
| | B3 | GCTCACTTAGCAGGACAGTCTA | 127 |
| | FIP | AGCTCCGGCAGAGGATGGTCAG | 128 |
| | (F1c + F2) | GGTTCTTCTCTCCAAGC | |
| | BIP | CCACCTAACGCAGAAACCTCCTAT | 129 |
| | (B1c + B2) | GTTATAGCGACAGCACCAA | |
| | LoopF | ACAGATTAGCAGGTTTCCCACC | 130 |
| | LoopB | GGGTGAGGGCTGTATACGC | 131 |
| | F2 | CAGGGTTCTTCTCTCCAAGC | 132 |
| | F1c | AGCTCCGGCAGAGGATGGT | 133 |
| | B2 | ATGTTATAGCGACAGCACCAA | 134 |
| | B1c | CCACCTAACGCAGAAACCTCCT | 135 |
| 5 | ETEC LT | | |
| | F3 | ACAACCTTGTGGTGCATGAT | 136 |
| | B3 | GCTCACTTAGCAGGACAGTCTA | 137 |
| | FIP | AGCTCCGGCAGAGGATGGTAATC | 138 |
| | (F1c + F2) | CAGGGTTCTTCTCTCCAA | |
| | BIP | CCACCTAACGCAGAAACCTCCTATG | 119 |
| | (B1c + B2) | TTATAGCGACAGCACCAA | |
| | LoopF | ACAGATTAGCAGGTTTCCCACC | 120 |
| | LoopB | GGGTGAGGGCTGTATACGC | 121 |
| | F2 | AATCCAGGGTTCTTCTCTCCAA | 122 |
| | F1c | AGCTCCGGCAGAGGATGGT | 123 |
| | B2 | ATGTTATAGCGACAGCACCAA | 124 |
| | B1c | CCACCTAACGCAGAAACCTCCT | 125 |
| 6 | ETEC LT | | |
| | F3 | TGTCAACCTCTGACTGATAGTC | 126 |
| | B3 | GATGTATTAGGCGTATACAGCC | 127 |
| | FIP | GCTTGGAGAGAAGAACCCTGGA | 128 |
| | (F1c + F2) | CCTCATTACAAGTATCACCTGT | |
| | BIP | GTGATCCGGTGGGAAACCTGCT | 129 |
| | (B1c + B2) | GAACGATTACATCGTAACAGG | |
| | LoopF | TCATCATGCACCACAAGGTTGT | 130 |
| | LoopB | TCCTCTGCCGGAGCTATATTCA | 131 |
| | F2 | CCTCATTACAAGTATCACCTGT | 132 |
| | F1c | GCTTGGAGAGAAGAACCCTGGA | 133 |
| | B2 | TGAACGATTACATCGTAACAGG | 134 |
| | B1c | GTGATCCGGTGGGAAACCTGC | 135 |
| 7 | ETEC LT | | |
| | F3 | ACCTGCTAATCTGTAACCATCC | 136 |
| | B3 | ACCGTGCTGACTCTAGACC | 137 |
| | FIP | AGGCGTATACAGCCCTCACCCTC | 138 |
| | (F1c + F2) | GTTCATCAATCACACCAA | |
| | BIP | ACTGTCCTGCTAAGTGAGCACTTT | 139 |
| | (B1c + B2) | ATGATCACGCGAGAGGAA | |
| | LoopF | TTCTGCGTTAGGTGGAATACCA | 140 |
| | LoopB | ATCTGACAAAGCCGGTTTGTG | 141 |
| | F2 | TCGTTCATCAATCACACCAA | 142 |
| | F1c | AGGCGTATACAGCCCTCACCC | 143 |
| | B2 | TTATGATCACGCGAGAGGAA | 144 |
| | B1c | ACTGTCCTGCTAAGTGAGCACT | 145 |
| 8 | ETEC LT | | |
| | F3 | TCTGCCGGAGCTATATTCAGAT | 146 |
| | B3 | ACCGTGCTGACTCTAGACC | 147 |
| | FIP | AGCGACAGCACCAAATATGTTTT | 148 |
| | (F1c + F2) | GGTATTCCACCTAACGCAGA | |
| | BIP | ACTGTCCTGCTAAGTGAGCACTTT | 149 |
| | (B1c + B2) | ATGATCACGCGAGAGGAA | |
| | LoopF | CAGCCCTCACCCATATGAACAG | 150 |
| | LoopB | ATCTGACAAAGCCGGTTTGTG | 151 |
| | F2 | TGGTATTCCACCTAACGCAGA | 152 |
| | F1c | AGCGACAGCACCAAATATGTTT | 153 |
| | B2 | TTATGATCACGCGAGAGGAA | 154 |
| | B1c | ACTGTCCTGCTAAGTGAGCACT | 155 |
| 9 | ETEC LT | | |
| | F3 | CCTCATTACAAGTATCACCTGT | 156 |
| | B3 | CTGCGTTAGGTGGAATACC | 157 |
| | FIP | GGTTTCCCACCGGATCACCACCTT | 158 |
| | (F1c + F2) | GTGGTGCATGATG | |
| | BIP | CCTCTGCCGGAGCTATATTCAGGGT | 159 |
| | (B1c + B2) | GTGATTGATGAACGATTAC | |
| | LoopF | TGGAGAGAAGAACCCTGGAT | 160 |
| | LoopB | ACCGGTCTCTATATTCCCTGT | 161 |
| | F2 | ACCTTGTGGTGCATGATG | 162 |
| | F1c | GGTTTCCCACCGGATCACC | 163 |
| | B2 | GGTGTGATTGATGAACGATTAC | 164 |
| | B1c | CCTCTGCCGGAGCTATATTCAG | 165 |
| 10 | ETEC LT | | |
| | F3 | CCTCATTACAAGTATCACCTGT | 166 |
| | B3 | CTGCGTTAGGTGGAATACC | 167 |
| | FIP | CAGGTTTCCCACCGGATCACACCTT | 168 |
| | (F1c + F2) | GTGGTGCATGATG | |
| | BIP | CCTCTGCCGGAGCTATATTCAGGGT | 169 |
| | (B1c + B2) | GTGATTGATGAACGATTAC | |
| | LoopF | TGGAGAGAAGAACCCTGGAT | 170 |
| | LoopB | ACCGGTCTCTATATTCCCTGT | 171 |
| | F2 | ACCTTGTGGTGCATGATG | 172 |
| | F1c | CAGGTTTCCCACCGGATCAC | 173 |
| | B2 | GGTGTGATTGATGAACGATTAC | 174 |
| | B1c | CCTCTGCCGGAGCTATATTCAG | 175 |
| 11 | ETEC LT | | |
| | F3 | CCTCATTACAAGTATCACCTGT | 176 |
| | B3 | CTGCGTTAGGTGGAATACC | 177 |
| | FIP | ACAGATTAGCAGGTTTCCCACCACCTT | 178 |
| | (F1c + F2) | GTGGTGCATGATG | |
| | BIP | CCTCTGCCGGAGCTATATTCAGGGTGT | 179 |
| | (B1c + B2) | GATTGATGAACGATTAC | |
| | LoopF | TGGAGAGAAGAACCCTGGAT | 180 |
| | LoopB | ACCGGTCTCTATATTCCCTGT | 181 |
| | F2 | ACCTTGTGGTGCATGATG | 182 |
| | F1c | ACAGATTAGCAGGTTTCCCACC | 183 |
| | B2 | GGTGTGATTGATGAACGATTAC | 184 |
| | B1c | CCTCTGCCGGAGCTATATTCAG | 185 |
| 12 | ETEC LT | | |
| | F3 | CCTCATTACAAGTATCACCTGT | 186 |
| | B3 | CTGCGTTAGGTGGAATACC | 187 |
| | FIP | GTTTCCCACCGGATCACCAAACCTT | 188 |
| | (F1c + F2) | GTGGTGCATGATG | |
| | BIP | CCTCTGCCGGAGCTATATTCAGGGT | 189 |
| | (B1c + B2) | GTGATTGATGAACGATTAC | |
| | LoopF | TGGAGAGAAGAACCCTGGAT | 190 |
| | LoopB | ACCGGTCTCTATATTCCCTGT | 191 |
| | F2 | ACCTTGTGGTGCATGATG | 192 |
| | F1c | GTTTCCCACCGGATCACCAA | 193 |
| | B2 | GGTGTGATTGATGAACGATTAC | 194 |
| | B1c | CCTCTGCCGGAGCTATATTCAG | 195 |
| 13 | ETEC LT | | |
| | F3 | CCTCATTACAAGTATCACCTGT | 196 |
| | B3 | CTGCGTTAGGTGGAATACC | 197 |
| | FIP | AGATTAGCAGGTTTCCCACCGACCTT | 198 |
| | (F1c + F2) | GTGGTGCATGATG | |
| | BIP | CCTCTGCCGGAGCTATATTCAGGGTGT | 199 |
| | (B1c + B2) | GATTGATGAACGATTAC | |
| | LoopF | TGGAGAGAAGAACCCTGGAT | 200 |
| | LoopB | ACCGGTCTCTATATTCCCTGT | 201 |
| | F2 | ACCTTGTGGTGCATGATG | 202 |
| | F1c | AGATTAGCAGGTTTCCCACCG | 203 |
| | B2 | GGTGTGATTGATGAACGATTAC | 204 |
| | B1c | CCTCTGCCGGAGCTATATTCAG | 205 |
| 14 | ETEC LT | | |
| | F3 | CCTCATTACAAGTATCACCTGT | 206 |
| | B3 | CTGCGTTAGGTGGAATACC | 207 |
| | FIP | TTTCCCACCGGATCACCAAGACCTT | 208 |
| | (F1c + F2) | GTGGTGCATGATG | |
| | BIP | CCTCTGCCGGAGCTATATTCAGGGT | 209 |
| | (B1c + B2) | GTGATTGATGAACGATTAC | |
| | LoopF | TGGAGAGAAGAACCCTGGAT | 210 |
| | LoopB | ACCGGTCTCTATATTCCCTGT | 211 |
| | F2 | ACCTTGTGGTGCATGATG | 212 |
| | F1c | TTTCCCACCGGATCACCAAG | 213 |
| | B2 | GGTGTGATTGATGAACGATTAC | 214 |
| | B1c | CCTCTGCCGGAGCTATATTCAG | 215 |
| 15 | ETEC LT | | |
| | F3 | CCTCATTACAAGTATCACCTGT | 216 |
| | B3 | GCGTTAGGTGGAATACCATAT | 217 |
| | FIP | GGTTTCCCACCGGATCACCACCTTGT | 218 |
| | (F1c + F2) | GGTGCATGATG | |
| | BIP | CCTCTGCCGGAGCTATATTCAGGGTGT | 219 |
| | (B1c + B2) | GATTGATGAACGATTAC | |
| | LoopF | TGGAGAGAAGAACCCTGGAT | 220 |
| | LoopB | ACCGGTCTCTATATTCCCTGT | 221 |
| | F2 | ACCTTGTGGTGCATGATG | 222 |
| | F1c | GGTTTCCCACCGGATCACC | 223 |
| | B2 | GGTGTGATTGATGAACGATTAC | 224 |
| | B1c | CCTCTGCCGGAGCTATATTCAG | 225 |
| 16 | ETEC LT | | |
| | F3 | GATGAATTTCCACAACCTTGTG | 226 |
| | B3 | CTGCGTTAGGTGGAATACC | 227 |
| | FIP | ACAGATTAGCAGGTTTCCCACCCATGAT | 228 |
| | (F1c + F2) | GAATCCAGGGTTCTT | |
| | BIP | CCTCTGCCGGAGCTATATTCAGGGTGT | 229 |
| | (B1c + B2) | GATTGATGAACGATTAC | |
| | LoopF | GGATCACCAAGCTTGGAGAG | 230 |
| | LoopB | ACCGGTCTCTATATTCCCTGT | 231 |
| | F2 | CATGATGAATCCAGGGTTCTT | 232 |
| | F1c | ACAGATTAGCAGGTTTCCCACC | 233 |
| | B2 | GGTGTGATTGATGAACGATTAC | 234 |
| | B1c | CCTCTGCCGGAGCTATATTCAG | 235 |
| 1 | ETEC STh | Accession no. NC_017724 | |
| | F3 | CGGGTGTGTGGAGGACTT | 236 |
| | B3 | GCTAAACCAGCAGGGTCTTC | 237 |
| | FIP | ATATTTGTGTGCGCCGTGGCT | 238 |
| | (F1c + F2) | CACCTCTTAGTCGTTCTTCAGC | |
| | BIP | ATAGCACCCGGTACAAGCAGG | 239 |
| | (B1c + B2) | ATGAAAGTAGTCCTGAAAGCATG | |
| | LoopF | CGCTGTTCTTCAACTGTGGAG | 240 |
| | LoopB | CACAATTCACAGCAGTAATTGC | 241 |
| | F2 | CACCTCTTAGTCGTTCTTCAGC | 242 |
| | F1c | ATATTTGTGTGCGCCGTGGCT | 243 |
| | B2 | TGAAAGTAGTCCTGAAAGCATG | 244 |
| | B1c | ATAGCACCCGGTACAAGCAGGA | 245 |
| 2 | ETEC STh | | |
| | F3 | CGGGTGTGTGGAGGACTT | 246 |
| | B3 | GCTAAACCAGCAGGGTCTTC | 247 |
| | FIP | ATATTTGTGTGCGCCGTGGCTCA | 248 |
| | (F1c + F2) | CCTCTTAGTCGTTCTTCAGC | |
| | BIP | TAGCACCCGGTACAAGCAGGATT | 249 |
| | (B1c + B2) | GAAAGTAGTCCTGAAAGCATG | |
| | LoopF | CGCTGTTCTTCAACTGTGGAG | 250 |
| | LoopB | CACAATTCACAGCAGTAATTGC | 251 |
| | F2 | CACCTCTTAGTCGTTCTTCAGC | 252 |
| | F1c | ATATTTGTGTGCGCCGTGGCT | 253 |
| | B2 | TGAAAGTAGTCCTGAAAGCATG | 254 |
| | B1c | TAGCACCCGGTACAAGCAGGAT | 255 |
| 3 | ETEC STh | | |
| | F3 | CGGGTGTGTGGAGGACTT | 256 |
| | B3 | GCTAAACCAGCAGGGTCTTC | 257 |
| | FIP | TATTTGTGTGCGCCGTGGCTG | 258 |
| | (F1c + F2) | CACCTCTTAGTCGTTCTTCAGC | |
| | BIP | ATAGCACCCGGTACAAGCAGG | 259 |
| | (B1c + B2) | ATGAAAGTAGTCCTGAAAGCATG | |
| | LoopF | CGCTGTTCTTCAACTGTGGAG | 260 |
| | LoopB | CACAATTCACAGCAGTAATTGC | 261 |
| | F2 | CACCTCTTAGTCGTTCTTCAGC | 262 |
| | F1c | TATTTGTGTGCGCCGTGGCTG | 263 |
| | B2 | TGAAAGTAGTCCTGAAAGCATG | 264 |
| | B1c | ATAGCACCCGGTACAAGCAGGA | 265 |
| 4 | ETEC STh | | |
| | F3 | CGGGTGTGTGGAGGACTT | 266 |
| | B3 | GCTAAACCAGCAGGGTCTTC | 267 |
| | FIP | ATTTGTGTGCGCCGTGGCCA | 268 |
| | (F1c + F2) | CCTCTTAGTCGTTCTTCAGC | |
| | BIP | ATAGCACCCGGTACAAGCAG | 269 |
| | (B1c + B2) | GATGAAAGTAGTCCTGAAAGCATG | |
| | LoopF | CGCTGTTCTTCAACTGTGGAG | 270 |
| | LoopB | CACAATTCACAGCAGTAATTGC | 271 |
| | F2 | CACCTCTTAGTCGTTCTTCAGC | 272 |
| | F1c | ATTTGTGTGCGCCGTGGC | 273 |
| | B2 | TGAAAGTAGTCCTGAAAGCATG | 274 |
| | B1c | ATAGCACCCGGTACAAGCAGGA | 275 |
| 5 | ETEC STh | | |
| | F3 | CGGGTGTGTGGAGGACTT | 276 |
| | B3 | CTAAACCAGCAGGGTCTTCAAA | 277 |
| | FIP | ATATTTGTGTGCGCCGTGGCTCA | 278 |
| | (F1c + F2) | CCTCTTAGTCGTTCTTCAGC | |
| | BIP | ATAGCACCCGGTACAAGCAGGAT | 279 |
| | (B1c + B2) | GAAAGTAGTCCTGAAAGCATG | |
| 28 | LoopF | CGCTGTTCTTCAACTGTGGAG | 280 |
| | LoopB | CACAATTCACAGCAGTAATTGC | 281 |
| | F2 | CACCTCTTAGTCGTTCTTCAGC | 282 |
| | F1c | ATATTTGTGTGCGCCGTGGCT | 283 |
| | B2 | TGAAAGTAGTCCTGAAAGCATG | 284 |
| | B1c | ATAGCACCCGGTACAAGCAGGA | 285 |
| 6 | ETEC STh | | |
| | F3 | CGGGTGTGTGGAGGACTT | 286 |
| | B3 | GCTAAACCAGCAGGGTCTTC | 287 |
| | FIP | TATTTGTGTGCGCCGTGGCTGGC | 288 |
| | (F1c + F2) | ACCTCTTAGTCGTTCTTCAGC | |
| | BIP | ATAGCACCCGGTACAAGCAGGAT | 289 |
| | (B1c + B2) | GAAAGTAGTCCTGAAAGCATG | |
| | LoopF | CGCTGTTCTTCAACTGTGGAG | 290 |
| | LoopB | CACAATTCACAGCAGTAATTGC | 291 |
| | F2 | CACCTCTTAGTCGTTCTTCAGC | 292 |
| | F1c | TATTTGTGTGCGCCGTGGCTGG | 293 |
| | B2 | TGAAAGTAGTCCTGAAAGCATG | 294 |
| | B1c | ATAGCACCCGGTACAAGCAGGA | 295 |
| 7 | ETEC STh | | |
| | F3 | CGGGTGTGTGGAGGACTT | 296 |
| | B3 | GCTAAACCAGCAGGGTCTTC | 297 |
| | FIP | TTTATATTTGTGTGCGCCGTGGCA | 298 |
| | (F1c + F2) | CCTCTTAGTCGTTCTTCAGC | |
| | BIP | GCACCCGGTACAAGCAGGATTGAA | 299 |
| | (B1c + B2) | AGTAGTCCTGAAAGCATG | |
| | LoopF | CGCTGTTCTTCAACTGTGGAG | 300 |
| | LoopB | CACAATTCACAGCAGTAATTGC | 301 |
| | F2 | CACCTCTTAGTCGTTCTTCAGC | 302 |
| | F1c | TTTATATTTGTGTGCGCCGTGG | 303 |
| | B2 | TGAAAGTAGTCCTGAAAGCATG | 304 |
| | B1c | GCACCCGGTACAAGCAGGAT | 305 |
| 8 | ETEC STh | | |
| | F3 | TGTGTGGAGGACTTTCACCT | 306 |
| | B3 | GCTAAACCAGCAGGGTCTTC | 307 |
| | FIP | ATTTGTGTGCGCCGTGGCTT | 308 |
| | (F1c + F2) | AGTCGTTCTTCAGCCTCCA | |
| | BIP | ATAGCACCCGGTACAAGCA | 309 |
| | (B1c + B2) | GGATGAAAGTAGTCCTGAAAGCATG | |
| | LoopF | TGGCGCTGTTCTTCAACTG | 310 |
| | LoopB | CACAATTCACAGCAGTAATTGC | 311 |
| | F2 | TTAGTCGTTCTTCAGCCTCCA | 312 |
| | F1c | ATTTGTGTGCGCCGTGGC | 313 |
| | B2 | TGAAAGTAGTCCTGAAAGCATG | 314 |
| | B1c | ATAGCACCCGGTACAAGCAGGA | 315 |
| 9 | ETEC STh | | |
| | F3 | TCCACAGTTGAAGAACAGC | 316 |
| | B3 | CTCTTCGTAGCGGAGAGT | 317 |
| | FIP | GCAATTACTGCTGTGAATTGTGC | 318 |
| | (F1c + F2) | GCACACAAATATAAAGGGAAC | |
| | BIP | TTGAAGACCCTGCTGGTTTAGCA | 319 |
| | (B1c + B2) | ATATTCGTGGACGACGT | |
| | LoopF | TTGTAATCCTGCTTGTACCG | 320 |
| | LoopB | ATCCTGAGCGAAAGGTGAAA | 321 |
| | F2 | CGCACACAAATATAAAGGGAAC | 322 |
| | F1c | GCAATTACTGCTGTGAATTGTG | 323 |
| | B2 | AATATTCGTGGACGACGT | 324 |
| | B1c | TTGAAGACCCTGCTGGTTTAGC | 325 |
| 10 | ETEC STh | | |
| | F3 | TCTTCAGCCTCCACAGTT | 326 |
| | B3 | CTCTTCGTAGCGGAGAGT | 327 |
| | FIP | GCAATTACTGCTGTGAATTGTGGA | 328 |
| | (F1c + F2) | AGAACAGCGCCAGCCA | |
| | BIP | TTGAAGACCCTGCTGGTTTAGCCGT | 329 |
| | (B1c + B2) | GTTTCGGAGGTAATATGAA | |
| | LoopF | TTGTAATCCTGCTTGTACCG | 330 |
| | LoopB | ATCCTGAGCGAAAGGTGAAA | 331 |
| | F2 | GAAGAACAGCGCCAGCCA | 332 |
| | F1c | GCAATTACTGCTGTGAATTGTG | 333 |
| | B2 | CGTGTTTCGGAGGTAATATGAA | 334 |
| | B1c | TTGAAGACCCTGCTGGTTTAGC | 335 |
| 11 | ETEC STh | | |
| | F3 | TCTTCAGCCTCCACAGTT | 336 |
| | B3 | CTCTTCGTAGCGGAGAGTA | 337 |
| | FIP | GCAATTACTGCTGTGAATTGTGGAA | 338 |
| | (F1c + F2) | GAACAGCGCCAGCCA | |
| | BIP | TTGAAGACCCTGCTGGTTTAGCCGT | 339 |
| | (B1c + B2) | GTTTCGGAGGTAATATGAA | |
| | LoopF | TTGTAATCCTGCTTGTACCG | 340 |
| | LoopB | ATCCTGAGCGAAAGGTGAAA | 341 |
| | F2 | GAAGAACAGCGCCAGCCA | 342 |
| | F1c | GCAATTACTGCTGTGAATTGTG | 343 |
| | B2 | CGTGTTTCGGAGGTAATATGAA | 344 |
| | B1c | TTGAAGACCCTGCTGGTTTAGC | 345 |
| 12 | ETEC STh | | |
| | F3 | TCCACAGTTGAAGAACAGC | 346 |
| | B3 | CTCTTCGTAGCGGAGAGT | 347 |
| | FIP | AGTAGCAATTACTGCTGTGACGCAC | 348 |
| | (F1c + F2) | ACAAATATAAAGGGAAC | |
| | BIP | GAAGACCCTGCTGGTTTAGCAATA | 349 |
| | (B1c + B2) | TTCGTGGACGACGT | |
| | LoopF | GTGTTGTAATCCTGCTTGTACC | 350 |
| | LoopB | ATCCTGAGCGAAAGGTGAAA | 351 |
| | F2 | CGCACACAAATATAAAGGGAAC | 352 |
| | F1c | AGTAGCAATTACTGCTGTGA | 353 |
| | B2 | AATATTCGTGGACGACGT | 354 |
| | B1c | GAAGACCCTGCTGGTTTAGC | 355 |
| 13 | ETEC STh | | |
| | F3 | TCTTCAGCCTCCACAGTT | 356 |
| | B3 | CTCTTCGTAGCGGAGAGT | 357 |
| | FIP | GTAGCAATTACTGCTGTGAAGAAGAA | 358 |
| | (F1c + F2) | CAGCGCCAGCCA | |
| | BIP | TTTGAAGACCCTGCTGGTTTAGCGTG | 359 |
| | (B1c + B2) | TTTCGGAGGTAATATGAA | |
| | LoopF | GTGTTGTAATCCTGCTTGTACC | 360 |
| | LoopB | CATCCTGAGCGAAAGGTGA | 361 |
| | F2 | GAAGAACAGCGCCAGCCA | 362 |
| | F1c | GTAGCAATTACTGCTGTGAA | 363 |
| | B2 | CGTGTTTCGGAGGTAATATGAA | 364 |
| | B1c | TTTGAAGACCCTGCTGGTTTAG | 365 |
| 14 | ETEC STh | | |
| | F3 | TCTTCAGCCTCCACAGTT | 366 |
| | B3 | CTCTTCGTAGCGGAGAGT | 367 |
| | FIP | TGTTGTAATCCTGCTTGTACCGGAAG | 368 |
| | (F1c + F2) | AACAGCGCCAGCCA | |
| | BIP | TTGAAGACCCTGCTGGTTTAGCCGTG | 369 |
| | (B1c + B2) | TTTCGGAGGTAATATGAA | |
| | LoopF | GTTCCCTTTATATTTGTGTGCG | 370 |
| | LoopB | ATCCTGAGCGAAAGGTGAAA | 371 |
| | F2 | GAAGAACAGCGCCAGCCA | 372 |
| | F1c | TGTTGTAATCCTGCTTGTACCG | 373 |
| | B2 | CGTGTTTCGGAGGTAATATGAA | 374 |
| | B1c | TTGAAGACCCTGCTGGTTTAGC | 375 |
| 15 | ETEC STh | | |
| | F3 | TCTTCAGCCTCCACAGTT | 366 |
| | B3 | CTCTTCGTAGCGGAGAGT | 367 |
| | FIP | GTGTTGTAATCCTGCTTGTACCGAAG | 376 |
| | (F1c + F2) | AACAGCGCCAGCCA | |
| | BIP | TTGAAGACCCTGCTGGTTTAGCCGTG | 369 |
| | (B1c + B2) | TTTCGGAGGTAATATGAA | |
| | LoopF | GTTCCCTTTATATTTGTGTGCG | 370 |
| | LoopB | ATCCTGAGCGAAAGGTGAAA | 371 |
| | F2 | GAAGAACAGCGCCAGCCA | 372 |
| | F1c | GTGTTGTAATCCTGCTTGTACC | 377 |
| | B2 | CGTGTTTCGGAGGTAATATGAA | 374 |
| | B1c | TTGAAGACCCTGCTGGTTTAGC | 375 |
| 16 | ETEC STh | | |
| | F3 | TCTTCAGCCTCCACAGTT | 366 |
| | B3 | CTCTTCGTAGCGGAGAGT | 367 |
| | FIP | TGTGTTGTAATCCTGCTTGT | 378 |
| | (F1c + F2) | ACGAAGAACAGCGCCAGCCA | |
| | BIP | TTGAAGACCCTGCTGGTTTAG | 369 |
| | (B1c + B2) | CCGTGTTTCGGAGGTAATATGAA | |
| | LoopF | GTTCCCTTTATATTTGTGTGCG | 370 |
| | LoopB | ATCCTGAGCGAAAGGTGAAA | 371 |
| | F2 | GAAGAACAGCGCCAGCCA | 372 |
| | F1c | TGTGTTGTAATCCTGCTTGTAC | 379 |
| | B2 | CGTGTTTCGGAGGTAATATGAA | 374 |
| | B1c | TTGAAGACCCTGCTGGTTTAGC | 375 |
| 1 | ETEC STP | Accession no. NC_017722 | |
| | F3 | TCAATACGGTTCTGA | 380 |
| | B3 | GTGTACCTCGACATA | 381 |
| | FIP | TTGTTGTAATCCTGCCCGAG | 382 |
| | (F1c + F2) | TCGCTTACTAT | |
| | BIP | TGAAGAGTCAAGTGAGCTA | 383 |
| | (B1c + B2) | ATGTTGGCAAT | |
| | LoopF | TGTGCTGGATGTTAT | 384 |
| | LoopB | CAGTTGACTGACTAA | 385 |
| | F2 | CGAGTCGCTTACTAT | 386 |
| | F1c | TTGTTGTAATCCTGCC | 387 |
| | B2 | GCTAATGTTGGCAAT | 388 |
| | B1c | TGAAGAGTCAAGTGA | 389 |
| 2 | ETEC STP | | |
| | F3 | CAATACGGTTCTGAC | 390 |
| | B3 | GTGTACCTCGACATA | 381 |
| | FIP | TTGTTGTAATCCTGCC | 391 |
| | (F1c + F2) | GAGTCGCTTACTATA | |
| | BIP | TGAAGAGTCAAGTGA | 383 |
| | (B1c + B2) | GCTAATGTTGGCAAT | |
| | LoopF | TGTGCTGGATGTTAT | 384 |
| | LoopB | CAGTTGACTGACTAA | 385 |
| | F2 | GAGTCGCTTACTATA | 392 |
| | F1c | TTGTTGTAATCCTGCC | 393 |
| | B2 | GCTAATGTTGGCAAT | 388 |
| | B1c | TGAAGAGTCAAGTGA | 389 |
| 3 | ETEC STP | | |
| | F3 | CAATACGGTTCTGAC | 394 |
| | B3 | GTGTACCTCGACATA | 395 |
| | FIP | TACTGCTGTGAACTTA | 396 |
| | (F1c + F2) | ATAACATCCAGCAC | |
| | BIP | TGAAGAGTCAAGTGA | 397 |
| | (B1c + B2) | GCTAATGTTGGCAAT | |
| | LoopF | TGTTGTAATCCTGCC | 398 |
| | LoopB | CAGTTGACTGACTAA | 399 |
| | F2 | AATAACATCCAGCAC | 400 |
| | F1c | TACTGCTGTGAACTT | 40 |
| | B2 | GCTAATGTTGGCAAT | 402 |
| | B1c | TGAAGAGTCAAGTGA | 403 |
| 4 | ETEC STp | | |
| | F3 | CAATACGGTTCTGAC | 404 |
| | B3 | GTGTACCTCGACATA | 405 |
| | FIP | TACTGCTGTGAACTTA | 406 |
| | (F1c + F2) | TAACATCCAGCACA | |
| | BIP | TGAAGAGTCAAGTGAG | 407 |
| | (B1c + B2) | CTAATGTTGGCAAT | |
| | LoopF | TGTTGTAATCCTGCC | 408 |
| | LoopB | CAGTTGACTGACTAA | 409 |
| | F2 | ATAACATCCAGCACA | 410 |
| | F1c | TACTGCTGTGAACTT | 411 |
| | B2 | GCTAATGTTGGCAAT | 412 |
| | B1c | TGAAGAGTCAAGTGA | 413 |
| 5 | ETEC STP | | |
| | F3 | TGAACATATCCAGGA | 414 |
| | B3 | GTGTACCTCGACATA | 415 |
| | FIP | TTGTTGTAATCCTGCCC | 416 |
| | (F1c + F2) | AATACGGTTCTGAC | |
| | BIP | TGAAGAGTCAAGTGAG | 417 |
| | (B1c + B2) | CTAATGTTGGCAAT | |
| | LoopF | TGTGCTGGATGTTAT | 418 |
| | LoopB | CAGTTGACTGACTAA | 419 |
| | F2 | CAATACGGTTCTGAC | 420 |
| | F1c | TTGTTGTAATCCTGCC | 421 |
| | B2 | GCTAATGTTGGCAAT | 422 |
| | B1c | TGAAGAGTCAAGTGA | 423 |
| 6 | ETEC STP | | |
| | F3 | ATCAATACGGTTCTG | 424 |
| | B3 | GTGTACCTCGACATA | 425 |
| | FIP | TTGTTGTAATCCTGCCA | 426 |
| | (F1c + F2) | CGAGTCGCTTACTA | |
| | BIP | TGAAGAGTCAAGTGAG | 427 |
| | (B1c + B2) | CTAATGTTGGCAAT | |
| | LoopF | TGTGCTGGATGTTAT | 428 |
| | LoopB | CAGTTGACTGACTAA | 429 |
| | F2 | ACGAGTCGCTTACTA | 430 |
| | F1c | TTGTTGTAATCCTGCC | 431 |
| | B2 | GCTAATGTTGGCAAT | 432 |
| | B1c | TGAAGAGTCAAGTGA | 433 |
| 7 | ETEC STp | | |
| | F3 | TGAACATATCCAGGA | 434 |
| | B3 | GTACCTCGACATATAAC | 435 |
| | FIP | TTGTTGTAATCCTGCCCAA | 436 |
| | (F1c + F2) | TACGGTTCTGAC | |
| | BIP | TGAAGAGTCAAGTGAGCT | 437 |
| | (B1c + B2) | AATGTTGGCAAT | |
| | LoopF | TGTGCTGGATGTTAT | 438 |
| | LoopB | CAGTTGACTGACTAA | 439 |
| | F2 | CAATACGGTTCTGAC | 440 |
| | F1c | TTGTTGTAATCCTGCC | 441 |
| | B2 | GCTAATGTTGGCAAT | 442 |
| | B1c | TGAAGAGTCAAGTGA | 443 |
| 8 | ETEC STP | | |
| | F3 | CAATACGGTTCTGAC | 444 |
| | B3 | GTGTACCTCGACATA | 445 |
| | FIP | TTACTGCTGTGAACTT | 446 |
| | (F1c + F2) | AACATCCAGCACAG | |
| | BIP | TGAAGAGTCAAGTGA | 447 |
| | (B1c + B2) | GCTAATGTTGGCAAT | |
| | LoopF | TTGTTGTAATCCTGC | 448 |
| | LoopB | CAGTTGACTGACTAA | 449 |
| | F2 | TAACATCCAGCACAG | 450 |
| | F1c | TTACTGCTGTGAACT | 45 |
| | B2 | GCTAATGTTGGCAAT | 452 |
| | B1c | TGAAGAGTCAAGTGA | 453 |
| 9 | ETEC STP | | |
| | F3 | TGTACTCGCTGATCG | 454 |
| | B3 | CTGAATCACTTGACTCT | 455 |
| | FIP | TCCTGGATATGTTCAAT | 456 |
| | (F1c + F2) | GACTGAACAATGTGGAGC | |
| | BIP | TATCAATACGGTTCTGAC | 457 |
| | (B1c + B2) | GATACTGCTGTGAACTT | |
| | LoopF | GTAACGCAGCCACTT | 458 |
| | LoopB | GCACAGGCAGGATTA | 459 |
| | F2 | TGAACAATGTGGAGC | 460 |
| | F1c | TCCTGGATATGTTCAATGAC | 461 |
| | B2 | TACTGCTGTGAACTT | 462 |
| | B1c | TATCAATACGGTTCTGACGA | 463 |
| 10 | ETEC STp | | |
| | F3 | AGCGGTGGTGAACAT | 464 |
| | B3 | CTGAATCACTTGACTCT | 465 |
| | FIP | CCTGGATATGTTCAATGACGT | 466 |
| | (F1c + F2) | GTACTCGCTGATCG | |
| | BIP | TATCAATACGGTTCTGACGAT | 467 |
| | (B1c + B2) | ACTGCTGTGAACTT | |
| | LoopF | CTCCACATTGTTCAGAC | 468 |
| | LoopB | GCACAGGCAGGATTA | 469 |
| | F2 | TGTACTCGCTGATCG | 470 |
| | F1c | CCTGGATATGTTCAATGACG | 471 |
| | B2 | TACTGCTGTGAACTT | 472 |
| | B1c | TATCAATACGGTTCTGACGA | 473 |
| 11 | ETEC STP | | |
| | F3 | AGCGGTGGTGAACAT | 474 |
| | B3 | ACTGAATCACTTGACTC | 475 |
| | FIP | CCTGGATATGTTCAATGACGT | 476 |
| | (F1c + F2) | GTACTCGCTGATCG | |
| | BIP | TATCAATACGGTTCTGACGAT | 477 |
| | (B1c + B2) | ACTGCTGTGAACTT | |
| | LoopF | CTCCACATTGTTCAGAC | 478 |
| | LoopB | GCACAGGCAGGATTA | 479 |
| | F2 | TGTACTCGCTGATCG | 480 |
| | F1c | CCTGGATATGTTCAATGACG | 481 |
| | B2 | TACTGCTGTGAACTT | 482 |
| | B1c | TATCAATACGGTTCTGACGA | 483 |
| 12 | ETEC STp | | |
| | F3 | AGCGGTGGTGAACAT | 484 |
| | B3 | CTGAATCACTTGACTCT | 485 |
| | FIP | TCCTGGATATGTTCAATGACT | 486 |
| | (F1c + F2) | GTACTCGCTGATCG | |
| | BIP | TATCAATACGGTTCTGACGAT | 487 |
| | (B1c + B2) | ACTGCTGTGAACTT | |
| | LoopF | CTCCACATTGTTCAGAC | 488 |
| | LoopB | GCACAGGCAGGATTA | 489 |
| | F2 | TGTACTCGCTGATCG | 490 |
| | F1c | TCCTGGATATGTTCAATGAC | 491 |
| | B2 | TACTGCTGTGAACTT | 492 |
| | B1c | TATCAATACGGTTCTGACGA | 493 |
| 13 | ETEC STP | | |
| | F3 | AGCGGTGGTGAACAT | 494 |
| | B3 | CTGAATCACTTGACTCT | 495 |
| | FIP | CGTAACGCAGCCACTTGTACT | 496 |
| | (F1c + F2) | CGCTGATCG | |
| | BIP | TATCAATACGGTTCTGACGAT | 497 |
| | (B1c + B2) | ACTGCTGTGAACTT | |
| | LoopF | CTCCACATTGTTCAGAC | 498 |
| | LoopB | GCACAGGCAGGATTA | 499 |
| | F2 | TGTACTCGCTGATCG | 500 |
| | F1c | CGTAACGCAGCCACT | 501 |
| | B2 | TACTGCTGTGAACTT | 502 |
| | B1c | TATCAATACGGTTCTGACGA | 503 |
| 14 | ETEC STP | | |
| | F3 | AGCGGTGGTGAACAT | 504 |
| | B3 | CTGAATCACTTGACTCT | 505 |
| | FIP | GTAACGCAGCCACTTTGTACT | 506 |
| | (F1c + F2) | CGCTGATCG | |
| | BIP | TATCAATACGGTTCTGACGAT | 507 |
| | (B1c + B2) | ACTGCTGTGAACTT | |
| | LoopF | CTCCACATTGTTCAGAC | 508 |
| | LoopB | GCACAGGCAGGATTA | 509 |
| | F2 | TGTACTCGCTGATCG | 510 |
| | F1c | GTAACGCAGCCACTT | 511 |
| | B2 | TACTGCTGTGAACTT | 512 |
| | B1c | TATCAATACGGTTCTGACGA | 513 |
| 15 | ETEC STP | | |
| | F3 | AGCGGTGGTGAACAT | 514 |
| | B3 | CTGAATCACTTGACTCT | 515 |
| | FIP | GCCAATCCTGGATATGTTCTG | 516 |
| | (F1c + F2) | TACTCGCTGATCG | |
| | BIP | TATCAATACGGTTCTGACGATA | 517 |
| | (B1c + B2) | CTGCTGTGAACTT | |
| | LoopF | CTCCACATTGTTCAGAC | 518 |
| | LoopB | GCACAGGCAGGATTA | 519 |
| | F2 | TGTACTCGCTGATCG | 520 |
| | F1c | GCCAATCCTGGATATGTTC | 521 |
| | B2 | TACTGCTGTGAACTT | 522 |
| | B1c | TATCAATACGGTTCTGACGA | 523 |
| 16 | ETEC STp | | |
| | F3 | ACATGCCGTCTGAAC | 524 |
| | B3 | CTGAATCACTTGACTCT | 525 |
| | FIP | TCCTGGATATGTTCAATGAC | 526 |
| | (F1c + F2) | AATGTGGAGCCAGAA | |
| | BIP | TATCAATACGGTTCTGACGA | 527 |
| | (B1c + B2) | TACTGCTGTGAACTT | |
| | LoopF | GTAACGCAGCCACTT | 528 |
| | LoopB | GCACAGGCAGGATTA | 529 |
| | F2 | AATGTGGAGCCAGAA | 530 |
| | F1c | TCCTGGATATGTTCAATGAC | 531 |
| | B2 | TACTGCTGTGAACTT | 532 |
| | B1c | TATCAATACGGTTCTGACGA | 533 |
| 1 | Shigella | | |
| | ipaH | Accession no. M76444 | |
| | F3 | AATTCTGGAGGACATTGC | 534 |
| | B3 | TACGCTTCAGTACAGCA | 535 |
| | FIP | GGCAGTGGAGAGCTGAAGGGAT | 536 |
| | (F1c + F2) | GAGATAGAAGTCTACCT | |
| | BIP | CGCACTGCCGAAGCTATGGAGAA | 537 |
| | (B1c + B2) | CCAGTCCGTAA | |
| | LoopF | CGAGCATGGTCTGGAAG | 538 |
| | LoopB | CAGAAGCCGTGAAGAGAAT | 539 |
| | F2 | GGATGAGATAGAAGTCTACCT | 540 |
| | F1c | GGCAGTGGAGAGCTGAAG | 541 |
| | B2 | GGAGAACCAGTCCGTAA | 542 |
| | B1c | CGCACTGCCGAAGCTAT | 543 |
| 2 | Shigella | | |
| | ipaH | | |
| | F3 | TGTATCACAGATATGGCATG | 544 |
| | B3 | GCGCCGGTATCATTATC | 545 |
| | FIP | CCATGCAGCGACCTGTTTTCCGAT | 546 |
| | (F1c + F2) | ACCGTCTCTG | |
| | BIP | TTCGCTGTTGCTGCTGATATTGTT | 547 |
| | (B1c + B2) | CCATGTGAGCG | |
| | LoopF | CGGAATCCGGAGGTATTG | 548 |
| | LoopB | TGAGAGCTGTGAGGACC | 549 |
| | F2 | TTCCGATACCGTCTCTG | 550 |
| | F1c | CCATGCAGCGACCTGTT | 551 |
| | B2 | ATTGTTCCATGTGAGCG | 552 |
| | B1c | TTCGCTGTTGCTGCTGAT | 553 |
| 3 | Shigella | | |
| | ipaH | | |
| | F3 | GGGAGTGACAGCAAATG | 554 |
| | B3 | GTTCAGTCTCACGCATC | 555 |
| | FIP | GCGGTCAGCTTCCGTACTTACGGA | 556 |
| | (F1c + F2) | CTGGTTCTCC | |
| | BIP | GCAGAAGAGCAGAAGTATGAGAT | 557 |
| | (B1c + B2) | CAGACCTGATGCTTTCAG | |
| | LoopF | TCAGTACAGCATGCCATG | 558 |
| | LoopB | TGGAGAATGAGTACTCTCAGA | 559 |
| | F2 | TTACGGACTGGTTCTCC | 560 |
| | F1c | GCGGTCAGCTTCCGTAC | 561 |
| | B2 | CAGACCTGATGCTTTCAG | 562 |
| | B1c | GCAGAAGAGCAGAAGTATGAGAT | 563 |
| 4 | Shigella | | |
| | ipaH | | |
| | F3 | CTTCGACAGCAGTCTTTC | 564 |
| | B3 | CAGTGGAGAGCTGAAGT | 565 |
| | FIP | GCGCCGGTATCATTATCGACGCTCA | 566 |
| | (F1c + F2) | CATGGAACAAT | |
| | BIP | CCTCGAAATTCTGGAGGACATTAGA | 567 |
| | (B1c + B2) | CTTCTATCTCATCCACA | |
| | LoopF | CCTTCTGATGCCTGATGG | 568 |
| | LoopB | GGATAAAGTCAGAACTCTCCAT | 569 |
| | F2 | CGCTCACATGGAACAAT | 570 |
| | F1c | GCGCCGGTATCATTATCGA | 571 |
| | B2 | AGACTTCTATCTCATCCACA | 572 |
| | B1c | CCTCGAAATTCTGGAGGACATT | 573 |
| 5 | Shigella | | |
| | ipaH | | |
| | F3 | CTTCGACAGCAGTCTTTC | 574 |
| | B3 | CAGTGGAGAGCTGAAGT | 575 |
| | FIP | AGCGCCGGTATCATTATCGCTCACAT | 576 |
| | (F1c + F2) | GGAACAATCTCC | |
| | BIP | CCTCGAAATTCTGGAGGACATTAGA | 577 |
| | (B1c + B2) | CTTCTATCTCATCCACA | |
| | LoopF | CCTTCTGATGCCTGATGG | 578 |
| | LoopB | GGATAAAGTCAGAACTCTCCAT | 579 |
| | F2 | CTCACATGGAACAATCTCC | 580 |
| | F1c | AGCGCCGGTATCATTATCG | 581 |
| | B2 | AGACTTCTATCTCATCCACA | 582 |
| | B1c | CCTCGAAATTCTGGAGGACATT | 583 |
| 6 | Shigella | | |
| | ipaH | | |
| | F3 | CTTCGACAGCAGTCTTTC | 584 |
| | B3 | CAGTGGAGAGCTGAAGT | 585 |
| | FIP | AGCGCCGGTATCATTATCGGCTCACA | 586 |
| | (F1c + F2) | TGGAACAATCT | |
| | BIP | CCTCGAAATTCTGGAGGACATTAGAC | 587 |
| | (B1c + B2) | TTCTATCTCATCCACA | |
| | LoopF | CCTTCTGATGCCTGATGG | 588 |
| | LoopB | GGATAAAGTCAGAACTCTCCAT | 589 |
| | F2 | GCTCACATGGAACAATCT | 590 |
| | F1c | AGCGCCGGTATCATTATCG | 591 |
| | B2 | AGACTTCTATCTCATCCACA | 592 |
| | B1c | CCTCGAAATTCTGGAGGACATT | 593 |
| 7 | Shigella | | |
| | ipaH | | |
| | F3 | TGAAGAGCATGCCAACAC | 594 |
| | B3 | GCCTTCTGATGCCTGATG | 595 |
| | FIP | TCCGCAGAGGCACTGAGTCTCTGCA | 596 |
| | (F1c + F2) | CGCAATACCTC | |
| | BIP | TCTTTCGCTGTTGCTGCTGATAGATT | 597 |
| | (B1c + B2) | GTTCCATGTGAGCG | |
| | LoopF | GCGACCTGTTCACGGAAT | 598 |
| | LoopB | GCCACTGAGAGCTGTGAG | 599 |
| | F2 | CTCTGCACGCAATACCTC | 600 |
| | F1c | TCCGCAGAGGCACTGAGT | 601 |
| | B2 | AGATTGTTCCATGTGAGCG | 602 |
| | B1c | TCTTTCGCTGTTGCTGCTGAT | 603 |
| 8 | Shigella | | |
| | ipaH | | |
| | F3 | TGAACATGAAGAGCATGCC | 604 |
| | B3 | GCCTTCTGATGCCTGATG | 605 |
| | FIP | TCCGCAGAGGCACTGAGTGTCTCT | 606 |
| | (F1c + F2) | GCACGCAATACC | |
| | BIP | TCTTTCGCTGTTGCTGCTGATAGA | 607 |
| | (B1c + B2) | TTGTTCCATGTGAGCG | |
| | LoopF | GCGACCTGTTCACGGAAT | 608 |
| | LoopB | GCCACTGAGAGCTGTGAG | 609 |
| | F2 | GTCTCTGCACGCAATACC | 610 |
| | F1c | TCCGCAGAGGCACTGAGT | 611 |
| | B2 | AGATTGTTCCATGTGAGCG | 612 |
| | B1c | TCTTTCGCTGTTGCTGCTGAT | 613 |
| 9 | Shigella | | |
| | ipaH | | |
| | F3 | AGCTTCGACAGCAGTCTT | 614 |
| | B3 | GCCAGGTAGACTTCTATCTCA | 615 |
| | FIP | TGATGCCTGATGGACCAGGACACT | 616 |
| | (F1c + F2) | GAGAGCTGTGAGGA | |
| | BIP | CGATAATGATACCGGCGCTCTGGG | 617 |
| | (B1c + B2) | CAATGTCCTCCAGAATT | |
| | LoopF | CCGGAGATTGTTCCATGTGA | 618 |
| | LoopB | GGAAATGTTCCGCCTCGA | 619 |
| | F2 | CACTGAGAGCTGTGAGGA | 620 |
| | F1c | TGATGCCTGATGGACCAGGA | 621 |
| | B2 | GGCAATGTCCTCCAGAATT | 622 |
| | B1c | CGATAATGATACCGGCGCTCTG | 623 |
| 10 | Shigella | | |
| | ipaH | | |
| | F3 | TGAACATGAAGAGCATGCC | 624 |
| | B3 | GCCTTCTGATGCCTGATG | 625 |
| | FIP | TCCGCAGAGGCACTGAGTTCTCTG | 626 |
| | (F1c + F2) | CACGCAATACCT | |
| | BIP | TCTTTCGCTGTTGCTGCTGATAGA | 627 |
| | (B1c + B2) | TTGTTCCATGTGAGCG | |
| | LoopF | GCGACCTGTTCACGGAAT | 628 |
| | LoopB | GCCACTGAGAGCTGTGAG | 629 |
| | F2 | TCTCTGCACGCAATACCT | 630 |
| | F1c | TCCGCAGAGGCACTGAGT | 631 |
| | B2 | AGATTGTTCCATGTGAGCG | 632 |
| | B1c | TCTTTCGCTGTTGCTGCTGAT | 633 |
| 11 | Shigella | | |
| | ipaH | | |
| | F3 | CTCTCCATTTTGTGGATGAGAT | 634 |
| | B3 | CGTACGCTTCAGTACAGC | 19 |
| | FIP | CTTCACGGCAGTGGAGAGCAAGT | 635 |
| | (F1c + F2) | CTACCTGGCCTTCC | |
| | BIP | TATGGCGTGTCGGGAGTGATTCA | 21 |
| | (B1c + B2) | TTCTCTTCACGGCTTC | |
| | LoopF | TCTCTGCGAGCATGGTCT | 636 |
| | LoopB | CACTGCCGAAGCTATGGT | 23 |
| | F2 | AAGTCTACCTGGCCTTCC | 637 |
| | F1c | CTTCACGGCAGTGGAGAGC | 25 |
| | B2 | TTCATTCTCTTCACGGCTTC | 26 |
| | B1c | TATGGCGTGTCGGGAGTGA | 27 |
| 12 | Shigella | | |
| | ipaH | | |
| | F3 | CCGTGACAGCATGGTTCC | 638 |
| | B3 | CACGGTCCTCACAGCTCT | 639 |
| | FIP | AGGTATTGCGTGCAGAGACGGTAT | 640 |
| | (F1c + F2) | GAAGAGCATGCCAACACC | |
| | BIP | AACAGGTCGCTGCATGGCTGCATC | 641 |
| | (B1c + B2) | AGCAGCAACAGCGA | |
| | LoopF | AAGGCGGTCAAGGAACGC | 642 |
| | LoopB | CGGAGCTTCGACAGCAGTC | 643 |
| | F2 | ATGAAGAGCATGCCAACACC | 644 |
| | F1c | AGGTATTGCGTGCAGAGACGGT | 645 |
| | B2 | CATCAGCAGCAACAGCGA | 646 |
| | B1c | AACAGGTCGCTGCATGGCTG | 647 |
| 13 | Shigella | | |
| | ipaH | | |
| | F3 | GATTCCGTGAACAGGTCGC | 648 |
| | B3 | TTCTCTGCGAGCATGGTCT | 649 |
| | FIP | CCGGAGATTGTTCCATGTGAGCGT | 650 |
| | (F1c + F2) | GTTGCTGCTGATGCCAC | |
| | BIP | AATGATACCGGCGCTCTGCTCTGC | 651 |
| | (B1c + B2) | AATGTCCTCCAGAATTTCGA | |
| | LoopF | ACACGGTCCTCACAGCTCT | 652 |
| | LoopB | CCTGGGCAGGGAAATGTTCC | 653 |
| | F2 | TGTTGCTGCTGATGCCAC | 654 |
| | F1c | CCGGAGATTGTTCCATGTGAGCG | 655 |
| | B2 | GCAATGTCCTCCAGAATTTCGA | 656 |
| | B1c | AATGATACCGGCGCTCTGCTCT | 657 |
| 14 | Shigella | | |
| | ipaH | | |
| | F3 | TCTGCGGAGCTTCGACA | 658 |
| | B3 | TTCTCTGCGAGCATGGTCT | 659 |
| | FIP | TGATGCCTGATGGACCAGGAGGAT | 660 |
| | (F1c + F2) | GCCACTGAGAGCTGTGA | |
| | BIP | AATGATACCGGCGCTCTGCTCTGC | 661 |
| | (B1c + B2) | AATGTCCTCCAGAATTTCGA | |
| | LoopF | ATGTGAGCGCGACACGG | 662 |
| | LoopB | CCTGGGCAGGGAAATGTTCC | 663 |
| | F2 | ATGCCACTGAGAGCTGTGA | 664 |
| | F1c | TGATGCCTGATGGACCAGGAGG | 665 |
| | B2 | GCAATGTCCTCCAGAATTTCGA | 666 |
| | B1c | AATGATACCGGCGCTCTGCTCT | 667 |
| 15 | Shigella | | |
| | ipaH | | |
| | F3 | CGTGAACAGGTCGCTGC | 668 |
| | B3 | TTCTCTGCGAGCATGGTCT | 669 |
| | FIP | TGATGCCTGATGGACCAGGAGGT | 670 |
| | (F1c + F2) | GATGCCACTGAGAGCTGT | |
| | BIP | AATGATACCGGCGCTCTGCTCTGC | 671 |
| | (B1c + B2) | AATGTCCTCCAGAATTTCGA | |
| | LoopF | ATGTGAGCGCGACACGG | 672 |
| | LoopB | CCTGGGCAGGGAAATGTTCC | 673 |
| | F2 | TGATGCCACTGAGAGCTGT | 674 |
| | F1c | TGATGCCTGATGGACCAGGAGG | 675 |
| | B2 | GCAATGTCCTCCAGAATTTCGA | 676 |
| | B1c | AATGATACCGGCGCTCTGCTCT | 677 |
| 16 | Shigella | | |
| | ipaH | | |
| | F3 | TGTTGCTGCTGATGCCAC | 678 |
| | B3 | TTCTCTGCGAGCATGGTCT | 679 |
| | FIP | TGATGCCTGATGGACCAGGAGGAG | 680 |
| | (F1c + F2) | AGCTGTGAGGACCGTG | |
| | BIP | AATGATACCGGCGCTCTGCTCTGC | 681 |
| | (B1c + B2) | AATGTCCTCCAGAATTTCGA | |
| | LoopF | CCGGAGATTGTTCCATGTGAGC | 682 |
| | LoopB | CCTGGGCAGGGAAATGTTCC | 683 |
| | F2 | AGAGCTGTGAGGACCGTG | 684 |
| | F1c | TGATGCCTGATGGACCAGGAGG | 685 |
| | B2 | GCAATGTCCTCCAGAATTTCGA | 686 |
| | B1c | AATGATACCGGCGCTCTGCTCT | 687 |
| 17 | Shigella | | |
| | ipaH | | |
| | F3 | GATTCCGTGAACAGGTCGC | 688 |
| | B3 | TTCTCTGCGAGCATGGTCT | 689 |
| | FIP | CCGGAGATTGTTCCATGTGAGCGTC | 690 |
| | (F1c + F2) | GCTGTTGCTGCTGATG | |
| | BIP | AATGATACCGGCGCTCTGCTCTGCAA | 691 |
| | (B1c + B2) | TGTCCTCCAGAATTTCGA | |
| | LoopF | ACACGGTCCTCACAGCTCT | 692 |
| | LoopB | CCTGGGCAGGGAAATGTTCC | 693 |
| | F2 | TCGCTGTTGCTGCTGATG | 694 |
| | F1c | CCGGAGATTGTTCCATGTGAGCG | 695 |
| | B2 | GCAATGTCCTCCAGAATTTCGA | 696 |
| | B1c | AATGATACCGGCGCTCTGCTCT | 697 |
| 18 | Shigella | | |
| | ipaH | | |
| | F3 | AACATGAAGAGCATGCCAACACC | 698 |
| | B3 | AGCAGAGCGCCGGTATCAT | 699 |
| | FIP | GTCGAAGCTCCGCAGAGGCACTTCTCTG | 700 |
| | (F1c + F2) | CACGCAATACCTCCG | |
| | BIP | CGCTGTTGCTGCTGATGCCACTGCGGAG | 701 |
| | (B1c + B2) | ATTGTTCCATGTGAGCG | |
| | LoopF | GCAGCGACCTGTTCACGGAATC | 702 |
| | LoopB | AGAGCTGTGAGGACCGTGTCG | 703 |
| | F2 | TCTCTGCACGCAATACCTCCG | 704 |
| | F1c | GTCGAAGCTCCGCAGAGGCACT | 705 |
| | B2 | CGGAGATTGTTCCATGTGAGCG | 706 |
| | B1c | CGCTGTTGCTGCTGATGCCACTG | 707 |
| 19 | Shigella | | |
| | ipaH | | |
| | F3 | TGTTCCGCCTCGAAATTCTGGA | 708 |
| | B3 | TTCCGTACGCTTCAGTACAGCAT | 709 |
| | FIP | TCCCGACACGCCATAGAAACGCAACCTGG | 710 |
| | (F1c + F2) | CCTTCCAGACCATG | |
| | BIP | ACAGCAAATGACCTCCGCACTGCCCAGAG | 711 |
| | (B1c + B2) | GGAGAACCAGTCCG | |
| | LoopF | ACGGCAGTGGAGAGCTGAAGT | 712 |
| | LoopB | CGAAGCTATGGTCAGAAGCCGTG | 713 |
| | F2 | ACCTGGCCTTCCAGACCATG | 714 |
| | F1c | TCCCGACACGCCATAGAAACGCA | 715 |
| | B2 | CCAGAGGGAGAACCAGTCCG | 716 |
| | B1c | ACAGCAAATGACCTCCGCACTGC | 717 |
| 20 | Shigella | | |
| | ipaH | | |
| | F3 | TGTTCCGCCTCGAAATTCTGGA | 718 |
| | B3 | TTCCGTACGCTTCAGTACAGCAT | 719 |
| | FIP | TCCCGACACGCCATAGAAACGCATGGCC | 720 |
| | (F1c + F2) | TTCCAGACCATGCT | |
| | BIP | ACAGCAAATGACCTCCGCACTGCCCAGA | 721 |
| | (B1c + B2) | GGGAGAACCAGTCCG | |
| | LoopF | ACGGCAGTGGAGAGCTGAAGT | 722 |
| | LoopB | CGAAGCTATGGTCAGAAGCCGTG | 723 |
| | F2 | TGGCCTTCCAGACCATGCT | 724 |
| | F1c | TCCCGACACGCCATAGAAACGCA | 725 |
| | B2 | CCAGAGGGAGAACCAGTCCG | 726 |
| | B1c | ACAGCAAATGACCTCCGCACTGC | 727 |
| 21 | Shigella | | |
| | ipaH | | |
| | F3 | TGTTCCGCCTCGAAATTCTGGA | 728 |
| | B3 | TTCCGTACGCTTCAGTACAGCAT | 729 |
| | FIP | TCCCGACACGCCATAGAAACGCACTGG | 730 |
| | (F1c + F2) | CCTTCCAGACCATGCT | |
| | BIP | ACAGCAAATGACCTCCGCACTGCCCAGA | 731 |
| | (B1c + B2) | GGGAGAACCAGTCCG | |
| | LoopF | ACGGCAGTGGAGAGCTGAAGT | 732 |
| | LoopB | CGAAGCTATGGTCAGAAGCCGTG | 733 |
| | F2 | CTGGCCTTCCAGACCATGCT | 734 |
| | F1c | TCCCGACACGCCATAGAAACGCA | 735 |
| | B2 | CCAGAGGGAGAACCAGTCCG | 736 |
| | B1c | ACAGCAAATGACCTCCGCACTGC | 737 |
| 22 | Shigella | | |
| | ipaH | | |
| | F3 | CCGCCTCGAAATTCTGGAGGA | 738 |
| | B3 | TTCCGTACGCTTCAGTACAGCAT | 739 |
| | FIP | TCCCGACACGCCATAGAAACGCACTTCC | 740 |
| | (F1c + F2) | AGACCATGCTCGCAGA | |
| | BIP | ACAGCAAATGACCTCCGCACTGCCCAGA | 741 |
| | (B1c + B2) | GGGAGAACCAGTCCG | |
| | LoopF | ACGGCAGTGGAGAGCTGAAGT | 742 |
| | LoopB | CGAAGCTATGGTCAGAAGCCGTG | 743 |
| | F2 | CTTCCAGACCATGCTCGCAGA | 744 |
| | F1c | TCCCGACACGCCATAGAAACGCA | 745 |
| | B2 | CCAGAGGGAGAACCAGTCCG | 746 |
| | B1c | ACAGCAAATGACCTCCGCACTGC | 747 |
| 23 | Shigella | | |
| | ipaH | | |
| | F3 | TCCGCCTCGAAATTCTGGAGG | 748 |
| | B3 | TTCCGTACGCTTCAGTACAGCAT | 749 |
| | FIP | TCCCGACACGCCATAGAAACGCACCTT | 750 |
| | (F1c + F2) | CCAGACCATGCTCGCA | |
| | BIP | ACAGCAAATGACCTCCGCACTGCCCAG | 751 |
| | (B1c + B2) | AGGGAGAACCAGTCCG | |
| | LoopF | ACGGCAGTGGAGAGCTGAAGT | 752 |
| | LoopB | CGAAGCTATGGTCAGAAGCCGTG | 753 |
| | F2 | CCTTCCAGACCATGCTCGCA | 754 |
| | F1c | TCCCGACACGCCATAGAAACGCA | 755 |
| | B2 | CCAGAGGGAGAACCAGTCCG | 756 |
| | B1c | ACAGCAAATGACCTCCGCACTGC | 757 |
| 24 | Shigella | | |
| | ipaH | | |
| | F3 | ACCGTCTCTGCACGCAAT | 758 |
| | B3 | AGAGCAGAGCGCCGGTATC | 759 |
| | FIP | GCTCCGCAGAGGCACTGAGTTTTTTT | 760 |
| | (F1c + F2) | ACCTCCGGATTCCGTGAAC | |
| | BIP | TGCCACTGAGAGCTGTGAGGACTTTTT | 761 |
| | (B1c + B2) | CTGATGCCTGATGGACCAGG | |
| | LoopF | TTC CAG CCA TGC AGC GAC C | 762 |
| | LoopB | GTCGCGCTCACATGGAACA | 763 |
| | F2 | ACCTCCGGATTCCGTGAAC | 764 |
| | F1c | GCTCCGCAGAGGCACTGAGTTTTTTT | 765 |
| | B2 | CTGATGCCTGATGGACCAGG | 766 |
| | B1c | TGCCACTGAGAGCTGTGAGGACTTTTT | 767 |
| 25 | Shigella | | |
| | ipaH | | |
| | F3 | AGCTGACTGACGAGGTACTG | 768 |
| | B3 | AGTTGCACGCCGTAAATCC | 769 |
| | FIP | TCCGGTCTGCGGTTTATGCTT | 770 |
| | (F1c + F2) | GTCTGAAAACGGCTCACGA | |
| | BIP | ACTCCGGAAAAACTGTGACCCG | 771 |
| | (B1c + B2) | GCCGGTATTGAGCGAGGA | |
| | LoopF | TGCGACGTGATTATGAATGGTG | 772 |
| | LoopB | CGGACCTTAACAACAACCCGTAAA | 773 |
| | F2 | GTCTGAAAACGGCTCACGA | 774 |
| | F1c | TCCGGTCTGCGGTTTATGCTT | 775 |
| | B2 | GCCGGTATTGAGCGAGGA | 776 |
| | B1c | ACTCCGGAAAAACTGTGACCCG | 777 |
| 26 | Shigella | | |
| | ipaH | | |
| | F3 | ATACCGTCTCTGCACGCA | 778 |
| | B3 | TGATGGACCAGGAGGGTT | 779 |
| | FIP | AAGCTCCGCAGAGGCACTGA | 780 |
| | (F1c + F2) | TACCTCCGGATTCCGTGAAC | |
| | BIP | AGCAGTCTTTCGCTGTTGCTGC | 781 |
| | (B1c + B2) | TCCGGAGATTGTTCCATGTG | |
| | LoopF | AGCCATGCAGCGACCT | 782 |
| | LoopB | GATGCCACTGAGAGCTGTGAGG | 783 |
| | F2 | TACCTCCGGATTCCGTGAAC | 784 |
| | F1c | AAGCTCCGCAGAGGCACTGA | 785 |
| | B2 | TCCGGAGATTGTTCCATGTG | 786 |
| | B1c | AGCAGTCTTTCGCTGTTGCTGC | 787 |
| 27 | Shigella | | |
| | ipaH | | |
| | F3 | GCATGCCAACACCTTTTCC | 788 |
| | B3 | TGATGGACCAGGAGGGTT | 789 |
| | FIP | CGACCTGTTCACGGAATCCGG | 790 |
| | (F1c + F2) | CTTGACCGCCTTTCCGATAC | |
| | BIP | AGCAGTCTTTCGCTGTTGCTGC | 791 |
| | (B1c + B2) | TCCGGAGATTGTTCCATGTG | |
| | LoopF | AGGTATTGCGTGCAGAGACG | 792 |
| | LoopB | ACTGAGAGCTGTGAGGACC | 793 |
| | F2 | CTTGACCGCCTTTCCGATAC | 794 |
| | F1c | CGACCTGTTCACGGAATCCGG | 795 |
| | B2 | TCCGGAGATTGTTCCATGTG | 796 |
| | B1c | AGCAGTCTTTCGCTGTTGCTGC | 797 |
| 1 | Cholera | | |
| | CtxA | Accession no. AF452584 | |
| | F3 | TGGATGGTATCGAGTTCAT | 798 |
| | B3 | GATTGGTATTCGTCAAGGAA | 799 |
| | FIP | CTCCAAGCTCTATGCTCCGAAC | 800 |
| | (F1c + F2) | TTAGATATTGCTCCAGC | |
| | BIP | AGAGCCGTGGATTCATCATGCG | 801 |
| | (B1c + B2) | CAAGTATTACTCATCGAT | |
| | LoopF | CCTGCCAATCCATAACCAT | 802 |
| | LoopB | GTTGTGGGAATGCTCCA | 803 |
| | F2 | AACTTAGATATTGCTCCAGC | 804 |
| | F1c | CTCCAAGCTCTATGCTCCG | 805 |
| | B2 | CGCAAGTATTACTCATCGAT | 806 |
| | B1c | AGAGCCGTGGATTCATCATG | 807 |
| 2 | Cholera | | |
| | CtxA | | |
| | F3 | AGATTCTAGACCTCCTGATG | 808 |
| | B3 | GTGCAGTGGCTATAACATAT | 809 |
| | FIP | CTGAGTTCCTCTTGCATGATCAAA | 810 |
| | (F1c + F2) | GAGGACAGAATGAGTACT | |
| | BIP | CGGGATTTGTTAGGCACGAGGGC | 811 |
| | (B1c + B2) | ACTTCTCAAACTAAT | |
| | LoopF | TTCATTTGAGTACCTCGGTC | 812 |
| | LoopB | TGATGGATATGTTTCCACCTC | 813 |
| | F2 | AAGAGGACAGAATGAGTACT | 814 |
| | F1c | CTGAGTTCCTCTTGCATGATCA | 815 |
| | B2 | GGGCACTTCTCAAACTAAT | 816 |
| | B1c | CGGGATTTGTTAGGCACGA | 817 |
| 3 | Cholera | | |
| | CtxA | | |
| | F3 | CAGGTGGTCTTATGCCA | 818 |
| | B3 | GTGCAGTGGCTATAACATAT | 819 |
| | FIP | CTGAGTTCCTCTTGCATGATCAAGA | 820 |
| | (F1c + F2) | GGACAGAATGAGTACTT | |
| | BIP | CGGGATTTGTTAGGCACGAGGGCA | 821 |
| | (B1c + B2) | CTTCTCAAACTAAT | |
| | LoopF | TTCATTTGAGTACCTCGGTC | 822 |
| | LoopB | TGATGGATATGTTTCCACCTC | 823 |
| | F2 | AGAGGACAGAATGAGTACTT | 824 |
| | F1c | CTGAGTTCCTCTTGCATGATCA | 825 |
| | B2 | GGGCACTTCTCAAACTAAT | 826 |
| | B1c | CGGGATTTGTTAGGCACGA | 827 |
| 4 | Cholera | | |
| | CtxA | | |
| | F3 | TGGATGGTATCGAGTTCAT | 828 |
| | B3 | GATTGGTATTCGTCAAGGAA | 829 |
| | FIP | CTCCAAGCTCTATGCTCCGAACTT | 830 |
| | (F1c + F2) | AGATATTGCTCCAGC | |
| | BIP | AGAGCCGTGGATTCATCATGCGC | 831 |
| | (B1c + B2) | AAGTATTACTCATCGAT | |
| | LoopF | CTGCCAATCCATAACCATCTGCT | 832 |
| | LoopB | GGTTGTGGGAATGCTCCAAGAT | 833 |
| | F2 | AACTTAGATATTGCTCCAGC | 834 |
| | F1c | CTCCAAGCTCTATGCTCCG | 835 |
| | B2 | CGCAAGTATTACTCATCGAT | 836 |
| | B1c | AGAGCCGTGGATTCATCATG | 837 |
| 5 | Cholera | | |
| | CtxA | | |
| | F3 | ATATCGGGCAGATTCTAGA | 838 |
| | B3 | GTTTGACCCACTAAGTGG | 29 |
| | FIP | TTTGAGTACCTCGGTCAAAGTACCC | 839 |
| | (F1c + F2) | TCCTGATGAAATAAAGCA | |
| | BIP | TGATCATGCAAGAGGAACTCAGATT | 31 |
| | (B1c + B2) | GAGGTGGAAACATATCC | |
| | LoopF | TCTGTCCTCTTGGCATAAGACCA | 32 |
| | LoopB | CGGGATTTGTTAGGCACGATGA | 33 |
| | F2 | CCTCCTGATGAAATAAAGCA | 840 |
| | F1c | TTTGAGTACCTCGGTCAAAGTAC | 35 |
| | B2 | ATTGAGGTGGAAACATATCC | 36 |
| | B1c | TGATCATGCAAGAGGAACTCAG | 37 |
| 6 | Cholera | | |
| | CtxA | | |
| | F3 | GCAGATTCTAGACCTCCTGA | 841 |
| | B3 | GGTGCAGTGGCTATAACATAT | 842 |
| | FIP | CGTCTGAGTTCCTCTTGCATGATCTG | 843 |
| | (F1c + F2) | CCAAGAGGACAGAATGA | |
| | BIP | GGCACGATGATGGATATGTTTCCAG | 844 |
| | (B1c + B2) | ACCAGACAATATAGTTTGACCC | |
| | LoopF | TTTGAGTACCTCGGTCAAAGTAC | 35 |
| | LoopB | CAATTAGTTTGAGAAGTGCCCAC | 845 |
| | F2 | TGCCAAGAGGACAGAATGA | 846 |
| | F1c | CGTCTGAGTTCCTCTTGCATGATC | 847 |
| | B2 | GACCAGACAATATAGTTTGACCC | 848 |
| | B1c | GGCACGATGATGGATATGTTTCCA | 849 |
| 7 | Cholera | | |
| | CtxA | | |
| | F3 | GCAGATTCTAGACCTCCTGA | 850 |
| | B3 | TTGGGTGCAGTGGCTATA | 851 |
| | FIP | CGTCTGAGTTCCTCTTGCATGATCTGCC | 852 |
| | (F1c + F2) | AAGAGGACAGAATGA | |
| | BIP | GGCACGATGATGGATATGTTTCCAGACC | 853 |
| | (B1c + B2) | AGACAATATAGTTTGACCC | |
| | LoopF | TTTGAGTACCTCGGTCAAAGTAC | 854 |
| | LoopB | CAATTAGTTTGAGAAGTGCCCAC | 855 |
| | F2 | TGCCAAGAGGACAGAATGA | 856 |
| | F1c | CGTCTGAGTTCCTCTTGCATGATC | 857 |
| | B2 | GACCAGACAATATAGTTTGACCC | 858 |
| | B1c | GGCACGATGATGGATATGTTTCCA | 859 |
| 8 | Cholera | | |
| | CtxA | | |
| | F3 | AGCAGTCAGGTGGTCTTAT | 860 |
| | B3 | GGTGCAGTGGCTATAACATAT | 861 |
| | FIP | CGTCTGAGTTCCTCTTGCATGATCGCC | 862 |
| | (F1c + F2) | AAGAGGACAGAATGAG | |
| | BIP | GGCACGATGATGGATATGTTTCCAGA | 863 |
| | (B1c + B2) | CCAGACAATATAGTTTGACCC | |
| | LoopF | CATTTGAGTACCTCGGTCAAAGTA | 864 |
| | LoopB | CAATTAGTTTGAGAAGTGCCCAC | 865 |
| | F2 | GCCAAGAGGACAGAATGAG | 866 |
| | F1c | CGTCTGAGTTCCTCTTGCATGATC | 867 |
| | B2 | GACCAGACAATATAGTTTGACCC | 868 |
| | B1c | GGCACGATGATGGATATGTTTCCA | 869 |
| 9 | Cholera | | |
| | CtxA | | |
| | F3 | GCAGATTCTAGACCTCCTGA | 870 |
| | B3 | CTTGTTCATCTGGATGAGGAC | 871 |
| | FIP | CGTCTGAGTTCCTCTTGCATGATCAGG | 872 |
| | (F1c + F2) | TGGTCTTATGCCAAGA | |
| | BIP | GGCACGATGATGGATATGTTTCCAGGT | 873 |
| | (B1c + B2) | GCAGTGGCTATAACATAT | |
| | LoopF | TGAGTACCTCGGTCAAAGTACTCA | 874 |
| | LoopB | AGTTTGAGAAGTGCCCACTTAGTG | 875 |
| | F2 | AGGTGGTCTTATGCCAAGA | 876 |
| | F1c | CGTCTGAGTTCCTCTTGCATGATC | 877 |
| | B2 | GGTGCAGTGGCTATAACATAT | 878 |
| | B1c | GGCACGATGATGGATATGTTTCCA | 869 |
| 10 | Cholera | | |
| | CtxA | | |
| | F3 | GGCACGATGATGGATATGTT | 879 |
| | B3 | GATGAATCCACGGCTCTTC | 880 |
| | FIP | TGGAATCCCACCTAAAGCAGAAACATAT | 881 |
| | (F1c + F2) | GTTATAGCCACTGCACC | |
| | BIP | AGATATTGCTCCAGCAGCAGATGTCCAA | 882 |
| | (B1c + B2) | GCTCTATGCTCCG | |
| | LoopF | TCATCTGGATGAGGACTGTATGCC | 883 |
| | LoopB | GTTATGGATTGGCAGGTTTCCCT | 884 |
| | F2 | ATATGTTATAGCCACTGCACC | 885 |
| | F1c | TGGAATCCCACCTAAAGCAGAAAC | 886 |
| | B2 | TCCAAGCTCTATGCTCCG | 887 |
| | B1c | AGATATTGCTCCAGCAGCAGATG | 888 |
| 11 | Cholera | | |
| | CtxA | | |
| | F3 | AGGTGGTCTTATGCCAAGA | 889 |
| | B3 | GGAAACCTGCCAATCCATAA | 890 |
| | FIP | GTTGGGTGCAGTGGCTATAACATGGC | 891 |
| | (F1c + F2) | ACGATGATGGATATGTT | |
| | BIP | GGCATACAGTCCTCATCCAGATGACG | 892 |
| | (B1c + B2) | ATACCATCCATATATTTGGGAG | |
| | LoopF | CACTAAGTGGGCACTTCTCAAACT | 893 |
| | LoopB | GTTTCTGCTTTAGGTGGGATTCCA | 894 |
| | F2 | GGCACGATGATGGATATGTT | 895 |
| | F1c | GTTGGGTGCAGTGGCTATAACAT | 896 |
| | B2 | CGATACCATCCATATATTTGGGAG | 897 |
| | B1c | GGCATACAGTCCTCATCCAGATGA | 898 |
| 12 | Cholera | | |
| | CtxA | | |
| | F3 | TCAACCTTTATGATCATGCAAGAGG | 899 |
| | B3 | CGATGTAATTGTTCATCAAGCACC | 900 |
| | FIP | GACCCACTAAGTGGGCACTTCTCACTCA | 901 |
| | (F1c + F2) | GACGGGATTTGTTAGGC | |
| | BIP | AGCCACTGCACCCAACATGTTTAACAAT | 902 |
| | (B1c + B2) | CCCACCTAAAGCAGAAACTTC | |
| | LoopF | GAGGTGGAAACATATCCATCATCGT | 903 |
| | LoopB | GCATACAGTCCTCATCCAGATGAAC | 904 |
| | F2 | CTCAGACGGGATTTGTTAGGC | 905 |
| | F1c | GACCCACTAAGTGGGCACTTCTCA | 906 |
| | B2 | AATCCCACCTAAAGCAGAAACTTC | 907 |
| | B1c | AGCCACTGCACCCAACATGTTTAAC | 908 |
| 13 | Cholera | | |
| | CtxA | | |
| | F3 | TCAACCTTTATGATCATGCAAGAGG | 909 |
| | B3 | CGATGTAATTGTTCATCAAGCACC | 910 |
| | FIP | TGACCCACTAAGTGGGCACTTCTCCT | 911 |
| | (F1c + F2) | CAGACGGGATTTGTTAGGC | |
| | BIP | AGCCACTGCACCCAACATGTTTAACAA | 912 |
| | (B1c + B2) | TCCCACCTAAAGCAGAAACTTC | |
| | LoopF | GAGGTGGAAACATATCCATCATCGT | 913 |
| | LoopB | GCATACAGTCCTCATCCAGATGAAC | 914 |
| | F2 | CTCAGACGGGATTTGTTAGGC | 915 |
| | F1c | TGACCCACTAAGTGGGCACTTCTC | 916 |
| | B2 | AATCCCACCTAAAGCAGAAACTTC | 917 |
| | B1c | AGCCACTGCACCCAACATGTTTAAC | 918 |
| 14 | Cholera | | |
| | CtxA | | |
| | F3 | TCAACCTTTATGATCATGCAAGAGG | 909 |
| | B3 | ACGATGTAATTGTTCATCAAGCACC | 919 |
| | FIP | GACCCACTAAGTGGGCACTTCTCACTC | 920 |
| | (F1c + F2) | AGACGGGATTTGTTAGGC | |
| | BIP | AGCCACTGCACCCAACATGTTTAACAAT | 912 |
| | (B1c + B2) | CCCACCTAAAGCAGAAACTTC | |
| | LoopF | GAGGTGGAAACATATCCATCATCGT | 913 |
| | LoopB | GCATACAGTCCTCATCCAGATGAAC | 914 |
| | F2 | CTCAGACGGGATTTGTTAGGC | 915 |
| | F1c | GACCCACTAAGTGGGCACTTCTCA | 921 |
| | B2 | AATCCCACCTAAAGCAGAAACTTC | 917 |
| | B1c | AGCCACTGCACCCAACATGTTTAAC | 918 |
| 15 | Cholera | | |
| | CtxA | | |
| | F3 | AGTCAGGTGGTCTTATGCCAAGAG | 922 |
| | B3 | CCATCTGCTGCTGGAGCAATATCTA | 923 |
| | FIP | TGACCCACTAAGTGGGCACTTCTCAT | 924 |
| | (F1c + F2) | CATGCAAGAGGAACTCAGACGG | |
| | BIP | GCCACTGCACCCAACATGTTTAACGT | 925 |
| | (B1c + B2) | GGAATCCCACCTAAAGCAGAAACT | |
| | LoopF | ACATATCCATCATCGTGCCTAACAA | 926 |
| | LoopB | GGCATACAGTCCTCATCCAGATGAA | 927 |
| | F2 | TCATGCAAGAGGAACTCAGACGG | 928 |
| | F1c | TGACCCACTAAGTGGGCACTTCTCA | 929 |
| | B2 | TGGAATCCCACCTAAAGCAGAAACT | 930 |
| | B1c | GCCACTGCACCCAACATGTTTAACG | 931 |
| 16 | Cholera | | |
| | CtxA | | |
| | F3 | AGTCAGGTGGTCTTATGCCAAGAG | 922 |
| | B3 | TCTGCTGCTGGAGCAATATCTAAGT | 932 |
| | FIP | TGACCCACTAAGTGGGCACTTCTCAT | 924 |
| | (F1c + F2) | CATGCAAGAGGAACTCAGACGG | |
| | BIP | GCCACTGCACCCAACATGTTTAACGT | 925 |
| | (B1c + B2) | GGAATCCCACCTAAAGCAGAAACT | |
| | LoopF | ACATATCCATCATCGTGCCTAACAA | 926 |
| | LoopB | GGCATACAGTCCTCATCCAGATGAA | 927 |
| | F2 | TCATGCAAGAGGAACTCAGACGG | 928 |
| | F1c | TGACCCACTAAGTGGGCACTTCTCA | 929 |
| | B2 | TGGAATCCCACCTAAAGCAGAAACT | 930 |
| | B1c | GCCACTGCACCCAACATGTTTAACG | 931 |
| 17 | Cholera | | |
| | CtxA | | |
| | F3 | AGTCAGGTGGTCTTATGCCAAGAG | 933 |
| | B3 | CCATCTGCTGCTGGAGCAATATCTA | 934 |
| | FIP | GACCCACTAAGTGGGCACTTCTCAAT | 935 |
| | (F1c + F2) | CATGCAAGAGGAACTCAGACGG | |
| | BIP | GCCACTGCACCCAACATGTTTAACGT | 936 |
| | (B1c + B2) | GGAATCCCACCTAAAGCAGAAACT | |
| | LoopF | ACATATCCATCATCGTGCCTAACAA | 937 |
| | LoopB | GGCATACAGTCCTCATCCAGATGAA | 938 |
| | F2 | TCATGCAAGAGGAACTCAGACGG | 939 |
| | F1c | GACCCACTAAGTGGGCACTTCTCAA | 940 |
| | B2 | TGGAATCCCACCTAAAGCAGAAACT | 941 |
| | B1c | GCCACTGCACCCAACATGTTTAACG | 942 |
| 18 | Cholera | | |
| | CtxA | | |
| | F3 | GATATTGCTCCAGCAGCA | 943 |
| | B3 | AACTTTAGATTGGTATTCGTCAA | 944 |
| | FIP | GTGCATGATGAATCCACGGCGATGG | 945 |
| | (F1c + F2) | TTATGGATTGGCAGG | |
| | BIP | GGTTGTGGGAATGCTCCAAGTTACA | 946 |
| | (B1c + B2) | CCTAGACTTTGGGTT | |
| | LoopF | CTCTATGCTCCGGAGGGAAA | 947 |
| | LoopB | CATCGATGAGTAATACTTGCGATGA | 948 |
| | F2 | GATGGTTATGGATTGGCAGG | 949 |
| | F1c | GTGCATGATGAATCCACGGC | 950 |
| | B2 | TTACACCTAGACTTTGGGTT | 951 |
| | B1c | GGTTGTGGGAATGCTCCAAG | 952 |
| 1 | Cholera O1 | Accession no. X59554 | |
| | F3 | TCTTCTGCTACCAGTGG | 953 |
| | B3 | ATTCAAGTGGAGCACTTG | 954 |
| | FIP | ACACCTCCTGCATAACTCTTGGTTAG | 955 |
| | (F1c + F2) | ATAAGGTAACCGCTC | |
| | BIP | TATGGATATTGATCCGACAAGCCGG | 956 |
| | (B1c + B2) | CGAAGTTTAGGTAACC | |
| | LoopF | CCATTGCTCGATGCTGT | 957 |
| | LoopB | AATGCCACTAACCTTGGG | 958 |
| | F2 | GTTAGATAAGGTAACCGCTC | 959 |
| | F1c | ACACCTCCTGCATAACTCTTG | 960 |
| | B2 | GGCGAAGTTTAGGTAACC | 961 |
| | B1c | TATGGATATTGATCCGACAAGCC | 962 |
| 2 | Cholera O1 | | |
| | F3 | CCTTGGTGTGATTGAAGAA | 963 |
| | B3 | AGCTTCTAATGGTTGGTTAG | 964 |
| | FIP | AAGTGGCTTATACGATGGCTTAAGCA | 965 |
| | (F1c + F2) | GAAGTAAGAGGCT | |
| | BIP | CTCAACAAGAAGAGAGGTTGACTAGA | 966 |
| | (B1c + B2) | TGCGGACATAGTATCA | |
| | LoopF | TCTCAGACATAACATCACCAC | 967 |
| | LoopB | ATCTACCACTCACCGATATTTC | 968 |
| | F2 | AAGCAGAAGTAAGAGGCT | 969 |
| | F1c | AAGTGGCTTATACGATGGCTT | 970 |
| | B2 | AGATGCGGACATAGTATCA | 971 |
| | B1c | CTCAACAAGAAGAGAGGTTGACT | 972 |
| 3 | Cholera O1 | | |
| | F3 | CGTGATGAATCGAACCTAG | 973 |
| | B3 | TGGCTTATACGATGGCT | 974 |
| | FIP | ATCACACCAAGGTCATCTGTAAGGTA | 975 |
| | (F1c + F2) | GGCTTACTTGAGTTTGT | |
| | BIP | TGTGAGTGTGGTAAAGCTGGAGCCT | 976 |
| | (B1c + B2) | CTTACTTCTGCTT | |
| | LoopF | CGGATATGAATGCGGTAGT | 977 |
| | LoopB | AAGTCATTGGACGAGCAA | 978 |
| | F2 | GTAGGCTTACTTGAGTTTGT | 979 |
| | F1c | ATCACACCAAGGTCATCTGTAAG | 980 |
| | B2 | AGCCTCTTACTTCTGCTT | 981 |
| | B1c | TGTGAGTGTGGTAAAGCTGG | 982 |
| 4 | Cholera O1 | | |
| | F3 | TCTTCTGCTACCAGTGG | 983 |
| | B3 | ATTCAAGTGGAGCACTTG | 984 |
| | FIP | ACACCTCCTGCATAACTCTTGGTTAGAT | 985 |
| | (F1c + F2) | AAGGTAACCGCTC | |
| | BIP | ACAAGCCCAAATGCCACTGATGTTGAG | 986 |
| | (B1c + B2) | GCGAAGTT | |
| | LoopF | CCATTGCTCGATGCTGTCGA | 987 |
| | LoopB | GCTCGTATTGCGGCGGTAA | 43 |
| | F2 | GTTAGATAAGGTAACCGCTC | 988 |
| | F1c | ACACCTCCTGCATAACTCTTG | 989 |
| | B2 | GATGTTGAGGCGAAGTT | 990 |
| | B1c | ACAAGCCCAAATGCCACT | 991 |
| 5 | Cholera O1 | | |
| | F3 | GTTAGATAAGGTAACCGCTC | 992 |
| | B3 | TCACTCGCAAGTGAATTC | 993 |
| | FIP | GGCTTGTCGGATCAATATCCATAGCAAG | 994 |
| | (F1c + F2) | AGTTATGCAGGAG | |
| | BIP | CTCGTATTGCGGCGGTAAACTTGGGCTA | 995 |
| | (B1c + B2) | TCAGCAT | |
| | LoopF | AACGGGCGACGTTTAGGC | 996 |
| | LoopB | AACTTCGCCTCAACATCGAAGT | 997 |
| | F2 | GCAAGAGTTATGCAGGAG | 998 |
| | F1c | GGCTTGTCGGATCAATATCCATA | 999 |
| | B2 | ACTTGGGCTATCAGCAT | 1000 |
| | B1c | CTCGTATTGCGGCGGTAA | 1001 |
| 6 | Cholera O1 | | |
| | F3 | CACGGTGATGACTTATTAGG | 1002 |
| | B3 | CACACTAGAGTCAGTTGC | 1003 |
| | FIP | TTTCTTTGGAAGGCCACTTACACAATGC | 1004 |
| | (F1c + F2) | TGATGTGCGTA | |
| | BIP | ATCCTCTTTGGACCTAAGCTTTCCTGATT | 1005 |
| | (B1c + B2) | GCCTTGTCAGAG | |
| | LoopF | GACTCGATTGCCTCTCGTCC | 1006 |
| | LoopB | TTGTTGGCAGCGATGCTCTAG | 1007 |
| | F2 | CAATGCTGATGTGCGTA | 1008 |
| | F1c | TTTCTTTGGAAGGCCACTTACA | 1009 |
| | B2 | CTGATTGCCTTGTCAGAG | 1010 |
| | B1c | ATCCTCTTTGGACCTAAGCTTTC | 1011 |
| 7 | Cholera O1 | | |
| | F3 | TCGGTAAGCGATGATACGA | 1012 |
| | B3 | CGATGTTGAGGCGAAGTT | 1013 |
| | FIP | CCATTGCTCGATGCTGTCGACAATCTT | 1014 |
| | (F1c + F2) | CTGCTACCAGTGG | |
| | BIP | AAGAGTTATGCAGGAGGTGTTGGGCTT | 1015 |
| | (B1c + B2) | GTCGGATCAATATCCAT | |
| | LoopF | CGAGCGGTTACCTTATCTAACAA | 1016 |
| | LoopB | CCTAAACGTCGCCCGTT | 1017 |
| | F2 | CAATCTTCTGCTACCAGTGG | 1018 |
| | F1c | CCATTGCTCGATGCTGTCGA | 1019 |
| | B2 | GCTTGTCGGATCAATATCCAT | 1020 |
| | B1c | AAGAGTTATGCAGGAGGTGTTGG | 1021 |
| 8 | Cholera O1 | | |
| | F3 | GCGTACCCAGTACCATATTG | 1022 |
| | B3 | ATTCAAGTGGAGCACTTGG | 1023 |
| | FIP | CCCAACACCTCCTGCATAACTCAGATAA | 1024 |
| | (F1c + F2) | GGTAACCGCTCGT | |
| | BIP | AAGCCCAAATGCCACTAACCTTCGATGTT | 1025 |
| | (B1c + B2) | GAGGCGAAGTT | |
| | LoopF | TTGCCATTGCTCGATGCT | 1026 |
| | LoopB | CTCGTATTGCGGCGGTAA | 1027 |
| | F2 | AGATAAGGTAACCGCTCGT | 1028 |
| | F1c | CCCAACACCTCCTGCATAACTC | 1029 |
| | B2 | CGATGTTGAGGCGAAGTT | 1030 |
| | B1c | AAGCCCAAATGCCACTAACCTT | 1031 |
| 9 | Cholera O1 | | |
| | F3 | CGAGCAATGGCAAGAGTT | 1032 |
| | B3 | TAGGCAATTGAAACGAGATCC | 1033 |
| | FIP | ACTTCGATGTTGAGGCGAAGTTGACAAG | 1034 |
| | (F1c + F2) | CCCAAATGCCA | |
| | BIP | GCCCAAGTGCTCCACTTGAAATGAGCGG | 1035 |
| | (B1c + B2) | CTCTTCACT | |
| | LoopF | TTACCGCCGCAATACGAG | 1036 |
| | LoopB | TGAACATCTGAATTCACTTGCG | 1037 |
| | F2 | GACAAGCCCAAATGCCA | 1038 |
| | F1c | ACTTCGATGTTGAGGCGAAGTT | 1039 |
| | B2 | ATGAGCGGCTCTTCACT | 1040 |
| | B1c | GCCCAAGTGCTCCACTTGAA | 1041 |
| 10 | Cholera O1 | | |
| | F3 | TCGGTAAGCGATGATACGA | 1042 |
| | B3 | CGATGTTGAGGCGAAGTT | 1043 |
| | FIP | CTCGATGCTGTCGACGAGCAAGCTTCAAT | 1044 |
| | (F1c + F2) | CTTCTGCTACC | |
| | BIP | GCAAGAGTTATGCAGGAGGTGTTGCTTGT | 1045 |
| | (B1c + B2) | CGGATCAATATCCAT | |
| | LoopF | ATGGTACTGGGTACGCCACT | 1046 |
| | LoopB | GCCTAAACGTCGCCCGTT | 1047 |
| | F2 | AAGCTTCAATCTTCTGCTACC | 1048 |
| | F1c | CTCGATGCTGTCGACGAGC | 1049 |
| | B2 | GCTTGTCGGATCAATATCCAT | 1050 |
| | B1c | GCAAGAGTTATGCAGGAGGTGTT | 1051 |
| 11 | Cholera O1 | | |
| | F3 | CAATCTTCTGCTACCAGTGG | 1052 |
| | B3 | ATTCAAGTGGAGCACTTGG | 1053 |
| | FIP | CCCAACACCTCCTGCATAACTCAGATAAGGT | 1054 |
| | (F1c + F2) | AACCGCTCGT | |
| | BIP | AAGCCCAAATGCCACTAACCTTCGATGTTGA | 1055 |
| | (B1c + B2) | GGCGAAGTT | |
| | LoopF | TTGCCATTGCTCGATGCTGT | 1056 |
| | LoopB | GCTCGTATTGCGGCGGTAA | 43 |
| | F2 | AGATAAGGTAACCGCTCGT | 1057 |
| | F1c | CCCAACACCTCCTGCATAACTC | 1058 |
| | B2 | CGATGTTGAGGCGAAGTT | 1059 |
| | B1c | AAGCCCAAATGCCACTAACCTT | 1060 |
| 12 | Cholera O1 | | |
| | F3 | CAATCTTCTGCTACCAGTGG | 1052 |
| | B3 | ATTCAAGTGGAGCACTTGG | 1053 |
| | FIP | CCCAACACCTCCTGCATAACTCTAGA | 1061 |
| | (F1c + F2) | TAAGGTAACCGCTCGT | |
| | BIP | AAGCCCAAATGCCACTAACCTTCGAT | 1055 |
| | (B1c + B2) | GTTGAGGCGAAGTT | |
| | LoopF | TTGCCATTGCTCGATGCTGT | 1056 |
| | LoopB | GCTCGTATTGCGGCGGTAA | 43 |
| | F2 | TAGATAAGGTAACCGCTCGT | 1062 |
| | F1c | CCCAACACCTCCTGCATAACTC | 1058 |
| | B2 | CGATGTTGAGGCGAAGTT | 1059 |
| | B1c | AAGCCCAAATGCCACTAACCTT | 1060 |
| 13 | Cholera O1 | | |
| | F3 | CGTCGACAGCATCGAGC | 1063 |
| | B3 | CTTCACTCGCAAGTGAATTCAGA | 1064 |
| | FIP | GGCATTTGGGCTTGTCGGATCAAG | 1065 |
| | (F1c + F2) | CAAGAGTTATGCAGGAGGTG | |
| | BIP | GGCTCGTATTGCGGCGGTAAAGCA | 1066 |
| | (B1c + B2) | CTTGGGCTATCAGCATC | |
| | LoopF | AACGGGCGACGTTTAGGC | 1067 |
| | LoopB | AACTTCGCCTCAACATCGAAGT | 1068 |
| | F2 | GCAAGAGTTATGCAGGAGGTG | 1069 |
| | F1c | GGCATTTGGGCTTGTCGGATCAA | 1070 |
| | B2 | GCACTTGGGCTATCAGCATC | 1071 |
| | B1c | GGCTCGTATTGCGGCGGTAAA | 1072 |
| 14 | Cholera O1 | | |
| | F3 | ACAGCATCGAGCAATGGC | 1073 |
| | B3 | CTTCACTCGCAAGTGAATTCAGA | 1064 |
| | FIP | GTGGCATTTGGGCTTGTCGGATGA | 1074 |
| | (F1c + F2) | GTTATGCAGGAGGTGTTGG | |
| | BIP | GGCTCGTATTGCGGCGGTAAAGCA | 1066 |
| | (B1c + B2) | CTTGGGCTATCAGCATC | |
| | LoopF | AACGGGCGACGTTTAGGC | 1067 |
| | LoopB | AACTTCGCCTCAACATCGAAGT | 1068 |
| | F2 | GAGTTATGCAGGAGGTGTTGG | 1075 |
| | F1c | GTGGCATTTGGGCTTGTCGGAT | 1076 |
| | B2 | GCACTTGGGCTATCAGCATC | 1071 |
| | B1c | GGCTCGTATTGCGGCGGTAAA | 1072 |
| 15 | Cholera O1 | | |
| | F3 | AAGCTTCAATCTTCTGCTACCAG | 1077 |
| | B3 | CTTCACTCGCAAGTGAATTCAGA | 1064 |
| | FIP | GGCATTTGGGCTTGTCGGATCAAG | 1078 |
| | (F1c + F2) | GCAAGAGTTATGCAGGAGG | |
| | BIP | GGCTCGTATTGCGGCGGTAAAGCA | 1066 |
| | (B1c + B2) | CTTGGGCTATCAGCATC | |
| | LoopF | AACGGGCGACGTTTAGGC | 1067 |
| | LoopB | AACTTCGCCTCAACATCGAAGT | 1068 |
| | F2 | GGCAAGAGTTATGCAGGAGG | 1079 |
| | F1c | GGCATTTGGGCTTGTCGGATCAA | 1080 |
| | B2 | GCACTTGGGCTATCAGCATC | 1071 |
| | B1c | GGCTCGTATTGCGGCGGTAAA | 1072 |
| 16 | Cholera O1 | | |
| | F3 | TCTTCTGCTACCAGTGGCGTAC | 38 |
| | B3 | AGTGGAGCACTTGGGCTATCAG | 1081 |
| | FIP | TGCAAAACGGGCGACGTTTAGGCT | 40 |
| | (F1c + F2) | CGTCGACAGCATCGAGCA | |
| | BIP | TGATCCGACAAGCCCAAATGCCACT | 1082 |
| | (B1c + B2) | CGATGTTGAGGCGAAGTTTAGGT | |
| | LoopF | AACACCTCCTGCATAACTCTTGC | 42 |
| | LoopB | GCTCGTATTGCGGCGGTAA | 43 |
| | F2 | TCGTCGACAGCATCGAGCA | 44 |
| | F1c | TGCAAAACGGGCGACGTTTAGGC | 45 |
| | B2 | TCGATGTTGAGGCGAAGTTTAGGT | 46 |
| | B1c | TGATCCGACAAGCCCAAATGCCAC | 47 |
| 17 | Cholera O1 | | |
| | F3 | TCTTCTGCTACCAGTGGCGTAC | 38 |
| | B3 | TTCAAGTGGAGCACTTGGGCTA | 39 |
| | FIP | GGGCGACGTTTAGGCCCCAACATC | 1083 |
| | (F1c + F2) | GTCGACAGCATCGAGCA | |
| | BIP | TGATCCGACAAGCCCAAATGCCAC | 1084 |
| | (B1c + B2) | TCGATGTTGAGGCGAAGTTTAGGT | |
| | LoopF | CCTCCTGCATAACTCTTGCCAT | 1085 |
| | LoopB | GCTCGTATTGCGGCGGTAA | 43 |
| | F2 | TCGTCGACAGCATCGAGCA | 44 |
| | F1c | GGGCGACGTTTAGGCCCCAACA | 1086 |
| | B2 | TCGATGTTGAGGCGAAGTTTAGGT | 46 |
| | B1c | TGATCCGACAAGCCCAAATGCCAC | 47 |
| 18 | Cholera O1 | | |
| | F3 | TCTTCTGCTACCAGTGGCGTAC | 38 |
| | B3 | TTCAAGTGGAGCACTTGGGCTA | 39 |
| | FIP | TGCAAAACGGGCGACGTTTAGGCT | 40 |
| | (F1c + F2) | CGTCGACAGCATCGAGCA | |
| | BIP | TGATCCGACAAGCCCAAATGCCACT | 41 |
| | (B1c + B2) | CGATGTTGAGGCGAAGTTTAGGT | |
| | LoopF | ACACCTCCTGCATAACTCTTGCCA | 1087 |
| | LoopB | GGCTCGTATTGCGGCGGTAA | 1088 |
| | F2 | TCGTCGACAGCATCGAGCA | 44 |
| | F1c | TGCAAAACGGGCGACGTTTAGGC | 45 |
| | B2 | TCGATGTTGAGGCGAAGTTTAGGT | 46 |
| | B1c | TGATCCGACAAGCCCAAATGCCAC | 47 |
| 19 | Cholera O1 | | |
| | F3 | TCTTCTGCTACCAGTGGCGTAC | 38 |
| | B3 | TTCAAGTGGAGCACTTGGGCTA | 39 |
| | FIP | TGCAAAACGGGCGACGTTTAGGCA | 1089 |
| | (F1c + F2) | CCGCTCGTCGACAGCA | |
| | BIP | TGATCCGACAAGCCCAAATGCCACT | 41 |
| | (B1c + B2) | CGATGTTGAGGCGAAGTTTAGGT | |
| | LoopF | ACACCTCCTGCATAACTCTTGCCA | 1090 |
| | LoopB | GGCTCGTATTGCGGCGGTAA | 1091 |
| | F2 | ACCGCTCGTCGACAGCA | 1092 |
| | F1c | TGCAAAACGGGCGACGTTTAGGC | 45 |
| | B2 | TCGATGTTGAGGCGAAGTTTAGGT | 46 |
| | B1c | TGATCCGACAAGCCCAAATGCCAC | 47 |
| 20 | Cholera O1 | | |
| | F3 | TCTTCTGCTACCAGTGGCGTAC | 38 |
| | B3 | TTCAAGTGGAGCACTTGGGCTA | 39 |
| | FIP | TGCAAAACGGGCGACGTTTAGGCT | 1093 |
| | (F1c + F2) | AACCGCTCGTCGACAGCAT | |
| | BIP | TGATCCGACAAGCCCAAATGCCACT | 41 |
| | (B1c + B2) | CGATGTTGAGGCGAAGTTTAGGT | |
| | LoopF | ACACCTCCTGCATAACTCTTGCCA | 1094 |
| | LoopB | GGCTCGTATTGCGGCGGTAA | 1095 |
| | F2 | TAACCGCTCGTCGACAGCAT | 1096 |
| | F1c | TGCAAAACGGGCGACGTTTAGGC | 45 |
| | B2 | TCGATGTTGAGGCGAAGTTTAGGT | 46 |
| | B1c | TGATCCGACAAGCCCAAATGCCAC | 47 |
| 21 | Cholera O1 | | |
| | F3 | TTTCTATGATGGTTGTTGGCAGCGA | 1097 |
| | B3 | TTTGCTTGCCCAATGTCTGGTACT | 1098 |
| | FIP | CGGAGAAGCGCAAGCAAACTGATTTTT | 1099 |
| | (F1c + F2) | GGCAATCAGAAAGTTGATTCGTCGG | |
| | BIP | GAAGGGCGGTCTAATAACACCTAAA | 1100 |
| | (B1c + B2) | TTTTGAGTTGGTAAGCGTACAAGAGCCT | |
| | LoopF | GAACACACTAGAGTCAGTTGCAGC | 1101 |
| | LoopB | GAGTTTGCAGAGAAGCTTGCCTCAG | 1102 |
| | F2 | GGCAATCAGAAAGTTGATTCGTCGG | 1103 |
| | F1c | CGGAGAAGCGCAAGCAAACTGAT | 1104 |
| | B2 | GAGTTGGTAAGCGTACAAGAGCCT | 1105 |
| | B1c | GAAGGGCGGTCTAATAACACCTAAA | 1106 |
| 1 | S. Typhi | Accession no. AL513382 | |
| | F3 | GTTGAACTGAGTCTGGC | 1107 |
| | B3 | GACAGGCGATTGCTAAC | 1108 |
| | FIP | AACAACCTGCAGCGTGTGAGTCG | 1109 |
| | (F1c + F2) | AGGTCAGACTG | |
| | BIP | CGCCTTCAGTGGTCTGCCATCAAA | 1110 |
| | (B1c + B2) | GGTCTGACTCAG | |
| | LoopF | CGGTTCAGTCTGCGAAT | 1111 |
| | LoopB | CAATGGAGATACCGTCGTT | 1112 |
| | F2 | GAGTCGAGGTCAGACTG | 1113 |
| | F1c | AACAACCTGCAGCGTGT | 1114 |
| | B2 | CATCAAAGGTCTGACTCAG | 1115 |
| | B1c | CGCCTTCAGTGGTCTGC | 1116 |
| 2 | S. Typhi | | |
| | F3 | CTGCAGGTTGTTGTTGA | 1117 |
| | B3 | AATACAAACAGCCTGTCG | 1118 |
| | FIP | AGGCGATTGCTAACCGTTTGGA | 1119 |
| | (F1c + F2) | GATACCGTCGTTAG | |
| | BIP | GATACGCAGACCGGAAGACAT | 1120 |
| | (B1c + B2) | AACCTGAACAAATCCCAG | |
| | LoopF | AGGTCTGACTCAGGCTT | 1121 |
| | LoopB | AACGCTCGATAGCAGTG | 1122 |
| | F2 | TGGAGATACCGTCGTTAG | 1123 |
| | F1c | AGGCGATTGCTAACCGTT | 1124 |
| | B2 | ATAACCTGAACAAATCCCAG | 1125 |
| | B1c | GATACGCAGACCGGAAGAC | 1126 |
| 3 | S. Typhi | | |
| | F3 | CCAAGGCAGCATCAATT | 48 |
| | B3 | TTATTGATGGTGGCTATGC | 1127 |
| | FIP | TCTGAAGTTGTTACTGCTACCGG | 1128 |
| | (F1c + F2) | CCTGTTCTGAAGTTATGT | |
| | BIP | GCCACCAAATTTCACAGCTCCAGG | 1129 |
| | (B1c + B2) | TGCAATTACTGCTAA | |
| | LoopF | ACTTAGCAAGCGACCTTGACAA | 1130 |
| | LoopB | TTGAGCAACGCCAGTACCATC | 1131 |
| | F2 | GCCTGTTCTGAAGTTATGT | 54 |
| | F1c | TCTGAAGTTGTTACTGCTACCG | 55 |
| | B2 | CAGGTGCAATTACTGCTAA | 56 |
| | B1c | GCCACCAAATTTCACAGCTC | 57 |
| 4 | S. Typhi | | |
| | F3 | GAGTCGAGGTCAGACTG | 1132 |
| | B3 | GTCTGCGTATCAACAGC | 1133 |
| | FIP | CAACAACAACCTGCAGCGGAGTTA | 1134 |
| | (F1c + F2) | GTACCATTCGCAG | |
| | BIP | ATACCGTCGTTAGCGTTACGGACA | 1135 |
| | (B1c + B2) | GGCGATTGCTAAC | |
| | LoopF | CGTGAACTGGCGGTTCAGT | 1136 |
| | LoopB | TGAGTCAGACCTTTGATGTTCGC | 1137 |
| | F2 | GAGTTAGTACCATTCGCAG | 1138 |
| | F1c | CAACAACAACCTGCAGCG | 1139 |
| | B2 | GACAGGCGATTGCTAAC | 1140 |
| | B1c | ATACCGTCGTTAGCGTTACG | 1141 |
| 5 | S. Typhi | | |
| | F3 | GAGTTAGTACCATTCGCAG | 1142 |
| | B3 | ATAACCTGAACAAATCCCAG | 1143 |
| | FIP | CGTAACGCTAACGACGGTATCTG | 1144 |
| | (F1c + F2) | CAGGTTGTTGTTGA | |
| | BIP | ACGGTTAGCAATCGCCTGGTTTG | 1145 |
| | (B1c + B2) | TCTTCCGGTCTG | |
| | LoopF | CCATTGCGCAGACCACTGA | 1146 |
| | LoopB | TCCTGCCGCATCGTCTTTC | 1147 |
| | F2 | CTGCAGGTTGTTGTTGA | 1148 |
| | F1c | CGTAACGCTAACGACGGTAT | 1149 |
| | B2 | GTTTGTCTTCCGGTCTG | 1150 |
| | B1c | ACGGTTAGCAATCGCCTG | 1151 |
| 6 | S. Typhi | | |
| | F3 | CGCCGTTGAACTGAGTC | 1152 |
| | B3 | CGGTCTGCGTATCAACAG | 1153 |
| | FIP | TGCAGCGTGTGCGTGAACCTGGA | 1154 |
| | (F1c + F2) | TGGAGTCGAGG | |
| | BIP | TCAGTGGTCTGCGCAATGGAGGT | 1155 |
| | (B1c + B2) | CTGACTCAGGCTTC | |
| | LoopF | GTTCAGTCTGCGAATGGTACT | 1156 |
| | LoopB | ATACCGTCGTTAGCGTTACG | 1157 |
| | F2 | CCTGGATGGAGTCGAGG | 1158 |
| | F1c | TGCAGCGTGTGCGTGAA | 1159 |
| | B2 | AGGTCTGACTCAGGCTTC | 1160 |
| | B1c | TCAGTGGTCTGCGCAATGG | 1161 |
| 7 | S. Typhi | | |
| | F3 | CCTGGATGGAGTCGAGG | 1162 |
| | B3 | CGGTCTGCGTATCAACAG | 1153 |
| | FIP | CCATTGCGCAGACCACTGACTGAA | 1163 |
| | (F1c + F2) | CCGCCAGTTCAC | |
| | BIP | GATACCGTCGTTAGCGTTACGGCA | 1164 |
| | (B1c + B2) | GGCGATTGCTAACCG | |
| | LoopF | AACAACCTGCAGCGTGT | 1165 |
| | LoopB | CCTGAGTCAGACCTTTGATGTT | 1166 |
| | F2 | CTGAACCGCCAGTTCAC | 1167 |
| | F1c | CCATTGCGCAGACCACTGA | 1168 |
| | B2 | CAGGCGATTGCTAACCG | 1169 |
| | B1c | GATACCGTCGTTAGCGTTACGG | 1170 |
| 8 | S. Typhi | | |
| | F3 | CGCTGCAGGTTGTTGTT | 1171 |
| | B3 | ATACAAACAGCCTGTCGC | 1172 |
| | FIP | GCAGGACAGGCGATTGCTAACCGT | 1173 |
| | (F1c + F2) | CGTTAGCGTTACG | |
| | BIP | GTTGATACGCAGACCGGAAGACAT | 1174 |
| | (B1c + B2) | AACCTGAACAAATCCCAGTC | |
| | LoopF | AACATCAAAGGTCTGACTCAGG | 1175 |
| | LoopB | ACGCTCGATAGCAGTGC | 1176 |
| | F2 | CCGTCGTTAGCGTTACG | 1177 |
| | F1c | GCAGGACAGGCGATTGCTAA | 1178 |
| | B2 | ATAACCTGAACAAATCCCAGTC | 1179 |
| | B1c | GTTGATACGCAGACCGGAAGAC | 1180 |
| 9 | S. Typhi | | |
| | F3 | CGCCGTTGAACTGAGTC | 1181 |
| | B3 | CGGTCTGCGTATCAACAG | 1182 |
| | FIP | TGCAGCGTGTGCGTGAACCTGGAT | 1183 |
| | (F1c + F2) | GGAGTCGAGG | |
| | BIP | TCAGTGGTCTGCGCAATGGAGGTC | 1184 |
| | (B1c + B2) | TGACTCAGGCTTC | |
| | LoopF | GGCGGTTCAGTCTGCGAAT | 1185 |
| | LoopB | GATACCGTCGTTAGCGTTACGG | 1186 |
| | F2 | CCTGGATGGAGTCGAGG | 1187 |
| | F1c | TGCAGCGTGTGCGTGAA | 1188 |
| | B2 | AGGTCTGACTCAGGCTTC | 1189 |
| | B1c | TCAGTGGTCTGCGCAATGG | 1190 |
| 10 | S. Typhi | | |
| | F3 | GGCATCTTGGACATTAAGCTTA | 1191 |
| | B3 | CTAACGACGGTATCTCCATTG | 1192 |
| | FIP | GACTCAGTTCAACGGCGTGAATTGG | 1193 |
| | (F1c + F2) | CACCAACCTGGAT | |
| | BIP | CCTGGATGGAGTCGAGGTCAGTGAA | 1194 |
| | (B1c + B2) | CTGGCGGTTCAG | |
| | LoopF | TGGCGCAGGACAACACC | 1195 |
| | LoopB | TGGGAGTTAGTACCATTCGCAGA | 1196 |
| | F2 | TTGGCACCAACCTGGAT | 1197 |
| | F1c | GACTCAGTTCAACGGCGTGAA | 1198 |
| | B2 | GTGAACTGGCGGTTCAG | 1199 |
| | B1c | CCTGGATGGAGTCGAGGTCA | 1200 |
| 11 | S. Typhi | | |
| | F3 | CCTGGATGGAGTCGAGG | 1201 |
| | B3 | CGGTCTGCGTATCAACAG | 1202 |
| | FIP | CCATTGCGCAGACCACTGACTGAACC | 1203 |
| | (F1c + F2) | GCCAGTTCAC | |
| | BIP | GATACCGTCGTTAGCGTTACGGCAGG | 1204 |
| | (B1c + B2) | CGATTGCTAACCG | |
| | LoopF | ACAACAACCTGCAGCGTGT | 1205 |
| | LoopB | TGAGTCAGACCTTTGATGTTCGC | 1206 |
| | F2 | CTGAACCGCCAGTTCAC | 1207 |
| | F1c | CCATTGCGCAGACCACTGA | 1208 |
| | B2 | CAGGCGATTGCTAACCG | 1209 |
| | B1c | GATACCGTCGTTAGCGTTACGG | 1210 |
| 12 | S. Typhi | | |
| | F3 | GTAGGCATCTTGGACATTAAGCT | 1211 |
| | B3 | CTAACGACGGTATCTCCATTGC | 1212 |
| | FIP | CGTGTATCCGGCCAGACTCAGTCGTT | 1213 |
| | (F1c + F2) | GGCACCAACCTGG | |
| | BIP | CGCTGGGTGATTTCAGCCTGGGTGAA | 1214 |
| | (B1c + B2) | CTGGCGGTTCAGTC | |
| | LoopF | TGGCGCAGGACAACACC | 1215 |
| | LoopB | ATGGAGTCGAGGTCAGACTGG | 1216 |
| | F2 | CGTTGGCACCAACCTGG | 1217 |
| | F1c | CGTGTATCCGGCCAGACTCAGT | 1218 |
| | B2 | GTGAACTGGCGGTTCAGTC | 1219 |
| | B1c | CGCTGGGTGATTTCAGCCTGG | 1220 |
| 13 | S. Typhi | | |
| | F3 | CGCCGTTGAACTGAGTCTG | 1221 |
| | B3 | GGTCTGCGTATCAACAGCG | 1222 |
| | FIP | ATCAACAACAACCTGCAGCGTGTGGA | 1223 |
| | (F1c + F2) | GTCGAGGTCAGACTGG | |
| | BIP | CCTTCAGTGGTCTGCGCAATGGCGAA | 1224 |
| | (B1c + B2) | CATCAAAGGTCTGACTCAG | |
| | LoopF | GGCGGTTCAGTCTGCGAAT | 1225 |
| | LoopB | GATACCGTCGTTAGCGTTACGG | 1226 |
| | F2 | GGAGTCGAGGTCAGACTGG | 1227 |
| | F1c | ATCAACAACAACCTGCAGCGTGT | 1228 |
| | B2 | CGAACATCAAAGGTCTGACTCAG | 1229 |
| | B1c | CCTTCAGTGGTCTGCGCAATGG | 1230 |
| 14 | S. Typhi | | |
| | F3 | CGCCAGGACTTTCACGC | 1231 |
| | B3 | GGTCTGCGTATCAACAGCG | 1222 |
| | FIP | CTGCAGCGTGTGCGTGAACTTCAGCCT | |
| | (F1c + F2) | GGATGGAGTCG | |
| | BIP | CCTTCAGTGGTCTGCGCAATGGCGAAC | 1224 |
| | (B1c + B2) | ATCAAAGGTCTGACTCAG | |
| | LoopF | GGCGGTTCAGTCTGCGAAT | 1225 |
| | LoopB | GATACCGTCGTTAGCGTTACGG | 1226 |
| | F2 | TCAGCCTGGATGGAGTCG | 1232 |
| | F1c | CTGCAGCGTGTGCGTGAACT | 1233 |
| | B2 | CGAACATCAAAGGTCTGACTCAG | 1229 |
| | B1c | CCTTCAGTGGTCTGCGCAATGG | 1230 |
| 15 | S. Typhi | | |
| | F3 | TCACGCCGTTGAACTGAGTCT | 1234 |
| | B3 | AGACGATGCGGCAGGACA | 1235 |
| | FIP | AACAACCTGCAGCGTGTGCGTGCTGGAT | 1236 |
| | (F1c + F2) | GGAGTCGAGGTCAGAC | |
| | BIP | GCCTTCAGTGGTCTGCGCAATGGTACCG | 1237 |
| | (B1c + B2) | CGAACATCAAAGGTCTGA | |
| | LoopF | GGCGGTTCAGTCTGCGAAT | 1225 |
| | LoopB | GATACCGTCGTTAGCGTTACGG | 1226 |
| | F2 | CTGGATGGAGTCGAGGTCAGAC | 1238 |
| | F1c | AACAACCTGCAGCGTGTGCGTG | 1239 |
| | B2 | TACCGCGAACATCAAAGGTCTGA | 1240 |
| | B1c | GCCTTCAGTGGTCTGCGCAATGG | 1241 |
| 16 | S. Typhi | | |
| | F3 | TCCTGCGCCAGGACTTTCA | 1242 |
| | B3 | AGACGATGCGGCAGGACA | 1235 |
| | FIP | CTGCAGCGTGTGCGTGAACTGGCAGCCT | 1243 |
| | (F1c + F2) | GGATGGAGTCGAGG | |
| | BIP | GCCTTCAGTGGTCTGCGCAATGGCCGCG | 1244 |
| | (B1c + B2) | AACATCAAAGGTCTGAC | |
| | LoopF | CGGTTCAGTCTGCGAATGGT | 1245 |
| | LoopB | GATACCGTCGTTAGCGTTACGG | 1226 |
| | F2 | CAGCCTGGATGGAGTCGAGG | 1246 |
| | F1c | CTGCAGCGTGTGCGTGAACTGG | 1247 |
| | B2 | CCGCGAACATCAAAGGTCTGAC | 1248 |
| | B1c | GCCTTCAGTGGTCTGCGCAATGG | 1249 |
| 17 | S. Typhi | | |
| | F3 | CTGCGCCAGGACTTTCACG | 1250 |
| | B3 | AGACGATGCGGCAGGACA | 1235 |
| | FIP | CTGCAGCGTGTGCGTGAACTGGGCCT | 1251 |
| | (F1c + F2) | GGATGGAGTCGAGGTC | |
| | BIP | GCCTTCAGTGGTCTGCGCAATGGTACCG | 1252 |
| | (B1c + B2) | CGAACATCAAAGGTCTGA | |
| | LoopF | CGGTTCAGTCTGCGAATGGT | 1245 |
| | LoopB | GATACCGTCGTTAGCGTTACGG | 1226 |
| | F2 | GCCTGGATGGAGTCGAGGTC | 1253 |
| | F1c | CTGCAGCGTGTGCGTGAACTGG | 1247 |
| | B2 | TACCGCGAACATCAAAGGTCTGA | 1254 |
| | B1c | GCCTTCAGTGGTCTGCGCAATGG | 1249 |
| 18 | S. Typhi | | |
| | F3 | TCACGCCGTTGAACTGAGTCT | 1255 |
| | B3 | GCAGGACAGGCGATTGCTAAC | 1256 |
| | FIP | CAACAACCTGCAGCGTGTGCGTCTGGAT | 1257 |
| | (F1c + F2) | GGAGTCGAGGTCAGAC | |
| | BIP | GCCTTCAGTGGTCTGCGCAATGGTACCG | 1258 |
| | (B1c + B2) | CGAACATCAAAGGTCTGA | |
| | LoopF | GAACTGGCGGTTCAGTCTGCG | 1259 |
| | LoopB | TCGTTAGCGTTACGGGAAGCCT | 1260 |
| | F2 | CTGGATGGAGTCGAGGTCAGAC | 1261 |
| | F1c | CAACAACCTGCAGCGTGTGCGT | 1262 |
| | B2 | TACCGCGAACATCAAAGGTCTGA | 1263 |
| | B1c | GCCTTCAGTGGTCTGCGCAATGG | 1264 |
| 19 | S. Typhi | | |
| | F3 | TCCTGCGCCAGGACTTTCA | 1265 |
| | B3 | GCAGGACAGGCGATTGCTAAC | 1256 |
| | FIP | CCTGCAGCGTGTGCGTGAACTGCAGCCT | 1266 |
| | (F1c + F2) | GGATGGAGTCGAGG | |
| | BIP | GCCTTCAGTGGTCTGCGCAATGGCCGCG | 1267 |
| | (B1c + B2) | AACATCAAAGGTCTGAC | |
| | LoopF | GCGGTTCAGTCTGCGAATGGTAC | 1268 |
| | LoopB | TCGTTAGCGTTACGGGAAGCCT | 1269 |
| | F2 | CAGCCTGGATGGAGTCGAGG | 1270 |
| | F1c | CCTGCAGCGTGTGCGTGAACTG | 1271 |
| | B2 | CCGCGAACATCAAAGGTCTGAC | 1272 |
| | B1c | GCCTTCAGTGGTCTGCGCAATGG | 1264 |
| 20 | S. Typhi | | |
| | F3 | CTGCGCCAGGACTTTCACG | 1273 |
| | B3 | GCAGGACAGGCGATTGCTAAC | 1256 |
| | FIP | CCTGCAGCGTGTGCGTGAACTGGCCT | 1274 |
| | (F1c + F2) | GGATGGAGTCGAGGTC | |
| | BIP | GCCTTCAGTGGTCTGCGCAATGGTACC | 1275 |
| | (B1c + B2) | GCGAACATCAAAGGTCTGA | |
| | LoopF | GCGGTTCAGTCTGCGAATGGTAC | 1268 |
| | LoopB | TCGTTAGCGTTACGGGAAGCCT | 1269 |
| | F2 | GCCTGGATGGAGTCGAGGTC | 1276 |
| | F1c | CCTGCAGCGTGTGCGTGAACTG | 1271 |
| | B2 | TACCGCGAACATCAAAGGTCTGA | 1277 |
| | B1c | GCCTTCAGTGGTCTGCGCAATGG | 1264 |
| 21 | S. Typhi | | |
| | F3 | GACAGGCGATTGCTAACCGT | 1278 |
| | B3 | CGTTCAGGCGCTGGGTGAT | 1279 |
| | FIP | TCAGCGCGCCTTCAGTGGTCTTTTTGTCTG | 1280 |
| | (F1c + F2) | ACTCAGGCTTCCCGT | |
| | BIP | CCTGCAGCGTGTGCGTGAACTGTTTTAGCC | 1281 |
| | (B1c + B2) | TGGATGGAGTCGAGGT | |
| | LoopF | GCGCAATGGAGATACCGTCGTT | 1282 |
| | LoopB | TGCGAATGGTACTAACTCCCAGTCTG | 1283 |
| | F2 | GTCTGACTCAGGCTTCCCGT | 1284 |
| | F1c | TCAGCGCGCCTTCAGTGGTCT | 1285 |
| | B2 | AGCCTGGATGGAGTCGAGGT | 1286 |
| | B1c | CCTGCAGCGTGTGCGTGAACTG | 1287 |
| | Norovirus | | |
| | NG1 | Accession no. NC 017722 | |
| | F3 | GATGGCAGGCCATGTTCC | 1288 |
| | B3 | ACAGGATCCATTGCAAGAGG | 1289 |
| | FIP | ACGAATTCGGGCAGAAGATCGCTTTTC | 1290 |
| | (F1c + F2) | TGGATGCGCTTCCATGA | |
| | BIP | TGATGATGGCGTCTAAGGACGCTTTTT | 1291 |
| | (B1c + B2) | CCGGTACCAACTGACCA | |
| | LoopF | TCCTGTCCACAATCCGAGG | 1292 |
| | LoopB | CAAGCGTGGATGGCGCTAG | 1293 |
| | F2 | CTGGATGCGCTTCCATGA | 1294 |
| | F1c | ACGAATTCGGGCAGAAGATCGC | 1295 |
| | B2 | TCCGGTACCAACTGACCA | 1296 |
| | B1c | TGATGATGGCGTCTAAGGACGC | 1297 |
| | Norovirus | | |
| | NG1 | | |
| | F3 | TTTACGTGCCCAGACAAG | 1298 |
| | B3 | AATAGCGGCACCAACAAC | 1299 |
| | FIP | CAAAGCTGGGAGCCAGATTG-AGCCAAT | 1300 |
| | (F1c + F2) | GTTCAGATGGATG | |
| | BIP | GTCGAATGACGCCAACCCAT-GCCATAA | 1301 |
| | (B1c + B2) | CCTCATTATTGACCT | |
| | LoopF | CCCACGTGCTCAGATCTGAGA | 1302 |
| | LoopB | TCCGCAGCCAACCTCGT | 1303 |
| | F2 | AGCCAATGTTCAGATGGATG | 1304 |
| | F1c | CAAAGCTGGGAGCCAGATTG | 1305 |
| | B2 | GCCATAACCTCATTATTGACCT | 1306 |
| | B1c | GTCGAATGACGCCAACCCAT | 1307 |
| 1 | Norovirus | | |
| | NG2 | Accession no. X86557 | |
| | F3 | GACAAGAGCCAATGTTCA | 1308 |
| | B3 | TCTAATCCAGGGGTCAATT | 1309 |
| | FIP | TCGACGCCATCTTCATTCACTGGATGA | 1310 |
| | (F1c + F2) | GATTCTCAGATCT | |
| | BIP | CATCTGATGGGTCCGCAGCCAGAGCC | 1311 |
| | (B1c + B2) | ATAACCTCAT | |
| | LoopF | AAAGCTGGGAGCCAGATT | 1312 |
| | LoopB | TCGTCCCAGAGGTCAATA | 1313 |
| | F2 | TGGATGAGATTCTCAGATCT | 1314 |
| | F1c | TCGACGCCATCTTCATTCAC | 1315 |
| | B2 | CCAGAGCCATAACCTCAT | 1316 |
| | B1c | CATCTGATGGGTCCGCAG | 1317 |
| 2 | Norovirus | | |
| | NG2 | | |
| | F3 | CAAGAGCCAATGTTCAGAT | 1318 |
| | B3 | TCTAATCCAGGGGTCAATT | 1309 |
| | FIP | ATTCGACGCCATCTTCATTCAGGATGA | 1319 |
| | (F1c + F2) | GATTCTCAGATCTG | |
| | BIP | CATCTGATGGGTCCGCAGCCAGAGCC | 1311 |
| | (B1c + B2) | ATAACCTCAT | |
| | LoopF | AAAGCTGGGAGCCAGATT | 1312 |
| | LoopB | TCGTCCCAGAGGTCAATA | 1313 |
| | F2 | GGATGAGATTCTCAGATCTG | 1320 |
| | F1c | ATTCGACGCCATCTTCATTCA | 1321 |
| | B2 | CCAGAGCCATAACCTCAT | 1316 |
| | B1c | CATCTGATGGGTCCGCAG | 1317 |
| 3 | Norovirus | | |
| | NG2 | | |
| | F3 | GATTTTTACGTGCCCAGA | 1322 |
| | B3 | TCTAATCCAGGGGTCAATT | 1309 |
| | FIP | TTCACAAAGCTGGGAGCCCAATGTTCA | 1323 |
| | (F1c + F2) | GATGGATGAGA | |
| | BIP | CATCTGATGGGTCCGCAGCCAGAGCC | 1311 |
| | (B1c + B2) | ATAACCTCAT | |
| | LoopF | CACGTGCTCAGATCTGAG | 1324 |
| | LoopB | TCGTCCCAGAGGTCAATA | 1313 |
| | F2 | CAATGTTCAGATGGATGAGA | 1325 |
| | F1c | TTCACAAAGCTGGGAGCC | 1326 |
| | B2 | CCAGAGCCATAACCTCAT | 1316 |
| | B1c | CATCTGATGGGTCCGCAG | 1317 |
| 4 | Norovirus | | |
| | NG2 | | |
| | F3 | ACAAGAGCCAATGTTCAG | 1327 |
| | B3 | TCTAATCCAGGGGTCAATT | 1309 |
| | FIP | TCGACGCCATCTTCATTCACTGGATGAG | 1328 |
| | (F1c + F2) | ATTCTCAGATCT | |
| | BIP | CATCTGATGGGTCCGCAGCCAGAGCCAT | 1311 |
| | (B1c + B2) | AACCTCAT | |
| | LoopF | CTGGGAGCCAGATTGCGATC | 1329 |
| | LoopB | AACCTCGTCCCAGAGGTCAAT | 1330 |
| | F2 | TGGATGAGATTCTCAGATCT | 1331 |
| | F1c | TCGACGCCATCTTCATTCAC | 1332 |
| | B2 | CCAGAGCCATAACCTCAT | 1316 |
| | B1c | CATCTGATGGGTCCGCAG | 1317 |
| 5 | Norovirus | | |
| | NG2 | | |
| | F3 | CAAGAGCCAATGTTCAGAT | 1333 |
| | B3 | TCTAATCCAGGGGTCAATT | 1309 |
| | FIP | ATTCGACGCCATCTTCATTCAGGATGAG | 1334 |
| | (F1c + F2) | ATTCTCAGATCTG | |
| | BIP | CATCTGATGGGTCCGCAGCCAGAGCCA | 1311 |
| | (B1c + B2) | TAACCTCAT | |
| | LoopF | CTGGGAGCCAGATTGCGATC | 1329 |
| | LoopB | AACCTCGTCCCAGAGGTCAAT | 1330 |
| | F2 | GGATGAGATTCTCAGATCTG | 1335 |
| | F1c | ATTCGACGCCATCTTCATTCA | 1336 |
| | B2 | CCAGAGCCATAACCTCAT | 1316 |
| | B1c | CATCTGATGGGTCCGCAG | 1317 |
| 6 | Norovirus | | |
| | NG2 | | |
| | F3 | GACAAGAGCCAATGTTCA | 1337 |
| | B3 | TCTAATCCAGGGGTCAATT | 1309 |
| | FIP | GACGCCATCTTCATTCACAAAGGATGGAT | 1338 |
| | (F1c + F2) | GAGATTCTCAGATC | |
| | BIP | CATCTGATGGGTCCGCAGCCAGAGCCA | 1311 |
| | (B1c + B2) | TAACCTCAT | |
| | LoopF | CTGGGAGCCAGATTGCGATC | 1329 |
| | LoopB | AACCTCGTCCCAGAGGTCAAT | 1330 |
| | F2 | GATGGATGAGATTCTCAGATC | 1339 |
| | F1c | GACGCCATCTTCATTCACAAAG | 1340 |
| | B2 | CCAGAGCCATAACCTCAT | 1316 |
| | B1c | CATCTGATGGGTCCGCAG | 1317 |
| 7 | Norovirus | | |
| | NG2 | | |
| | F3 | CAGACAAGAGCCAATGTTCA | 1341 |
| | B3 | CAATAGCGGCACCAACAA | 1342 |
| | FIP | CGTCATTCGACGCCATCTTCAGATTCTCA | 1343 |
| | (F1c + F2) | GATCTGAGCACG | |
| | BIP | CATCTGATGGGTCCGCAGCTCCAGAGCC | 1344 |
| | (B1c + B2) | ATAACCTCATTA | |
| | LoopF | TTCACAAAGCTGGGAGCC | 1345 |
| | LoopB | CCTCGTCCCAGAGGTCAA | 1346 |
| | F2 | GATTCTCAGATCTGAGCACG | 74 |
| | F1c | CGTCATTCGACGCCATCTTCA | 75 |
| | B2 | TCCAGAGCCATAACCTCATTA | 76 |
| | B1c | CATCTGATGGGTCCGCAGC | 77 |
| 8 | Norovirus | | |
| | NG2 | | |
| | F3 | TTACGTGCCCAGACAAGA | 1347 |
| | B3 | CTCCAGAGCCATAACCTCA | 1348 |
| | FIP | CTGGGAGCCAGATTGCGATCGCCAAT | 1349 |
| | (F1c + F2) | GTTCAGATGGATGA | |
| | BIP | TGAAGATGGCGTCGAATGACGATTGA | 1350 |
| | (B1c + B2) | CCTCTGGGACGAG | |
| | LoopF | ACGTGCTCAGATCTGAGAATC | 1351 |
| | LoopB | CATCTGATGGGTCCGCAG | 1352 |
| | F2 | GCCAATGTTCAGATGGATGA | 1353 |
| | F1c | CTGGGAGCCAGATTGCGATC | 72 |
| | B2 | ATTGACCTCTGGGACGAG | 1354 |
| | B1c | TGAAGATGGCGTCGAATGACG | 1355 |
| 9 | Norovirus | | |
| | NG2 | | |
| | F3 | TACGTGCCCAGACAAGAG | 1356 |
| | B3 | CTCCAGAGCCATAACCTCA | 1348 |
| | FIP | CTGGGAGCCAGATTGCGATCCCAATGTT | 1357 |
| | (F1c + F2) | CAGATGGATGAGAT | |
| | BIP | TGAAGATGGCGTCGAATGACGATTGA | 1350 |
| | (B1c + B2) | CCTCTGGGACGAG | |
| | LoopF | TCCCACGTGCTCAGATCT | 1358 |
| | LoopB | CATCTGATGGGTCCGCAG | 1352 |
| | F2 | CCAATGTTCAGATGGATGAGAT | 1359 |
| | F1c | CTGGGAGCCAGATTGCGATC | 72 |
| | B2 | ATTGACCTCTGGGACGAG | 1354 |
| | B1c | TGAAGATGGCGTCGAATGACG | 1355 |
| 10 | Norovirus | | |
| | NG2 | | |
| | F3 | TTACGTGCCCAGACAAGA | 1347 |
| | B3 | CTCCAGAGCCATAACCTCA | 1348 |
| | FIP | CTGGGAGCCAGATTGCGATCGCCAAT | 1349 |
| | (F1c + F2) | GTTCAGATGGATGA | |
| | BIP | TGAAGATGGCGTCGAATGACGATTGA | 1350 |
| | (B1c + B2) | CCTCTGGGACGAG | |
| | LoopF | CCTCCCACGTGCTCAGATCT | 1360 |
| | LoopB | CATCTGATGGGTCCGCAGC | 77 |
| | F2 | GCCAATGTTCAGATGGATGA | 1361 |
| | F1c | CTGGGAGCCAGATTGCGATC | 72 |
| | B2 | ATTGACCTCTGGGACGAG | 1354 |
| | B1c | TGAAGATGGCGTCGAATGACG | 1355 |
| 11 | Norovirus | | |
| | NG2 | | |
| | F3 | TACGTGCCCAGACAAGAG | 1362 |
| | B3 | CTCCAGAGCCATAACCTCA | 1348 |
| | FIP | CTGGGAGCCAGATTGCGATCCCAATGTT | 1357 |
| | (F1c + F2) | CAGATGGATGAGAT | |
| | BIP | TGAAGATGGCGTCGAATGACGATTGACC | 1350 |
| | (B1c + B2) | TCTGGGACGAG | |
| | LoopF | CCTCCCACGTGCTCAGATCT | 1360 |
| | LoopB | CATCTGATGGGTCCGCAGC | 77 |
| | F2 | CCAATGTTCAGATGGATGAGAT | 1363 |
| | F1c | CTGGGAGCCAGATTGCGATC | 72 |
| | B2 | ATTGACCTCTGGGACGAG | 1354 |
| | B1c | TGAAGATGGCGTCGAATGACG | 1355 |
| 12 | Norovirus | | |
| | NG2 | | |
| | F3 | GCCCAGACAAGAGCCAATG | 1364 |
| | B3 | CCAGGGGTCAATTACGTTTTGT | 1365 |
| | FIP | CCCATCAGATGGGTTGGCGTCCGATCG | 1366 |
| | (F1c + F2) | CAATCTGGCTCC | |
| | BIP | AGCCAACCTCGTCCCAGAGGCCACAGG | 1367 |
| | (B1c + B2) | TGCCGCAATAG | |
| | LoopF | TCGACGCCATCTTCATTCACAA | 1368 |
| | LoopB | CTCTGGAGCCCGTTGTTGG | 1369 |
| | F2 | CGATCGCAATCTGGCTCC | 1370 |
| | F1c | CCCATCAGATGGGTTGGCGTC | 1371 |
| | B2 | CCACAGGTGCCGCAATAG | 1372 |
| | B1c | AGCCAACCTCGTCCCAGAGG | 1373 |
| 13 | Norovirus | | |
| | NG2 | | |
| | F3 | GCCCAGACAAGAGCCAATG | 1364 |
| | B3 | TCCAGGGGTCAATTACGTTTTG | 1374 |
| | FIP | CCCATCAGATGGGTTGGCGTCCGATCG | 1375 |
| | (F1c + F2) | CAATCTGGCTCC | |
| | BIP | AGCCAACCTCGTCCCAGAGGCCACAGGT | 1367 |
| | (B1c + B2) | GCCGCAATAG | |
| | LoopF | TCGACGCCATCTTCATTCACAA | 1368 |
| | LoopB | CTCTGGAGCCCGTTGTTGG | 1369 |
| | F2 | CGATCGCAATCTGGCTCC | 1370 |
| | F1c | CCCATCAGATGGGTTGGCGTC | 1371 |
| | B2 | CCACAGGTGCCGCAATAG | 1372 |
| | B1c | AGCCAACCTCGTCCCAGAGG | 1373 |
| 14 | Norovirus | | |
| | NG2 | | |
| | F3 | GCCCAGACAAGAGCCAATG | 1364 |
| | B3 | CCAGGGGTCAATTACGTTTTGT | 1365 |
| | FIP | CCCATCAGATGGGTTGGCGTCACGAT | 1376 |
| | (F1c + F2) | CGCAATCTGGCTCC | |
| | BIP | AGCCAACCTCGTCCCAGAGGCCACAGG | 1367 |
| | (B1c + B2) | TGCCGCAATAG | |
| | LoopF | TCGACGCCATCTTCATTCACAA | 1368 |
| | LoopB | CTCTGGAGCCCGTTGTTGG | 1369 |
| | F2 | CGATCGCAATCTGGCTCC | 1370 |
| | F1c | CCCATCAGATGGGTTGGCGTCA | 1377 |
| | B2 | CCACAGGTGCCGCAATAG | 1372 |
| | B1c | AGCCAACCTCGTCCCAGAGG | 1373 |
| 15 | Norovirus | | |
| | NG2 | | |
| | F3 | GCCCAGACAAGAGCCAATGTTC | 1378 |
| | B3 | TTTGTTGGCCCGCCACAG | 1379 |
| | FIP | GCGGACCCATCAGATGGGTTGGCAAT | 1380 |
| | (F1c + F2) | CTGGCTCCCAGCTTTGTG | |
| | BIP | GCCAACCTCGTCCCAGAGGTCACGCAA | 1381 |
| | (B1c + B2) | TAGCGGCACCAACA | |
| | LoopF | CGTCATTCGACGCCATCTTCA | 75 |
| | LoopB | AATGAGGTTATGGCTCTGGAGC | 1382 |
| | F2 | CAATCTGGCTCCCAGCTTTGTG | 1383 |
| | F1c | GCGGACCCATCAGATGGGTTGG | 1384 |
| | B2 | CGCAATAGCGGCACCAACA | 1385 |
| | B1c | GCCAACCTCGTCCCAGAGGTCA | 1386 |
| 16 | Norovirus | | |
| | NG2 | | |
| | F3 | GCCCAGACAAGAGCCAATGTTC | 1378 |
| | B3 | TTTTGTTGGCCCGCCACAG | 1379 |
| | FIP | GCGGACCCATCAGATGGGTTGGCAATCT | 1380 |
| | (F1c + F2) | GGCTCCCAGCTTTGTG | |
| | BIP | GCCAACCTCGTCCCAGAGGTCACGCAAT | 1381 |
| | (B1c + B2) | AGCGGCACCAACA | |
| | LoopF | CGTCATTCGACGCCATCTTCA | 75 |
| | LoopB | AATGAGGTTATGGCTCTGGAGC | 1382 |
| | F2 | CAATCTGGCTCCCAGCTTTGTG | 1383 |
| | F1c | GCGGACCCATCAGATGGGTTGG | 1384 |
| | B2 | CGCAATAGCGGCACCAACA | 1385 |
| | B1c | GCCAACCTCGTCCCAGAGGTCA | 1386 |
| 17 | Norovirus | | |
| | NG2 | | |
| | F3 | GCCCAGACAAGAGCCAATGTTC | 1378 |
| | B3 | GTTTTGTTGGCCCGCCACA | 1387 |
| | FIP | GCGGACCCATCAGATGGGTTGGCAATCT | 1380 |
| | (F1c + F2) | GGCTCCCAGCTTTGTG | |
| | BIP | GCCAACCTCGTCCCAGAGGTCACGCAAT | 1381 |
| | (B1c + B2) | AGCGGCACCAACA | |
| | LoopF | CGTCATTCGACGCCATCTTCA | 75 |
| | LoopB | AATGAGGTTATGGCTCTGGAGC | 1382 |
| | F2 | CAATCTGGCTCCCAGCTTTGTG | 1383 |
| | F1c | GCGGACCCATCAGATGGGTTGG | 1384 |
| | B2 | CGCAATAGCGGCACCAACA | 1385 |
| | B1c | GCCAACCTCGTCCCAGAGGTCA | 1386 |
| 18 | Norovirus | | |
| | NG2 | | |
| | F3 | TGGATGAGATTCTCAGATCTGAGCA | 1388 |
| | B3 | TCCAGGGGTCAATTACGTTTTGTTG | 1389 |
| | FIP | GACCCATCAGATGGGTTGGCGTCATT | 1390 |
| | (F1c + F2) | GGGAGGGCGATCGCAATC | |
| | BIP | CAGCCAACCTCGTCCCAGAGGTCACA | 1391 |
| | (B1c + B2) | GGTGCCGCAATAGCG | |
| | LoopF | TCTTCATTCACAAAGCTGGGAGCC | 1392 |
| | LoopB | TGGAGCCCGTTGTTGGTGC | 1393 |
| | F2 | TGGGAGGGCGATCGCAATC | 1394 |
| | F1c | GACCCATCAGATGGGTTGGCGTCAT | 1395 |
| | B2 | CACAGGTGCCGCAATAGCG | 1396 |
| | B1c | CAGCCAACCTCGTCCCAGAGGT | 1397 |
| 19 | Norovirus | | |
| | NG2 | | |
| | F3 | ACGTGCCCAGACAAGAGC | 1398 |
| | B3 | GCTCCAGAGCCATAACCTCAT | 1399 |
| | FIP | GCTGGGAGCCAGATTGCGATTTTTATGTT | 1400 |
| | (F1c + F2) | CAGATGGATGAGATTCTCAG | |
| | BIP | GATGGCGTCGAATGACGCCATTTTATTGA | 1401 |
| | (B1c + B2) | CCTCTGGGACGAGG | |
| | LoopF | CCTCCCACGTGCTCAGA | 1402 |
| | LoopB | ACCCATCTGATGGGTCCGCA | 1403 |
| | F2 | ATGTTCAGATGGATGAGATTCTCAG | 1404 |
| | F1c | GCTGGGAGCCAGATTGCGAT | 1405 |
| | B2 | ATTGACCTCTGGGACGAGG | 1406 |
| | B1c | GATGGCGTCGAATGACGCCA | 1407 |

Sample. In some aspects, the sample can be a sample from a subject such as blood, stool, sputum, oropharyngeal, nasopharyngeal, pap smear, urine, or saliva sample. In some aspects, the sample can be a water sample or an environmental sample. In some aspects, the sample can be a sample from a food such as a fruit, meat, vegetable, grain, etc. In some aspects, the sample can be obtained using, for example, a rectal swab, blood or stool spotted on paper (e.g., filter paper, protein saver card), oropharyngeal and nasopharyngeal swabs, pap smear or sputum.

Subject. In some aspects, the subject was exposed or is suspected of being exposed to one or more pathogenic microorganisms. In some aspects, the subject has one or more signs or symptoms of a pathogenic microorganism. In some aspects, the subject has diarrhea, fever, vomiting, abdominal pain, blood in stool or a combination thereof. In some aspects, for HPV, the subject has cervical or oropharyngeal cancer. In some aspects, for Mycobacterium tuberculosis or leprosy, the subject has warts. In some aspects, the subject can be asymptomatic and still carry or be positive for one or more pathogenic microorganisms.

### Kits

Disclosed herein are kits for detecting a target microorganism in a sample. In some aspects, the kits can comprise: a lysis buffer; a filter; a lyophilized buffer; loop mediated isothermal amplification (LAMP) reagents; and one or more primer sets specific to the DNA or RNA of the target microorganism. In some aspects, the LAMP reagents can be lyophilized. In some aspects, the one or more primer sets can be lyophilized. In some aspects, the LAMP reagents and the one or more primer sets can be present in a plurality of microfuge tubes. In some aspects, the filter can comprise a LAMP inhibitor control DNA. In some aspects, the kit further comprises a device for performing the amplification and/or detection of the target microorganism as provided herein.

In some aspects, the target microorganism can be a virus, a bacteriophage, or a bacteria. In some aspects, the target microorganism can be E. coli, Shigella spp, Vibro cholerae, non-cholera Vibro spp, Campylobacter spp, Mycobacterium spp, Salmonella spp, an enteric virus, a coronavirus, or human papillomavirus. In some aspects, the target microorganism can be a parasite. In some aspects, the parasite can be Cryptosporidium, Entamoeba histolytica, Giardia lamblia, and Plasmodium.

In some aspects, the one or more primer sets can be specific for one or more of heat labile toxin (LT) gene, heat stable toxin (STh, and STp) gene, eae gene, bfpA gene, aaiC gene, aatA gene, CVD432 gene, F1845 gene, stx1 gene, stx2 gene, rfbO157 gene, invasion plasmid gene (ipaH), cholera toxin A (ctxA) gene, O1 lipopolysaccharide (O1rfb) gene, O139 gene, 16S gene, IS 6110 gene, MPB 64 gene, 16 S RNA gene, rpoB gene, FliC flagellar gene, invA gene, norovirus G1 gene, norovirus G2 gene, RdRp gene, capsid gene, NSP3 gene, hexon gene, ORF-1 gene, NS5 gene, C gene, E gene, M gene, N gene, S gene, L1 gene, E6 gene, or E7 gene.

The kits can also comprise suitable instructions (e.g., written and/or provided as audio-, visual-, or audiovisual material). The kits can further comprise one or more of the following: instructions, transfer pipettes, microfuge tubes, plastic sticks (stool pisck for solid stool) syringes, a sterile container, delivery devices, tube caps, slides, solid supports, and buffers or other control reagents.

### EXAMPLES

### Example 1. Development of a Simple, Rapid and Sensitive Diagnostic Assay for Enterotoxigenic E. coli and Shigella Spp Applicable to Endemic Countries

Enterotoxigenic E. coli (ETEC) and Shigella spp (Shigella) are complex pathogens and the diagnostic assays currently used to detect these pathogens are either elaborate or complex which are difficult to apply in the resource poor settings where these diseases are endemic.

To address this gap, a rapid and simple diagnostic assay “Rapid LAMP based Diagnostic Test (RLDT)” was developed. Described herein is a sample preparation method using fecal samples, lyophilized reaction strips combined with a loop mediated isothermal amplification platform, ETEC (LT, STh and STp genes) and Shigella (ipaH gene) detection is made simple, rapid (<60 minutes) and inexpensive. ES-RLDT includes 6 primers targeting one gene, making it more specific. This assay is mostly electricity and cold chain free. Moreover, ES-RLDT requires minimal equipment. To avoid any end user's bias, a battery operated, hand-held reader is used to read the ES-RLDT results. The results can be read as positive/negative or as real time amplification depending on the end user's need. The performance specifications of ES-RLDT assay including analytical sensitivity and specificity were evaluated using fecal samples spiked with ETEC and Shigella strains. The limit of detection was 9×104 CFU/gm of stool for LT, STh, and STp and 4×103 CFU/gm of stool for ipaH gene which corresponds to about 23 CFU and 1 CFU respectively per 25 uL reaction within 40 minutes.

ES-RLDT is a diagnostic assay for ETEC and Shigella which is simple and rapid and at the same time sufficiently sensitive. ES-RLDT can be applicable to the resource poor endemic settings and has the potential to address the current gaps in the diagnostic assays of ETEC and Shigella.

Materials and Methods. Optimization of ES-RLDT Assay. The targets for ES-RLDT were selected as heat labile toxin (LT), heat stable toxin (STh, and STp) genes for detection of ETEC and invasion plasmid gene (ipaH) for Shigella. The primers were designed using the online software Primer Explorer V5 (https://primerexplorer.jp/e/). A set of 3 primer pairs, including two outer primers (forward primer F3 and backward primer B3), two inner primers (forward inner primer FIP and backward inner primer BIP), and two loop primers (forward loop primer LF and backward loop primer LB), were selected. The primers were assessed for specificity before use in ES-RLDT assays by analysis using the Basic Local Alignment Search Tool (BLAST) of the National Center for Biotechnology Information (NCBI), against sequences in GenBank. The primer sequences, concentrations, and GenBank IDs are shown in Table 8.

TABLE 8
Primer Sequences used in ES-RLDT assays.

| Primer | | Primer | |
|---|---|---|---|
| Name | Sequence (5′-3′) | Concentration | Reference |
| ETEC | | | |
| LT | GenBank: FN649414.1 | | |
| F3 | ATCGTGTTAATTTTGGTGTGATTG (SEQ ID NO: 1) | 0.8 μM | This |
| | | | Study |
| B3 | CTGGGTCTCCTCATTACAAGT (SEQ ID NO: 2) | 0.8 μM | This |
| | | | Study |
| FIP | AACCATCCTCTGCCGGAGCTATATTGAACGATT | 1.6 μM | This |
| | ACATCGTAACAGGGAAT (SEQ ID NO: 3) | | Study |
| BIP | TTCCCACCGGATCACCAAGCTGTTCTTGATGAA | 1.6 μM | This |
| | TCTCCACAACCTT (SEQ ID NO: 4) | | Study |
| LF | GATTTCTGTAATACCGGTCTCTAT (SEQ ID NO: 5) | 0.8 μM | This |
| | | | Study |
| LB | AGAAGAACCCTGGATTCATCATGC (SEQ ID NO: 6) | 0.8 μM | This |
| | | | Study |
| ETEC | | | |
| STp | GenBank: FN649414.1 | | |
| F3 | GCAAAATCCGTTTAACTAATCTCAA (SEQ ID NO: 7) | 0.8 μM | This |
| | | | Study |
| B3 | ACAGCAGTAAAATGTGTTGTTCAT (SEQ ID NO: 8) | 0.8 μM | This |
| | | | Study |
| FIP | AAGAGGGGAAAGATAATACAGAAATTTTTTAAA | 1.6 μM | This |
| | CAACATGACGGGAGGT (SEQ ID NO: 9) | | Study |
| BIP | TAGTCAGTCAACTGAATCACTTGATTTTTCTGTTG | 1.6 μM | This |
| | TTTTTTACAACATCACACT (SEQ ID NO: 10) | | Study |
| LF | GCCAACATTAGCTTTTTCATG (SEQ ID NO:11) | 0.8 μM | This |
| | | | Study |
| LB | CAAAAGAGAAAATTACATTAGAGAC (SEQ ID NO: | 0.8 μM | This |
| | 12) | | Study |
| ETEC | | | |
| STh | GenBank: FN649414.1 | | |
| F3 | CTCAGGATGCTAAACCAGT (SEQ ID NO: 13) | 0.2 μM | Yano et al |
| | | | 2007¹⁷ |
| B3 | CAGAACAAATATAAAGGGAACTGTT (SEQ ID NO: 14) | 0.2 μM | Yano et al |
| | | | 2007 |
| FIP | TCATGCTTTCAGGACCACTTTTATTGAGTCTTCAAAA | 1.6 μM | Yano et al |
| | GAAAAAATCACACT (SEQ ID NO: 15) | | 2007 |
| BIP | AGTAGCAATTACTGCTGTGAATTGTCCCTTTATATTA | 1.6 μM | Yano et al |
| | TTAATAGCACCCG (SEQ ID NO: 16) | | 2007 |
| LB | GTTGTAATCCTGCTTGT (SEQ ID NO: 17) | 0.8 μM | Yano et al |
| | | | 2007 |
| Shigella | | | |
| ipaH | GenBank: M76444.1 | | |
| F3 | AATTCTGGAGGACATTGCC (SEQ ID NO: 18) | 0.2 μM | This |
| | | | Study |
| B3 | CGTACGCTTCAGTACAGC (SEQ ID NO: 19) | 0.2 μM | This |
| | | | Study |
| FIP | CTTCACGGCAGTGGAGAGCTGAGATAGAA | 1.6 μM | This |
| | GTCTACCTGGC (SEQ ID NO: 20) | | Study |
| BIP | TATGGCGTGTCGGGAGTGATTCATTCTCTT | 1.6 μM | This |
| | CACGGCTTC (SEQ ID NO: 21) | | Study |
| LF | CTCTGCGAGCATGGTCTG (SEQ ID NO: 22) | 0.8 μM | This |
| | | | Study |
| LB | CACTGCCGAAGCTATGGT (SEQ ID NO: 23) | 0.8 μM | This |
| | | | Study |

A loop mediated isothermal amplification (LAMP) based assay was developed for detection of ETEC and Shigella using OmniAmp 2× Isothermal Master Mix (Lucigen, WI). The final concentrations of the reaction mixtures were 1× OmniAmp Master Mix, 2 mM FionaGreen dye (Marker Gene, OR), 1×LAMP primer mix, and 5 μL of DNA, brought to volume (25 μL) with DNase-RNase-free water. Initial optimization experiments were performed on a Step One Plus Real-Time PCR System (Applied Biosystems, CA). Amplification was monitored by the detection of FionaGreen fluorescence and quantified by the instrument software. To calculate the time to result, threshold was set as 3000 or 4000 RFU. The reaction temperature was determined using a gradient of 68° C.-74° C. Later experiments were performed using AmpliFire Isothermal Fluorometer (Douglas Scientific, MN currently Agdia Inc., IN). The ETEC primers were tested for any cross reactivity against ctxA gene of Vibrio cholera. The ipaH primer was tested against S. flexneri, S. sonnei, S. dysenteriae, and S. boydii to confirm these primers can detect all of these Shigella spp.

Development of a Simple and Rapid Sample Preparation Method. To make ES-RLDT assay appropriate to use in the low resource settings, a simple and rapid sample preparation method directly from the stool samples was developed. Heat lysis was performed by diluting sample into an extraction buffer followed by incubation at 90° C. for 5 min. After incubation, lysates were used as template in ES-RLDT reactions. M13mp18 plasmid (New England BioLabs, MA) was included as reaction inhibitor control in the extraction buffer.

ES-RLDT with Lyophilized Reagents. The ES-RLDT assay is developed as a kit. The kit includes reaction strips and accessories for sample preparation. Each reaction strip has 8 microfuge tubes (0.2 ml) with the LAMP master mix targeting LT, STh, STp and ipaH genes, with one target in each tube. In addition, a tube for reaction inhibitor control is added per sample. To allow ambient storage, complete 1× OmniAmp-LAMP formulation along with 10% trehalose (Sigma-Aldrich, MO), were dispensed into the microfuge tubes (Denville). The mixture was lyophilized at JHU core laboratory, using a Labconco FreeZone bench-top lyophilizer (Labconco Corporation, MO). After lyophilization, tubes were capped and packed in light resistant bags with desiccant pouch. Lyophilized ES-RLDT reactions were rehydrated with template, into a total volume of 25 μl and incubated and detected in AmpliFire fluorometer reader. The reader has the ability to incubate and read eight samples simultaneously (one strip). This device is optimized for isothermal chemistry and allows real time monitoring of amplification. It offers a touch screen interface, data storage and portability (hand-held) with a rechargeable battery. The algorithm can be set depending on the end user's need either for in depth analysis of the amplification using the detection curves or for binary +/− results. The assay programs are coded in bar codes and scanned to include in the machine.

Performance Testing of ES-RLDT Assay. For analytical sensitivity and specificity of ES-RLDT, ETEC strain H10407 (LT+STh+STp+) and Shigella flexneri 2a 2457T were used as ETEC and Shigella positive strains, respectively. Both the strains were obtained from Walter Reed Army Institute of Research (WRAIR) The naïve stool samples used were from donors negative for ETEC and Shigella. The strains were cultured in LB broth and incubated at 37° C. for approximately 6-7 hours. The number of Colony Forming Units (CFU) was determined by optical density as well as by quantitative plate counts from the culture. The naïve stool sample was aliquoted and spiked with 10-fold serially diluted cultures of ETEC or Shigella. The spiked stool samples as well as the naïve stool samples were processed as described before. To determine specificity, ES-RLDT was tested using a range of positive and negative strains of E. coli, Shigella spp as well as other enteric pathogens like Vibrio cholerae, Campylobacter spp, and Salmonella typhi.

The ES-RLDT assay was evaluated for repeatability, reproducibility, accuracy, matrix inhibition and linearity directly from spiked serially diluted stool samples and lyophilized strips. The lyophilized RLDT strips were also tested for stability at ambient temperatures.

To compare sensitivity of ES-RLDT assay with qPCR the naïve stool sample was spiked with 10-fold serially diluted cultures of ETEC and Shigella separately. DNA was extracted from the spiked stool using QIAamp DNA Stool Mini Kit (Qiagen Valencia, CA). Purified 2.5 uL DNA from each spiked sample dilution was used for both ES-RLDT and qPCR (Lothigius A, et al. J Appl Microbiol. 2008; 104(4):1128-36).

Statistical Analysis. The coefficient of variations (CV) were calculated as the ratio of the standard deviation to the mean (average). The linearity was determined by plotting the log time to result (TTR) values against CFU/gm of stool and Pearson correlation coefficient was calculated.

Results. Optimization of Reaction Temperature and Time. For determination of optimum temperature, reactions were performed using ETEC and Shigella strains at the temperatures between 68° C. to 74° C. in 1° C. increments for 30 to 60 minutes in 10 minutes increments. Optimum time to result (TTR), sensitivity, and specific amplification were obtained when the reaction was performed at 71° C. for 40 minutes. Analyzing the results with considering TTR, relative fluorescence units (RFU), specificity and sensitivity the threshold was set at 4000 RFU. Subsequent reactions were performed at 71° C. for 40 minutes, unless otherwise noted.

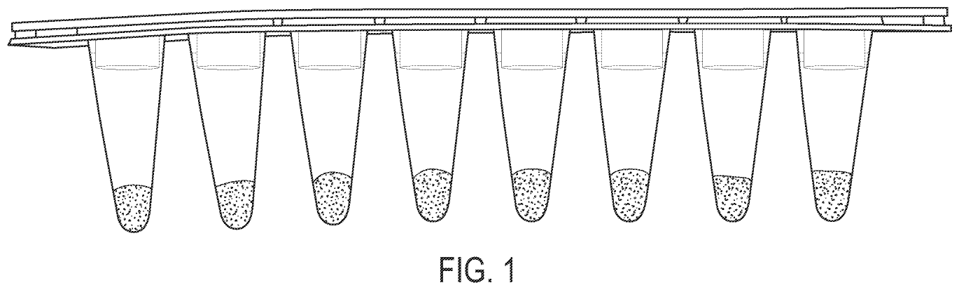

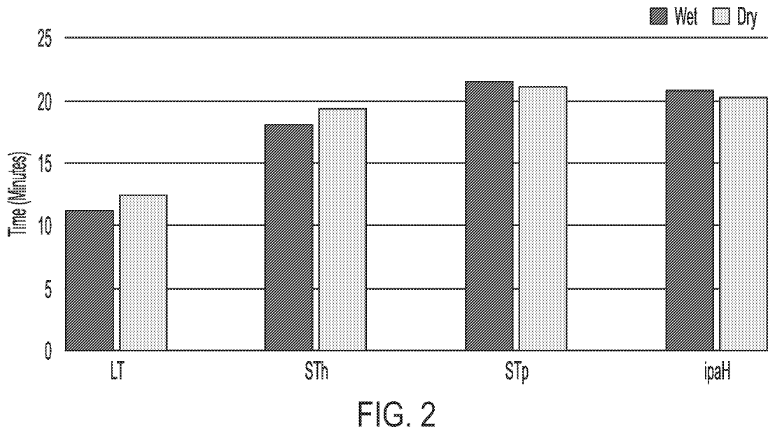

ES-RLDT with lyophilized reagents. A lyophilized formulation for the ES-RLDT reagents (FIG. 1) was compared with wet reagents for detection of ETEC and Shigella targets. No diminution of TTR or sensitivity was seen with the dried formulation compared to wet (FIG. 2).

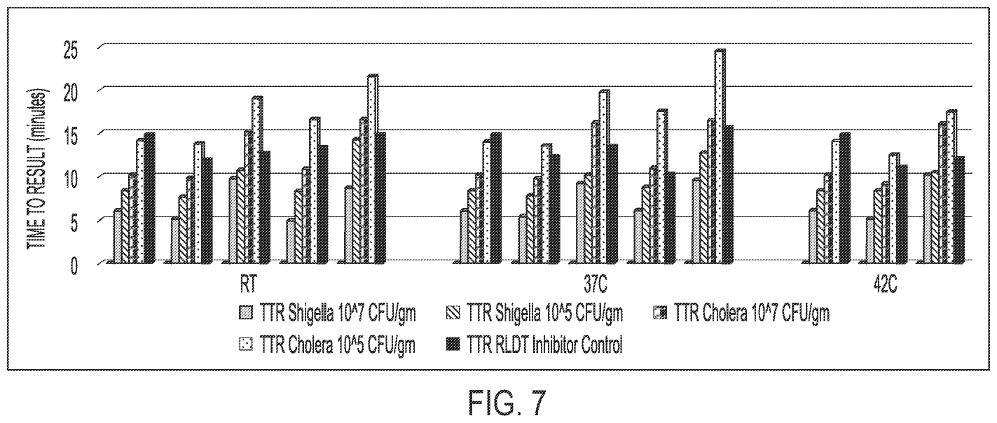

To evaluate stability of the dry formulations of RLDT, the lyophilized ES-RLDT reagents were incubated at room temperature (˜23° C.), 37° C., and 42° C. and assayed by ES-RLDT. The dry ES-RLDT formulation was stable at 23° C. and 37° C. for 90 days. The dried reagents were stable and functional at 42° C. for 60 days although it did show higher TTRs in the later months. FIG. 7 also shows that the lyophilized RLDT assay strips and reagents were stable for at least one year.

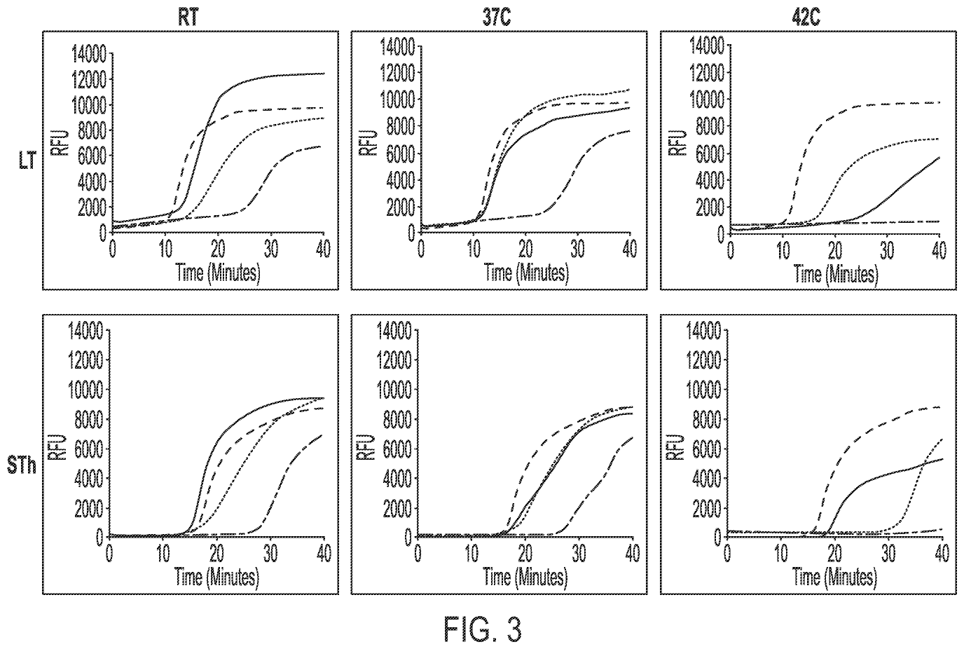

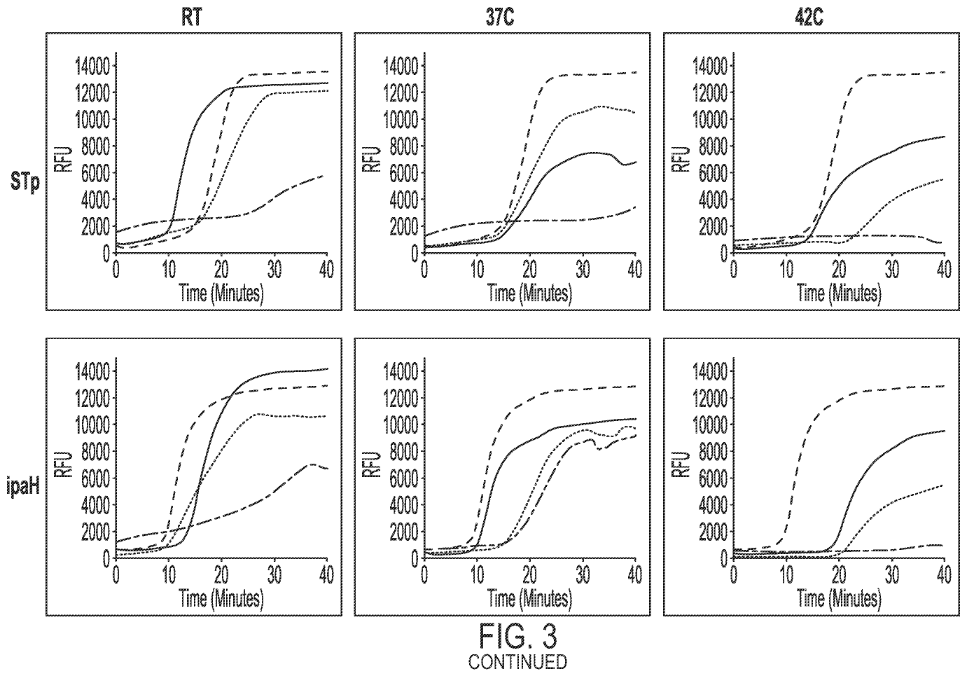

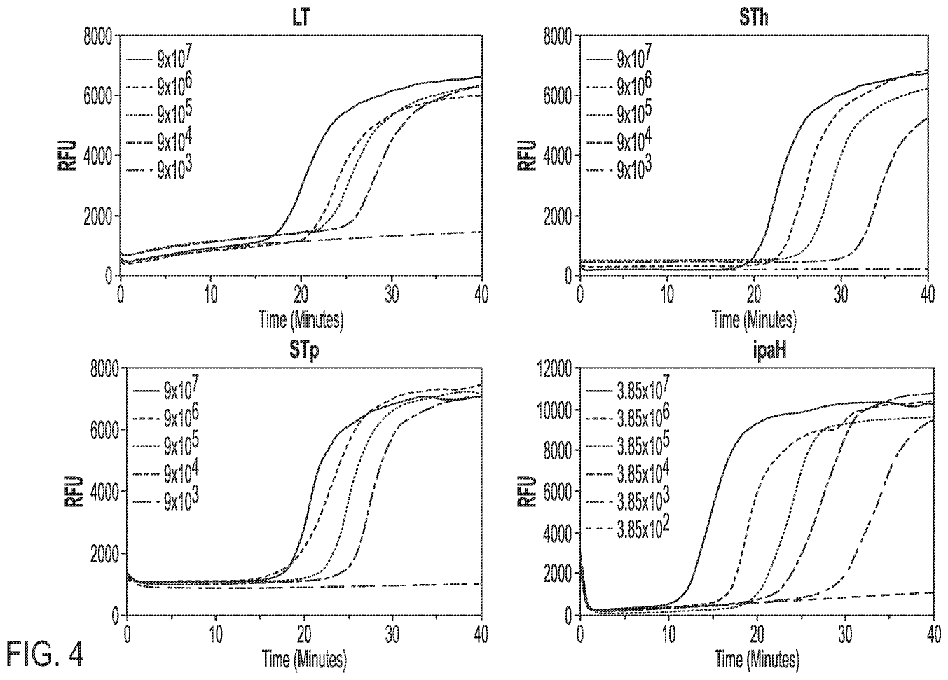

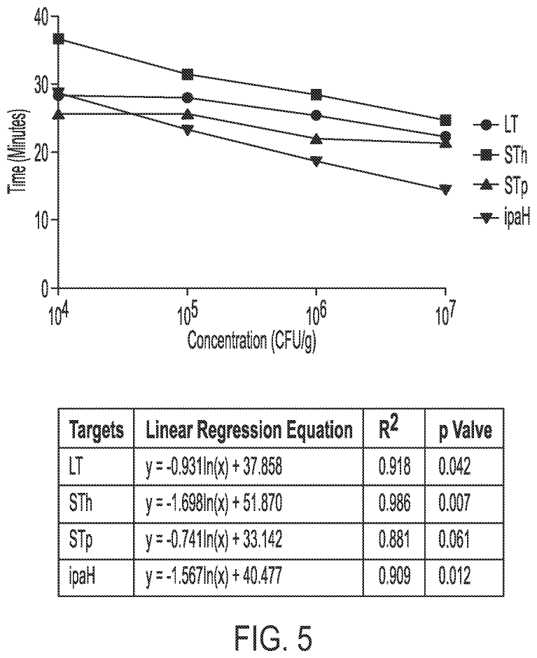

Analytical Sensitivity and Specificity of ES-RLDT The 10-fold serial dilutions of ETEC and Shigella spiked stools ranged from 102 to 108 CFU of organisms per gram of stool were run 10 times using our rapid sample preparation method and lyophilized reaction strips. The lowest detection limit (LOD) was 9×104 CFU/gm of stool for LT, STh, and STp and 3.85×103 CFU/gm of stool for the ipaH gene which corresponds to about 23 CFU and 1 CFU per 25 uL reaction respectively within 40 minutes (FIG. 3). LOD was defined as the lowest concentration at which the target could be detected in the 10 runs with spiked samples. Linearity was established by averaging the TTR over three runs and plotting the results. The TTR increased consistently as the concentration of the bacteria in the samples decreased. The R² linearity values were between 0.88 to 0.99 (FIG. 5).

No amplification was observed with naive stool or stool samples spiked with pathogens other than the targets, suggesting that the assay is specific to ETEC and Shigella (Table 9). The ipaH gene could detect S. flexneri 2a, S. dysenteriae, S. sonnei and S. boydii. The reaction inhibitor control was positive in every run.

TABLE 9
Specificity of ES-RLDT.

| Pathogen/Strain | Source | LT | STh | STp | ipaH |
|---|---|---|---|---|---|
| ETEC H10407 | WRAIR | + | + | + | − |
| ETEC B7A | WRAIR | + | + | + | − |
| ETEC E24377A | WRAIR | + | + | − | − |
| E. coli 25922 | ATCC | − | − | − | − |
| ETEC 335093 | Bangladesh | + | − | + | − |
| ETEC 335140 | Bangladesh | − | + | + | − |
| ETEC 335152 | Bangladesh | + | − | − | − |
| S. flexneri 2a 2457T | WRAIR | − | − | − | + |
| S. dysenteriae AMC 43-A-1 | ATCC | − | − | − | + |
| S. boydii AMC 4006 | ATCC | − | − | − | + |
| S. sonnei | ATCC | − | − | − | + |
| V. Cholera O1 N16961 | ATCC | − | − | − | − |
| V. Cholera O1 14035 | ATCC | − | − | − | − |
| V. Cholera O139 51394 | ATCC | − | − | − | − |
| Salmonella typhi | ATCC | − | − | − | − |
| Campylobacter jejuni | ATCC | − | − | − | − |
| Campylobacter coli | ATCC | − | − | − | − |

Repeatability was tested with ten repeats of two samples, respectively, spiked with a high (10⁷) and a low (10⁵) concentration of each target (see, Table 10). Reproducibility was tested with 10 identically spiked samples for each concentration, high and low, that were assayed over 5 days. Positive results were defined when the amplification RFU reached the threshold within 40 minutes of the reaction and the assay inhibitor control was positive. Both high and low dilutions met the criteria for positive results for 10000 of the repeatability and reproducibility assays. Analytical accuracy was evaluated with reference samples. The accuracy (sensitivity and specificity) for detecting both ETEC and Shigella targets were 100%. Stool is a difficult substrate to use for extraction and amplification because of the presence of a variety of inhibitors, which can vary between samples. Matrix inhibition was tested using three lots of stool from healthy donors spiked with positive controls of ETEC and Shigella and extracted and amplified in duplicates. No significant difference was observed among the three lots for any of the assays and the results were as expected.

TABLE 10
Analytical performance of RLDT.

| Test | | Acceptance | | Results |
|---|---|---|---|---|
| performed | Test Method | Criteria | Targets | (Pass/Fail) |
| Limit of | LOD is defined as number of | Assay | ETEC | 9 × 10⁴ CFU/g |
| Detection | copies per gram of stool that were | inhibitor | LT | m of stool |
| (LOD) | consistently 100% detectable with | control is | STh | |
| | 10 distinct | positive. | STp | |
| | extractions/amplifications. | Positive | Shigella | 4 × 10³ |
| | | results are | ipaH | CFU/gm of |
| | | defined | | stool |
| Repeatability | Tested with five repeats of two | when the | ETEC | |
| | samples respectively spiked with a | amplification | LT | 20/20 |
| | high and a low concentration of | reach the | | (100%) |
| | each ETEC and Shigella. | threshold | STh | 20/20 |
| | | within 40 | | (100%) |
| | | minutes of | STp | 20/20 |
| | | the reaction. | | (100%) |
| | | | Shigella | |
| | | | ipaH | 20/20 |
| | | | | (100%) |
| Reproducibility | Tested with 10 identically spiked | | ETEC | |
| | samples for each concentration | | LT | 20/20 |
| | (two concentrations, high and low, | | | (100%) |
| | were interrogated) that were | | STh | 20/20 |
| | extracted and assayed over 5 days. | | | (100%) |
| | | | STp | 20/20 |
| | | | | (100%) |
| | | | Shigella | |
| | | | ipaH | 20/20 |
| | | | | (100%) |
| Accuracy | ES-RLDT was tested using | | ETEC | |
| | reference samples as well as a | | LT | 100% |
| | range of positive and negative | | STh | 100% |
| | strains of enteric pathogens. | | STp | 100% |
| | | | Shigella | |
| | | | ipaH | 100% |
| Matrix | Three different lots of stool from | | ETEC | |
| Inhibition | healthy donors were spiked with | | LT | 6/6 |
| | high and low concentrations of | | | (100%) |
| | ETEC or Shigella and tested. | | STh | 6/6 |
| | | | | (100%) |
| | | | STp | 6/6 |
| | | | | (100%) |
| | | | Shigella | |
| | | | ipaH | 6/6 |
| | | | | (100%) |

To understand if ES-RLDT could be used as a semi-quantitative assay, the % CV of the TTR values of ETEC and Shigella target genes were analyzed during repeatability and reproducibility experiments. The TTR values of the target genes had repeatability, within-run variance from 2.78% to 9.43% and reproducibility, between-run variance from 4.41% to 12.90% (Table 11).

TABLE 11
CV of ES-RLDT.

| TTR CV (%) | |
|---|---|
| Repeatability | Reproducibility |

| Targets | High | Low | High | Low |
|---|---|---|---|---|
| LT | 4.06 | 2.78 | 6.94 | 12.38 |
| STh | 3.54 | 6.67 | 4.41 | 10.5 |
| STp | 3.34 | 8.51 | 7.71 | 12.54 |
| ipaH | 8.89 | 11.18 | 9.71 | 12.90 |

Sensitivity of ES-RLDT compared to qPCR. The sensitivity of ETEC and Shigella detection by ES-RLDT was compared to that of qPCR, using purified DNA from 10-fold serial dilutions ranging from 10⁰ to 10⁷ CFU/gm of stool of spiked stool of ETEC or Shigella. The sensitivity of both the assays were similar and could detect till <10 bacteria/gm of stool.

Advantages of ES-RLDT over current diagnostics assays of ETEC and Shigella. Ease of use: ES-RLDT is performed directly from the stool samples with minimum treatment. Using the rapid sample preparation and lyophilized kit the assay is simple. Assay can be performed with minimum training. The assay results can be read as +/− using a handheld reader. RLDT is mostly electricity free as its use the battery-operated reader. Lyophilized tubes can be stored at ambient temperature and thus can avoid maintaining cold chain. Rapid: The assay will take ˜50 minutes from stool to end result and thus, ETEC and Shigella colonies can be isolated from the positive samples for further characterization. Specificity: As 6 primers are used for detecting each target, ES-RLDT has high specificity. Equipment: ES-RLDT requires a heat block and a reader. Waste: Will generate minimum biohazard wastes.

Discussion ES-RLDT is a rapid and simple nucleic acid amplification based diagnostic assay for ETEC and Shigella which is suitable to the laboratories and clinics of the resource poor endemic countries. Considering the ultimate goal for this assay as a point-of-use diagnostic tool for areas with limited access to adequate equipment and infrastructure, it was important that the ES-RLDT assay be optimized for maximum ease of use and ability for ambient storage. Stool is a challenging substrate to use for extraction of DNA and amplification because of the presence of a variety of inhibitors, which can vary between samples. Therefore, in designing this test, competing challenges were confronted to make the sample processing procedure simple but at the same time sensitive and specific. A simple and rapid sample preparation method directly from the stool was developed which resulted in a LOD of 10⁴ CFU/gm of stool for Shigella and 10⁵ CFU/gm of stool for ETEC which are equivalent to 1 and 23 copies per reaction tube, respectively. This LOD is either lower (for Shigella) or same (for ETEC) as reported for detection of ETEC and Shigella genes using the TaqMan Array Card for enteropathogen detections (Liu J, et al. J Clin Microbiol. 2013. 51(2): 472-480) that has been used in the reanalysis of the samples from the Global Enteric Multicenter Study (GEMS) (Liu J, et al. Lancet. 2016 388(10051):1291-30116) and the multisite birth cohort study (MAL-ED) (James A Platts-Mills, et al. Lancet Glob Health. 2018 December; 6(12): e1309-e1318). Of note, the TaqMan Array card uses purified DNA and RLDT is performed directly from the stool. The sensitivity of RLDT was similar to quantitative PCR when both of the assays were performed with purified DNA from stool, establishes that the assay method in RLDT did not affect the sensitivity of the assay.

The reagents were lyophilized including primers and dye, made RLDT as dry format which is stable in ambient temperatures. These modifications avoid handling of individual reagents as well as requirement of maintaining cold chain which makes RLDT applicable to endemic countries where improved laboratories/clinics are not available.

Similar to LAMP, the ES-RLDT results can be read by naked eye or using UV illuminator. However, this may create end user's bias when using the assay in the field by technicians with minimum training. To address this difficulty, a handheld battery powered equipment, Amplifier, which can read the results as positive or negative, is recommended. This equipment will add cost to the assay but is a one-time primary cost and would outweigh the cons of end user's bias.

Since RLDT assay takes about 50 minutes from stool to end result and, thus, ETEC and Shigella positive stool samples can be cultured to isolate colonies for downstream characterization of the strains, using serotyping for both ETEC and Shigella, colonization factors typing of ETEC, susceptibility testing to antibiotics and whole genome sequencing. Using ES-RLDT as a screening tool has a huge advantage during disease surveillance or ETEC and Shigella vaccine phase III trials in the endemic countries, as would largely minimize the time, workload and cost.

Although RLDT is a qualitative test, a linear relation between the TTR of RLDT and CFU or copy numbers of the bacteria per gram of stool was observed. Thus, the TTR in RLDT might be able to semi-quantify the number of the target bacteria in stool.

RLDT is based on LAMP technology which was first developed by Notomi et al (Notomi T., et al. (2000). Nucleic Acids Res. 28:E63 10.1093/nar/28.12.e63). In recent years, several LAMP based assays have been developed in the laboratories for the rapid diagnosis of infectious pathogens including ETEC (Yano A, et al. J Microbiol Methods. 2007 68(2):414-20; Liu W, et al. J Microbiol Methods. 2019 June; 161:47-55; and Yang W, et al. Biosci Trends. 2014 8(6):316-21) and Shigella (Wang Y, et al. Front Microbiol. 2015 Dec. 14; 6:1400; Liew P S, et al. Trop Biomed. 2014 December; 31(4):709-20; Soli K W, et al. Diagn Microbiol Infect Dis. 2013 December; 77(4):321-3; Shao Y, et al. Int J Food Microbiol. 2011 Aug. 2; 148(2):75-9; and Zhang L, et al. Front Microbiol. 2018; 9: 94). Stool is a complex sample to extract DNA and amplify because of the presence of inhibitors. The LAMP assays previously developed to detect enteric pathogens from stool are either from isolated colonies or isolating purified DNA with commercial kits or using complex process which are not feasible in the resource poor endemic settings. In addition, these assays require to maintain cold chain which is difficult to achieve in these settings. ES-RLDT have addressed these issues and adapted to be applicable to the endemic settings where it is much needed.

In conclusion, ES-RLDT assay described in this study has advantages, including rapid results, simple operation procedures, easy readout of the results as well as having a better sensitivity compared to culture methods and colony-based PCR and equivalent sensitivity to the detection of ETEC and Shigella using quantitative PCR. In addition, ES-RLDT is mostly electricity and cold chain free. Together, these qualities make RLDT easy to scale up and appropriate to use in the endemic settings.

### Example 2. Field Evaluation of a Novel, Rapid Diagnostic Assay RLDT, and Molecular Epidemiology of Enterotoxigenic E. coli Among Zambian Children Presenting with Diarrhea

Enterotoxigenic Escherichia coli (ETEC) is one of the top aetiologic agents of diarrhea in children under the age of 5 in low-middle income countries (LMICs). The lack of point of care diagnostic tools for routine ETEC diagnosis results in limited data regarding the actual burden and epidemiology in the endemic areas. Rapid LAMP based Diagnostic Test (RLDT) was evaluated for its ability to detect ETEC in stool as a point of care diagnostic assay in a resource-limited setting.

A cross-sectional study of 324 randomly selected stool samples from children under 5 presenting with moderate to severe diarrhea (MSD) was carried out. The samples were collected between November 2012 and September 2013 at selected health facilities in Zambia. The RLDT was evaluated by targeting three ETEC toxin genes [heat labile toxin (LT) and heat stable toxins (STh, and STp)]. Quantitative PCR was used to evaluate the diagnostic sensitivity and specificity of RLDT for detection of ETEC.

The study included 50.6% of participants that were female. The overall prevalence of ETEC was 19.8% by qPCR and 19.4% by RLDT. The children between 12 to 59 months had the highest prevalence of 22%. The study determined ETEC toxin distribution was LT (49%), ST (34%) and LT/ST (16%). The sensitivity and specificity of the RLDT compared to qPCR using a Ct 35 as the cutoff, were 90.7% and 97.5% for LT, 85.2% and 99.3% for STh and 100% and 99.7% for STp, respectively.

The results show that RLDT is sensitive and specific as well as easy to implement in the endemic countries. Being rapid and simple, the RLDT also is a tool for point-of-care testing at the health facilities and laboratories in the resource-limited settings. ETEC is a top cause of diarrheal diseases in low and middle income countries. The advancement of molecular diagnosis has made it possible to accurately detect ETEC in endemic areas. However, the complexity, infrastructure and cost implication of these tests has made it a challenge to routinely incorporate them in health facilities in endemic settings. The ETEC RLDT is a molecular tool that can be used to screen for ETEC in resource limited settings. Described herein, is the performance of the RLDT against a qPCR. The findings demonstrate that the ETEC RLDT performs comparable to the qPCR.

Enterotoxigenic Escherichia coli (ETEC) is one of the top ten causes of diarrhea (Troeger C, et al. The Lancet Infectious Diseases. 2018; 18: 1211-1228) with an estimated 75 million diarrhea episodes annually in children under the age of 5 years. It is also responsible for an estimated 18,700 deaths (9,900-30,659), accounting for ˜4.2% (2.2-6.8) of total diarrhea-related deaths (Khalil I A, et al. The Lancet Infectious Diseases. 2018; 18: 1229-1240). Diarrhea is also associated with long-term consequences of poor growth and cognitive development among children (Khalil I, et al. Enterotoxigenic Escherichia coli (ETEC) vaccines: Priority activities to enable product development, licensure, and global access. Vaccine. 2021; and Anderson J D, et al. The Lancet Global Health. 2019; 7: e321-e330). The ETEC disease burden estimates are reportedly lower than the actual cases in endemic areas due to limited diagnostic capacity (Liu J, et al. The Lancet. 2016; 388: 1291-1301). In low- and middle-income countries (LMICs), diarrhea remains a wet season disease with enteric pathogens like ETEC playing a fundamental role in warmer and wetter summer months (Levine M M, et al. The Global Enteric Multicenter Study (GEMS): Impetus, rationale, and genesis. Clinical Infectious Diseases. 2012; 55; and Paredes-Paredes, M, et al. Journal of Travel Medicine. 2011; 18: 475, pp. 121-125). It is important to understand the seasonality of ETEC in the region to inform policymakers on prevention and control strategies.

To accurately diagnose ETEC, one needs to first culture stool, isolate E. coli colonies and then test if the bacterium produces toxins (LT, STh, and STp) through the use of phenotypic assays such as dot blotting or through the use of conventional PCR. Quantitative PCR (qPCR) is performed with purified DNA from stool; although more sensitive; however, it is technology dependent and difficult to perform without well-equipped laboratories (Croxen M A, et al. Clinical microbiology reviews. 2013; 26: 822-880). The complex nature of the diagnosis leads to (i) long turnaround time, which in turn promotes presumptive treatment that could lead to Antimicrobial Resistance (AMR) (Tribble D R. Journal of travel medicine. 2017; 24: S6-S12; and Bokhary H, et al. Tropical Medicine and Infectious Disease. 2021; 6: 11) and (ii) increase in cost and labor needed for the detection of ETEC, resulting in ETEC not being routinely tested in resource-limited settings.

The complexity of these diagnostic methods results in the underestimation of the burden of ETEC because countries where the infection is endemic cannot afford the infrastructure and expertise required for this (Lanata C F, Black R E. The Lancet Infectious diseases. 2018; 18: 1165-116). To develop an effective program for control and prevention, accurate burden data is important (Fleckenstein J M, Kuhlmann F M. Current infectious disease reports. 2019; 21:9). In resource-limited settings, there is a need for a simple, readily available method that can be used to detect ETEC in minimally equipped laboratories and health settings.

In this study, RLDT was field evaluated in Zambia and compared with qPCR, using previously collected stool samples. The prevalence of ETEC infections among the Zambian children presenting with moderate to severe diarrhea (MSD) is also described as well as asymptomatic cases, at the outpatient clinics.

Materials and Methods. Study design. This was a retrospective study using 324 randomly selected samples from 1500 stored stool samples collected at various health facilities in the Lusaka district of Zambia. These samples were collected between November 2012 to September 2013 from a rotavirus vaccine effectiveness study (Beres et al. Clinical Infectious Diseases. 2016; 62: S175-S182). Clinical information including diarrhea severity, social demographic data were collected from study participants.

Randomization of stool samples before selection. An independent statistician was tasked to randomly select 324 retrospectively collected samples. This set of samples represented stool samples with equal distribution of sex and under 5 age groups. The statistician also stratified the participant samples with a 2:1 ratio of symptomatic and asymptomatic diarrhea cases.

Laboratory Assays. Sample Processing Collection and Storage. Samples with collected clinical information of moderate to severe diarrhea and asymptomatic representation were sorted, separated and stored at −80° C. before testing.

ETEC-RLDT Assay. RLDT assays were conducted directly from the frozen stool samples using the RLDT kit. In short, samples were added to a sample processing tube with lysis buffer followed by heat lysis. The processed samples were then added to the ETEC RLDT lyophilized reaction tube (LRT) strips. Each strip consisted of 8 tubes, organized as two reaction tubes each for LT, STh and STp genes. One reaction tube was added as the RLDT inhibitor control (Connor S, et al. Evaluation of a novel, simple, sensitive and rapid fieldable detection assay for ETEC and Shigella spp from stool samples. (PNTD-D-21-01199R1). The strips were run for 40 minutes in a real time fluorometer reader (Agdia Inc, IN, USA). The results were read as positive/negative by the reader.

qPCR Assay. Nucleic Acid extraction: About 100-150 mg of bulk stool were added to SK38 bead tubes (Bertin Technologies, Montigny, France) containing lysis buffer (bioMérieux, Marcy I'Etoile, France). The stool suspension was vortexed for 5 minutes, allowed to stand at room temperature for 10 to 15 minutes, then centrifuged at 14,000 rpm for 2 minutes to pellet stool material. About 200 μl of the supernatant were transferred into a nuclease-free 1.5 ml microcentrifuge tube for extracting nucleic acid using a QIAamp DNA Mini Kit (Qiagen, Hilden, Germany) according to the manufacturer's instructions. qPCR Amplification: The 25 μl reaction mixtures contained 12.5 ul Quantitech SYBR Green Master mix (Qiagen, Hilden, Germany), 1 uM primer mix 5 ul, PCR grade water 5 μl (Invitrogen, USA) and 2.5 ul of samples. PCR was carried out for 40 cycles of 95° C. for 15 s and 60° C. for 1 min (Bolin I, et al. Journal of Clinical Microbiology. 2006; 44: 3872-3877). qPCR cycling conditions were run on the RotogeneQ platform (Qiagen, Hilden Germany). Cut-off for the determination of ETEC positives was set as Ct35 as was done in previous studies (Liu J, et al. The Lancet. 2016; 388: 1291-1301). Each sample was run at a minimum in duplicate, and results were averaged. Chakraborty et al previously has established the limit of detection (LOD) of RLDT for ETEC genes LT, STh and STp using stool samples spiked with reference ETEC strain (PNTD-D-21-01199R1, PNTD-D-21-01198R1). The LOD was 9×10⁴ CFU/g of stool which corresponds to qPCR Ct of 28.2, 28.6 and 30.07 for LT, STh and STp respectively). Therefore, we also evaluated the performance of the RLDT using this LOD (Ct 28) as the cut off (Table 14).

Diarrhea (symptomatic) was defined as the primary caregiver reporting that the child had three or more loose stools within 24 hours. An asymptomatic case was defined as a child presenting to a health facility with other non-diarrhea complications.

Statistical analysis. A minimum sample size of 324 with an assumed ETEC prevalence of 40.7% (Chisenga C C, et al. Pediatric Infect Dis. 2018; 3: 8) produces a two-sided 95% sensitivity confidence interval with a width of 12% when the sample sensitivity is at least 85% and the two-sided 95% specificity confidence interval with a width of 5% when sample specificity is at least 0.95%. Summary statistics were calculated for baseline variables. Proportions and median (IQR) were used to express categorical and continuous variables. A Chi-square test was used to determine the association between ETEC positivity and baseline characteristics.

Statistical analysis significance was set at p-value <0.05 and data were analysed using Stata version 16.0 (StataCorp LLC, College Station, Texas). The correlation of ETEC monthly positivity frequencies was assessed to determine seasonality.

A sample was considered positive for ETEC, when at least one of the ETEC genes, LT,STh or STp was positive. To compare RLDT with qPCR, a Ct value cut off of 35 was used. Any sample with Ct value of 35 or less by qPCR was considered as true positive.

To avoid incorrectly determining some samples to be false positive by RLDT, samples with Ct-values greater than 35 detectable by both qPCR and RLDT were also included as true positive. RLDT was compared with qPCR with the Ct value cut off of 28.

Social demographics and prevalence. A total of 324 samples with a mean age of about 30 months were included in the analysis, 50.9% were female, 28.4% were asymptomatic, with 3.10% of the symptomatic cases presenting with severe disease according to a modified versikari severity scoring (Fleckenstein J M, Kuhlmann F M. Current infectious disease reports. 2019; 21: 9). Overall, ETEC prevalence was about 19% with both the assays, RLDT and qPCR and the highest prevalence was observed in children between 12-59 months of about 22% (Table 12).

TABLE 12
Baseline characteristics by qPCR/RLDT positivity

| | Total | Positive | | Positive | |
|---|---|---|---|---|---|
| | samples | by | | by | |
| | tested n | RLDT | | qPCR | |
| | (%) | n (%) | p value | n (%) | p value |
| Age | | | | | |
| <12 months | 159 (49.1) | 26 (16.4) | 0.41 | 26 (16.4) | 0.44 |
| 12-23 months | 37 (11.4) | 9 (24.3) | | 8 (21.6) | |
| 24-59 months | 98 (30.2) | 21 (21.4) | | 22 (22.4) | |
| missing * | 30 (9.3) | 7 (23.3) | | 8 (26.7) | |
| Sex | | 63 | | 19.75 | |
| Male | 152 (46.9) | 34 (22.4) | 0.29 | 34 (22.4) | 0.35 |
| Female | 165 (50.9) | 29 (17.6) | | 30 (18.2) | |
| missing * | 7 (2.2) | 0 (0) | | 0 (0) | |
| Symptomatic | | | | | |
| No | 92(28.4) | 12(13) | 0.07 | 13(14.1) | 0.09 ² |
| Yes | 227(70.1) | 51(22.5) | | 51(22.5) | |
| missing * | 5(1.5) | 0(0) | | 0(0) | |
| Severity | | | | | |
| Mild/ | 287 (88.6) | 50 (17.4) | 1.0 ¹ | 51 (17.8) | 0.70 ¹ |
| Moderate | | | | | |
| Severe | 10 (3.1) | 1 (10) | | 2 (20) | |
| missing * | 27 (8.3) | 12 (44.4) | | 11 (40.7) | |
| Wash | | | | | |
| Adequate | 213 (65.7) | 40 (18.8) | 0.15 | 40 (18.8) | 0.70 |
| Inadequate | 62 (19.1) | 11 (17.7) | | 13 (21) | |
| missing | 49 (15.1) | 12 (24.5) | | 11 (22.4) | |
| Total | 324 (100) | 63 (19.4) | | 64 (19.8) | |

NOTE:
Chi square test was used to compare the association of baseline characteristics such as age, sex,
Note:
diarrhea severity and wash data against RLDT and qPCR ETEC positivity. P values less than 0.05 showing a statistically significant difference.
* Statistical significance (P < 0.05).

Performance of the RLDT against qPCR. The performance of the RLDT against qPCR is shown in Table 13. The prevalence of ETEC was 19.8% by qPCR and 19.4% by RLDT. The evaluation of the LT, STh and STp toxin genes sensitivity and specificity of the RLDT using a Ct 35 value cut-off 299 with a 95% confidence interval were, 90.7% (77.9-97.4) and 97.5% (94.8-99); (85.2% 300 (66.3-95.8) and 99.3% (97.5-99.9); and 100% (59.0-100) and 99.7% (98.3-100)), respectively. With the Ct cut off of 28, the sensitivity and specificity were higher (Table 14).

TABLE 13
Performance of RLDT against qPCR using a cut off of Ct35

| | Number of | Samples | Samples | | | | |
|---|---|---|---|---|---|---|---|
| | samples | positive | positive | False | False | Sensitivity | Specificity |
| Ct <= 35** | tested | by RLDT | by qPCR | positive | negative | (95% CI) | (95% CI) |
| LT | 319 | 46 | 43 | 7 | 4 | 90.7 (77.9-97.4) | 97.5 (94.8-99.0) |
| STp | 324 | 8 | 7 | 1 | 0 | 100 (59.0-100.0) | 99.7 (98.3-100.0) |
| STh | 317 | 25 | 27 | 2 | 4 | 85.2 (66.3-95.8) | 99.3 (97.5-99.9) |

Note:
35** a Ct value cutoff for both qPCR and the ETEC RLDT,
CI = Confidence Interval

TABLE 14
Performance of RLDT against qPCR using a cut off of Ct28

| | | number | | |
|---|---|---|---|---|
| Cut off | | positive | Sensitivity | Specificity |
| CT <=28 * | n | (%) | (95% CI) | (95% CI) |
| Lt | 319 | 37 (11.6) | 97.3 (85.8-99.9) | 96.5 (93.6-98.3) |
| Stp | 324 | 7 (2.2) | 100 (59-100) | 99.7 (98.3-100) |
| Sth | 316 | 24 (7.6) | 95.8 (78.9-99.9) | 99.3 (97.6-99.9) |

Note:
28** a Ct value cut off for both qPCR and the ETEC RLDT,
CI = Confidence Interval

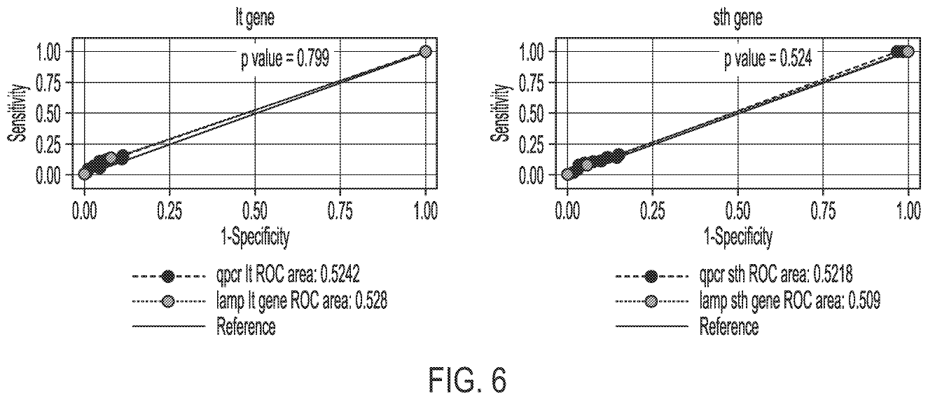

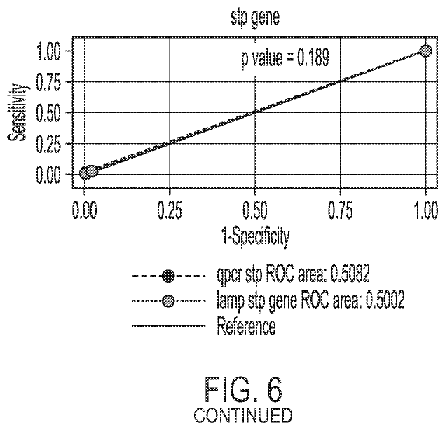

Performance of RLDT against qPCR by the clinical representation and AUC analysis. The performance of the RLDT against qPCR by the participants' clinical representation is shown in Table 15. The evaluation of symptomatic participants of the LT, STh and STp toxin genes sensitivity and specificity of the RLDT using the CT value cutoff of 35 with a 95% confidence interval was 91.4% (76.9-99.7), and 96.8% (93.2-98.8), 85.7% (63.7-97.0) and 99% (96.5-99.9) and 100% (54.1-100) and 100% (98.3-100), respectively. Similar results observed when asymptomatic cases were evaluated for sensitivity and specificity of LT, STh and STp (87.5% (47.4-99.7) and 98.8% (93.5-100), (83.3% (35.9-99.6) and 100% (95.8-100)) and (100% (2.5-100) and 98.9% (94-100), respectively. A comparison of the ETEC RLDT to qPCR tests for each target gene using Area Under the Curve (AUC) analysis to evaluate the performance of the two instruments. From the analysis, no significant difference was found between the ETEC RLDT to qPCR (FIG. 6).

TABLE 15
Performance of RLDT against qPCR by the clinical state of participants

| | | Number of | Samples | Samples | | | | |
|---|---|---|---|---|---|---|---|---|
| | | samples | positive | positive | False | False | Sensitivity | Specificity |
| Ct <= 35** | Clinical Status | tested | by RLDT | by qPCR | positive | negative | (95% CI) | (95% CI) |
| LT | Asymptomatic | 92 | 8 | 8 | 1 | 1 | 87.5 (47.4-99.7) | 98.8 (93.5-100) |
| | Symptomatic | 224 | 38 | 35 | 6 | 3 | 91.4 (76.9-98.2) | 96.8 (93.2-98.8) |
| STp | Asymptomatic | 92 | 2 | 1 | 1 | 0 | 100.0 (2.5-100.0) | 98.9 (94.0-100.0) |
| | Symptomatic | 227 | 6 | 6 | 0 | 0 | 100 (54.1-100.0) | 100.0 (98.3-100.0) |
| STh | Asymptomatic | 91 | 5 | 6 | 0 | 1 | 83.3 (35.9-99.6) | 100 (95.8-100.0) |
| | Symptomatic | 223 | 20 | 21 | 2 | 3 | 85.7 (63.7-97.0) | 99.0 (96.5-99.9) |

Note:
35** a Ct value cutoff for both qPCR and the ETEC RLDT,
CI = Confidence Interval

ETEC toxin gene distribution. ETEC expressing the heat Labile toxin (LT) had a frequency of 49% being the dominant expressed gene, followed by 34% of strains expressing the Heat stable toxin (ST) genes. The frequency of ETEC expressing the combination of both LT/ST toxins was 16%.

Seasonality. A seasonal trend of ETEC was observed over 12 months with high positivity rates between December and February (warm, rainy season) and a minor peak between April and May (dry season).

Discussion. This study is the first field evaluation of ETEC RLDT and establishes that it performed equally as the qPCR, as demonstrated by the specificity, sensitivity and AUC curves for each toxin gene LT, STh and STp. The performance of the RLDT was similar among ETEC positive diarrhea and asymptomatic cases. These findings are important as they support the use of the RLDT for screening for ETEC among children presenting with diarrhea at health facilities. In addition, its turnaround time and simplicity (not requiring skilled laboratory personnel for testing and results interpretation) makes it an effective method for resource-limited settings. The RLDT can also be implemented in these countries for ETEC disease surveillance which is important for obtaining meaningful disease burden data to inform policymakers and healthcare professionals for developing control and prevention programs. Similar studies which aimed at assessing LAMP platforms sensitivity and specificity against qPCR for the detection of Mycoplasma pneumonia (Ishiguro N, et al. Clinical laboratory. 2015; 61:603-606) and Leptospira spp (Suwancharoen D, et al. The Journal of veterinary medical science. 2016; 78: 1299-1302) found that both the LAMP assays had good sensitivity and specificity (99.1% and 100.0%) and (96.8% and 97.0%), respectively. These studies also concluded that the LAMP platforms are easy to use and comparable to the qPCR, as shown in this study.

It was also determined that across the stratified age groups, children between 12 to 59 months were at the highest risk of getting ETEC infection with prevalence of ˜22%. The overall prevalence of ETEC under 5 years old, in this study was ˜19%. The isolation rates of ETEC in this study is similar to previous studies that have reported the prevalence of ETEC in developing countries from Bangladesh, Turkey, Peru, Mexico, Egypt, Argentina, India, Nicaragua, and Tunisia which indicated a rate of 18-38% in children (Qadri F, et al. Clinical microbiology reviews. 2005; 18: 465-483; Al-Gallas N, et al. The American journal of tropical medicine and hygiene. 2007; 77: 571-582; Hien B T T, et al. Journal of clinical microbiology, 2008; 46: 996-1004; Bueris V, et al. Memorias do Instituto Oswaldo Cruz. 2007; 102: 839-844; and Işeri L, et al. Brazilian journal of microbiology: [publication of the Brazilian Society for Microbiology]. 2011; 42: 243-247). However, the ETEC prevalence in the study described herein was lower than what was reported in a previous study (40.7%) conducted in Zambia (Chisenga C C, et al. Pediatric Infect Dis. 2018; 3: 8) using Luminex Magpix GPP panel which uses x-TAG technology. This could be attributed to the different in testing platforms, technology and sensitivity of the assays.

The seasonal prevalence observed in this study is similar to what was reported in Kenya (Shah M, et al. Tropical Medicine and Health. 2016; 44: 1-8) which reported the seasonal variation of enteric bacterial pathogens among the hospitalized children with diarrhea. ETEC infections were found all year round with an increase during the warm rainy season and dry seasons (Shah M, et al. Tropical Medicine and Health. 2016; 44: 1-8). This information is important to inform policymakers and healthcare professionals to develop control and prevention programs including when to deploy the ETEC vaccines.

It was also found that in Lusaka, Zambia, among the circulating ETEC strains, the LT-ETEC strains was the highest followed by ST-ETEC and LT+ST-ETEC strains. Six percent of the ETEC strains were STp-ETEC. A similar distribution of toxin genes among ETEC strains was reported from Bolivia (LT 70%, LT+STh 23% and STh 7%) (Rodas C, et al. Brazilian Journal of Infectious Diseases. 2011; 15: 132-137). Michelo et al, also reported similar results, LT+STh being the most common toxin combination and LT+STh+STp being the least common in Zambia (Simuyandi M, et al. Arch Microbiol Immunology. 2019; 03). This suggests that vaccines such as ETVAX could be effective for this population and region.

Conclusion. The results demonstrated that the RLDT performed comparable to the qPCR assay. Additionally, the observed specificity and sensitivity are high evidencing that the RLDT can be used in a field setting to rapidly detect ETEC among patients presenting with diarrhea in the health facilities. This study provides support for using the method disclosed herein as a broader application of the RLDT as a simple and rapid diagnostic test for ETEC in the endemic countries where such simple assays are urgently needed. The results also show that LT-ETEC and ST-ETTEC strains were highly prevalent and ETEC positivity was highest in the warm rainy season.

### Example 3. Field Evaluation of RLDT for Detection of Shigella and Enterotoxigenic E. coli in India

Study design: Stool samples from 405 patients with diarrhea (including dysentery) under 5 years of age, seeking care in either the Infectious disease Hospital or BC Roy Children Hospital, Kolkata, India were tested in the study for ETEC and Shigella using RLDT, qPCR and culture.

Methods: OPCR: DNA was isolated from stool samples using a bead beater to disrupt cells. The cell slurry was centrifuged, and the supernatant was processed using the Qiagen QIAamp DNA stool extraction kit. The stool samples were screened using qPCR for detecting LT, STh STp and ipaH genes.

Culture followed by Colony PCR for ETEC: The stool samples were cultured on MacConkey agar. For the detection of ETEC, 5 lactose fermenting colonies from each sample were inoculated and stored separately in 1.5% nutrient agar in microfuge tubes. E. coli isolates were tested using multiplex PCR assay, targeting 3 toxin genes. For each 25 μl PCR mixture, boiled template is mixed with 15 μl of Master mix containing PCR buffer, MgCl2, dNTP, specific primers (Invitrogen), and Taq polymerase. The amplification was done as 94° C. for 5 min denaturation followed by 40 cycles at 94° C. (30 s), 55° C. (30 s), and 72° C. (60 s) and final elongation at 72° C. (60 s).

Culture method for Shigella: As followed at NICED, stool specimens were cultured onto XLD and HEA agar. The Shigella like colonies were selected for further biochemical analysis (TSI, LIA, Citrate, MIO, Indole) and confirmed serologically by slide agglutination using commercially purchased antisera (Denka Seiken Co. Ltd).

RLDT: Stool samples were processed and tested using the RLDT kits.

The results are shown in Tables 16 and 17.

TABLE 16
Sensitivity and specificity of Shigella RLDT comparing with qPCR and culture in India

| | | | | | | | |
|---|---|---|---|---|---|---|---|
| | | | Prevalence | | | | |
| | Total | Prevalence | by qPCR | False | False | | |
| | samples | by RLDT | or Culture | positive | negative | Sensitivity | Specificity |
| Targets | screened | (%) | (%) | (%) | (%) | (%) | (%) |

QPCR vs RLDT

| | | | | | | | | | |
|---|---|---|---|---|---|---|---|---|---|
| ipaH | 405 | 85 (21%) | 91 | (22.5%) | 0 | (0%) | 6 (6.6%) | 93.4 | 100 |

Culture vs RLDT

| | | | | | | | | | |
|---|---|---|---|---|---|---|---|---|---|
| ipaH | 405 | 85 (21%) | 35 | (8.6%) | 51 | (13.8%) | 1 (2.9%) | 97.1 | 86.2 |

TABLE 17
Sensitivity and specificity of ETEC RLDT comparing with qPCR and culture in India

| | | | | | | | |
|---|---|---|---|---|---|---|---|
| | | | Prevalence | | | | |
| | Total | Prevalence | by qPCR | False | False | | |
| | samples | by RLDT | or Culture | positive | negative | Sensitivity | Specificity |
| Targets | screened | (%) | (%) | (%) | (%) | (%) | (%) |

QPCR vs RLDT

| | | | | | | | | | | | |
|---|---|---|---|---|---|---|---|---|---|---|---|
| ETEC | 404 | 68 | (16.8%) | 58 | (14.4%) | 11 | (3.2%) | 1 | (1.7%) | 98.3 | 96.8 |
| Total | | | | | | | | | | | |
| LT | 404 | 47 | (11.6%) | 43 | (10.6%) | 6 | (1.7%) | 2 | (4.7%) | 95.3 | 98.3 |
| STh | 404 | 22 | (5.4%) | 24 | (5.9%) | 0 | (0%) | 2 | (8.3%) | 91.7 | 100 |
| STp | 404 | 26 | (6.4%) | 18 | (4.5%) | 8 | (2.1%) | 0 | (0%) | 100 | 97.9 |

Culture Vs RLDT

| | | | | | | | | | | | |
|---|---|---|---|---|---|---|---|---|---|---|---|
| ETEC | 405 | 68 | (16.8%) | 15 | (3.7%) | 54 | (13.8%) | 1 | (6.7%) | 93.3 | 86.2 |
| Total | | | | | | | | | | | |
| ETEC | 405 | 47 | (11.6%) | 8 | (2%) | 39 | (9.8%) | 0 | (0%) | 100 | 90.2 |
| LT | | | | | | | | | | | |
| ETEC | 405 | 44 | (10.9%) | 12 | (3%) | 33 | (8.4%) | 1 | (8.3%) | 91.7 | 91.6 |
| ST | | | | | | | | | | | |

Note:
Culture for ETEC and Shigella has been reported to be much less sensitive than molecular tests.

1. A method of detecting a target microorganism in a sample, the method comprising:
a) obtaining or having obtained a sample from a subject, wherein the sample comprises or is suspected of comprising the target microorganism;
b) contacting the sample with a lysis solution to form a mixture;
c) filtering the mixture of b) through a filter to form a filtered mixture, wherein the filtered mixture comprises DNA or RNA of the target microorganism;
d) contacting the filtered mixture of c) with loop mediated isothermal amplification (LAMP) reagents and one or more primer sets specific to the DNA or RNA of the target microorganism, wherein the LAMP reagents and the one or more primer sets are lyophilized;
e) amplifying the DNA or RNA of the target microorganism, thereby producing one or more amplicons; and
f) detecting the presence or absence of the one or more amplicons;
wherein the presence of the one or more of the amplicons indicates the presence of the target microorganism.

2. The method of claim 1, wherein the one or more primer sets in step d) are specific for one or more genes specific to the target microorganism.

3. The method of claim 1, wherein the one or more primer sets in step d) are specific for one or more of heat labile toxin (LT) gene, heat stable toxin (STh, and STp) gene, eae gene, bfpA gene, aaiC gene, aatA gene, CVD432 gene, F1845 gene, stx1 gene, stx2 gene, rfbO157 gene, invasion plasmid gene (ipaH), cholera toxin A (ctxA) gene, O1 lipopolysaccharide (O1rfb) gene, O139 gene, 16S gene, IS 6110 gene, MPB 64 gene, 16 S RNA gene, rpoB gene, FliC flagellar gene, invA gene, norovirus G1 gene, norovirus G2 gene, RdRp gene, capsid gene, NSP3 gene, hexon gene, ORF-1 gene, E gene, M gene, N gene, S gene, L1 gene, E6 gene, or E7 gene.

4. The method of claim 1, wherein the filter comprises a LAMP inhibitor control DNA and wherein the filtered mixture of step c) comprises DNA or RNA of the target microorganism and the LAMP inhibitor control DNA.

5. (canceled)

6. The method of claim 1, wherein the target microorganism is E. coli, Shigella spp, Vibro cholerae, non-cholera Vibro spp, Campylobacter spp, Mycobacterium spp, Salmonella spp, an enteric virus, a coronavirus, or human papillomavirus.

7. The method of claim 6, wherein the E. coli is an enterotoxigenic E. coli, an enteropathogenic E. coli, an enteroaggregative E. coli, an enteroinvasive E. coli, an enterohemorrhagic E. coli, a shiga toxin-producing E. coli, a verocytotoxin-producing E. coli or a diffusely adherent E. coli.

8.-17. (canceled)

18. The method of claim 6, wherein the Mycobacterium spp is M. tuberculosis.

19. The method of claim 18, wherein the one or more primer sets of step d) are specific to the IS 6110 gene, the MPB 64 gene, the 16 S rRNA gene, or the rpoB gene.

20. The method of claim 6, wherein the Salmonella spp is S. typhi or S. paratyphi.

21. (canceled)

22. The method of claim 6, wherein the enteric virus is norovirus, sapovirus, astrovirus, rotavirus, or adenovirus.

23.-27. (canceled)

28. The method of claim 6, wherein the coronavirus is SARS-CoV-2.

29. The method of claim 28, wherein the one or more primer sets of step d) are specific to the ORF-1 gene, the E gene, the M gene, the N gene and the S gene.

30. The method of claim 6, wherein the Shigella spp is S. flexneri, S. sonnei, S. dysenteriae, or S. boydii.

31. The method of claim 30, wherein the one or more primer sets of step d) are specific to the invasion plasmid gene (ipaH).

32. The method of claim 6, wherein the target microorganism is the human papillomavirus and wherein the one or more primer sets of step d) are specific to the L1 gene, E6 gene or E7 gene.

33. (canceled)

34. The method ofany of claim 1, wherein the sample is a blood, stool, sputum, oropharyngeal, nasopharyngeal, pap smear, or saliva sample.

35. A method of detecting pathogenic E. coli in a sample, the method comprising:
a) obtaining or having obtained a sample from a subject, wherein the sample comprises or is suspected of comprising the pathogenic E. coli;
b) contacting the sample with a lysis solution to form a mixture;
c) filtering the mixture of b) through a filter to form a filtered mixture, wherein the filtered mixture comprises DNA or RNA of the pathogenic E. coli; and
d) contacting the filtered mixture of c) with loop mediated isothermal amplification (LAMP) reagents and one or more primer sets specific to the DNA or RNA of the pathogenic E. coli, wherein the LAMP reagents and the one or more primer sets are lyophilized;
e) amplifying DNA or RNA of the pathogenic E. coli, thereby producing one or more amplicons; and
f) detecting the presence or absence of the one or more amplicons;
wherein the presence of the one or more amplicons indicates the presence of the pathogenic E. coli.

36.-59. (canceled)

60. A kit for detecting a target microorganism in a sample, the kit comprising: a lysis buffer; a filter; a lyophilized buffer; loop mediated isothermal amplification (LAMP) reagents; and one or more primer sets specific to the DNA or RNA of the target microorganism, wherein the LAMP reagents and the one or more primer sets are lyophilized.

61. (canceled)

62. The kit of claim 60, wherein the one or more primer sets are specific for one or more of the heat labile toxin (LT) gene, heat stable toxin (STh, and STp) gene, eae gene, bfpA gene, aaiC gene, aatA gene, CVD432 gene, F1845 gene, stx1 gene, stx2 gene, rfbO157 gene, invasion plasmid gene (ipaH), cholera toxin A (ctxA) gene, O1 lipopolysaccharide (O1rfb) gene, O139 gene, 16S gene, IS 6110 gene, MPB 64 gene, 16 S RNA gene, rpoB gene, FliC flagellar gene, invA gene, norovirus G1 gene, norovirus G2 gene, RdRp gene, capsid gene, NSP3 gene, hexon gene, ORF-1 gene, E gene, M gene, N gene, S gene, L1 gene, E6 gene, or E7 gene.

63. The kit of claim 60, wherein the filter comprises a LAMP inhibitor control DNA.


SEQUENCE LISTING (1407 sequences)

SEQ ID NO: 1 - DNA, 24 bp, Artificial Sequence
  Synthetic construct
atcgtgttaattttggtgtgattg

SEQ ID NO: 2 - DNA, 21 bp, Artificial Sequence
  Synthetic construct
ctgggtctcctcattacaagt

SEQ ID NO: 3 - DNA, 50 bp, Artificial Sequence
  Synthetic construct
aaccatcctctgccggagctatattgaacgattacatcgtaacagggaat

SEQ ID NO: 4 - DNA, 46 bp, Artificial Sequence
  Synthetic construct
ttcccaccggatcaccaagctgttcttgatgaatctccacaacctt

SEQ ID NO: 5 - DNA, 24 bp, Artificial Sequence
  Synthetic construct
gatttctgtaataccggtctctat

SEQ ID NO: 6 - DNA, 24 bp, Artificial Sequence
  Synthetic construct
agaagaaccctggattcatcatgc

SEQ ID NO: 7 - DNA, 25 bp, Artificial Sequence
  Synthetic construct
gcaaaatccgtttaactaatctcaa

SEQ ID NO: 8 - DNA, 24 bp, Artificial Sequence
  Synthetic construct
acagcagtaaaatgtgttgttcat

SEQ ID NO: 9 - DNA, 49 bp, Artificial Sequence
  Synthetic construct
aagaggggaaagataatacagaaattttttaaacaacatgacgggaggt

SEQ ID NO: 10 - DNA, 54 bp, Artificial Sequence
  Synthetic construct
tagtcagtcaactgaatcacttgatttttctgttgttttttacaacatcacact

SEQ ID NO: 11 - DNA, 21 bp, Artificial Sequence
  Synthetic construct
gccaacattagctttttcatg

SEQ ID NO: 12 - DNA, 25 bp, Artificial Sequence
  Synthetic construct
caaaagagaaaattacattagagac

SEQ ID NO: 13 - DNA, 19 bp, Artificial Sequence
  Synthetic construct
ctcaggatgctaaaccagt

SEQ ID NO: 14 - DNA, 25 bp, Artificial Sequence
  Synthetic construct
cagaacaaatataaagggaactgtt

SEQ ID NO: 15 - DNA, 51 bp, Artificial Sequence
  Synthetic construct
tcatgctttcaggaccacttttattgagtcttcaaaagaaaaaatcacact

SEQ ID NO: 16 - DNA, 50 bp, Artificial Sequence
  Synthetic construct
agtagcaattactgctgtgaattgtccctttatattattaatagcacccg

SEQ ID NO: 17 - DNA, 17 bp, Artificial Sequence
  Synthetic construct
gttgtaatcctgcttgt

SEQ ID NO: 18 - DNA, 19 bp, Artificial Sequence
  Synthetic construct
aattctggaggacattgcc

SEQ ID NO: 19 - DNA, 18 bp, Artificial Sequence
  Synthetic construct
cgtacgcttcagtacagc

SEQ ID NO: 20 - DNA, 40 bp, Artificial Sequence
  Synthetic construct
cttcacggcagtggagagctgagatagaagtctacctggc

SEQ ID NO: 21 - DNA, 39 bp, Artificial Sequence
  Synthetic construct
tatggcgtgtcgggagtgattcattctcttcacggcttc

SEQ ID NO: 22 - DNA, 18 bp, Artificial Sequence
  Synthetic construct
ctctgcgagcatggtctg

SEQ ID NO: 23 - DNA, 18 bp, Artificial Sequence
  Synthetic construct
cactgccgaagctatggt

SEQ ID NO: 24 - DNA, 21 bp, Artificial Sequence
  Synthetic construct
tgagatagaagtctacctggc

SEQ ID NO: 25 - DNA, 19 bp, Artificial Sequence
  Synthetic construct
cttcacggcagtggagagc

SEQ ID NO: 26 - DNA, 20 bp, Artificial Sequence
  Synthetic construct
ttcattctcttcacggcttc

SEQ ID NO: 27 - DNA, 19 bp, Artificial Sequence
  Synthetic construct
tatggcgtgtcgggagtga

SEQ ID NO: 28 - DNA, 21 bp, Artificial Sequence
  Synthetic construct
gttatatcgggcagattctag

SEQ ID NO: 29 - DNA, 18 bp, Artificial Sequence
  Synthetic construct
gtttgacccactaagtgg

SEQ ID NO: 30 - DNA, 43 bp, Artificial Sequence
  Synthetic construct
tttgagtacctcggtcaaagtacacctcctgatgaaataaagc

SEQ ID NO: 31 - DNA, 42 bp, Artificial Sequence
  Synthetic construct
tgatcatgcaagaggaactcagattgaggtggaaacatatcc

SEQ ID NO: 32 - DNA, 23 bp, Artificial Sequence
  Synthetic construct
tctgtcctcttggcataagacca

SEQ ID NO: 33 - DNA, 22 bp, Artificial Sequence
  Synthetic construct
cgggatttgttaggcacgatga

SEQ ID NO: 34 - DNA, 20 bp, Artificial Sequence
  Synthetic construct
acctcctgatgaaataaagc

SEQ ID NO: 35 - DNA, 23 bp, Artificial Sequence
  Synthetic construct
tttgagtacctcggtcaaagtac

SEQ ID NO: 36 - DNA, 20 bp, Artificial Sequence
  Synthetic construct
attgaggtggaaacatatcc

SEQ ID NO: 37 - DNA, 22 bp, Artificial Sequence
  Synthetic construct
tgatcatgcaagaggaactcag

SEQ ID NO: 38 - DNA, 22 bp, Artificial Sequence
  Synthetic construct
tcttctgctaccagtggcgtac

SEQ ID NO: 39 - DNA, 22 bp, Artificial Sequence
  Synthetic construct
ttcaagtggagcacttgggcta

SEQ ID NO: 40 - DNA, 42 bp, Artificial Sequence
  Synthetic construct
tgcaaaacgggcgacgtttaggctcgtcgacagcatcgagca

SEQ ID NO: 41 - DNA, 48 bp, Artificial Sequence
  Synthetic construct
tgatccgacaagcccaaatgccactcgatgttgaggcgaagtttaggt

SEQ ID NO: 42 - DNA, 23 bp, Artificial Sequence
  Synthetic construct
aacacctcctgcataactcttgc

SEQ ID NO: 43 - DNA, 19 bp, Artificial Sequence
  Synthetic construct
gctcgtattgcggcggtaa

SEQ ID NO: 44 - DNA, 19 bp, Artificial Sequence
  Synthetic construct
tcgtcgacagcatcgagca

SEQ ID NO: 45 - DNA, 23 bp, Artificial Sequence
  Synthetic construct
tgcaaaacgggcgacgtttaggc

SEQ ID NO: 46 - DNA, 24 bp, Artificial Sequence
  Synthetic construct
tcgatgttgaggcgaagtttaggt

SEQ ID NO: 47 - DNA, 24 bp, Artificial Sequence
  Synthetic construct
tgatccgacaagcccaaatgccac

SEQ ID NO: 48 - DNA, 17 bp, Artificial Sequence
  Synthetic construct
ccaaggcagcatcaatt

SEQ ID NO: 49 - DNA, 19 bp, Artificial Sequence
  Synthetic construct
tctatgccgctacatatga

SEQ ID NO: 50 - DNA, 41 bp, Artificial Sequence
  Synthetic construct
tctgaagttgttactgctaccggcctgttctgaagttatgt

SEQ ID NO: 51 - DNA, 39 bp, Artificial Sequence
  Synthetic construct
gccaccaaatttcacagctccaggtgcaattactgctaa

SEQ ID NO: 52 - DNA, 18 bp, Artificial Sequence
  Synthetic construct
cttagcaagcgaccttga

SEQ ID NO: 53 - DNA, 17 bp, Artificial Sequence
  Synthetic construct
ttgagcaacgccagtac

SEQ ID NO: 54 - DNA, 19 bp, Artificial Sequence
  Synthetic construct
gcctgttctgaagttatgt

SEQ ID NO: 55 - DNA, 22 bp, Artificial Sequence
  Synthetic construct
tctgaagttgttactgctaccg

SEQ ID NO: 56 - DNA, 19 bp, Artificial Sequence
  Synthetic construct
caggtgcaattactgctaa

SEQ ID NO: 57 - DNA, 20 bp, Artificial Sequence
  Synthetic construct
gccaccaaatttcacagctc

SEQ ID NO: 58 - DNA, 17 bp, Artificial Sequence
  Synthetic construct
tgtggacaggagatcgc

SEQ ID NO: 59 - DNA, 17 bp, Artificial Sequence
  Synthetic construct
acttgtccagcagtcgc

SEQ ID NO: 60 - DNA, 42 bp, Artificial Sequence
  Synthetic construct
tagcgccatccacgcttgattttcttctgcccgaattcgtaa

SEQ ID NO: 61 - DNA, 43 bp, Artificial Sequence
  Synthetic construct
ctggtcagttggtaccggagttttctgtcgaagaacctgctac

SEQ ID NO: 62 - DNA, 21 bp, Artificial Sequence
  Synthetic construct
cgtccttagacgccatcatca

SEQ ID NO: 63 - DNA, 21 bp, Artificial Sequence
  Synthetic construct
gcttctgaccctcttgcaatg

SEQ ID NO: 64 - DNA, 19 bp, Artificial Sequence
  Synthetic construct
cttctgcccgaattcgtaa

SEQ ID NO: 65 - DNA, 19 bp, Artificial Sequence
  Synthetic construct
tagcgccatccacgcttga

SEQ ID NO: 66 - DNA, 19 bp, Artificial Sequence
  Synthetic construct
ctgtcgaagaacctgctac

SEQ ID NO: 67 - DNA, 20 bp, Artificial Sequence
  Synthetic construct
ctggtcagttggtaccggag

SEQ ID NO: 68 - DNA, 20 bp, Artificial Sequence
  Synthetic construct
cagacaagagccaatgttca

SEQ ID NO: 69 - DNA, 18 bp, Artificial Sequence
  Synthetic construct
caatagcggcaccaacaa

SEQ ID NO: 70 - DNA, 41 bp, Artificial Sequence
  Synthetic construct
cgtcattcgacgccatcttcagattctcagatctgagcacg

SEQ ID NO: 71 - DNA, 40 bp, Artificial Sequence
  Synthetic construct
catctgatgggtccgcagctccagagccataacctcatta

SEQ ID NO: 72 - DNA, 20 bp, Artificial Sequence
  Synthetic construct
ctgggagccagattgcgatc

SEQ ID NO: 73 - DNA, 20 bp, Artificial Sequence
  Synthetic construct
aacctcgtcccagaggtcaa

SEQ ID NO: 74 - DNA, 20 bp, Artificial Sequence
  Synthetic construct
gattctcagatctgagcacg

SEQ ID NO: 75 - DNA, 21 bp, Artificial Sequence
  Synthetic construct
cgtcattcgacgccatcttca

SEQ ID NO: 76 - DNA, 21 bp, Artificial Sequence
  Synthetic construct
tccagagccataacctcatta

SEQ ID NO: 77 - DNA, 19 bp, Artificial Sequence
  Synthetic construct
catctgatgggtccgcagc

SEQ ID NO: 78 - DNA, 23 bp, Artificial Sequence
  Synthetic construct
ctgcttaacacaagttgagtagg

SEQ ID NO: 79 - DNA, 20 bp, Artificial Sequence
  Synthetic construct
ttccttaggtaccgtcagaa

SEQ ID NO: 80 - DNA, 52 bp, Artificial Sequence
  Synthetic construct
ggaccgtgtctcagttccagtgtgacggatgagactatatagtatcagctag

SEQ ID NO: 81 - DNA, 49 bp, Artificial Sequence
  Synthetic construct
cgggaggcagcagtagggaatattgctaagaaaaggagtttacgctccg

SEQ ID NO: 82 - DNA, 23 bp, Artificial Sequence
  Synthetic construct
gttaagcgtcatagccttggtaa

SEQ ID NO: 83 - DNA, 18 bp, Artificial Sequence
  Synthetic construct
gcgtggaggatgacactt

SEQ ID NO: 84 - DNA, 42 bp, Artificial Sequence
  Synthetic construct
acagcaccgccacctatagtagaagcttttttaaactagggc

SEQ ID NO: 85 - DNA, 46 bp, Artificial Sequence
  Synthetic construct
aggcagcagaacttacgcattgagtttgaaaaaacattctacctct

SEQ ID NO: 86 - DNA, 22 bp, Artificial Sequence
  Synthetic construct
gcaagacaatattattgatcgc

SEQ ID NO: 87 - DNA, 18 bp, Artificial Sequence
  Synthetic construct
ctttcacaggctgcactt

SEQ ID NO: 88 - DNA, 22 bp, Artificial Sequence
  Synthetic construct
ctagctgctactacagaaccac

SEQ ID NO: 89 - DNA, 20 bp, Artificial Sequence
  Synthetic construct
catcaagcttcacaaggaaa

SEQ ID NO: 90 - DNA, 46 bp, Artificial Sequence
  Synthetic construct
aagagataaacaccatgatcccagtcatgaatgagcttactttagc

SEQ ID NO: 91 - DNA, 44 bp, Artificial Sequence
  Synthetic construct
gcggcaaagacttatgataaagctaccgccattcctaaaacaag

SEQ ID NO: 92 - DNA, 18 bp, Artificial Sequence
  Synthetic construct
tgggagcgtttttgatct

SEQ ID NO: 93 - DNA, 18 bp, Artificial Sequence
  Synthetic construct
aatcaaactcaccgccat

SEQ ID NO: 94 - DNA, 20 bp, Artificial Sequence
  Synthetic construct
ccactacagcaaaggtgatg

SEQ ID NO: 95 - DNA, 19 bp, Artificial Sequence
  Synthetic construct
ccacgatagcctttatgga

SEQ ID NO: 96 - DNA, 22 bp, Artificial Sequence
  Synthetic construct
actgattgccgcaattgaattg

SEQ ID NO: 97 - DNA, 22 bp, Artificial Sequence
  Synthetic construct
gactatcagtcagaggttgaca

SEQ ID NO: 98 - DNA, 42 bp, Artificial Sequence
  Synthetic construct
agcggcgcaacatttcaggtttggtctcggtcagatatgtga

SEQ ID NO: 99 - DNA, 41 bp, Artificial Sequence
  Synthetic construct
ttgcctgccatcgattccgttctctatgtgcatacggagct

SEQ ID NO: 100 - DNA, 22 bp, Artificial Sequence
  Synthetic construct
ccgggcagtcaacatatagact

SEQ ID NO: 101 - DNA, 22 bp, Artificial Sequence
  Synthetic construct
tgtgttgcgatattccgaacat

SEQ ID NO: 102 - DNA, 22 bp, Artificial Sequence
  Synthetic construct
ttggtctcggtcagatatgtga

SEQ ID NO: 103 - DNA, 20 bp, Artificial Sequence
  Synthetic construct
agcggcgcaacatttcaggt

SEQ ID NO: 104 - DNA, 21 bp, Artificial Sequence
  Synthetic construct
tctctatgtgcatacggagct

SEQ ID NO: 105 - DNA, 20 bp, Artificial Sequence
  Synthetic construct
ttgcctgccatcgattccgt

SEQ ID NO: 106 - DNA, 22 bp, Artificial Sequence
  Synthetic construct
cctcattacaagtatcacctgt

SEQ ID NO: 107 - DNA, 22 bp, Artificial Sequence
  Synthetic construct
gctcacttagcaggacagtcta

SEQ ID NO: 108 - DNA, 40 bp, Artificial Sequence
  Synthetic construct
agctccggcagaggatggttgtggtgcatgatgaatccag

SEQ ID NO: 109 - DNA, 43 bp, Artificial Sequence
  Synthetic construct
ccacctaacgcagaaacctcctatgttatagcgacagcaccaa

SEQ ID NO: 110 - DNA, 18 bp, Artificial Sequence
  Synthetic construct
ccaccggatcaccaagct

SEQ ID NO: 111 - DNA, 19 bp, Artificial Sequence
  Synthetic construct
gggtgagggctgtatacgc

SEQ ID NO: 112 - DNA, 21 bp, Artificial Sequence
  Synthetic construct
tgtggtgcatgatgaatccag

SEQ ID NO: 113 - DNA, 19 bp, Artificial Sequence
  Synthetic construct
agctccggcagaggatggt

SEQ ID NO: 114 - DNA, 21 bp, Artificial Sequence
  Synthetic construct
atgttatagcgacagcaccaa

SEQ ID NO: 115 - DNA, 22 bp, Artificial Sequence
  Synthetic construct
ccacctaacgcagaaacctcct

SEQ ID NO: 116 - DNA, 21 bp, Artificial Sequence
  Synthetic construct
aaccttgtggtgcatgatgaa

SEQ ID NO: 117 - DNA, 22 bp, Artificial Sequence
  Synthetic construct
gctcacttagcaggacagtcta

SEQ ID NO: 118 - DNA, 40 bp, Artificial Sequence
  Synthetic construct
agctccggcagaggatggttccagggttcttctctccaag

SEQ ID NO: 119 - DNA, 43 bp, Artificial Sequence; identical to SEQ ID NO: 109
  Synthetic construct

SEQ ID NO: 120 - DNA, 22 bp, Artificial Sequence
  Synthetic construct
acagattagcaggtttcccacc

SEQ ID NO: 121 - DNA, 19 bp, Artificial Sequence
  Synthetic construct
gggtgagggctgtatacgc

SEQ ID NO: 122 - DNA, 21 bp, Artificial Sequence
  Synthetic construct
tccagggttcttctctccaag

SEQ ID NO: 123 - DNA, 19 bp, Artificial Sequence
  Synthetic construct
agctccggcagaggatggt

SEQ ID NO: 124 - DNA, 21 bp, Artificial Sequence
  Synthetic construct
atgttatagcgacagcaccaa

SEQ ID NO: 125 - DNA, 22 bp, Artificial Sequence
  Synthetic construct
ccacctaacgcagaaacctcct

SEQ ID NO: 126 - DNA, 22 bp, Artificial Sequence
  Synthetic construct
aaccttgtggtgcatgatgaat

SEQ ID NO: 127 - DNA, 22 bp, Artificial Sequence
  Synthetic construct
gctcacttagcaggacagtcta

SEQ ID NO: 128 - DNA, 39 bp, Artificial Sequence
  Synthetic construct
agctccggcagaggatggtcagggttcttctctccaagc

SEQ ID NO: 129 - DNA, 43 bp, Artificial Sequence; identical to SEQ ID NO: 109
  Synthetic construct

SEQ ID NO: 130 - DNA, 22 bp, Artificial Sequence
  Synthetic construct
acagattagcaggtttcccacc

SEQ ID NO: 131 - DNA, 19 bp, Artificial Sequence
  Synthetic construct
gggtgagggctgtatacgc

SEQ ID NO: 132 - DNA, 20 bp, Artificial Sequence
  Synthetic construct
cagggttcttctctccaagc

SEQ ID NO: 133 - DNA, 19 bp, Artificial Sequence
  Synthetic construct
agctccggcagaggatggt

SEQ ID NO: 134 - DNA, 21 bp, Artificial Sequence
  Synthetic construct
atgttatagcgacagcaccaa

SEQ ID NO: 135 - DNA, 22 bp, Artificial Sequence
  Synthetic construct
ccacctaacgcagaaacctcct

SEQ ID NO: 136 - DNA, 20 bp, Artificial Sequence
  Synthetic construct
acaaccttgtggtgcatgat

SEQ ID NO: 137 - DNA, 22 bp, Artificial Sequence
  Synthetic construct
gctcacttagcaggacagtcta

SEQ ID NO: 138 - DNA, 41 bp, Artificial Sequence
  Synthetic construct
agctccggcagaggatggtaatccagggttcttctctccaa

SEQ ID NO: 139 - DNA, 42 bp, Artificial Sequence
  Synthetic construct
actgtcctgctaagtgagcactttatgatcacgcgagaggaa

SEQ ID NO: 140 - DNA, 22 bp, Artificial Sequence
  Synthetic construct
ttctgcgttaggtggaatacca

SEQ ID NO: 141 - DNA, 21 bp, Artificial Sequence
  Synthetic construct
atctgacaaagccggtttgtg

SEQ ID NO: 142 - DNA, 20 bp, Artificial Sequence
  Synthetic construct
tcgttcatcaatcacaccaa

SEQ ID NO: 143 - DNA, 21 bp, Artificial Sequence
  Synthetic construct
aggcgtatacagccctcaccc

SEQ ID NO: 144 - DNA, 20 bp, Artificial Sequence
  Synthetic construct
ttatgatcacgcgagaggaa

SEQ ID NO: 145 - DNA, 22 bp, Artificial Sequence
  Synthetic construct
actgtcctgctaagtgagcact

SEQ ID NO: 146 - DNA, 22 bp, Artificial Sequence
  Synthetic construct
tctgccggagctatattcagat

SEQ ID NO: 147 - DNA, 19 bp, Artificial Sequence
  Synthetic construct
accgtgctgactctagacc

SEQ ID NO: 148 - DNA, 43 bp, Artificial Sequence
  Synthetic construct
agcgacagcaccaaatatgttttggtattccacctaacgcaga

SEQ ID NO: 149 - DNA, 42 bp, Artificial Sequence; identical to SEQ ID NO: 139
  Synthetic construct

SEQ ID NO: 150 - DNA, 22 bp, Artificial Sequence
  Synthetic construct
cagccctcacccatatgaacag

SEQ ID NO: 151 - DNA, 21 bp, Artificial Sequence
  Synthetic construct
atctgacaaagccggtttgtg

SEQ ID NO: 152 - DNA, 21 bp, Artificial Sequence
  Synthetic construct
tggtattccacctaacgcaga

SEQ ID NO: 153 - DNA, 22 bp, Artificial Sequence
  Synthetic construct
agcgacagcaccaaatatgttt

SEQ ID NO: 154 - DNA, 20 bp, Artificial Sequence
  Synthetic construct
ttatgatcacgcgagaggaa

SEQ ID NO: 155 - DNA, 22 bp, Artificial Sequence
  Synthetic construct
actgtcctgctaagtgagcact

SEQ ID NO: 156 - DNA, 22 bp, Artificial Sequence
  Synthetic construct
cctcattacaagtatcacctgt

SEQ ID NO: 157 - DNA, 19 bp, Artificial Sequence
  Synthetic construct
ctgcgttaggtggaatacc

SEQ ID NO: 158 - DNA, 37 bp, Artificial Sequence
  Synthetic construct
ggtttcccaccggatcaccaccttgtggtgcatgatg

SEQ ID NO: 159 - DNA, 44 bp, Artificial Sequence
  Synthetic construct
cctctgccggagctatattcagggtgtgattgatgaacgattac

SEQ ID NO: 160 - DNA, 20 bp, Artificial Sequence
  Synthetic construct
tggagagaagaaccctggat

SEQ ID NO: 161 - DNA, 21 bp, Artificial Sequence
  Synthetic construct
accggtctctatattccctgt

SEQ ID NO: 162 - DNA, 18 bp, Artificial Sequence
  Synthetic construct
accttgtggtgcatgatg

SEQ ID NO: 163 - DNA, 19 bp, Artificial Sequence
  Synthetic construct
ggtttcccaccggatcacc

SEQ ID NO: 164 - DNA, 22 bp, Artificial Sequence
  Synthetic construct
ggtgtgattgatgaacgattac

SEQ ID NO: 165 - DNA, 22 bp, Artificial Sequence
  Synthetic construct
cctctgccggagctatattcag

SEQ ID NO: 166 - DNA, 22 bp, Artificial Sequence
  Synthetic construct
cctcattacaagtatcacctgt

SEQ ID NO: 167 - DNA, 19 bp, Artificial Sequence
  Synthetic construct
ctgcgttaggtggaatacc

SEQ ID NO: 168 - DNA, 38 bp, Artificial Sequence
  Synthetic construct
caggtttcccaccggatcacaccttgtggtgcatgatg

SEQ ID NO: 169 - DNA, 44 bp, Artificial Sequence; identical to SEQ ID NO: 159
  Synthetic construct

SEQ ID NO: 170 - DNA, 20 bp, Artificial Sequence
  Synthetic construct
tggagagaagaaccctggat

SEQ ID NO: 171 - DNA, 21 bp, Artificial Sequence
  Synthetic construct
accggtctctatattccctgt

SEQ ID NO: 172 - DNA, 18 bp, Artificial Sequence
  Synthetic construct
accttgtggtgcatgatg

SEQ ID NO: 173 - DNA, 20 bp, Artificial Sequence
  Synthetic construct
caggtttcccaccggatcac

SEQ ID NO: 174 - DNA, 22 bp, Artificial Sequence
  Synthetic construct
ggtgtgattgatgaacgattac

SEQ ID NO: 175 - DNA, 22 bp, Artificial Sequence
  Synthetic construct
cctctgccggagctatattcag

SEQ ID NO: 176 - DNA, 22 bp, Artificial Sequence
  Synthetic construct
cctcattacaagtatcacctgt

SEQ ID NO: 177 - DNA, 19 bp, Artificial Sequence
  Synthetic construct
ctgcgttaggtggaatacc

SEQ ID NO: 178 - DNA, 40 bp, Artificial Sequence
  Synthetic construct
acagattagcaggtttcccaccaccttgtggtgcatgatg

SEQ ID NO: 179 - DNA, 44 bp, Artificial Sequence; identical to SEQ ID NO: 159
  Synthetic construct

SEQ ID NO: 180 - DNA, 20 bp, Artificial Sequence
  Synthetic construct
tggagagaagaaccctggat

SEQ ID NO: 181 - DNA, 21 bp, Artificial Sequence
  Synthetic construct
accggtctctatattccctgt

SEQ ID NO: 182 - DNA, 18 bp, Artificial Sequence
  Synthetic construct
accttgtggtgcatgatg

SEQ ID NO: 183 - DNA, 22 bp, Artificial Sequence
  Synthetic construct
acagattagcaggtttcccacc

SEQ ID NO: 184 - DNA, 22 bp, Artificial Sequence
  Synthetic construct
ggtgtgattgatgaacgattac

SEQ ID NO: 185 - DNA, 22 bp, Artificial Sequence
  Synthetic construct
cctctgccggagctatattcag

SEQ ID NO: 186 - DNA, 22 bp, Artificial Sequence
  Synthetic construct
cctcattacaagtatcacctgt

SEQ ID NO: 187 - DNA, 19 bp, Artificial Sequence
  Synthetic construct
ctgcgttaggtggaatacc

SEQ ID NO: 188 - DNA, 38 bp, Artificial Sequence
  Synthetic construct
gtttcccaccggatcaccaaaccttgtggtgcatgatg

SEQ ID NO: 189 - DNA, 44 bp, Artificial Sequence; identical to SEQ ID NO: 159
  Synthetic construct

SEQ ID NO: 190 - DNA, 20 bp, Artificial Sequence
  Synthetic construct
tggagagaagaaccctggat

SEQ ID NO: 191 - DNA, 21 bp, Artificial Sequence
  Synthetic construct
accggtctctatattccctgt

SEQ ID NO: 192 - DNA, 18 bp, Artificial Sequence
  Synthetic construct
accttgtggtgcatgatg

SEQ ID NO: 193 - DNA, 20 bp, Artificial Sequence
  Synthetic construct
gtttcccaccggatcaccaa

SEQ ID NO: 194 - DNA, 22 bp, Artificial Sequence
  Synthetic construct
ggtgtgattgatgaacgattac

SEQ ID NO: 195 - DNA, 22 bp, Artificial Sequence
  Synthetic construct
cctctgccggagctatattcag

SEQ ID NO: 196 - DNA, 22 bp, Artificial Sequence
  Synthetic construct
cctcattacaagtatcacctgt

SEQ ID NO: 197 - DNA, 19 bp, Artificial Sequence
  Synthetic construct
ctgcgttaggtggaatacc

SEQ ID NO: 198 - DNA, 39 bp, Artificial Sequence
  Synthetic construct
agattagcaggtttcccaccgaccttgtggtgcatgatg

SEQ ID NO: 199 - DNA, 44 bp, Artificial Sequence; identical to SEQ ID NO: 159
  Synthetic construct

SEQ ID NO: 200 - DNA, 20 bp, Artificial Sequence
  Synthetic construct
tggagagaagaaccctggat

SEQ ID NO: 201 - DNA, 21 bp, Artificial Sequence
  Synethtic construct
accggtctctatattccctgt

SEQ ID NO: 202 - DNA, 18 bp, Artificial Sequence
  Synthetic construct
accttgtggtgcatgatg

SEQ ID NO: 203 - DNA, 21 bp, Artificial Sequence
  Synthetic construct
agattagcaggtttcccaccg

SEQ ID NO: 204 - DNA, 22 bp, Artificial Sequence
  Synthetic construct
ggtgtgattgatgaacgattac

SEQ ID NO: 205 - DNA, 22 bp, Artificial Sequence
  Synthetic construct
cctctgccggagctatattcag

SEQ ID NO: 206 - DNA, 22 bp, Artificial Sequence
  Synthetic construct
cctcattacaagtatcacctgt

SEQ ID NO: 207 - DNA, 19 bp, Artificial Sequence
  Synthetic construct
ctgcgttaggtggaatacc

SEQ ID NO: 208 - DNA, 38 bp, Artificial Sequence
  Synthetic construct
tttcccaccggatcaccaagaccttgtggtgcatgatg

SEQ ID NO: 209 - DNA, 44 bp, Artificial Sequence; identical to SEQ ID NO: 159
  Synthetic construct

SEQ ID NO: 210 - DNA, 20 bp, Artificial Sequence
  Synthetic construct
tggagagaagaaccctggat

SEQ ID NO: 211 - DNA, 21 bp, Artificial Sequence
  Synthetic construct
accggtctctatattccctgt

SEQ ID NO: 212 - DNA, 18 bp, Artificial Sequence
  Synthetic construct
accttgtggtgcatgatg

SEQ ID NO: 213 - DNA, 20 bp, Artificial Sequence
  Synthetic construct
tttcccaccggatcaccaag

SEQ ID NO: 214 - DNA, 22 bp, Artificial Sequence
  Synthetic construct
ggtgtgattgatgaacgattac

SEQ ID NO: 215 - DNA, 22 bp, Artificial Sequence
  Synthetic construct
cctctgccggagctatattcag

SEQ ID NO: 216 - DNA, 22 bp, Artificial Sequence
  Synthetic construct
cctcattacaagtatcacctgt

SEQ ID NO: 217 - DNA, 21 bp, Artificial Sequence
  Synthetic construct
gcgttaggtggaataccatat

SEQ ID NO: 218 - DNA, 37 bp, Artificial Sequence; identical to SEQ ID NO: 158
  Synthetic construct

SEQ ID NO: 219 - DNA, 44 bp, Artificial Sequence; identical to SEQ ID NO: 159
  Synthetic construct

SEQ ID NO: 220 - DNA, 20 bp, Artificial Sequence
  Synthetic construct
tggagagaagaaccctggat

SEQ ID NO: 221 - DNA, 21 bp, Artificial Sequence
  Synthetic construct
accggtctctatattccctgt

SEQ ID NO: 222 - DNA, 18 bp, Artificial Sequence
  Synthetic construct
accttgtggtgcatgatg

SEQ ID NO: 223 - DNA, 19 bp, Artificial Sequence
  Synthetic construct
ggtttcccaccggatcacc

SEQ ID NO: 224 - DNA, 22 bp, Artificial Sequence
  Synthetic construct
ggtgtgattgatgaacgattac

SEQ ID NO: 225 - DNA, 22 bp, Artificial Sequence
  Synthetic construct
cctctgccggagctatattcag

SEQ ID NO: 226 - DNA, 22 bp, Artificial Sequence
  Synthetic construct
gatgaatttccacaaccttgtg

SEQ ID NO: 227 - DNA, 19 bp, Artificial Sequence
  Synthetic construct
ctgcgttaggtggaatacc

SEQ ID NO: 228 - DNA, 43 bp, Artificial Sequence
  Synthetic construct
acagattagcaggtttcccacccatgatgaatccagggttctt

SEQ ID NO: 229 - DNA, 44 bp, Artificial Sequence; identical to SEQ ID NO: 159
  Synthetic construct

SEQ ID NO: 230 - DNA, 20 bp, Artificial Sequence
  Synthetic construct
ggatcaccaagcttggagag

SEQ ID NO: 231 - DNA, 21 bp, Artificial Sequence
  Synthetic construct
accggtctctatattccctgt

SEQ ID NO: 232 - DNA, 21 bp, Artificial Sequence
  Synthetic construct
catgatgaatccagggttctt

SEQ ID NO: 233 - DNA, 22 bp, Artificial Sequence
  Synthetic construct
acagattagcaggtttcccacc

SEQ ID NO: 234 - DNA, 22 bp, Artificial Sequence
  Synthetic construct
ggtgtgattgatgaacgattac

SEQ ID NO: 235 - DNA, 22 bp, Artificial Sequence
  Synthetic construct
cctctgccggagctatattcag

SEQ ID NO: 236 - DNA, 18 bp, Artificial Sequence
  Synthetic construct
cgggtgtgtggaggactt

SEQ ID NO: 237 - DNA, 20 bp, Artificial Sequence
  Synthetic construct
gctaaaccagcagggtcttc

SEQ ID NO: 238 - DNA, 43 bp, Artificial Sequence
  Synthetic construct
atatttgtgtgcgccgtggctcacctcttagtcgttcttcagc

SEQ ID NO: 239 - DNA, 44 bp, Artificial Sequence
  Synthetic construct
atagcacccggtacaagcaggatgaaagtagtcctgaaagcatg

SEQ ID NO: 240 - DNA, 21 bp, Artificial Sequence
  Synthetic construct
cgctgttcttcaactgtggag

SEQ ID NO: 241 - DNA, 22 bp, Artificial Sequence
  Synthetic construct
cacaattcacagcagtaattgc

SEQ ID NO: 242 - DNA, 22 bp, Artificial Sequence
  Synthetic construct
cacctcttagtcgttcttcagc

SEQ ID NO: 243 - DNA, 21 bp, Artificial Sequence
  Synthetic construct
atatttgtgtgcgccgtggct

SEQ ID NO: 244 - DNA, 22 bp, Artificial Sequence
  Synthetic construct
tgaaagtagtcctgaaagcatg

SEQ ID NO: 245 - DNA, 22 bp, Artificial Sequence
  Synthetic construct
atagcacccggtacaagcagga

SEQ ID NO: 246 - DNA, 18 bp, Artificial Sequence
  Synthetic construct
cgggtgtgtggaggactt

SEQ ID NO: 247 - DNA, 20 bp, Artificial Sequence
  Synthetic construct
gctaaaccagcagggtcttc

SEQ ID NO: 248 - DNA, 43 bp, Artificial Sequence; identical to SEQ ID NO: 238
  Synthetic construct

SEQ ID NO: 249 - DNA, 44 bp, Artificial Sequence
  Synthetic construct
tagcacccggtacaagcaggattgaaagtagtcctgaaagcatg

SEQ ID NO: 250 - DNA, 21 bp, Artificial Sequence
  Synthetic construct
cgctgttcttcaactgtggag

SEQ ID NO: 251 - DNA, 22 bp, Artificial Sequence
  Synthetic construct
cacaattcacagcagtaattgc

SEQ ID NO: 252 - DNA, 22 bp, Artificial Sequence
  Synthetic construct
cacctcttagtcgttcttcagc

SEQ ID NO: 253 - DNA, 21 bp, Artificial Sequence
  Synthetic construct
atatttgtgtgcgccgtggct

SEQ ID NO: 254 - DNA, 22 bp, Artificial Sequence
  Synthetic construct
tgaaagtagtcctgaaagcatg

SEQ ID NO: 255 - DNA, 22 bp, Artificial Sequence
  Synthetic construct
tagcacccggtacaagcaggat

SEQ ID NO: 256 - DNA, 18 bp, Artificial Sequence
  Synthetic construct
cgggtgtgtggaggactt

SEQ ID NO: 257 - DNA, 20 bp, Artificial Sequence
  Synthetic construct
gctaaaccagcagggtcttc

SEQ ID NO: 258 - DNA, 43 bp, Artificial Sequence
  Synthetic construct
tatttgtgtgcgccgtggctgcacctcttagtcgttcttcagc

SEQ ID NO: 259 - DNA, 44 bp, Artificial Sequence; identical to SEQ ID NO: 239
  Synthetic construct

SEQ ID NO: 260 - DNA, 21 bp, Artificial Sequence
  Synthetic construct
cgctgttcttcaactgtggag

SEQ ID NO: 261 - DNA, 22 bp, Artificial Sequence
  Synthetic construct
cacaattcacagcagtaattgc

SEQ ID NO: 262 - DNA, 22 bp, Artificial Sequence
  Synthetic construct
cacctcttagtcgttcttcagc

SEQ ID NO: 263 - DNA, 21 bp, Artificial Sequence
  Synthetic construct
tatttgtgtgcgccgtggctg

SEQ ID NO: 264 - DNA, 22 bp, Artificial Sequence
  Synthetic construct
tgaaagtagtcctgaaagcatg

SEQ ID NO: 265 - DNA, 22 bp, Artificial Sequence
  Synthetic construct
atagcacccggtacaagcagga

SEQ ID NO: 266 - DNA, 18 bp, Artificial Sequence
  Synthetic construct
cgggtgtgtggaggactt

SEQ ID NO: 267 - DNA, 20 bp, Artificial Sequence
  Synthetic construct
gctaaaccagcagggtcttc

SEQ ID NO: 268 - DNA, 40 bp, Artificial Sequence
  Synthetic construct
atttgtgtgcgccgtggccacctcttagtcgttcttcagc

SEQ ID NO: 269 - DNA, 44 bp, Artificial Sequence; identical to SEQ ID NO: 239
  Synthetic construct

SEQ ID NO: 270 - DNA, 21 bp, Artificial Sequence
  Synthetic construct
cgctgttcttcaactgtggag

SEQ ID NO: 271 - DNA, 22 bp, Artificial Sequence
  Synthetic construct
cacaattcacagcagtaattgc

SEQ ID NO: 272 - DNA, 22 bp, Artificial Sequence
  Synthetic construct
cacctcttagtcgttcttcagc

SEQ ID NO: 273 - DNA, 18 bp, Artificial Sequence
  Synthetic construct
atttgtgtgcgccgtggc

SEQ ID NO: 274 - DNA, 22 bp, Artificial Sequence
  Synthetic construct
tgaaagtagtcctgaaagcatg

SEQ ID NO: 275 - DNA, 22 bp, Artificial Sequence
  Synthetic construct
atagcacccggtacaagcagga

SEQ ID NO: 276 - DNA, 18 bp, Artificial Sequence
  Synthetic construct
cgggtgtgtggaggactt

SEQ ID NO: 277 - DNA, 22 bp, Artificial Sequence
  Synthetic construct
ctaaaccagcagggtcttcaaa

SEQ ID NO: 278 - DNA, 43 bp, Artificial Sequence; identical to SEQ ID NO: 238
  Synthetic construct

SEQ ID NO: 279 - DNA, 44 bp, Artificial Sequence; identical to SEQ ID NO: 239
  Synthetic construct

SEQ ID NO: 280 - DNA, 21 bp, Artificial Sequence
  Synthetic construct
cgctgttcttcaactgtggag

SEQ ID NO: 281 - DNA, 22 bp, Artificial Sequence
  Synthetic construct
cacaattcacagcagtaattgc

SEQ ID NO: 282 - DNA, 22 bp, Artificial Sequence
  Synthetic construct
cacctcttagtcgttcttcagc

SEQ ID NO: 283 - DNA, 21 bp, Artificial Sequence
  Synthetic construct
atatttgtgtgcgccgtggct

SEQ ID NO: 284 - DNA, 22 bp, Artificial Sequence
  Synthetic construct
tgaaagtagtcctgaaagcatg

SEQ ID NO: 285 - DNA, 22 bp, Artificial Sequence
  Synthetic construct
atagcacccggtacaagcagga

SEQ ID NO: 286 - DNA, 18 bp, Artificial Sequence
  Synthetic construct
cgggtgtgtggaggactt

SEQ ID NO: 287 - DNA, 20 bp, Artificial Sequence
  Synthetic construct
gctaaaccagcagggtcttc

SEQ ID NO: 288 - DNA, 44 bp, Artificial Sequence
  Synthetic construct
tatttgtgtgcgccgtggctggcacctcttagtcgttcttcagc

SEQ ID NO: 289 - DNA, 44 bp, Artificial Sequence; identical to SEQ ID NO: 239
  Synthetic construct

SEQ ID NO: 290 - DNA, 21 bp, Artificial Sequence
  Synthetic construct
cgctgttcttcaactgtggag

SEQ ID NO: 291 - DNA, 22 bp, Artificial Sequence
  Synthetic construct
cacaattcacagcagtaattgc

SEQ ID NO: 292 - DNA, 22 bp, Artificial Sequence
  Synthetic construct
cacctcttagtcgttcttcagc

SEQ ID NO: 293 - DNA, 22 bp, Artificial Sequence
  Synthetic construct
tatttgtgtgcgccgtggctgg

SEQ ID NO: 294 - DNA, 22 bp, Artificial Sequence
  Synthetic construct
tgaaagtagtcctgaaagcatg

SEQ ID NO: 295 - DNA, 22 bp, Artificial Sequence
  Synthetic construct
atagcacccggtacaagcagga

SEQ ID NO: 296 - DNA, 18 bp, Artificial Sequence
  Synthetic construct
cgggtgtgtggaggactt

SEQ ID NO: 297 - DNA, 20 bp, Artificial Sequence
  Synthetic construct
gctaaaccagcagggtcttc

SEQ ID NO: 298 - DNA, 44 bp, Artificial Sequence
  Synthetic construct
tttatatttgtgtgcgccgtggcacctcttagtcgttcttcagc

SEQ ID NO: 299 - DNA, 42 bp, Artificial Sequence
  Synthetic construct
gcacccggtacaagcaggattgaaagtagtcctgaaagcatg

SEQ ID NO: 300 - DNA, 21 bp, Artificial Sequence
  Synthetic construct
cgctgttcttcaactgtggag

SEQ ID NO: 301 - DNA, 22 bp, Artificial Sequence
  Synthetic construct
cacaattcacagcagtaattgc

SEQ ID NO: 302 - DNA, 22 bp, Artificial Sequence
  Synthetic construct
cacctcttagtcgttcttcagc

SEQ ID NO: 303 - DNA, 22 bp, Artificial Sequence
  Synthetic construct
tttatatttgtgtgcgccgtgg

SEQ ID NO: 304 - DNA, 22 bp, Artificial Sequence
  Synthetic construct
tgaaagtagtcctgaaagcatg

SEQ ID NO: 305 - DNA, 20 bp, Artificial Sequence
  Synthetic construct
gcacccggtacaagcaggat

SEQ ID NO: 306 - DNA, 20 bp, Artificial Sequence
  Synthetic construct
tgtgtggaggactttcacct

SEQ ID NO: 307 - DNA, 20 bp, Artificial Sequence
  Synthetic construct
gctaaaccagcagggtcttc

SEQ ID NO: 308 - DNA, 39 bp, Artificial Sequence
  Synthetic construct
atttgtgtgcgccgtggcttagtcgttcttcagcctcca

SEQ ID NO: 309 - DNA, 44 bp, Artificial Sequence; identical to SEQ ID NO: 239
  Synthetic construct

SEQ ID NO: 310 - DNA, 19 bp, Artificial Sequence
  Synthetic construct
tggcgctgttcttcaactg

SEQ ID NO: 311 - DNA, 22 bp, Artificial Sequence
  Synthetic construct
cacaattcacagcagtaattgc

SEQ ID NO: 312 - DNA, 21 bp, Artificial Sequence
  Synthetic construct
ttagtcgttcttcagcctcca

SEQ ID NO: 313 - DNA, 18 bp, Artificial Sequence
  Synthetic construct
atttgtgtgcgccgtggc

SEQ ID NO: 314 - DNA, 22 bp, Artificial Sequence
  Synthetic construct
tgaaagtagtcctgaaagcatg

SEQ ID NO: 315 - DNA, 22 bp, Artificial Sequence
  Synthetic construct
atagcacccggtacaagcagga

SEQ ID NO: 316 - DNA, 19 bp, Artificial Sequence
  Synthetic construct
tccacagttgaagaacagc

SEQ ID NO: 317 - DNA, 18 bp, Artificial Sequence
  Synthetic construct
ctcttcgtagcggagagt

SEQ ID NO: 318 - DNA, 44 bp, Artificial Sequence
  Synthetic construct
gcaattactgctgtgaattgtgcgcacacaaatataaagggaac

SEQ ID NO: 319 - DNA, 40 bp, Artificial Sequence
  Synthetic construct
ttgaagaccctgctggtttagcaatattcgtggacgacgt

SEQ ID NO: 320 - DNA, 20 bp, Artificial Sequence
  Synthetic construct
ttgtaatcctgcttgtaccg

SEQ ID NO: 321 - DNA, 20 bp, Artificial Sequence
  Synthetic construct
atcctgagcgaaaggtgaaa

SEQ ID NO: 322 - DNA, 22 bp, Artificial Sequence
  Synthetic construct
cgcacacaaatataaagggaac

SEQ ID NO: 323 - DNA, 22 bp, Artificial Sequence
  Synthetic construct
gcaattactgctgtgaattgtg

SEQ ID NO: 324 - DNA, 18 bp, Artificial Sequence
  Synthetic construct
aatattcgtggacgacgt

SEQ ID NO: 325 - DNA, 22 bp, Artificial Sequence
  Synthetic construct
ttgaagaccctgctggtttagc

SEQ ID NO: 326 - DNA, 18 bp, Artificial Sequence
  Synthetic construct
tcttcagcctccacagtt

SEQ ID NO: 327 - DNA, 18 bp, Artificial Sequence
  Synthetic construct
ctcttcgtagcggagagt

SEQ ID NO: 328 - DNA, 40 bp, Artificial Sequence
  Synthetic construct
gcaattactgctgtgaattgtggaagaacagcgccagcca

SEQ ID NO: 329 - DNA, 44 bp, Artificial Sequence
  Synthetic construct
ttgaagaccctgctggtttagccgtgtttcggaggtaatatgaa

SEQ ID NO: 330 - DNA, 20 bp, Artificial Sequence
  Synthetic construct
ttgtaatcctgcttgtaccg

SEQ ID NO: 331 - DNA, 20 bp, Artificial Sequence
  Synthetic construct
atcctgagcgaaaggtgaaa

SEQ ID NO: 332 - DNA, 18 bp, Artificial Sequence
  Synthetic construct
gaagaacagcgccagcca

SEQ ID NO: 333 - DNA, 22 bp, Artificial Sequence
  Synthetic construct
gcaattactgctgtgaattgtg

SEQ ID NO: 334 - DNA, 22 bp, Artificial Sequence
  Synthetic construct
cgtgtttcggaggtaatatgaa

SEQ ID NO: 335 - DNA, 22 bp, Artificial Sequence
  Synthetic construct
ttgaagaccctgctggtttagc

SEQ ID NO: 336 - DNA, 18 bp, Artificial Sequence
  Synthetic construct
tcttcagcctccacagtt

SEQ ID NO: 337 - DNA, 19 bp, Artificial Sequence
  Synthetic construct
ctcttcgtagcggagagta

SEQ ID NO: 338 - DNA, 40 bp, Artificial Sequence; identical to SEQ ID NO: 328
  Synthetic construct

SEQ ID NO: 339 - DNA, 44 bp, Artificial Sequence; identical to SEQ ID NO: 329
  Synthetic construct

SEQ ID NO: 340 - DNA, 20 bp, Artificial Sequence
  Synthetic construct
ttgtaatcctgcttgtaccg

SEQ ID NO: 341 - DNA, 20 bp, Artificial Sequence
  Synthetic construct
atcctgagcgaaaggtgaaa

SEQ ID NO: 342 - DNA, 18 bp, Artificial Sequence
  Synthetic construct
gaagaacagcgccagcca

SEQ ID NO: 343 - DNA, 22 bp, Artificial Sequence
  Synthetic construct
gcaattactgctgtgaattgtg

SEQ ID NO: 344 - DNA, 22 bp, Artificial Sequence
  Synthetic construct
cgtgtttcggaggtaatatgaa

SEQ ID NO: 345 - DNA, 22 bp, Artificial Sequence
  Synthetic construct
ttgaagaccctgctggtttagc

SEQ ID NO: 346 - DNA, 19 bp, Artificial Sequence
  Synthetic construct
tccacagttgaagaacagc

SEQ ID NO: 347 - DNA, 18 bp, Artificial Sequence
  Synthetic construct
ctcttcgtagcggagagt

SEQ ID NO: 348 - DNA, 42 bp, Artificial Sequence
  Synthetic construct
agtagcaattactgctgtgacgcacacaaatataaagggaac

SEQ ID NO: 349 - DNA, 38 bp, Artificial Sequence
  Synthetic construct
gaagaccctgctggtttagcaatattcgtggacgacgt

SEQ ID NO: 350 - DNA, 22 bp, Artificial Sequence
  Synthetic construct
gtgttgtaatcctgcttgtacc

SEQ ID NO: 351 - DNA, 20 bp, Artificial Sequence
  Synthetic construct
atcctgagcgaaaggtgaaa

SEQ ID NO: 352 - DNA, 22 bp, Artificial Sequence
  Synthetic construct
cgcacacaaatataaagggaac

SEQ ID NO: 353 - DNA, 20 bp, Artificial Sequence
  Synthetic construct
agtagcaattactgctgtga

SEQ ID NO: 354 - DNA, 18 bp, Artificial Sequence
  Synthetic construct
aatattcgtggacgacgt

SEQ ID NO: 355 - DNA, 20 bp, Artificial Sequence
  Synthetic construct
gaagaccctgctggtttagc

SEQ ID NO: 356 - DNA, 18 bp, Artificial Sequence
  Synthetic construct
tcttcagcctccacagtt

SEQ ID NO: 357 - DNA, 18 bp, Artificial Sequence
  Synthetic construct
ctcttcgtagcggagagt

SEQ ID NO: 358 - DNA, 38 bp, Artificial Sequence
  Synthetic construct
gtagcaattactgctgtgaagaagaacagcgccagcca

SEQ ID NO: 359 - DNA, 44 bp, Artificial Sequence
  Synthetic construct
tttgaagaccctgctggtttagcgtgtttcggaggtaatatgaa

SEQ ID NO: 360 - DNA, 22 bp, Artificial Sequence
  Synthetic construct
gtgttgtaatcctgcttgtacc

SEQ ID NO: 361 - DNA, 19 bp, Artificial Sequence
  Synthetic construct
catcctgagcgaaaggtga

SEQ ID NO: 362 - DNA, 18 bp, Artificial Sequence
  Synthetic construct
gaagaacagcgccagcca

SEQ ID NO: 363 - DNA, 20 bp, Artificial Sequence
  Synthetic construct
gtagcaattactgctgtgaa

SEQ ID NO: 364 - DNA, 22 bp, Artificial Sequence
  Synthetic construct
cgtgtttcggaggtaatatgaa

SEQ ID NO: 365 - DNA, 22 bp, Artificial Sequence
  Synthetic construct
tttgaagaccctgctggtttag

SEQ ID NO: 366 - DNA, 18 bp, Artificial Sequence
  Synthetic construct
tcttcagcctccacagtt

SEQ ID NO: 367 - DNA, 18 bp, Artificial Sequence
  Synthetic construct
ctcttcgtagcggagagt

SEQ ID NO: 368 - DNA, 40 bp, Artificial Sequence
  Synthetic construct
tgttgtaatcctgcttgtaccggaagaacagcgccagcca

SEQ ID NO: 369 - DNA, 44 bp, Artificial Sequence; identical to SEQ ID NO: 329
  Synthetic construct

SEQ ID NO: 370 - DNA, 22 bp, Artificial Sequence
  Synthetic construct
gttccctttatatttgtgtgcg

SEQ ID NO: 371 - DNA, 20 bp, Artificial Sequence
  Synthetic construct
atcctgagcgaaaggtgaaa

SEQ ID NO: 372 - DNA, 18 bp, Artificial Sequence
  Synthetic construct
gaagaacagcgccagcca

SEQ ID NO: 373 - DNA, 22 bp, Artificial Sequence
  Synthetic construct
tgttgtaatcctgcttgtaccg

SEQ ID NO: 374 - DNA, 22 bp, Artificial Sequence
  Synthetic construct
cgtgtttcggaggtaatatgaa

SEQ ID NO: 375 - DNA, 22 bp, Artificial Sequence
  Synthetic construct
ttgaagaccctgctggtttagc

SEQ ID NO: 376 - DNA, 40 bp, Artificial Sequence
  Synthetic construct
gtgttgtaatcctgcttgtaccgaagaacagcgccagcca

SEQ ID NO: 377 - DNA, 22 bp, Artificial Sequence
  Synthetic construct
gtgttgtaatcctgcttgtacc

SEQ ID NO: 378 - DNA, 40 bp, Artificial Sequence
  Synthetic construct
tgtgttgtaatcctgcttgtacgaagaacagcgccagcca

SEQ ID NO: 379 - DNA, 22 bp, Artificial Sequence
  Synthetic construct
tgtgttgtaatcctgcttgtac

SEQ ID NO: 380 - DNA, 15 bp, Artificial Sequence
  Synthetic construct
tcaatacggttctga

SEQ ID NO: 381 - DNA, 15 bp, Artificial Sequence
  Synthetic construct
gtgtacctcgacata

SEQ ID NO: 382 - DNA, 31 bp, Artificial Sequence
  Synthetic construct
ttgttgtaatcctgcccgagtcgcttactat

SEQ ID NO: 383 - DNA, 30 bp, Artificial Sequence
  Synthetic construct
tgaagagtcaagtgagctaatgttggcaat

SEQ ID NO: 384 - DNA, 15 bp, Artificial Sequence
  Synthetic construct
tgtgctggatgttat

SEQ ID NO: 385 - DNA, 15 bp, Artificial Sequence
  Synthetic construct
cagttgactgactaa

SEQ ID NO: 386 - DNA, 15 bp, Artificial Sequence
  Synthetic construct
cgagtcgcttactat

SEQ ID NO: 387 - DNA, 16 bp, Artificial Sequence
  Synthetic construct
ttgttgtaatcctgcc

SEQ ID NO: 388 - DNA, 15 bp, Artificial Sequence
  Synthetic construct
gctaatgttggcaat

SEQ ID NO: 389 - DNA, 15 bp, Artificial Sequence
  Synthetic construct
tgaagagtcaagtga

SEQ ID NO: 390 - DNA, 15 bp, Artificial Sequence
  Synthetic construct
caatacggttctgac

SEQ ID NO: 391 - DNA, 31 bp, Artificial Sequence
  Synthetic construct
ttgttgtaatcctgccgagtcgcttactata

SEQ ID NO: 392 - DNA, 15 bp, Artificial Sequence
  Synthetic construct
gagtcgcttactata

SEQ ID NO: 393 - DNA, 16 bp, Artificial Sequence
  Synthetic construct
ttgttgtaatcctgcc

SEQ ID NO: 394 - DNA, 15 bp, Artificial Sequence
  Synthetic construct
caatacggttctgac

SEQ ID NO: 395 - DNA, 15 bp, Artificial Sequence
  Synthetic construct
gtgtacctcgacata

SEQ ID NO: 396 - DNA, 30 bp, Artificial Sequence
  Synthetic construct
tactgctgtgaacttaataacatccagcac

SEQ ID NO: 397 - DNA, 30 bp, Artificial Sequence; identical to SEQ ID NO: 383
  Synthetic construct

SEQ ID NO: 398 - DNA, 15 bp, Artificial Sequence
  Synthetic construct
tgttgtaatcctgcc

SEQ ID NO: 399 - DNA, 15 bp, Artificial Sequence
  Synthetic construct
cagttgactgactaa

SEQ ID NO: 400 - DNA, 15 bp, Artificial Sequence
  Synthetic construct
aataacatccagcac

SEQ ID NO: 401 - DNA, 15 bp, Artificial Sequence
  Synthetic construct
tactgctgtgaactt

SEQ ID NO: 402 - DNA, 15 bp, Artificial Sequence
  Synthetic construct
gctaatgttggcaat

SEQ ID NO: 403 - DNA, 15 bp, Artificial Sequence
  Synthetic construct
tgaagagtcaagtga

SEQ ID NO: 404 - DNA, 15 bp, Artificial Sequence
  Synthetic construct
caatacggttctgac

SEQ ID NO: 405 - DNA, 15 bp, Artificial Sequence
  Synthetic construct
gtgtacctcgacata

SEQ ID NO: 406 - DNA, 30 bp, Artificial Sequence
  Synthetic construc
tactgctgtgaacttataacatccagcaca

SEQ ID NO: 407 - DNA, 30 bp, Artificial Sequence; identical to SEQ ID NO: 383
  Synthetic construct

SEQ ID NO: 408 - DNA, 15 bp, Artificial Sequence
  Synthetic construct
tgttgtaatcctgcc

SEQ ID NO: 409 - DNA, 15 bp, Artificial Sequence
  Synthetic construct
cagttgactgactaa

SEQ ID NO: 410 - DNA, 15 bp, Artificial Sequence
  Synthetic construct
ataacatccagcaca

SEQ ID NO: 411 - DNA, 15 bp, Artificial Sequence
  Synthetic construct
tactgctgtgaactt

SEQ ID NO: 412 - DNA, 15 bp, Artificial Sequence
  Synthetic construct
gctaatgttggcaat

SEQ ID NO: 413 - DNA, 15 bp, Artificial Sequence
  Synthetic construct
tgaagagtcaagtga

SEQ ID NO: 414 - DNA, 15 bp, Artificial Sequence
  Synthetic construct
tgaacatatccagga

SEQ ID NO: 415 - DNA, 15 bp, Artificial Sequence
  Synthetic construct
gtgtacctcgacata

SEQ ID NO: 416 - DNA, 31 bp, Artificial Sequence
  Synthetic construct
ttgttgtaatcctgcccaatacggttctgac

SEQ ID NO: 417 - DNA, 30 bp, Artificial Sequence; identical to SEQ ID NO: 383
  Synthetic construct

SEQ ID NO: 418 - DNA, 15 bp, Artificial Sequence
  Synthetic construct
tgtgctggatgttat

SEQ ID NO: 419 - DNA, 15 bp, Artificial Sequence
  Synthetic construct
cagttgactgactaa

SEQ ID NO: 420 - DNA, 15 bp, Artificial Sequence
  Synthetic construct
caatacggttctgac

SEQ ID NO: 421 - DNA, 16 bp, Artificial Sequence
  Synthetic construct
ttgttgtaatcctgcc

SEQ ID NO: 422 - DNA, 15 bp, Artificial Sequence
  Synthetic construct
gctaatgttggcaat

SEQ ID NO: 423 - DNA, 15 bp, Artificial Sequence
  Synthetic construct
tgaagagtcaagtga

SEQ ID NO: 424 - DNA, 15 bp, Artificial Sequence
  Synthetic construct
atcaatacggttctg

SEQ ID NO: 425 - DNA, 15 bp, Artificial Sequence
  Synthetic construct
gtgtacctcgacata

SEQ ID NO: 426 - DNA, 31 bp, Artificial Sequence
  Synthetic construct
ttgttgtaatcctgccacgagtcgcttacta

SEQ ID NO: 427 - DNA, 30 bp, Artificial Sequence; identical to SEQ ID NO: 383
  Synthetic construct

SEQ ID NO: 428 - DNA, 15 bp, Artificial Sequence
  Synthetic construct
tgtgctggatgttat

SEQ ID NO: 429 - DNA, 15 bp, Artificial Sequence
  Synthetic construct
cagttgactgactaa

SEQ ID NO: 430 - DNA, 15 bp, Artificial Sequence
  Synthetic construct
acgagtcgcttacta

SEQ ID NO: 431 - DNA, 16 bp, Artificial Sequence
  Synthetic construct
ttgttgtaatcctgcc

SEQ ID NO: 432 - DNA, 15 bp, Artificial Sequence
  Synthetic construct
gctaatgttggcaat

SEQ ID NO: 433 - DNA, 15 bp, Artificial Sequence
  Synthetic construct
tgaagagtcaagtga

SEQ ID NO: 434 - DNA, 15 bp, Artificial Sequence
  Synthetic construct
tgaacatatccagga

SEQ ID NO: 435 - DNA, 17 bp, Artificial Sequence
  Synthetic construct
gtacctcgacatataac

SEQ ID NO: 436 - DNA, 31 bp, Artificial Sequence; identical to SEQ ID NO: 416
  Synthetic construct

SEQ ID NO: 437 - DNA, 30 bp, Artificial Sequence; identical to SEQ ID NO: 383
  Synthetic construct

SEQ ID NO: 438 - DNA, 15 bp, Artificial Sequence
  Synthetic construct
tgtgctggatgttat

SEQ ID NO: 439 - DNA, 15 bp, Artificial Sequence
  Synthetic construct
cagttgactgactaa

SEQ ID NO: 440 - DNA, 15 bp, Artificial Sequence
  Synthetic construct
caatacggttctgac

SEQ ID NO: 441 - DNA, 16 bp, Artificial Sequence
  Synthetic construct
ttgttgtaatcctgcc

SEQ ID NO: 442 - DNA, 15 bp, Artificial Sequence
  Synthetic construct
gctaatgttggcaat

SEQ ID NO: 443 - DNA, 15 bp, Artificial Sequence
  Synthetic construct
tgaagagtcaagtga

SEQ ID NO: 444 - DNA, 15 bp, Artificial Sequence
  Synthetic construct
caatacggttctgac

SEQ ID NO: 445 - DNA, 15 bp, Artificial Sequence
  Synthetic construct
gtgtacctcgacata

SEQ ID NO: 446 - DNA, 30 bp, Artificial Sequence
  Synthetic construct
ttactgctgtgaacttaacatccagcacag

SEQ ID NO: 447 - DNA, 30 bp, Artificial Sequence; identical to SEQ ID NO: 383
  Synthetic construct

SEQ ID NO: 448 - DNA, 15 bp, Artificial Sequence
  Synthetic construct
ttgttgtaatcctgc

SEQ ID NO: 449 - DNA, 15 bp, Artificial Sequence
  Synthetic construct
cagttgactgactaa

SEQ ID NO: 450 - DNA, 15 bp, Artificial Sequence
  Synthetic construct
taacatccagcacag

SEQ ID NO: 451 - DNA, 15 bp, Artificial Sequence
  Synthetic construct
ttactgctgtgaact

SEQ ID NO: 452 - DNA, 15 bp, Artificial Sequence
  Synthetic construct
gctaatgttggcaat

SEQ ID NO: 453 - DNA, 15 bp, Artificial Sequence
  Synthetic construct
tgaagagtcaagtga

SEQ ID NO: 454 - DNA, 15 bp, Artificial Sequence
  Synthetic construct
tgtactcgctgatcg

SEQ ID NO: 455 - DNA, 17 bp, Artificial Sequence
  Synthetic construct
ctgaatcacttgactct

SEQ ID NO: 456 - DNA, 35 bp, Artificial Sequence
  Synthetic construct
tcctggatatgttcaatgactgaacaatgtggagc

SEQ ID NO: 457 - DNA, 35 bp, Artificial Sequence
  Synthetic construct
tatcaatacggttctgacgatactgctgtgaactt

SEQ ID NO: 458 - DNA, 15 bp, Artificial Sequence
  Synthetic construct
gtaacgcagccactt

SEQ ID NO: 459 - DNA, 15 bp, Artificial Sequence
  Synthetic construct
gcacaggcaggatta

SEQ ID NO: 460 - DNA, 15 bp, Artificial Sequence
  Synthetic construct
tgaacaatgtggagc

SEQ ID NO: 461 - DNA, 20 bp, Artificial Sequence
  Synthetic construct
tcctggatatgttcaatgac

SEQ ID NO: 462 - DNA, 15 bp, Artificial Sequence
  Synthetic construct
tactgctgtgaactt

SEQ ID NO: 463 - DNA, 20 bp, Artificial Sequence
  Synthetic construct
tatcaatacggttctgacga

SEQ ID NO: 464 - DNA, 15 bp, Artificial Sequence
  Synthetic construct
agcggtggtgaacat

SEQ ID NO: 465 - DNA, 17 bp, Artificial Sequence
  Synthetic construct
ctgaatcacttgactct

SEQ ID NO: 466 - DNA, 35 bp, Artificial Sequence
  Synthetic construct
cctggatatgttcaatgacgtgtactcgctgatcg

SEQ ID NO: 467 - DNA, 35 bp, Artificial Sequence; identical to SEQ ID NO: 457
  Synthetic construct

SEQ ID NO: 468 - DNA, 17 bp, Artificial Sequence
  Synthetic construct
ctccacattgttcagac

SEQ ID NO: 469 - DNA, 15 bp, Artificial Sequence
  Synthetic construct
gcacaggcaggatta

SEQ ID NO: 470 - DNA, 15 bp, Artificial Sequence
  Synthetic construct
tgtactcgctgatcg

SEQ ID NO: 471 - DNA, 20 bp, Artificial Sequence
  Synthetic construct
cctggatatgttcaatgacg

SEQ ID NO: 472 - DNA, 15 bp, Artificial Sequence
  Synthetic construct
tactgctgtgaactt

SEQ ID NO: 473 - DNA, 20 bp, Artificial Sequence
  Synthetic construct
tatcaatacggttctgacga

SEQ ID NO: 474 - DNA, 15 bp, Artificial Sequence
  Synthetic construct
agcggtggtgaacat

SEQ ID NO: 475 - DNA, 17 bp, Artificial Sequence
  Synthetic construct
actgaatcacttgactc

SEQ ID NO: 476 - DNA, 35 bp, Artificial Sequence; identical to SEQ ID NO: 466
  Synthetic construct

SEQ ID NO: 477 - DNA, 35 bp, Artificial Sequence; identical to SEQ ID NO: 457
  Synthetic construct

SEQ ID NO: 478 - DNA, 17 bp, Artificial Sequence
  Synthetic construct
ctccacattgttcagac

SEQ ID NO: 479 - DNA, 15 bp, Artificial Sequence
  Synthetic construct
gcacaggcaggatta

SEQ ID NO: 480 - DNA, 15 bp, Artificial Sequence
  Synthetic construct
tgtactcgctgatcg

SEQ ID NO: 481 - DNA, 20 bp, Artificial Sequence
  Synthetic construct
cctggatatgttcaatgacg

SEQ ID NO: 482 - DNA, 15 bp, Artificial Sequence
  Synthetic construct
tactgctgtgaactt

SEQ ID NO: 483 - DNA, 20 bp, Artificial Sequence
  Synthetic construct
tatcaatacggttctgacga

SEQ ID NO: 484 - DNA, 15 bp, Artificial Sequence
  Synthetic construct
agcggtggtgaacat

SEQ ID NO: 485 - DNA, 17 bp, Artificial Sequence
  Synthetic construct
ctgaatcacttgactct

SEQ ID NO: 486 - DNA, 35 bp, Artificial Sequence
  Synthetic construct
tcctggatatgttcaatgactgtactcgctgatcg

SEQ ID NO: 487 - DNA, 35 bp, Artificial Sequence; identical to SEQ ID NO: 457
  Synthetic construct

SEQ ID NO: 488 - DNA, 17 bp, Artificial Sequence
  Synthetic construct
ctccacattgttcagac

SEQ ID NO: 489 - DNA, 15 bp, Artificial Sequence
  Synthetic construct
gcacaggcaggatta

SEQ ID NO: 490 - DNA, 15 bp, Artificial Sequence
  Synthetic construct
tgtactcgctgatcg

SEQ ID NO: 491 - DNA, 20 bp, Artificial Sequence
  Synthetic construct
tcctggatatgttcaatgac

SEQ ID NO: 492 - DNA, 15 bp, Artificial Sequence
  Synthetic construct
tactgctgtgaactt

SEQ ID NO: 493 - DNA, 20 bp, Artificial Sequence
  Synthetic construct
tatcaatacggttctgacga

SEQ ID NO: 494 - DNA, 15 bp, Artificial Sequence
  Synthetic construct
agcggtggtgaacat

SEQ ID NO: 495 - DNA, 17 bp, Artificial Sequence
  Synthetic construct
ctgaatcacttgactct

SEQ ID NO: 496 - DNA, 30 bp, Artificial Sequence
  Synthetic construct
cgtaacgcagccacttgtactcgctgatcg

SEQ ID NO: 497 - DNA, 35 bp, Artificial Sequence; identical to SEQ ID NO: 457
  Synthetic construct

SEQ ID NO: 498 - DNA, 17 bp, Artificial Sequence
  Synthetic construct
ctccacattgttcagac

SEQ ID NO: 499 - DNA, 15 bp, Artificial Sequence
  Synthetic construct
gcacaggcaggatta

SEQ ID NO: 500 - DNA, 15 bp, Artificial Sequence
  Synthetic construct
tgtactcgctgatcg

SEQ ID NO: 501 - DNA, 15 bp, Artificial Sequence
  Synthetic construct
cgtaacgcagccact

SEQ ID NO: 502 - DNA, 15 bp, Artificial Sequence
  Synthetic construct
tactgctgtgaactt

SEQ ID NO: 503 - DNA, 20 bp, Artificial Sequence
  Synthetic construct
tatcaatacggttctgacga

SEQ ID NO: 504 - DNA, 15 bp, Artificial Sequence
  Synthetic construct
agcggtggtgaacat

SEQ ID NO: 505 - DNA, 17 bp, Artificial Sequence
  Synthetic construct
ctgaatcacttgactct

SEQ ID NO: 506 - DNA, 30 bp, Artificial Sequence
  Synthetic construct
gtaacgcagccactttgtactcgctgatcg

SEQ ID NO: 507 - DNA, 35 bp, Artificial Sequence; identical to SEQ ID NO: 457
  Synthetic construct

SEQ ID NO: 508 - DNA, 17 bp, Artificial Sequence
  Synthetic construct
ctccacattgttcagac

SEQ ID NO: 509 - DNA, 15 bp, Artificial Sequence
  Synthetic construct
gcacaggcaggatta

SEQ ID NO: 510 - DNA, 15 bp, Artificial Sequence
  Synthetic construct
tgtactcgctgatcg

SEQ ID NO: 511 - DNA, 15 bp, Artificial Sequence
  Synthetic construct
gtaacgcagccactt

SEQ ID NO: 512 - DNA, 15 bp, Artificial Sequence
  Synthetic construct
tactgctgtgaactt

SEQ ID NO: 513 - DNA, 20 bp, Artificial Sequence
  Synthetic construct
tatcaatacggttctgacga

SEQ ID NO: 514 - DNA, 15 bp, Artificial Sequence
  Synthetic construct
agcggtggtgaacat

SEQ ID NO: 515 - DNA, 17 bp, Artificial Sequence
  Synthetic construct
ctgaatcacttgactct

SEQ ID NO: 516 - DNA, 34 bp, Artificial Sequence
  Synthetic construct
gccaatcctggatatgttctgtactcgctgatcg

SEQ ID NO: 517 - DNA, 35 bp, Artificial Sequence; identical to SEQ ID NO: 457
  Synthetic construct

SEQ ID NO: 518 - DNA, 17 bp, Artificial Sequence
  Synthetic construct
ctccacattgttcagac

SEQ ID NO: 519 - DNA, 15 bp, Artificial Sequence
  Synthetic construct
gcacaggcaggatta

SEQ ID NO: 520 - DNA, 15 bp, Artificial Sequence
  Synthetic construct
tgtactcgctgatcg

SEQ ID NO: 521 - DNA, 19 bp, Artificial Sequence
  Synthetic construct
gccaatcctggatatgttc

SEQ ID NO: 522 - DNA, 15 bp, Artificial Sequence
  Synthetic construct
tactgctgtgaactt

SEQ ID NO: 523 - DNA, 20 bp, Artificial Sequence
  Synthetic construct
tatcaatacggttctgacga

SEQ ID NO: 524 - DNA, 15 bp, Artificial Sequence
  Synthetic construct
acatgccgtctgaac

SEQ ID NO: 525 - DNA, 17 bp, Artificial Sequence
  Synthetic construct
ctgaatcacttgactct

SEQ ID NO: 526 - DNA, 35 bp, Artificial Sequence
  Synthetic construct
tcctggatatgttcaatgacaatgtggagccagaa

SEQ ID NO: 527 - DNA, 35 bp, Artificial Sequence; identical to SEQ ID NO: 457
  Synthetic construct

SEQ ID NO: 528 - DNA, 15 bp, Artificial Sequence
  Synthetic construct
gtaacgcagccactt

SEQ ID NO: 529 - DNA, 15 bp, Artificial Sequence
  Synthetic construct
gcacaggcaggatta

SEQ ID NO: 530 - DNA, 15 bp, Artificial Sequence
  Synthetic construct
aatgtggagccagaa

SEQ ID NO: 531 - DNA, 20 bp, Artificial Sequence
  Synthetic construct
tcctggatatgttcaatgac

SEQ ID NO: 532 - DNA, 15 bp, Artificial Sequence
  Synthetic construct
tactgctgtgaactt

SEQ ID NO: 533 - DNA, 20 bp, Artificial Sequence
  Synthetic construct
tatcaatacggttctgacga

SEQ ID NO: 534 - DNA, 18 bp, Artificial Sequence
  Synthetic construct
aattctggaggacattgc

SEQ ID NO: 535 - DNA, 17 bp, Artificial Sequence
  Synthetic construct
tacgcttcagtacagca

SEQ ID NO: 536 - DNA, 39 bp, Artificial Sequence
  Synthetic construct
ggcagtggagagctgaagggatgagatagaagtctacct

SEQ ID NO: 537 - DNA, 34 bp, Artificial Sequence
  Synthetic construct
cgcactgccgaagctatggagaaccagtccgtaa

SEQ ID NO: 538 - DNA, 17 bp, Artificial Sequence
  Synthetic construct
cgagcatggtctggaag

SEQ ID NO: 539 - DNA, 19 bp, Artificial Sequence
  Synthetic construct
cagaagccgtgaagagaat

SEQ ID NO: 540 - DNA, 21 bp, Artificial Sequence
  Synthetic construct
ggatgagatagaagtctacct

SEQ ID NO: 541 - DNA, 18 bp, Artificial Sequence
  Synthetic construct
ggcagtggagagctgaag

SEQ ID NO: 542 - DNA, 17 bp, Artificial Sequence
  Synthetic construct
ggagaaccagtccgtaa

SEQ ID NO: 543 - DNA, 17 bp, Artificial Sequence
  Synthetic construct
cgcactgccgaagctat

SEQ ID NO: 544 - DNA, 20 bp, Artificial Sequence
  Synthetic construct
tgtatcacagatatggcatg

SEQ ID NO: 545 - DNA, 17 bp, Artificial Sequence
  Synthetic construct
gcgccggtatcattatc

SEQ ID NO: 546 - DNA, 34 bp, Artificial Sequence
  Synthetic construct
ccatgcagcgacctgttttccgataccgtctctg

SEQ ID NO: 547 - DNA, 35 bp, Artificial Sequence
  Synthetic construct
ttcgctgttgctgctgatattgttccatgtgagcg

SEQ ID NO: 548 - DNA, 18 bp, Artificial Sequence
  Synthetic construct
cggaatccggaggtattg

SEQ ID NO: 549 - DNA, 17 bp, Artificial Sequence
  Synthetic construct
tgagagctgtgaggacc

SEQ ID NO: 550 - DNA, 17 bp, Artificial Sequence
  Synthetic construct
ttccgataccgtctctg

SEQ ID NO: 551 - DNA, 17 bp, Artificial Sequence
  Synthetic construct
ccatgcagcgacctgtt

SEQ ID NO: 552 - DNA, 18 bp, Artificial Sequence
  Synthetic construct
ttcgctgttgctgctgat

SEQ ID NO: 553 - DNA, 18 bp, Artificial Sequence
  Synthetic construct
ttcgctgttgctgctgat

SEQ ID NO: 554 - DNA, 17 bp, Artificial Sequence
  Synthetic construct
gggagtgacagcaaatg

SEQ ID NO: 555 - DNA, 17 bp, Artificial Sequence
  Synthetic construct
gttcagtctcacgcatc

SEQ ID NO: 556 - DNA, 34 bp, Artificial Sequence
  Synthetic construct
gcggtcagcttccgtacttacggactggttctcc

SEQ ID NO: 557 - DNA, 41 bp, Artificial Sequence
  Synthetic construct
gcagaagagcagaagtatgagatcagacctgatgctttcag

SEQ ID NO: 558 - DNA, 18 bp, Artificial Sequence
  Synthetic construct
tcagtacagcatgccatg

SEQ ID NO: 559 - DNA, 21 bp, Artificial Sequence
  Synthetic construct
tggagaatgagtactctcaga

SEQ ID NO: 560 - DNA, 17 bp, Artificial Sequence
  Synthetic construct
ttacggactggttctcc

SEQ ID NO: 561 - DNA, 17 bp, Artificial Sequence
  Synthetic construct
gcggtcagcttccgtac

SEQ ID NO: 562 - DNA, 18 bp, Artificial Sequence
  Synthetic construct
cagacctgatgctttcag

SEQ ID NO: 563 - DNA, 23 bp, Artificial Sequence
  Synthetic construct
gcagaagagcagaagtatgagat

SEQ ID NO: 564 - DNA, 18 bp, Artificial Sequence
  Synthetic construct
cttcgacagcagtctttc

SEQ ID NO: 565 - DNA, 17 bp, Artificial Sequence
  Synthetic construct
cagtggagagctgaagt

SEQ ID NO: 566 - DNA, 36 bp, Artificial Sequence
  Synthetic construct
gcgccggtatcattatcgacgctcacatggaacaat

SEQ ID NO: 567 - DNA, 42 bp, Artificial Sequence
  Synthetic construct
cctcgaaattctggaggacattagacttctatctcatccaca

SEQ ID NO: 568 - DNA, 18 bp, Artificial Sequence
  Synthetic construct
ccttctgatgcctgatgg

SEQ ID NO: 569 - DNA, 22 bp, Artificial Sequence
  Synthetic construct
ggataaagtcagaactctccat

SEQ ID NO: 570 - DNA, 17 bp, Artificial Sequence
  Synthetic construct
cgctcacatggaacaat

SEQ ID NO: 571 - DNA, 19 bp, Artificial Sequence
  Synthetic construct
gcgccggtatcattatcga

SEQ ID NO: 572 - DNA, 20 bp, Artificial Sequence
  Synthetic construct
agacttctatctcatccaca

SEQ ID NO: 573 - DNA, 22 bp, Artificial Sequence
  Synthetic construct
cctcgaaattctggaggacatt

SEQ ID NO: 574 - DNA, 18 bp, Artificial Sequence
  Synthetic construct
cttcgacagcagtctttc

SEQ ID NO: 575 - DNA, 17 bp, Artificial Sequence
  Synthetic construct
cagtggagagctgaagt

SEQ ID NO: 576 - DNA, 38 bp, Artificial Sequence
  Synthetic construct
agcgccggtatcattatcgctcacatggaacaatctcc

SEQ ID NO: 577 - DNA, 42 bp, Artificial Sequence; identical to SEQ ID NO: 567
  Synthetic construct

SEQ ID NO: 578 - DNA, 18 bp, Artificial Sequence
  Synthetic construct
ccttctgatgcctgatgg

SEQ ID NO: 579 - DNA, 22 bp, Artificial Sequence
  Synthetic construct
ggataaagtcagaactctccat

SEQ ID NO: 580 - DNA, 19 bp, Artificial Sequence
  Synthetic construct
ctcacatggaacaatctcc

SEQ ID NO: 581 - DNA, 19 bp, Artificial Sequence
  Synthetic construct
agcgccggtatcattatcg

SEQ ID NO: 582 - DNA, 20 bp, Artificial Sequence
  Synthetic construct
agacttctatctcatccaca

SEQ ID NO: 583 - DNA, 22 bp, Artificial Sequence
  Synthetic construct
cctcgaaattctggaggacatt

SEQ ID NO: 584 - DNA, 18 bp, Artificial Sequence
  Synthetic construct
cttcgacagcagtctttc

SEQ ID NO: 585 - DNA, 17 bp, Artificial Sequence
  Synthetic construct
cagtggagagctgaagt

SEQ ID NO: 586 - DNA, 37 bp, Artificial Sequence
  Synthetic construct
agcgccggtatcattatcggctcacatggaacaatct

SEQ ID NO: 587 - DNA, 42 bp, Artificial Sequence; identical to SEQ ID NO: 567
  Synthetic construct

SEQ ID NO: 588 - DNA, 18 bp, Artificial Sequence
  Synthetic construct
ccttctgatgcctgatgg

SEQ ID NO: 589 - DNA, 22 bp, Artificial Sequence
  Synthetic construct
ggataaagtcagaactctccat

SEQ ID NO: 590 - DNA, 18 bp, Artificial Sequence
  Synthetic construrct
gctcacatggaacaatct

SEQ ID NO: 591 - DNA, 19 bp, Artificial Sequence
  Synthetic construct
agcgccggtatcattatcg

SEQ ID NO: 592 - DNA, 20 bp, Artificial Sequence
  Synthetic construct
agacttctatctcatccaca

SEQ ID NO: 593 - DNA, 22 bp, Artificial Sequence
  Synthetic construct
cctcgaaattctggaggacatt

SEQ ID NO: 594 - DNA, 18 bp, Artificial Sequence
  Synthetic construct
tgaagagcatgccaacac

SEQ ID NO: 595 - DNA, 18 bp, Artificial Sequence
  Synthetic construct
gccttctgatgcctgatg

SEQ ID NO: 596 - DNA, 36 bp, Artificial Sequence
  Synthetic construct
tccgcagaggcactgagtctctgcacgcaatacctc

SEQ ID NO: 597 - DNA, 40 bp, Artificial Sequence
  Synthetic construct
tctttcgctgttgctgctgatagattgttccatgtgagcg

SEQ ID NO: 598 - DNA, 18 bp, Artificial Sequence
  Synthetic construct
gcgacctgttcacggaat

SEQ ID NO: 599 - DNA, 18 bp, Artificial Sequence
  Synthetic construct
gccactgagagctgtgag

SEQ ID NO: 600 - DNA, 18 bp, Artificial Sequence
  Synthetic construct
ctctgcacgcaatacctc

SEQ ID NO: 601 - DNA, 18 bp, Artificial Sequence
  Synthetic construct
tccgcagaggcactgagt

SEQ ID NO: 602 - DNA, 19 bp, Artificial Sequence
  Synthetic construct
agattgttccatgtgagcg

SEQ ID NO: 603 - DNA, 21 bp, Artificial Sequence
  Synthetic construct
tctttcgctgttgctgctgat

SEQ ID NO: 604 - DNA, 19 bp, Artificial Sequence
  Synthetic construct
tgaacatgaagagcatgcc

SEQ ID NO: 605 - DNA, 18 bp, Artificial Sequence
  Synthetic construct
gccttctgatgcctgatg

SEQ ID NO: 606 - DNA, 36 bp, Artificial Sequence
  Synthetic construct
tccgcagaggcactgagtgtctctgcacgcaatacc

SEQ ID NO: 607 - DNA, 40 bp, Artificial Sequence; identical to SEQ ID NO: 597
  Synthetic construct

SEQ ID NO: 608 - DNA, 18 bp, Artificial Sequence
  Synthetic construct
gcgacctgttcacggaat

SEQ ID NO: 609 - DNA, 18 bp, Artificial Sequence
  Synthetic construct
gccactgagagctgtgag

SEQ ID NO: 610 - DNA, 18 bp, Artificial Sequence
  Synthetic construct
gtctctgcacgcaatacc

SEQ ID NO: 611 - DNA, 18 bp, Artificial Sequence
  Synthetic construct
tccgcagaggcactgagt

SEQ ID NO: 612 - DNA, 19 bp, Artificial Sequence
  Synthetic construct
agattgttccatgtgagcg

SEQ ID NO: 613 - DNA, 21 bp, Artificial Sequence
  Synthetic construct
tctttcgctgttgctgctgat

SEQ ID NO: 614 - DNA, 18 bp, Artificial Sequence
  Synthetic construct
agcttcgacagcagtctt

SEQ ID NO: 615 - DNA, 21 bp, Artificial Sequence
  Synthetic construct
gccaggtagacttctatctca

SEQ ID NO: 616 - DNA, 38 bp, Artificial Sequence
  Synthetic construct
tgatgcctgatggaccaggacactgagagctgtgagga

SEQ ID NO: 617 - DNA, 41 bp, Artificial Sequence
  Synthetic construct
cgataatgataccggcgctctgggcaatgtcctccagaatt

SEQ ID NO: 618 - DNA, 20 bp, Artificial Sequence
  Synthetic construct
ccggagattgttccatgtga

SEQ ID NO: 619 - DNA, 18 bp, Artificial Sequence
  Synthetic construct
ggaaatgttccgcctcga

SEQ ID NO: 620 - DNA, 18 bp, Artificial Sequence
  Synthetic construct
cactgagagctgtgagga

SEQ ID NO: 621 - DNA, 20 bp, Artificial Sequence
  Synthetic construct
tgatgcctgatggaccagga

SEQ ID NO: 622 - DNA, 19 bp, Artificial Sequence
  Synthetic construct
ggcaatgtcctccagaatt

SEQ ID NO: 623 - DNA, 22 bp, Artificial Sequence
  Synthetic construct
cgataatgataccggcgctctg

SEQ ID NO: 624 - DNA, 19 bp, Artificial Sequence
  Synthetic construct
tgaacatgaagagcatgcc

SEQ ID NO: 625 - DNA, 18 bp, Artificial Sequence
  Synthetic construct
gccttctgatgcctgatg

SEQ ID NO: 626 - DNA, 36 bp, Artificial Sequence
  Synthetic construct
tccgcagaggcactgagttctctgcacgcaatacct

SEQ ID NO: 627 - DNA, 40 bp, Artificial Sequence; identical to SEQ ID NO: 597
  Synthetic construct

SEQ ID NO: 628 - DNA, 18 bp, Artificial Sequence
  Synthetic construct
gcgacctgttcacggaat

SEQ ID NO: 629 - DNA, 18 bp, Artificial Sequence
  Synthetic construct
gccactgagagctgtgag

SEQ ID NO: 630 - DNA, 18 bp, Artificial Sequence
  Synthetic construct
tctctgcacgcaatacct

SEQ ID NO: 631 - DNA, 18 bp, Artificial Sequence
  Synthetic construct
tccgcagaggcactgagt

SEQ ID NO: 632 - DNA, 19 bp, Artificial Sequence
  Synthetic construct
agattgttccatgtgagcg

SEQ ID NO: 633 - DNA, 21 bp, Artificial Sequence
  Synthetic construct
tctttcgctgttgctgctgat

SEQ ID NO: 634 - DNA, 22 bp, Artificial Sequence
  Synthetic construct
ctctccattttgtggatgagat

SEQ ID NO: 635 - DNA, 23 bp, Artificial Sequence
  Synthetic construct
cttcacggcagtggagagcaagt

SEQ ID NO: 636 - DNA, 18 bp, Artificial Sequence
  Synthetic construct
tctctgcgagcatggtct

SEQ ID NO: 637 - DNA, 18 bp, Artificial Sequence
  Synthetic construct
aagtctacctggccttcc

SEQ ID NO: 638 - DNA, 18 bp, Artificial Sequence
  Synthetic construct
ccgtgacagcatggttcc

SEQ ID NO: 639 - DNA, 18 bp, Artificial Sequence
  Synthetic construct
cacggtcctcacagctct

SEQ ID NO: 640 - DNA, 42 bp, Artificial Sequence
  Synthetic construct
aggtattgcgtgcagagacggtatgaagagcatgccaacacc

SEQ ID NO: 641 - DNA, 38 bp, Artificial Sequence
  Synthetic construct
aacaggtcgctgcatggctgcatcagcagcaacagcga

SEQ ID NO: 642 - DNA, 18 bp, Artificial Sequence
  Synthetic construct
aaggcggtcaaggaacgc

SEQ ID NO: 643 - DNA, 19 bp, Artificial Sequence
  Synthetic construct
cggagcttcgacagcagtc

SEQ ID NO: 644 - DNA, 20 bp, Artificial Sequence
  Synthetic construct
atgaagagcatgccaacacc

SEQ ID NO: 645 - DNA, 22 bp, Artificial Sequence
  Synthetic construct
aggtattgcgtgcagagacggt

SEQ ID NO: 646 - DNA, 18 bp, Artificial Sequence
  Synthetic construct
catcagcagcaacagcga

SEQ ID NO: 647 - DNA, 20 bp, Artificial Sequence
  Synthetic construct
aacaggtcgctgcatggctg

SEQ ID NO: 648 - DNA, 19 bp, Artificial Sequence
  Synthetic construct
gattccgtgaacaggtcgc

SEQ ID NO: 649 - DNA, 19 bp, Artificial Sequence
  Synthetic construct
ttctctgcgagcatggtct

SEQ ID NO: 650 - DNA, 41 bp, Artificial Sequence
  Synthetic construct
ccggagattgttccatgtgagcgtgttgctgctgatgccac

SEQ ID NO: 651 - DNA, 44 bp, Artificial Sequence
  Synthetic construct
aatgataccggcgctctgctctgcaatgtcctccagaatttcga

SEQ ID NO: 652 - DNA, 19 bp, Artificial Sequence
  Synthetic construct
acacggtcctcacagctct

SEQ ID NO: 653 - DNA, 20 bp, Artificial Sequence
  Synthetic construct
cctgggcagggaaatgttcc

SEQ ID NO: 654 - DNA, 18 bp, Artificial Sequence
  Synthetic construct
tgttgctgctgatgccac

SEQ ID NO: 655 - DNA, 22 bp, Artificial Sequence
  Synthetic construct
gcaatgtcctccagaatttcga

SEQ ID NO: 656 - DNA, 22 bp, Artificial Sequence
  Synthetic construct
gcaatgtcctccagaatttcga

SEQ ID NO: 657 - DNA, 22 bp, Artificial Sequence
  Synthetic construct
aatgataccggcgctctgctct

SEQ ID NO: 658 - DNA, 17 bp, Artificial Sequence
  Synthetic construct
tctgcggagcttcgaca

SEQ ID NO: 659 - DNA, 19 bp, Artificial Sequence
  Synthetic construct
ttctctgcgagcatggtct

SEQ ID NO: 660 - DNA, 41 bp, Artificial Sequence
  Synthetic construct
tgatgcctgatggaccaggaggatgccactgagagctgtga

SEQ ID NO: 661 - DNA, 44 bp, Artificial Sequence; identical to SEQ ID NO: 651
  Synthetic construct

SEQ ID NO: 662 - DNA, 17 bp, Artificial Sequence
  Synthetic construct
atgtgagcgcgacacgg

SEQ ID NO: 663 - DNA, 20 bp, Artificial Sequence
  Synthetic construct
cctgggcagggaaatgttcc

SEQ ID NO: 664 - DNA, 19 bp, Artificial Sequence
  Synthetic construct
atgccactgagagctgtga

SEQ ID NO: 665 - DNA, 22 bp, Artificial Sequence
  Synthetic construct
tgatgcctgatggaccaggagg

SEQ ID NO: 666 - DNA, 22 bp, Artificial Sequence
  Synthetic construct
gcaatgtcctccagaatttcga

SEQ ID NO: 667 - DNA, 22 bp, Artificial Sequence
  Synthetic construct
aatgataccggcgctctgctct

SEQ ID NO: 668 - DNA, 17 bp, Artificial Sequence
  Synthetic construct
cgtgaacaggtcgctgc

SEQ ID NO: 669 - DNA, 19 bp, Artificial Sequence
  Synthetic construct
ttctctgcgagcatggtct

SEQ ID NO: 670 - DNA, 41 bp, Artificial Sequence
  Synthetic construct
tgatgcctgatggaccaggaggtgatgccactgagagctgt

SEQ ID NO: 671 - DNA, 44 bp, Artificial Sequence; identical to SEQ ID NO: 651
  Synthetic construct

SEQ ID NO: 672 - DNA, 17 bp, Artificial Sequence
  Synthetic construct
atgtgagcgcgacacgg

SEQ ID NO: 673 - DNA, 20 bp, Artificial Sequence
  Synthetic construct
cctgggcagggaaatgttcc

SEQ ID NO: 674 - DNA, 19 bp, Artificial Sequence
  Synthetic construct
tgatgccactgagagctgt

SEQ ID NO: 675 - DNA, 22 bp, Artificial Sequence
  Synthetic construct
tgatgcctgatggaccaggagg

SEQ ID NO: 676 - DNA, 22 bp, Artificial Sequence
  Synthetic construct
gcaatgtcctccagaatttcga

SEQ ID NO: 677 - DNA, 22 bp, Artificial Sequence
  Synthetic construct
aatgataccggcgctctgctct

SEQ ID NO: 678 - DNA, 18 bp, Artificial Sequence
  Synthetic construct
tgttgctgctgatgccac

SEQ ID NO: 679 - DNA, 19 bp, Artificial Sequence
  Synthetic construct
ttctctgcgagcatggtct

SEQ ID NO: 680 - DNA, 40 bp, Artificial Sequence
  Synthetic construct
tgatgcctgatggaccaggaggagagctgtgaggaccgtg

SEQ ID NO: 681 - DNA, 44 bp, Artificial Sequence; identical to SEQ ID NO: 651
  Synthetic construct

SEQ ID NO: 682 - DNA, 22 bp, Artificial Sequence
  Synthetic construct
ccggagattgttccatgtgagc

SEQ ID NO: 683 - DNA, 20 bp, Artificial Sequence
  Synthetic construct
cctgggcagggaaatgttcc

SEQ ID NO: 684 - DNA, 18 bp, Artificial Sequence
  Synthetic construct
agagctgtgaggaccgtg

SEQ ID NO: 685 - DNA, 22 bp, Artificial Sequence
  Synthetic construct
tgatgcctgatggaccaggagg

SEQ ID NO: 686 - DNA, 22 bp, Artificial Sequence
  Synthetic construct
gcaatgtcctccagaatttcga

SEQ ID NO: 687 - DNA, 22 bp, Artificial Sequence
  Synthetic construct
aatgataccggcgctctgctct

SEQ ID NO: 688 - DNA, 19 bp, Artificial Sequence
  Synthetic construct
gattccgtgaacaggtcgc

SEQ ID NO: 689 - DNA, 19 bp, Artificial Sequence
  Synthetic construct
ttctctgcgagcatggtct

SEQ ID NO: 690 - DNA, 41 bp, Artificial Sequence
  Synthetic construct
ccggagattgttccatgtgagcgtcgctgttgctgctgatg

SEQ ID NO: 691 - DNA, 44 bp, Artificial Sequence; identical to SEQ ID NO: 651
  Synthetic construct

SEQ ID NO: 692 - DNA, 19 bp, Artificial Sequence
  Synthetic construct
acacggtcctcacagctct

SEQ ID NO: 693 - DNA, 20 bp, Artificial Sequence
  Synthetic construct
cctgggcagggaaatgttcc

SEQ ID NO: 694 - DNA, 18 bp, Artificial Sequence
  Synthetic construct
tcgctgttgctgctgatg

SEQ ID NO: 695 - DNA, 23 bp, Artificial Sequence
  Synthetic construct
ccggagattgttccatgtgagcg

SEQ ID NO: 696 - DNA, 22 bp, Artificial Sequence
  Synthetic construct
gcaatgtcctccagaatttcga

SEQ ID NO: 697 - DNA, 22 bp, Artificial Sequence
  Synthetic construct
aatgataccggcgctctgctct

SEQ ID NO: 698 - DNA, 23 bp, Artificial Sequence
  Synthetic construct
aacatgaagagcatgccaacacc

SEQ ID NO: 699 - DNA, 19 bp, Artificial Sequence
  Synthetic construct
agcagagcgccggtatcat

SEQ ID NO: 700 - DNA, 43 bp, Artificial Sequence
  Synthetic construct
gtcgaagctccgcagaggcacttctctgcacgcaatacctccg

SEQ ID NO: 701 - DNA, 45 bp, Artificial Sequence
  Synthetic construct
cgctgttgctgctgatgccactgcggagattgttccatgtgagcg

SEQ ID NO: 702 - DNA, 22 bp, Artificial Sequence
  Synthetic construct
gcagcgacctgttcacggaatc

SEQ ID NO: 703 - DNA, 21 bp, Artificial Sequence
  Synthetic construct
agagctgtgaggaccgtgtcg

SEQ ID NO: 704 - DNA, 21 bp, Artificial Sequence
  Synthetic construct
tctctgcacgcaatacctccg

SEQ ID NO: 705 - DNA, 22 bp, Artificial Sequence
  Synthetic construct
gtcgaagctccgcagaggcact

SEQ ID NO: 706 - DNA, 22 bp, Artificial Sequence
  Synthetic construct
cggagattgttccatgtgagcg

SEQ ID NO: 707 - DNA, 23 bp, Artificial Sequence
  Synthetic construct
cgctgttgctgctgatgccactg

SEQ ID NO: 708 - DNA, 22 bp, Artificial Sequence
  Synthetic construct
tgttccgcctcgaaattctgga

SEQ ID NO: 709 - DNA, 23 bp, Artificial Sequence
  Synthetic construct
ttccgtacgcttcagtacagcat

SEQ ID NO: 710 - DNA, 43 bp, Artificial Sequence
  Synthetic construct
tcccgacacgccatagaaacgcaacctggccttccagaccatg

SEQ ID NO: 711 - DNA, 43 bp, Artificial Sequence
  Synthetic construct
acagcaaatgacctccgcactgcccagagggagaaccagtccg

SEQ ID NO: 712 - DNA, 21 bp, Artificial Sequence
  Synthetic construct
acggcagtggagagctgaagt

SEQ ID NO: 713 - DNA, 23 bp, Artificial Sequence
  Synthetic construct
cgaagctatggtcagaagccgtg

SEQ ID NO: 714 - DNA, 23 bp, Artificial Sequence
  Synthetic construct
tcccgacacgccatagaaacgca

SEQ ID NO: 715 - DNA, 23 bp, Artificial Sequence
  Synthetic construct
tcccgacacgccatagaaacgca

SEQ ID NO: 716 - DNA, 20 bp, Artificial Sequence
  Synthetic construct
ccagagggagaaccagtccg

SEQ ID NO: 717 - DNA, 23 bp, Artificial Sequence
  Synthetic construct
acagcaaatgacctccgcactgc

SEQ ID NO: 718 - DNA, 22 bp, Artificial Sequence
  Synthetic construct
tgttccgcctcgaaattctgga

SEQ ID NO: 719 - DNA, 23 bp, Artificial Sequence
  Synthetic construct
ttccgtacgcttcagtacagcat

SEQ ID NO: 720 - DNA, 42 bp, Artificial Sequence
  Synthetic construct
tcccgacacgccatagaaacgcatggccttccagaccatgct

SEQ ID NO: 721 - DNA, 43 bp, Artificial Sequence; identical to SEQ ID NO: 711
  Synthetic construct

SEQ ID NO: 722 - DNA, 21 bp, Artificial Sequence
  Synthetic construct
acggcagtggagagctgaagt

SEQ ID NO: 723 - DNA, 23 bp, Artificial Sequence
  Synthetic construct
cgaagctatggtcagaagccgtg

SEQ ID NO: 724 - DNA, 19 bp, Artificial Sequence
  Synthetic construct
tggccttccagaccatgct

SEQ ID NO: 725 - DNA, 23 bp, Artificial Sequence
  Synthetic construct
tcccgacacgccatagaaacgca

SEQ ID NO: 726 - DNA, 20 bp, Artificial Sequence
  Synthetic construct
ccagagggagaaccagtccg

SEQ ID NO: 727 - DNA, 23 bp, Artificial Sequence
  Synthetic construct
acagcaaatgacctccgcactgc

SEQ ID NO: 728 - DNA, 22 bp, Artificial Sequence
  Synthetic construct
tgttccgcctcgaaattctgga

SEQ ID NO: 729 - DNA, 23 bp, Artificial Sequence
  Synthetic construct
ttccgtacgcttcagtacagcat

SEQ ID NO: 730 - DNA, 43 bp, Artificial Sequence
  Synthetic construct
tcccgacacgccatagaaacgcactggccttccagaccatgct

SEQ ID NO: 731 - DNA, 43 bp, Artificial Sequence; identical to SEQ ID NO: 711
  Synthetic construct

SEQ ID NO: 732 - DNA, 21 bp, Artificial Sequence
  Synthetic construct
acggcagtggagagctgaagt

SEQ ID NO: 733 - DNA, 23 bp, Artificial Sequence
  Synthtetic construct
cgaagctatggtcagaagccgtg

SEQ ID NO: 734 - DNA, 20 bp, Artificial Sequence
  Synthetic construct
ctggccttccagaccatgct

SEQ ID NO: 735 - DNA, 23 bp, Artificial Sequence
  Synthetic construct
tcccgacacgccatagaaacgca

SEQ ID NO: 736 - DNA, 20 bp, Artificial Sequence
  Synthetic construct
ccagagggagaaccagtccg

SEQ ID NO: 737 - DNA, 23 bp, Artificial Sequence
  Synthetic construct
acagcaaatgacctccgcactgc

SEQ ID NO: 738 - DNA, 21 bp, Artificial Sequence
  Synthetic construct
ccgcctcgaaattctggagga

SEQ ID NO: 739 - DNA, 23 bp, Artificial Sequence
  Synthetic construct
ttccgtacgcttcagtacagcat

SEQ ID NO: 740 - DNA, 44 bp, Artificial Sequence
  Synthetic construct
tcccgacacgccatagaaacgcacttccagaccatgctcgcaga

SEQ ID NO: 741 - DNA, 43 bp, Artificial Sequence; identical to SEQ ID NO: 711
  Synthetic construct

SEQ ID NO: 742 - DNA, 21 bp, Artificial Sequence
  Synthetic construct
acggcagtggagagctgaagt

SEQ ID NO: 743 - DNA, 23 bp, Artificial Sequence
  Synthetic construct
cgaagctatggtcagaagccgtg

SEQ ID NO: 744 - DNA, 21 bp, Artificial Sequence
  Synthetic construct
cttccagaccatgctcgcaga

SEQ ID NO: 745 - DNA, 23 bp, Artificial Sequence
  Synthetic construct
tcccgacacgccatagaaacgca

SEQ ID NO: 746 - DNA, 20 bp, Artificial Sequence
  Synthetic construct
ccagagggagaaccagtccg

SEQ ID NO: 747 - DNA, 23 bp, Artificial Sequence
  Synthetic construct
acagcaaatgacctccgcactgc

SEQ ID NO: 748 - DNA, 21 bp, Artificial Sequence
  Synthetic construct
tccgcctcgaaattctggagg

SEQ ID NO: 749 - DNA, 23 bp, Artificial Sequence
  Synthetic construct
ttccgtacgcttcagtacagcat

SEQ ID NO: 750 - DNA, 43 bp, Artificial Sequence
  Synthetic construct
tcccgacacgccatagaaacgcaccttccagaccatgctcgca

SEQ ID NO: 751 - DNA, 43 bp, Artificial Sequence; identical to SEQ ID NO: 711
  Synthetic construct

SEQ ID NO: 752 - DNA, 21 bp, Artificial Sequence
  Synthetic construct
acggcagtggagagctgaagt

SEQ ID NO: 753 - DNA, 23 bp, Artificial Sequence
  Synthetic construct
cgaagctatggtcagaagccgtg

SEQ ID NO: 754 - DNA, 20 bp, Artificial Sequence
  Synthetic construct
ccttccagaccatgctcgca

SEQ ID NO: 755 - DNA, 23 bp, Artificial Sequence
  Synthetic construct
tcccgacacgccatagaaacgca

SEQ ID NO: 756 - DNA, 20 bp, Artificial Sequence
  Synthetic construct
ccagagggagaaccagtccg

SEQ ID NO: 757 - DNA, 23 bp, Artificial Sequence
  Synthetic construct
acagcaaatgacctccgcactgc

SEQ ID NO: 758 - DNA, 18 bp, Artificial Sequence
  Synthetic construct
accgtctctgcacgcaat

SEQ ID NO: 759 - DNA, 19 bp, Artificial Sequence
  Synthetic construct
agagcagagcgccggtatc

SEQ ID NO: 760 - DNA, 45 bp, Artificial Sequence
  Synthetic construct
gctccgcagaggcactgagtttttttacctccggattccgtgaac

SEQ ID NO: 761 - DNA, 47 bp, Artificial Sequence
  Synthetic construct
tgccactgagagctgtgaggactttttctgatgcctgatggaccagg

SEQ ID NO: 762 - DNA, 19 bp, Artificial Sequence
  Synthetic construct
ttccagccatgcagcgacc

SEQ ID NO: 763 - DNA, 19 bp, Artificial Sequence
  Synthetic construct
gtcgcgctcacatggaaca

SEQ ID NO: 764 - DNA, 19 bp, Artificial Sequence
  Synthetic construct
acctccggattccgtgaac

SEQ ID NO: 765 - DNA, 26 bp, Artificial Sequence
  Synthetic construct
gctccgcagaggcactgagttttttt

SEQ ID NO: 766 - DNA, 20 bp, Artificial Sequence
  Synthetic construct
ctgatgcctgatggaccagg

SEQ ID NO: 767 - DNA, 27 bp, Artificial Sequence
  Synthetic construct
tgccactgagagctgtgaggacttttt

SEQ ID NO: 768 - DNA, 20 bp, Artificial Sequence
  Synthetic construct
agctgactgacgaggtactg

SEQ ID NO: 769 - DNA, 19 bp, Artificial Sequence
  Synthetic construct
agttgcacgccgtaaatcc

SEQ ID NO: 770 - DNA, 40 bp, Artificial Sequence
  Synthetic construct
tccggtctgcggtttatgcttgtctgaaaacggctcacga

SEQ ID NO: 771 - DNA, 40 bp, Artificial Sequence
  Synthetic construct
actccggaaaaactgtgacccggccggtattgagcgagga

SEQ ID NO: 772 - DNA, 22 bp, Artificial Sequence
  Synthetic construct
tgcgacgtgattatgaatggtg

SEQ ID NO: 773 - DNA, 24 bp, Artificial Sequence
  Synthetic construct
cggaccttaacaacaacccgtaaa

SEQ ID NO: 774 - DNA, 19 bp, Artificial Sequence
  Synthetic construct
gtctgaaaacggctcacga

SEQ ID NO: 775 - DNA, 21 bp, Artificial Sequence
  Synthetic construct
tccggtctgcggtttatgctt

SEQ ID NO: 776 - DNA, 18 bp, Artificial Sequence
  Synthetic construct
gccggtattgagcgagga

SEQ ID NO: 777 - DNA, 22 bp, Artificial Sequence
  Synthetic construct
actccggaaaaactgtgacccg

SEQ ID NO: 778 - DNA, 18 bp, Artificial Sequence
  Synthetic construct
ataccgtctctgcacgca

SEQ ID NO: 779 - DNA, 18 bp, Artificial Sequence
  Syntheic construct
tgatggaccaggagggtt

SEQ ID NO: 780 - DNA, 40 bp, Artificial Sequence
  Synthetic construct
aagctccgcagaggcactgatacctccggattccgtgaac

SEQ ID NO: 781 - DNA, 42 bp, Artificial Sequence
  Synthetic construct
agcagtctttcgctgttgctgctccggagattgttccatgtg

SEQ ID NO: 782 - DNA, 16 bp, Artificial Sequence
  Synthetic construct
agccatgcagcgacct

SEQ ID NO: 783 - DNA, 22 bp, Artificial Sequence
  Synthetic construct
gatgccactgagagctgtgagg

SEQ ID NO: 784 - DNA, 20 bp, Artificial Sequence
  Synthetic construct
tacctccggattccgtgaac

SEQ ID NO: 785 - DNA, 20 bp, Artificial Sequence
  Synthetic construct
aagctccgcagaggcactga

SEQ ID NO: 786 - DNA, 20 bp, Artificial Sequence
  Synthetic construct
tccggagattgttccatgtg

SEQ ID NO: 787 - DNA, 22 bp, Artificial Sequence
  Synthetic construct
agcagtctttcgctgttgctgc

SEQ ID NO: 788 - DNA, 19 bp, Artificial Sequence
  Synthetic construct
gcatgccaacaccttttcc

SEQ ID NO: 789 - DNA, 18 bp, Artificial Sequence
  Synthetic construct
tgatggaccaggagggtt

SEQ ID NO: 790 - DNA, 41 bp, Artificial Sequence
  Synthetic construct
cgacctgttcacggaatccggcttgaccgcctttccgatac

SEQ ID NO: 791 - DNA, 42 bp, Artificial Sequence; identical to SEQ ID NO: 781
  Synthetic construct

SEQ ID NO: 792 - DNA, 20 bp, Artificial Sequence
  Synthetic construct
aggtattgcgtgcagagacg

SEQ ID NO: 793 - DNA, 19 bp, Artificial Sequence
  Synthetic construct
actgagagctgtgaggacc

SEQ ID NO: 794 - DNA, 20 bp, Artificial Sequence
  Synthetic construct
cttgaccgcctttccgatac

SEQ ID NO: 795 - DNA, 21 bp, Artificial Sequence
  Synthetic construct
cgacctgttcacggaatccgg

SEQ ID NO: 796 - DNA, 20 bp, Artificial Sequence
  Synthetic construct
tccggagattgttccatgtg

SEQ ID NO: 797 - DNA, 22 bp, Artificial Sequence
  Synthetic construct
agcagtctttcgctgttgctgc

SEQ ID NO: 798 - DNA, 19 bp, Artificial Sequence
  Synthetic construct
tggatggtatcgagttcat

SEQ ID NO: 799 - DNA, 20 bp, Artificial Sequence
  Synthetic construct
gattggtattcgtcaaggaa

SEQ ID NO: 800 - DNA, 39 bp, Artificial Sequence
  Synthetic construct
ctccaagctctatgctccgaacttagatattgctccagc

SEQ ID NO: 801 - DNA, 40 bp, Artificial Sequence
  Synthetic construct
agagccgtggattcatcatgcgcaagtattactcatcgat

SEQ ID NO: 802 - DNA, 19 bp, Artificial Sequence
  Synthetic construct
cctgccaatccataaccat

SEQ ID NO: 803 - DNA, 17 bp, Artificial Sequence
  Synthetic construct
gttgtgggaatgctcca

SEQ ID NO: 804 - DNA, 20 bp, Artificial Sequence
  Synthetic construct
aacttagatattgctccagc

SEQ ID NO: 805 - DNA, 19 bp, Artificial Sequence
  Synthetic construct
ctccaagctctatgctccg

SEQ ID NO: 806 - DNA, 20 bp, Artificial Sequence
  Synthetic construct
cgcaagtattactcatcgat

SEQ ID NO: 807 - DNA, 20 bp, Artificial Sequence
  Synthetic construct
agagccgtggattcatcatg

SEQ ID NO: 808 - DNA, 20 bp, Artificial Sequence
  Synthetic construct
agattctagacctcctgatg

SEQ ID NO: 809 - DNA, 20 bp, Artificial Sequence
  Synthetic construct
gtgcagtggctataacatat

SEQ ID NO: 810 - DNA, 42 bp, Artificial Sequence
  Synthetic construct
ctgagttcctcttgcatgatcaaagaggacagaatgagtact

SEQ ID NO: 811 - DNA, 38 bp, Artificial Sequence
  Synthetic construct
cgggatttgttaggcacgagggcacttctcaaactaat

SEQ ID NO: 812 - DNA, 20 bp, Artificial Sequence
  Synthetic construct
ttcatttgagtacctcggtc

SEQ ID NO: 813 - DNA, 21 bp, Artificial Sequence
  Synthetic construct
tgatggatatgtttccacctc

SEQ ID NO: 814 - DNA, 20 bp, Artificial Sequence
  Synthetic construct
aagaggacagaatgagtact

SEQ ID NO: 815 - DNA, 22 bp, Artificial Sequence
  Synthetic construct
ctgagttcctcttgcatgatca

SEQ ID NO: 816 - DNA, 19 bp, Artificial Sequence
  Synthetic construct
gggcacttctcaaactaat

SEQ ID NO: 817 - DNA, 19 bp, Artificial Sequence
  Synthetic construct
cgggatttgttaggcacga

SEQ ID NO: 818 - DNA, 17 bp, Artificial Sequence
  Synthetic construct
caggtggtcttatgcca

SEQ ID NO: 819 - DNA, 20 bp, Artificial Sequence
  Synthetic construct
gtgcagtggctataacatat

SEQ ID NO: 820 - DNA, 42 bp, Artificial Sequence
  Synthetic construct
ctgagttcctcttgcatgatcaagaggacagaatgagtactt

SEQ ID NO: 821 - DNA, 38 bp, Artificial Sequence; identical to SEQ ID NO: 811
  Synthetic construct

SEQ ID NO: 822 - DNA, 20 bp, Artificial Sequence
  Synthetic construct
ttcatttgagtacctcggtc

SEQ ID NO: 823 - DNA, 21 bp, Artificial Sequence
  Synthetic construct
tgatggatatgtttccacctc

SEQ ID NO: 824 - DNA, 20 bp, Artificial Sequence
  Synthetic construct
agaggacagaatgagtactt

SEQ ID NO: 825 - DNA, 22 bp, Artificial Sequence
  Synthetic construct
ctgagttcctcttgcatgatca

SEQ ID NO: 826 - DNA, 19 bp, Artificial Sequence
  Synthetic construct
gggcacttctcaaactaat

SEQ ID NO: 827 - DNA, 19 bp, Artificial Sequence
  Synthetic construct
cgggatttgttaggcacga

SEQ ID NO: 828 - DNA, 19 bp, Artificial Sequence
  Synthetic construct
tggatggtatcgagttcat

SEQ ID NO: 829 - DNA, 20 bp, Artificial Sequence
  Synthetic construct
gattggtattcgtcaaggaa

SEQ ID NO: 830 - DNA, 39 bp, Artificial Sequence; identical to SEQ ID NO: 800
  Synthetic construct

SEQ ID NO: 831 - DNA, 40 bp, Artificial Sequence; identical to SEQ ID NO: 801
  Synthetic construct

SEQ ID NO: 832 - DNA, 23 bp, Artificial Sequence
  Synthetic construct
ctgccaatccataaccatctgct

SEQ ID NO: 833 - DNA, 22 bp, Artificial Sequence
  Synthetic construct
ggttgtgggaatgctccaagat

SEQ ID NO: 834 - DNA, 20 bp, Artificial Sequence
  Synthetic construct
aacttagatattgctccagc

SEQ ID NO: 835 - DNA, 19 bp, Artificial Sequence
  Synthetic construct
ctccaagctctatgctccg

SEQ ID NO: 836 - DNA, 20 bp, Artificial Sequence
  Synthetic construct
cgcaagtattactcatcgat

SEQ ID NO: 837 - DNA, 20 bp, Artificial Sequence
  Synthetic construct
agagccgtggattcatcatg

SEQ ID NO: 838 - DNA, 19 bp, Artificial Sequence
  Synthetic construct
atatcgggcagattctaga

SEQ ID NO: 839 - DNA, 43 bp, Artificial Sequence
  Synthetic construct
tttgagtacctcggtcaaagtaccctcctgatgaaataaagca

SEQ ID NO: 840 - DNA, 20 bp, Artificial Sequence
  Synthetic construct
cctcctgatgaaataaagca

SEQ ID NO: 841 - DNA, 20 bp, Artificial Sequence
  Synthetic construct
gcagattctagacctcctga

SEQ ID NO: 842 - DNA, 21 bp, Artificial Sequence
  Synthetic construct
ggtgcagtggctataacatat

SEQ ID NO: 843 - DNA, 43 bp, Artificial Sequence
  Synthetic construct
cgtctgagttcctcttgcatgatctgccaagaggacagaatga

SEQ ID NO: 844 - DNA, 47 bp, Artificial Sequence
  Synthetic construct
ggcacgatgatggatatgtttccagaccagacaatatagtttgaccc

SEQ ID NO: 845 - DNA, 23 bp, Artificial Sequence
  Synthetic construct
caattagtttgagaagtgcccac

SEQ ID NO: 846 - DNA, 19 bp, Artificial Sequence
  Synthetic construct
tgccaagaggacagaatga

SEQ ID NO: 847 - DNA, 24 bp, Artificial Sequence
  Synthetic construct
cgtctgagttcctcttgcatgatc

SEQ ID NO: 848 - DNA, 23 bp, Artificial Sequence
  Synthetic construct
gaccagacaatatagtttgaccc

SEQ ID NO: 849 - DNA, 24 bp, Artificial Sequence
  Synthetic construct
ggcacgatgatggatatgtttcca

SEQ ID NO: 850 - DNA, 20 bp, Artificial Sequence
  Synthetic construct
gcagattctagacctcctga

SEQ ID NO: 851 - DNA, 18 bp, Artificial Sequence
  Synthetic construct
ttgggtgcagtggctata

SEQ ID NO: 852 - DNA, 43 bp, Artificial Sequence; identical to SEQ ID NO: 843
  Synthetic construct

SEQ ID NO: 853 - DNA, 47 bp, Artificial Sequence; identical to SEQ ID NO: 844
  Synthetic construct

SEQ ID NO: 854 - DNA, 23 bp, Artificial Sequence
  Synthetic construct
tttgagtacctcggtcaaagtac

SEQ ID NO: 855 - DNA, 23 bp, Artificial Sequence
  Synthetic construct
caattagtttgagaagtgcccac

SEQ ID NO: 856 - DNA, 19 bp, Artificial Sequence
  Synthetic construct
tgccaagaggacagaatga

SEQ ID NO: 857 - DNA, 24 bp, Artificial Sequence
  Synthetic construct
cgtctgagttcctcttgcatgatc

SEQ ID NO: 858 - DNA, 23 bp, Artificial Sequence
  Synthetic construct
gaccagacaatatagtttgaccc

SEQ ID NO: 859 - DNA, 24 bp, Artificial Sequence
  Synthetic construct
ggcacgatgatggatatgtttcca

SEQ ID NO: 860 - DNA, 19 bp, Artificial Sequence
  Synthetic construct
agcagtcaggtggtcttat

SEQ ID NO: 861 - DNA, 21 bp, Artificial Sequence
  Synthetic construct
ggtgcagtggctataacatat

SEQ ID NO: 862 - DNA, 43 bp, Artificial Sequence
  Synthetic construct
cgtctgagttcctcttgcatgatcgccaagaggacagaatgag

SEQ ID NO: 863 - DNA, 47 bp, Artificial Sequence; identical to SEQ ID NO: 844
  Synthetic construct

SEQ ID NO: 864 - DNA, 24 bp, Artificial Sequence
  Synthetic construct
catttgagtacctcggtcaaagta

SEQ ID NO: 865 - DNA, 23 bp, Artificial Sequence
  Synthetic construct
caattagtttgagaagtgcccac

SEQ ID NO: 866 - DNA, 19 bp, Artificial Sequence
  Synthetic construct
gccaagaggacagaatgag

SEQ ID NO: 867 - DNA, 24 bp, Artificial Sequence
  Synthetic construct
cgtctgagttcctcttgcatgatc

SEQ ID NO: 868 - DNA, 23 bp, Artificial Sequence
  Synthetic construct
gaccagacaatatagtttgaccc

SEQ ID NO: 869 - DNA, 24 bp, Artificial Sequence
  Synthetic construct
ggcacgatgatggatatgtttcca

SEQ ID NO: 870 - DNA, 20 bp, Artificial Sequence
  Synthetic construct
gcagattctagacctcctga

SEQ ID NO: 871 - DNA, 21 bp, Artificial Sequence
  Synthetic construct
cttgttcatctggatgaggac

SEQ ID NO: 872 - DNA, 43 bp, Artificial Sequence
  Synthetic construct
cgtctgagttcctcttgcatgatcaggtggtcttatgccaaga

SEQ ID NO: 873 - DNA, 45 bp, Artificial Sequence
  Synthetic construct
ggcacgatgatggatatgtttccaggtgcagtggctataacatat

SEQ ID NO: 874 - DNA, 24 bp, Artificial Sequence
  Synthetic construct
tgagtacctcggtcaaagtactca

SEQ ID NO: 875 - DNA, 24 bp, Artificial Sequence
  Synthetic construct
agtttgagaagtgcccacttagtg

SEQ ID NO: 876 - DNA, 19 bp, Artificial Sequence
  Synthetic construct
aggtggtcttatgccaaga

SEQ ID NO: 877 - DNA, 24 bp, Artificial Sequence
  Synthetic construct
cgtctgagttcctcttgcatgatc

SEQ ID NO: 878 - DNA, 21 bp, Artificial Sequence
  Synthetic construct
ggtgcagtggctataacatat

SEQ ID NO: 879 - DNA, 20 bp, Artificial Sequence
  Synthetic construct
ggcacgatgatggatatgtt

SEQ ID NO: 880 - DNA, 19 bp, Artificial Sequence
  Synthetic construct
gatgaatccacggctcttc

SEQ ID NO: 881 - DNA, 45 bp, Artificial Sequence
  Synthetic
tggaatcccacctaaagcagaaacatatgttatagccactgcacc

SEQ ID NO: 882 - DNA, 41 bp, Artificial Sequence
  Synthetic construct
agatattgctccagcagcagatgtccaagctctatgctccg

SEQ ID NO: 883 - DNA, 24 bp, Artificial Sequence
  Synthetic construct
tcatctggatgaggactgtatgcc

SEQ ID NO: 884 - DNA, 23 bp, Artificial Sequence
  Synthetic construct
gttatggattggcaggtttccct

SEQ ID NO: 885 - DNA, 21 bp, Artificial Sequence
  Synthetic construct
atatgttatagccactgcacc

SEQ ID NO: 886 - DNA, 24 bp, Artificial Sequence
  Synthetic construct
tggaatcccacctaaagcagaaac

SEQ ID NO: 887 - DNA, 18 bp, Artificial Sequence
  Synthetic construct
tccaagctctatgctccg

SEQ ID NO: 888 - DNA, 23 bp, Artificial Sequence
  Synthetic construct
agatattgctccagcagcagatg

SEQ ID NO: 889 - DNA, 19 bp, Artificial Sequence
  Synthetic construct
aggtggtcttatgccaaga

SEQ ID NO: 890 - DNA, 20 bp, Artificial Sequence
  Synthetic construct
ggaaacctgccaatccataa

SEQ ID NO: 891 - DNA, 43 bp, Artificial Sequence
  Synthetic construct
gttgggtgcagtggctataacatggcacgatgatggatatgtt

SEQ ID NO: 892 - DNA, 48 bp, Artificial Sequence
  Synthetic construct
ggcatacagtcctcatccagatgacgataccatccatatatttgggag

SEQ ID NO: 893 - DNA, 24 bp, Artificial Sequence
  Synthetic construct
cactaagtgggcacttctcaaact

SEQ ID NO: 894 - DNA, 24 bp, Artificial Sequence
  Synthetic construct
gtttctgctttaggtgggattcca

SEQ ID NO: 895 - DNA, 20 bp, Artificial Sequence
  Synthetic construct
ggcacgatgatggatatgtt

SEQ ID NO: 896 - DNA, 23 bp, Artificial Sequence
  Synthetic construct
gttgggtgcagtggctataacat

SEQ ID NO: 897 - DNA, 24 bp, Artificial Sequence
  Synthetic construct
cgataccatccatatatttgggag

SEQ ID NO: 898 - DNA, 24 bp, Artificial Sequence
  Synthetic construct
ggcatacagtcctcatccagatga

SEQ ID NO: 899 - DNA, 25 bp, Artificial Sequence
  Synthetic construct
tcaacctttatgatcatgcaagagg

SEQ ID NO: 900 - DNA, 24 bp, Artificial Sequence
  Synthetic construct
cgatgtaattgttcatcaagcacc

SEQ ID NO: 901 - DNA, 45 bp, Artificial Sequence
  Synthetic construct
gacccactaagtgggcacttctcactcagacgggatttgttaggc

SEQ ID NO: 902 - DNA, 49 bp, Artificial Sequence
  Synthetic construct
agccactgcacccaacatgtttaacaatcccacctaaagcagaaacttc

SEQ ID NO: 903 - DNA, 25 bp, Artificial Sequence
  Synthetic construct
gaggtggaaacatatccatcatcgt

SEQ ID NO: 904 - DNA, 25 bp, Artificial Sequence
  Synthetic construct
gcatacagtcctcatccagatgaac

SEQ ID NO: 905 - DNA, 21 bp, Artificial Sequence
  Synthetic construct
ctcagacgggatttgttaggc

SEQ ID NO: 906 - DNA, 24 bp, Artificial Sequence
  Synthetic construct
gacccactaagtgggcacttctca

SEQ ID NO: 907 - DNA, 24 bp, Artificial Sequence
  Synthetic construct
aatcccacctaaagcagaaacttc

SEQ ID NO: 908 - DNA, 25 bp, Artificial Sequence
  Synthetic construct
agccactgcacccaacatgtttaac

SEQ ID NO: 909 - DNA, 25 bp, Artificial Sequence
  Synthetic construct
tcaacctttatgatcatgcaagagg

SEQ ID NO: 910 - DNA, 24 bp, Artificial Sequence
  Synthetic construct
cgatgtaattgttcatcaagcacc

SEQ ID NO: 911 - DNA, 45 bp, Artificial Sequence
  Synthetic construct
tgacccactaagtgggcacttctcctcagacgggatttgttaggc

SEQ ID NO: 912 - DNA, 49 bp, Artificial Sequence; identical to SEQ ID NO: 902
  Synthetic construct

SEQ ID NO: 913 - DNA, 25 bp, Artificial Sequence
  Synthetic construct
gaggtggaaacatatccatcatcgt

SEQ ID NO: 914 - DNA, 25 bp, Artificial Sequence
  Synthetic construct
gcatacagtcctcatccagatgaac

SEQ ID NO: 915 - DNA, 21 bp, Artificial Sequence
  Synthetic construct
ctcagacgggatttgttaggc

SEQ ID NO: 916 - DNA, 24 bp, Artificial Sequence
  Synthetic construct
tgacccactaagtgggcacttctc

SEQ ID NO: 917 - DNA, 24 bp, Artificial Sequence
  Synthetic construct
aatcccacctaaagcagaaacttc

SEQ ID NO: 918 - DNA, 25 bp, Artificial Sequence
  Synthetic construct
agccactgcacccaacatgtttaac

SEQ ID NO: 919 - DNA, 25 bp, Artificial Sequence
  Synthetic construct
acgatgtaattgttcatcaagcacc

SEQ ID NO: 920 - DNA, 45 bp, Artificial Sequence; identical to SEQ ID NO: 901
  Synthetic construct

SEQ ID NO: 921 - DNA, 24 bp, Artificial Sequence
  Synthetic construct
gacccactaagtgggcacttctca

SEQ ID NO: 922 - DNA, 24 bp, Artificial Sequence
  Synthetic construct
agtcaggtggtcttatgccaagag

SEQ ID NO: 923 - DNA, 25 bp, Artificial Sequence
  Synthetic construct
ccatctgctgctggagcaatatcta

SEQ ID NO: 924 - DNA, 48 bp, Artificial Sequence
  Synthetic construct
tgacccactaagtgggcacttctcatcatgcaagaggaactcagacgg

SEQ ID NO: 925 - DNA, 50 bp, Artificial Sequence
  Synthetic construct
gccactgcacccaacatgtttaacgtggaatcccacctaaagcagaaact

SEQ ID NO: 926 - DNA, 25 bp, Artificial Sequence
  Synthetic construct
acatatccatcatcgtgcctaacaa

SEQ ID NO: 927 - DNA, 25 bp, Artificial Sequence
  Synthetic construct
ggcatacagtcctcatccagatgaa

SEQ ID NO: 928 - DNA, 23 bp, Artificial Sequence
  Synthetic construct
tcatgcaagaggaactcagacgg

SEQ ID NO: 929 - DNA, 25 bp, Artificial Sequence
  Synthetic construct
tgacccactaagtgggcacttctca

SEQ ID NO: 930 - DNA, 25 bp, Artificial Sequence
  Synthetic construct
tggaatcccacctaaagcagaaact

SEQ ID NO: 931 - DNA, 25 bp, Artificial Sequence
  Synthetic construct
gccactgcacccaacatgtttaacg

SEQ ID NO: 932 - DNA, 25 bp, Artificial Sequence
  Synthetic construct
tctgctgctggagcaatatctaagt

SEQ ID NO: 933 - DNA, 24 bp, Artificial Sequence
  Synthetic construct
agtcaggtggtcttatgccaagag

SEQ ID NO: 934 - DNA, 25 bp, Artificial Sequence
  Synthetic construct
ccatctgctgctggagcaatatcta

SEQ ID NO: 935 - DNA, 48 bp, Artificial Sequence
  Synthetic construct
gacccactaagtgggcacttctcaatcatgcaagaggaactcagacgg

SEQ ID NO: 936 - DNA, 50 bp, Artificial Sequence; identical to SEQ ID NO: 925
  Synthetic construct

SEQ ID NO: 937 - DNA, 25 bp, Artificial Sequence
  Synthetic construct
acatatccatcatcgtgcctaacaa

SEQ ID NO: 938 - DNA, 25 bp, Artificial Sequence
  Synthetic construct
ggcatacagtcctcatccagatgaa

SEQ ID NO: 939 - DNA, 23 bp, Artificial Sequence
  Synthetic construct
tcatgcaagaggaactcagacgg

SEQ ID NO: 940 - DNA, 25 bp, Artificial Sequence
  Synthetic construct
gacccactaagtgggcacttctcaa

SEQ ID NO: 941 - DNA, 25 bp, Artificial Sequence
  Synthetic construct
tggaatcccacctaaagcagaaact

SEQ ID NO: 942 - DNA, 25 bp, Artificial Sequence
  Synthetic construct
gccactgcacccaacatgtttaacg

SEQ ID NO: 943 - DNA, 18 bp, Artificial Sequence
  Synthetic construct
gatattgctccagcagca

SEQ ID NO: 944 - DNA, 23 bp, Artificial Sequence
  Synthetic construct
aactttagattggtattcgtcaa

SEQ ID NO: 945 - DNA, 40 bp, Artificial Sequence
  Synthetic construct
gtgcatgatgaatccacggcgatggttatggattggcagg

SEQ ID NO: 946 - DNA, 40 bp, Artificial Sequence
  Synthetic construct
ggttgtgggaatgctccaagttacacctagactttgggtt

SEQ ID NO: 947 - DNA, 20 bp, Artificial Sequence
  Synthetic construct
ctctatgctccggagggaaa

SEQ ID NO: 948 - DNA, 25 bp, Artificial Sequence
  Synthetic construct
catcgatgagtaatacttgcgatga

SEQ ID NO: 949 - DNA, 20 bp, Artificial Sequence
  Synthetic construct
gatggttatggattggcagg

SEQ ID NO: 950 - DNA, 20 bp, Artificial Sequence
  Synthetic construct
gtgcatgatgaatccacggc

SEQ ID NO: 951 - DNA, 20 bp, Artificial Sequence
  Synthetic construct
ttacacctagactttgggtt

SEQ ID NO: 952 - DNA, 20 bp, Artificial Sequence
  Synthetic construct
ggttgtgggaatgctccaag

SEQ ID NO: 953 - DNA, 17 bp, Artificial Sequence
  Synthetic construct
tcttctgctaccagtgg

SEQ ID NO: 954 - DNA, 18 bp, Artificial Sequence
  Synthetic construct
attcaagtggagcacttg

SEQ ID NO: 955 - DNA, 41 bp, Artificial Sequence
  Synthetic construct
acacctcctgcataactcttggttagataaggtaaccgctc

SEQ ID NO: 956 - DNA, 41 bp, Artificial Sequence
  Synthetic construct
tatggatattgatccgacaagccggcgaagtttaggtaacc

SEQ ID NO: 957 - DNA, 17 bp, Artificial Sequence
  Synthetic construct
ccattgctcgatgctgt

SEQ ID NO: 958 - DNA, 18 bp, Artificial Sequence
  Synthetic construct
aatgccactaaccttggg

SEQ ID NO: 959 - DNA, 20 bp, Artificial Sequence
  Synthetic construct
gttagataaggtaaccgctc

SEQ ID NO: 960 - DNA, 21 bp, Artificial Sequence
  Synthetic construct
acacctcctgcataactcttg

SEQ ID NO: 961 - DNA, 18 bp, Artificial Sequence
  Synthetic construct
ggcgaagtttaggtaacc

SEQ ID NO: 962 - DNA, 23 bp, Artificial Sequence
  Synthetic construct
tatggatattgatccgacaagcc

SEQ ID NO: 963 - DNA, 19 bp, Artificial Sequence
  Synthetic construct
ccttggtgtgattgaagaa

SEQ ID NO: 964 - DNA, 20 bp, Artificial Sequence
  Synthetic construct
agcttctaatggttggttag

SEQ ID NO: 965 - DNA, 39 bp, Artificial Sequence
  Synthetic construct
aagtggcttatacgatggcttaagcagaagtaagaggct

SEQ ID NO: 966 - DNA, 42 bp, Artificial Sequence
  Synthetic construct
ctcaacaagaagagaggttgactagatgcggacatagtatca

SEQ ID NO: 967 - DNA, 21 bp, Artificial Sequence
  Synthetic construct
tctcagacataacatcaccac

SEQ ID NO: 968 - DNA, 22 bp, Artificial Sequence
  Synthetic construct
atctaccactcaccgatatttc

SEQ ID NO: 969 - DNA, 18 bp, Artificial Sequence
  Synthetic construct
aagcagaagtaagaggct

SEQ ID NO: 970 - DNA, 21 bp, Artificial Sequence
  Synthetic construct
aagtggcttatacgatggctt

SEQ ID NO: 971 - DNA, 19 bp, Artificial Sequence
  Synthetic construct
agatgcggacatagtatca

SEQ ID NO: 972 - DNA, 23 bp, Artificial Sequence
  Synthetic construct
ctcaacaagaagagaggttgact

SEQ ID NO: 973 - DNA, 19 bp, Artificial Sequence
  Synthetic construct
cgtgatgaatcgaacctag

SEQ ID NO: 974 - DNA, 17 bp, Artificial Sequence
  Synthetic construct
tggcttatacgatggct

SEQ ID NO: 975 - DNA, 43 bp, Artificial Sequence
  Synthetic construct
atcacaccaaggtcatctgtaaggtaggcttacttgagtttgt

SEQ ID NO: 976 - DNA, 38 bp, Artificial Sequence
  Synthetic construct
tgtgagtgtggtaaagctggagcctcttacttctgctt

SEQ ID NO: 977 - DNA, 19 bp, Artificial Sequence
  Synthetic construct
cggatatgaatgcggtagt

SEQ ID NO: 978 - DNA, 18 bp, Artificial Sequence
  Synthetic construct
aagtcattggacgagcaa

SEQ ID NO: 979 - DNA, 20 bp, Artificial Sequence
  Synthetic construct
gtaggcttacttgagtttgt

SEQ ID NO: 980 - DNA, 23 bp, Artificial Sequence
  Synthetic construct
atcacaccaaggtcatctgtaag

SEQ ID NO: 981 - DNA, 18 bp, Artificial Sequence
  Synthetic construct
agcctcttacttctgctt

SEQ ID NO: 982 - DNA, 20 bp, Artificial Sequence
  Synthetic construct
tgtgagtgtggtaaagctgg

SEQ ID NO: 983 - DNA, 17 bp, Artificial Sequence
  Synthetic construct
tcttctgctaccagtgg

SEQ ID NO: 984 - DNA, 18 bp, Artificial Sequence
  Synthetic construct
attcaagtggagcacttg

SEQ ID NO: 985 - DNA, 41 bp, Artificial Sequence; identical to SEQ ID NO: 955
  Synthetic construct

SEQ ID NO: 986 - DNA, 35 bp, Artificial Sequence
  Synthetic construct
acaagcccaaatgccactgatgttgaggcgaagtt

SEQ ID NO: 987 - DNA, 20 bp, Artificial Sequence
  Synthetic construct
ccattgctcgatgctgtcga

SEQ ID NO: 988 - DNA, 20 bp, Artificial Sequence
  Synthetic construct
gttagataaggtaaccgctc

SEQ ID NO: 989 - DNA, 21 bp, Artificial Sequence
  Synthetic construct
acacctcctgcataactcttg

SEQ ID NO: 990 - DNA, 17 bp, Artificial Sequence
  Synthetic construct
gatgttgaggcgaagtt

SEQ ID NO: 991 - DNA, 18 bp, Artificial Sequence
  Synthetic construct
acaagcccaaatgccact

SEQ ID NO: 992 - DNA, 20 bp, Artificial Sequence
  Synthetic construct
gttagataaggtaaccgctc

SEQ ID NO: 993 - DNA, 18 bp, Artificial Sequence
  Synthetic construct
tcactcgcaagtgaattc

SEQ ID NO: 994 - DNA, 41 bp, Artificial Sequence
  Synthetic construct
ggcttgtcggatcaatatccatagcaagagttatgcaggag

SEQ ID NO: 995 - DNA, 35 bp, Artificial Sequence
  Synthetic construct
ctcgtattgcggcggtaaacttgggctatcagcat

SEQ ID NO: 996 - DNA, 18 bp, Artificial Sequence
  Synthetic construct
aacgggcgacgtttaggc

SEQ ID NO: 997 - DNA, 22 bp, Artificial Sequence
  Synthetic construct
aacttcgcctcaacatcgaagt

SEQ ID NO: 998 - DNA, 18 bp, Artificial Sequence
  Synthetic construct
gcaagagttatgcaggag

SEQ ID NO: 999 - DNA, 23 bp, Artificial Sequence
  Synthetic construct
ggcttgtcggatcaatatccata

SEQ ID NO: 1000 - DNA, 17 bp, Artificial Sequence
  Synthetic construct
acttgggctatcagcat

SEQ ID NO: 1001 - DNA, 18 bp, Artificial Sequence
  Synthetic construct
ctcgtattgcggcggtaa

SEQ ID NO: 1002 - DNA, 20 bp, Artificial Sequence
  Synthetic construct
cacggtgatgacttattagg

SEQ ID NO: 1003 - DNA, 18 bp, Artificial Sequence
  Synthetic construct
cacactagagtcagttgc

SEQ ID NO: 1004 - DNA, 39 bp, Artificial Sequence
  Synthetic construct
tttctttggaaggccacttacacaatgctgatgtgcgta

SEQ ID NO: 1005 - DNA, 41 bp, Artificial Sequence
  Synthetic construct
atcctctttggacctaagctttcctgattgccttgtcagag

SEQ ID NO: 1006 - DNA, 20 bp, Artificial Sequence
  Synthetic construct
gactcgattgcctctcgtcc

SEQ ID NO: 1007 - DNA, 21 bp, Artificial Sequence
  Synthetic construct
ttgttggcagcgatgctctag

SEQ ID NO: 1008 - DNA, 17 bp, Artificial Sequence
  Synthetic construct
caatgctgatgtgcgta

SEQ ID NO: 1009 - DNA, 22 bp, Artificial Sequence
  Synthetic construct
tttctttggaaggccacttaca

SEQ ID NO: 1010 - DNA, 18 bp, Artificial Sequence
  Synthetic construct
ctgattgccttgtcagag

SEQ ID NO: 1011 - DNA, 23 bp, Artificial Sequence
  Synthetic construct
atcctctttggacctaagctttc

SEQ ID NO: 1012 - DNA, 19 bp, Artificial Sequence
  Synthetic construct
tcggtaagcgatgatacga

SEQ ID NO: 1013 - DNA, 18 bp, Artificial Sequence
  Synthetic construct
cgatgttgaggcgaagtt

SEQ ID NO: 1014 - DNA, 40 bp, Artificial Sequence
  Synthetic construct
ccattgctcgatgctgtcgacaatcttctgctaccagtgg

SEQ ID NO: 1015 - DNA, 44 bp, Artificial Sequence
  Synthetic construct
aagagttatgcaggaggtgttgggcttgtcggatcaatatccat

SEQ ID NO: 1016 - DNA, 23 bp, Artificial Sequence
  Synthetic construct
cgagcggttaccttatctaacaa

SEQ ID NO: 1017 - DNA, 17 bp, Artificial Sequence
  Synthetic construct
cctaaacgtcgcccgtt

SEQ ID NO: 1018 - DNA, 20 bp, Artificial Sequence
  Synthetic construct
caatcttctgctaccagtgg

SEQ ID NO: 1019 - DNA, 20 bp, Artificial Sequence
  Synthetic construct
ccattgctcgatgctgtcga

SEQ ID NO: 1020 - DNA, 21 bp, Artificial Sequence
  Synthetic construct
gcttgtcggatcaatatccat

SEQ ID NO: 1021 - DNA, 23 bp, Artificial Sequence
  Synthetic construct
aagagttatgcaggaggtgttgg

SEQ ID NO: 1022 - DNA, 20 bp, Artificial Sequence
  Synthetic construct
gcgtacccagtaccatattg

SEQ ID NO: 1023 - DNA, 19 bp, Artificial Sequence
  Synthetic construct
attcaagtggagcacttgg

SEQ ID NO: 1024 - DNA, 41 bp, Artificial Sequence
  Synthetic construct
cccaacacctcctgcataactcagataaggtaaccgctcgt

SEQ ID NO: 1025 - DNA, 40 bp, Artificial Sequence
  Synthetic construct
aagcccaaatgccactaaccttcgatgttgaggcgaagtt

SEQ ID NO: 1026 - DNA, 18 bp, Artificial Sequence
  Synthetic construct
ttgccattgctcgatgct

SEQ ID NO: 1027 - DNA, 18 bp, Artificial Sequence
  Synthetic construct
ctcgtattgcggcggtaa

SEQ ID NO: 1028 - DNA, 19 bp, Artificial Sequence
  Synthetic construct
agataaggtaaccgctcgt

SEQ ID NO: 1029 - DNA, 22 bp, Artificial Sequence
  Synthetic construct
cccaacacctcctgcataactc

SEQ ID NO: 1030 - DNA, 18 bp, Artificial Sequence
  Synthetic construct
cgatgttgaggcgaagtt

SEQ ID NO: 1031 - DNA, 22 bp, Artificial Sequence
  Synthetic construct
aagcccaaatgccactaacctt

SEQ ID NO: 1032 - DNA, 18 bp, Artificial Sequence
  Synthetic construct
cgagcaatggcaagagtt

SEQ ID NO: 1033 - DNA, 21 bp, Artificial Sequence
  Synthetic construct
taggcaattgaaacgagatcc

SEQ ID NO: 1034 - DNA, 39 bp, Artificial Sequence
  Synthetic construct
acttcgatgttgaggcgaagttgacaagcccaaatgcca

SEQ ID NO: 1035 - DNA, 37 bp, Artificial Sequence
  Synthetic construct
gcccaagtgctccacttgaaatgagcggctcttcact

SEQ ID NO: 1036 - DNA, 18 bp, Artificial Sequence
  Synthetic construct
ttaccgccgcaatacgag

SEQ ID NO: 1037 - DNA, 22 bp, Artificial Sequence
  Synthetic construct
tgaacatctgaattcacttgcg

SEQ ID NO: 1038 - DNA, 17 bp, Artificial Sequence
  Synthetic construct
gacaagcccaaatgcca

SEQ ID NO: 1039 - DNA, 22 bp, Artificial Sequence
  Synthetic construct
acttcgatgttgaggcgaagtt

SEQ ID NO: 1040 - DNA, 17 bp, Artificial Sequence
  Synthetic construct
atgagcggctcttcact

SEQ ID NO: 1041 - DNA, 20 bp, Artificial Sequence
  Synthetic construct
gcccaagtgctccacttgaa

SEQ ID NO: 1042 - DNA, 19 bp, Artificial Sequence
  Synthetic construct
tcggtaagcgatgatacga

SEQ ID NO: 1043 - DNA, 18 bp, Artificial Sequence
  Synthetic construct
cgatgttgaggcgaagtt

SEQ ID NO: 1044 - DNA, 40 bp, Artificial Sequence
  Synthetic construct
ctcgatgctgtcgacgagcaagcttcaatcttctgctacc

SEQ ID NO: 1045 - DNA, 44 bp, Artificial Sequence
  Synthetic construct
gcaagagttatgcaggaggtgttgcttgtcggatcaatatccat

SEQ ID NO: 1046 - DNA, 20 bp, Artificial Sequence
  Synthetic construct
atggtactgggtacgccact

SEQ ID NO: 1047 - DNA, 18 bp, Artificial Sequence
  Synthetic construct
gcctaaacgtcgcccgtt

SEQ ID NO: 1048 - DNA, 21 bp, Artificial Sequence
  Synthetic construct
aagcttcaatcttctgctacc

SEQ ID NO: 1049 - DNA, 19 bp, Artificial Sequence
  Synthetic construct
ctcgatgctgtcgacgagc

SEQ ID NO: 1050 - DNA, 21 bp, Artificial Sequence
  Synthetic construct
gcttgtcggatcaatatccat

SEQ ID NO: 1051 - DNA, 23 bp, Artificial Sequence
  Synthetic construct
gcaagagttatgcaggaggtgtt

SEQ ID NO: 1052 - DNA, 20 bp, Artificial Sequence
  Synthetic construct
caatcttctgctaccagtgg

SEQ ID NO: 1053 - DNA, 19 bp, Artificial Sequence
  Synthetic construct
attcaagtggagcacttgg

SEQ ID NO: 1054 - DNA, 41 bp, Artificial Sequence; identical to SEQ ID NO: 1024
  Synthetic construct

SEQ ID NO: 1055 - DNA, 40 bp, Artificial Sequence; identical to SEQ ID NO: 1025
  Synthetic construct

SEQ ID NO: 1056 - DNA, 20 bp, Artificial Sequence
  Synthetic construct
ttgccattgctcgatgctgt

SEQ ID NO: 1057 - DNA, 19 bp, Artificial Sequence
  Synthetic construct
agataaggtaaccgctcgt

SEQ ID NO: 1058 - DNA, 22 bp, Artificial Sequence
  Synthetic construct
cccaacacctcctgcataactc

SEQ ID NO: 1059 - DNA, 18 bp, Artificial Sequence
  Synthetic construct
cgatgttgaggcgaagtt

SEQ ID NO: 1060 - DNA, 22 bp, Artificial Sequence
  Synthetic construct
aagcccaaatgccactaacctt

SEQ ID NO: 1061 - DNA, 42 bp, Artificial Sequence
  Synthetic construct
cccaacacctcctgcataactctagataaggtaaccgctcgt

SEQ ID NO: 1062 - DNA, 20 bp, Artificial Sequence
  Synthetic construct
tagataaggtaaccgctcgt

SEQ ID NO: 1063 - DNA, 17 bp, Artificial Sequence
  Synthetic construct
cgtcgacagcatcgagc

SEQ ID NO: 1064 - DNA, 23 bp, Artificial Sequence
  Synthetic construct
cttcactcgcaagtgaattcaga

SEQ ID NO: 1065 - DNA, 44 bp, Artificial Sequence
  Synthetic construct
ggcatttgggcttgtcggatcaagcaagagttatgcaggaggtg

SEQ ID NO: 1066 - DNA, 41 bp, Artificial Sequence
  Synthetic construct
ggctcgtattgcggcggtaaagcacttgggctatcagcatc

SEQ ID NO: 1067 - DNA, 18 bp, Artificial Sequence
  Synthetic construct
aacgggcgacgtttaggc

SEQ ID NO: 1068 - DNA, 22 bp, Artificial Sequence
  Synthetic construct
aacttcgcctcaacatcgaagt

SEQ ID NO: 1069 - DNA, 21 bp, Artificial Sequence
  Synthetic construct
gcaagagttatgcaggaggtg

SEQ ID NO: 1070 - DNA, 23 bp, Artificial Sequence
  Synthetic construct
ggcatttgggcttgtcggatcaa

SEQ ID NO: 1071 - DNA, 20 bp, Artificial Sequence
  Synthetic construct
gcacttgggctatcagcatc

SEQ ID NO: 1072 - DNA, 21 bp, Artificial Sequence
  Synthetic construct
ggctcgtattgcggcggtaaa

SEQ ID NO: 1073 - DNA, 18 bp, Artificial Sequence
  Synthetic construct
acagcatcgagcaatggc

SEQ ID NO: 1074 - DNA, 43 bp, Artificial Sequence
  Synthetic construct
gtggcatttgggcttgtcggatgagttatgcaggaggtgttgg

SEQ ID NO: 1075 - DNA, 21 bp, Artificial Sequence
  Synthetic construct
gagttatgcaggaggtgttgg

SEQ ID NO: 1076 - DNA, 22 bp, Artificial Sequence
  Synthetic construct
gtggcatttgggcttgtcggat

SEQ ID NO: 1077 - DNA, 23 bp, Artificial Sequence
  Synthetic construct
aagcttcaatcttctgctaccag

SEQ ID NO: 1078 - DNA, 43 bp, Artificial Sequence
  Synthetic construct
ggcatttgggcttgtcggatcaaggcaagagttatgcaggagg

SEQ ID NO: 1079 - DNA, 20 bp, Artificial Sequence
  Synthetic construct
ggcaagagttatgcaggagg

SEQ ID NO: 1080 - DNA, 23 bp, Artificial Sequence
  Synthetic construct
ggcatttgggcttgtcggatcaa

SEQ ID NO: 1081 - DNA, 22 bp, Artificial Sequence
  Synthetic construct
agtggagcacttgggctatcag

SEQ ID NO: 1082 - DNA, 48 bp, Artificial Sequence; identical to SEQ ID NO: 41
  Synthetic construct

SEQ ID NO: 1083 - DNA, 41 bp, Artificial Sequence
  Synthetic construct
gggcgacgtttaggccccaacatcgtcgacagcatcgagca

SEQ ID NO: 1084 - DNA, 48 bp, Artificial Sequence; identical to SEQ ID NO: 41
  Synthetic construct

SEQ ID NO: 1085 - DNA, 22 bp, Artificial Sequence
  Synthetic construct
cctcctgcataactcttgccat

SEQ ID NO: 1086 - DNA, 22 bp, Artificial Sequence
  Synthetic construct
gggcgacgtttaggccccaaca

SEQ ID NO: 1087 - DNA, 24 bp, Artificial Sequence
  Synthetic construct
acacctcctgcataactcttgcca

SEQ ID NO: 1088 - DNA, 20 bp, Artificial Sequence
  Synthetic construct
ggctcgtattgcggcggtaa

SEQ ID NO: 1089 - DNA, 40 bp, Artificial Sequence
  Synthetic construct
tgcaaaacgggcgacgtttaggcaccgctcgtcgacagca

SEQ ID NO: 1090 - DNA, 24 bp, Artificial Sequence
  Synthetic construct
acacctcctgcataactcttgcca

SEQ ID NO: 1091 - DNA, 20 bp, Artificial Sequence
  Synthetic construct
ggctcgtattgcggcggtaa

SEQ ID NO: 1092 - DNA, 17 bp, Artificial Sequence
  Synthetic construct
accgctcgtcgacagca

SEQ ID NO: 1093 - DNA, 43 bp, Artificial Sequence
  Synthetic construct
tgcaaaacgggcgacgtttaggctaaccgctcgtcgacagcat

SEQ ID NO: 1094 - DNA, 24 bp, Artificial Sequence
  Synthetic construct
acacctcctgcataactcttgcca

SEQ ID NO: 1095 - DNA, 20 bp, Artificial Sequence
  Synthetic construct
ggctcgtattgcggcggtaa

SEQ ID NO: 1096 - DNA, 20 bp, Artificial Sequence
  Synthetic construct
taaccgctcgtcgacagcat

SEQ ID NO: 1097 - DNA, 25 bp, Artificial Sequence
  Synthetic construct
tttctatgatggttgttggcagcga

SEQ ID NO: 1098 - DNA, 24 bp, Artificial Sequence
  Synthetic construct
tttgcttgcccaatgtctggtact

SEQ ID NO: 1099 - DNA, 52 bp, Artificial Sequence
  Synthetic construct
cggagaagcgcaagcaaactgatttttggcaatcagaaagttgattcgtcgg

SEQ ID NO: 1100 - DNA, 53 bp, Artificial Sequence
  Synthetic construct
gaagggcggtctaataacacctaaattttgagttggtaagcgtacaagagcct

SEQ ID NO: 1101 - DNA, 24 bp, Artificial Sequence
  Synthetic construct
gaacacactagagtcagttgcagc

SEQ ID NO: 1102 - DNA, 25 bp, Artificial Sequence
  Synthetic construct
gagtttgcagagaagcttgcctcag

SEQ ID NO: 1103 - DNA, 25 bp, Artificial Sequence
  Synthetic construct
ggcaatcagaaagttgattcgtcgg

SEQ ID NO: 1104 - DNA, 23 bp, Artificial Sequence
  Synthetic construct
cggagaagcgcaagcaaactgat

SEQ ID NO: 1105 - DNA, 24 bp, Artificial Sequence
  Synthetic construct
gagttggtaagcgtacaagagcct

SEQ ID NO: 1106 - DNA, 25 bp, Artificial Sequence
  Synthetic construct
gaagggcggtctaataacacctaaa

SEQ ID NO: 1107 - DNA, 17 bp, Artificial Sequence
  Synthetic construct
gttgaactgagtctggc

SEQ ID NO: 1108 - DNA, 17 bp, Artificial Sequence
  Synthetic construct
gacaggcgattgctaac

SEQ ID NO: 1109 - DNA, 34 bp, Artificial Sequence
  Synthetic construct
aacaacctgcagcgtgtgagtcgaggtcagactg

SEQ ID NO: 1110 - DNA, 36 bp, Artificial Sequence
  Synthetic construct
cgccttcagtggtctgccatcaaaggtctgactcag

SEQ ID NO: 1111 - DNA, 17 bp, Artificial Sequence
  Synthetic construct
cggttcagtctgcgaat

SEQ ID NO: 1112 - DNA, 19 bp, Artificial Sequence
  Synthetic construct
caatggagataccgtcgtt

SEQ ID NO: 1113 - DNA, 17 bp, Artificial Sequence
  Synthetic construct
gagtcgaggtcagactg

SEQ ID NO: 1114 - DNA, 17 bp, Artificial Sequence
  Synthetic construct
aacaacctgcagcgtgt

SEQ ID NO: 1115 - DNA, 19 bp, Artificial Sequence
  Synthetic construct
catcaaaggtctgactcag

SEQ ID NO: 1116 - DNA, 17 bp, Artificial Sequence
  Synthetic construct
cgccttcagtggtctgc

SEQ ID NO: 1117 - DNA, 17 bp, Artificial Sequence
  Synthetic construct
ctgcaggttgttgttga

SEQ ID NO: 1118 - DNA, 18 bp, Artificial Sequence
  Synthetic construct
aatacaaacagcctgtcg

SEQ ID NO: 1119 - DNA, 36 bp, Artificial Sequence
  Synthetic construct
aggcgattgctaaccgtttggagataccgtcgttag

SEQ ID NO: 1120 - DNA, 39 bp, Artificial Sequence
  Synthetic construct
gatacgcagaccggaagacataacctgaacaaatcccag

SEQ ID NO: 1121 - DNA, 17 bp, Artificial Sequence
  Synthetic construct
aggtctgactcaggctt

SEQ ID NO: 1122 - DNA, 17 bp, Artificial Sequence
  Synthetic construct
aacgctcgatagcagtg

SEQ ID NO: 1123 - DNA, 18 bp, Artificial Sequence
  Synthetic construct
tggagataccgtcgttag

SEQ ID NO: 1124 - DNA, 18 bp, Artificial Sequence
  Synthetic construct
aggcgattgctaaccgtt

SEQ ID NO: 1125 - DNA, 20 bp, Artificial Sequence
  Synthetic construct
ataacctgaacaaatcccag

SEQ ID NO: 1126 - DNA, 19 bp, Artificial Sequence
  Synthetic construct
gatacgcagaccggaagac

SEQ ID NO: 1127 - DNA, 19 bp, Artificial Sequence
  Synthetic construct
ttattgatggtggctatgc

SEQ ID NO: 1128 - DNA, 41 bp, Artificial Sequence; identical to SEQ ID NO: 50
  Synthetic construct

SEQ ID NO: 1129 - DNA, 39 bp, Artificial Sequence; identical to SEQ ID NO: 51
  Synthetic construct

SEQ ID NO: 1130 - DNA, 22 bp, Artificial Sequence
  Synthetic construct
acttagcaagcgaccttgacaa

SEQ ID NO: 1131 - DNA, 21 bp, Artificial Sequence
  Synthetic construct
ttgagcaacgccagtaccatc

SEQ ID NO: 1132 - DNA, 17 bp, Artificial Sequence
  Synthetic construct
gagtcgaggtcagactg

SEQ ID NO: 1133 - DNA, 17 bp, Artificial Sequence
  Synthetic construct
gtctgcgtatcaacagc

SEQ ID NO: 1134 - DNA, 37 bp, Artificial Sequence
  Synthetic construct
caacaacaacctgcagcggagttagtaccattcgcag

SEQ ID NO: 1135 - DNA, 37 bp, Artificial Sequence
  Synthetic construct
ataccgtcgttagcgttacggacaggcgattgctaac

SEQ ID NO: 1136 - DNA, 19 bp, Artificial Sequence
  Synthetic construct
cgtgaactggcggttcagt

SEQ ID NO: 1137 - DNA, 23 bp, Artificial Sequence
  Synthetic construct
tgagtcagacctttgatgttcgc

SEQ ID NO: 1138 - DNA, 19 bp, Artificial Sequence
  Synthetic construct
gagttagtaccattcgcag

SEQ ID NO: 1139 - DNA, 18 bp, Artificial Sequence
  Synthetic construct
caacaacaacctgcagcg

SEQ ID NO: 1140 - DNA, 17 bp, Artificial Sequence
  Synthetic construct
gacaggcgattgctaac

SEQ ID NO: 1141 - DNA, 20 bp, Artificial Sequence
  Synthetic construct
ataccgtcgttagcgttacg

SEQ ID NO: 1142 - DNA, 19 bp, Artificial Sequence
  Synthetic construct
gagttagtaccattcgcag

SEQ ID NO: 1143 - DNA, 20 bp, Artificial Sequence
  Synthetic construct
ataacctgaacaaatcccag

SEQ ID NO: 1144 - DNA, 37 bp, Artificial Sequence
  Synthetic construct
cgtaacgctaacgacggtatctgcaggttgttgttga

SEQ ID NO: 1145 - DNA, 35 bp, Artificial Sequence
  Synthetic construct
acggttagcaatcgcctggtttgtcttccggtctg

SEQ ID NO: 1146 - DNA, 19 bp, Artificial Sequence
  Synthetic construct
ccattgcgcagaccactga

SEQ ID NO: 1147 - DNA, 19 bp, Artificial Sequence
  Synthetic construct
tcctgccgcatcgtctttc

SEQ ID NO: 1148 - DNA, 17 bp, Artificial Sequence
  Synthetic construct
ctgcaggttgttgttga

SEQ ID NO: 1149 - DNA, 20 bp, Artificial Sequence
  Synthetic construct
cgtaacgctaacgacggtat

SEQ ID NO: 1150 - DNA, 17 bp, Artificial Sequence
  Synthetic construct
gtttgtcttccggtctg

SEQ ID NO: 1151 - DNA, 18 bp, Artificial Sequence
  Synthetic construct
acggttagcaatcgcctg

SEQ ID NO: 1152 - DNA, 17 bp, Artificial Sequence
  Synthetic construct
cgccgttgaactgagtc

SEQ ID NO: 1153 - DNA, 18 bp, Artificial Sequence
  Synthetic construct
cggtctgcgtatcaacag

SEQ ID NO: 1154 - DNA, 34 bp, Artificial Sequence
  Synthetic construct
tgcagcgtgtgcgtgaacctggatggagtcgagg

SEQ ID NO: 1155 - DNA, 37 bp, Artificial Sequence
  Synthetic construct
tcagtggtctgcgcaatggaggtctgactcaggcttc

SEQ ID NO: 1156 - DNA, 21 bp, Artificial Sequence
  Synthetic construct
gttcagtctgcgaatggtact

SEQ ID NO: 1157 - DNA, 20 bp, Artificial Sequence
  Synthetic construct
ataccgtcgttagcgttacg

SEQ ID NO: 1158 - DNA, 17 bp, Artificial Sequence
  Synthetic construct
cctggatggagtcgagg

SEQ ID NO: 1159 - DNA, 17 bp, Artificial Sequence
  Synthetic construct
tgcagcgtgtgcgtgaa

SEQ ID NO: 1160 - DNA, 18 bp, Artificial Sequence
  Synthetic construct
aggtctgactcaggcttc

SEQ ID NO: 1161 - DNA, 19 bp, Artificial Sequence
  Synthetic construct
tcagtggtctgcgcaatgg

SEQ ID NO: 1162 - DNA, 17 bp, Artificial Sequence
  Synthetic construct
cctggatggagtcgagg

SEQ ID NO: 1163 - DNA, 36 bp, Artificial Sequence
  Synthetic construct
ccattgcgcagaccactgactgaaccgccagttcac

SEQ ID NO: 1164 - DNA, 39 bp, Artificial Sequence
  Synthetic construct
gataccgtcgttagcgttacggcaggcgattgctaaccg

SEQ ID NO: 1165 - DNA, 17 bp, Artificial Sequence
  Synthetic construct
aacaacctgcagcgtgt

SEQ ID NO: 1166 - DNA, 22 bp, Artificial Sequence
  Synthetic construct
cctgagtcagacctttgatgtt

SEQ ID NO: 1167 - DNA, 17 bp, Artificial Sequence
  Synthetic construct
ctgaaccgccagttcac

SEQ ID NO: 1168 - DNA, 19 bp, Artificial Sequence
  Synthetic construct
ccattgcgcagaccactga

SEQ ID NO: 1169 - DNA, 17 bp, Artificial Sequence
  Synthetic construct
caggcgattgctaaccg

SEQ ID NO: 1170 - DNA, 22 bp, Artificial Sequence
  Synthetic construct
gataccgtcgttagcgttacgg

SEQ ID NO: 1171 - DNA, 17 bp, Artificial Sequence
  Synthetic construct
cgctgcaggttgttgtt

SEQ ID NO: 1172 - DNA, 18 bp, Artificial Sequence
  Synthetic construct
atacaaacagcctgtcgc

SEQ ID NO: 1173 - DNA, 37 bp, Artificial Sequence
  Synthetic construct
gcaggacaggcgattgctaaccgtcgttagcgttacg

SEQ ID NO: 1174 - DNA, 44 bp, Artificial Sequence
  Synthetic construct
gttgatacgcagaccggaagacataacctgaacaaatcccagtc

SEQ ID NO: 1175 - DNA, 22 bp, Artificial Sequence
  Synthetic construct
aacatcaaaggtctgactcagg

SEQ ID NO: 1176 - DNA, 17 bp, Artificial Sequence
  Synthetic construct
acgctcgatagcagtgc

SEQ ID NO: 1177 - DNA, 17 bp, Artificial Sequence
  Synthetic construct
ccgtcgttagcgttacg

SEQ ID NO: 1178 - DNA, 20 bp, Artificial Sequence
  Synthetic construct
gcaggacaggcgattgctaa

SEQ ID NO: 1179 - DNA, 22 bp, Artificial Sequence
  Synthetic construct
ataacctgaacaaatcccagtc

SEQ ID NO: 1180 - DNA, 22 bp, Artificial Sequence
  Synthetic construct
gttgatacgcagaccggaagac

SEQ ID NO: 1181 - DNA, 17 bp, Artificial Sequence
  Synthetic construct
cgccgttgaactgagtc

SEQ ID NO: 1182 - DNA, 18 bp, Artificial Sequence
  Synthetic construct
cggtctgcgtatcaacag

SEQ ID NO: 1183 - DNA, 34 bp, Artificial Sequence; identical to SEQ ID NO: 1154
  Synthetic construct

SEQ ID NO: 1184 - DNA, 37 bp, Artificial Sequence; identical to SEQ ID NO: 1155
  Synthetic construct

SEQ ID NO: 1185 - DNA, 19 bp, Artificial Sequence
  Synthetic construct
ggcggttcagtctgcgaat

SEQ ID NO: 1186 - DNA, 22 bp, Artificial Sequence
  Synthetic construct
gataccgtcgttagcgttacgg

SEQ ID NO: 1187 - DNA, 17 bp, Artificial Sequence
  Synthetic construct
cctggatggagtcgagg

SEQ ID NO: 1188 - DNA, 17 bp, Artificial Sequence
  Synthetic construct
tgcagcgtgtgcgtgaa

SEQ ID NO: 1189 - DNA, 18 bp, Artificial Sequence
  Synthetic construct
aggtctgactcaggcttc

SEQ ID NO: 1190 - DNA, 19 bp, Artificial Sequence
  Synthetic construct
tcagtggtctgcgcaatgg

SEQ ID NO: 1191 - DNA, 22 bp, Artificial Sequence
  Synthetic construct
ggcatcttggacattaagctta

SEQ ID NO: 1192 - DNA, 21 bp, Artificial Sequence
  Synthetic construct
ctaacgacggtatctccattg

SEQ ID NO: 1193 - DNA, 38 bp, Artificial Sequence
  Synthetic construct
gactcagttcaacggcgtgaattggcaccaacctggat

SEQ ID NO: 1194 - DNA, 37 bp, Artificial Sequence
  Synthetic construct
cctggatggagtcgaggtcagtgaactggcggttcag

SEQ ID NO: 1195 - DNA, 17 bp, Artificial Sequence
  Synthetic construct
tggcgcaggacaacacc

SEQ ID NO: 1196 - DNA, 23 bp, Artificial Sequence
  Synthetic construct
tgggagttagtaccattcgcaga

SEQ ID NO: 1197 - DNA, 17 bp, Artificial Sequence
  Synthetic construct
ttggcaccaacctggat

SEQ ID NO: 1198 - DNA, 21 bp, Artificial Sequence
  Synthetic construct
gactcagttcaacggcgtgaa

SEQ ID NO: 1199 - DNA, 17 bp, Artificial Sequence
  Synthetic construct
gtgaactggcggttcag

SEQ ID NO: 1200 - DNA, 20 bp, Artificial Sequence
  Synthetic construct
cctggatggagtcgaggtca

SEQ ID NO: 1201 - DNA, 17 bp, Artificial Sequence
  Synthetic construct
cctggatggagtcgagg

SEQ ID NO: 1202 - DNA, 18 bp, Artificial Sequence
  Synthetic construct
cggtctgcgtatcaacag

SEQ ID NO: 1203 - DNA, 36 bp, Artificial Sequence; identical to SEQ ID NO: 1163
  Synthetic construct

SEQ ID NO: 1204 - DNA, 39 bp, Artificial Sequence; identical to SEQ ID NO: 1164
  Synthetic construct

SEQ ID NO: 1205 - DNA, 19 bp, Artificial Sequence
  Synthetic construct
acaacaacctgcagcgtgt

SEQ ID NO: 1206 - DNA, 23 bp, Artificial Sequence
  Synthetic construct
tgagtcagacctttgatgttcgc

SEQ ID NO: 1207 - DNA, 17 bp, Artificial Sequence
  Synthetic construct
ctgaaccgccagttcac

SEQ ID NO: 1208 - DNA, 19 bp, Artificial Sequence
  Synthetic construct
ccattgcgcagaccactga

SEQ ID NO: 1209 - DNA, 17 bp, Artificial Sequence
  Synthetic construct
caggcgattgctaaccg

SEQ ID NO: 1210 - DNA, 22 bp, Artificial Sequence
  Synthetic construct
gataccgtcgttagcgttacgg

SEQ ID NO: 1211 - DNA, 23 bp, Artificial Sequence
  Synthetic construct
gtaggcatcttggacattaagct

SEQ ID NO: 1212 - DNA, 22 bp, Artificial Sequence
  Synthetic construct
ctaacgacggtatctccattgc

SEQ ID NO: 1213 - DNA, 39 bp, Artificial Sequence
  Synthetic construct
cgtgtatccggccagactcagtcgttggcaccaacctgg

SEQ ID NO: 1214 - DNA, 40 bp, Artificial Sequence
  Synthetic construct
cgctgggtgatttcagcctgggtgaactggcggttcagtc

SEQ ID NO: 1215 - DNA, 17 bp, Artificial Sequence
  Synthetic construct
tggcgcaggacaacacc

SEQ ID NO: 1216 - DNA, 21 bp, Artificial Sequence
  Synthetic construct
atggagtcgaggtcagactgg

SEQ ID NO: 1217 - DNA, 17 bp, Artificial Sequence
  Synthetic construct
cgttggcaccaacctgg

SEQ ID NO: 1218 - DNA, 22 bp, Artificial Sequence
  Synthetic construct
cgtgtatccggccagactcagt

SEQ ID NO: 1219 - DNA, 19 bp, Artificial Sequence
  Synthetic construct
gtgaactggcggttcagtc

SEQ ID NO: 1220 - DNA, 21 bp, Artificial Sequence
  Synthetic construct
cgctgggtgatttcagcctgg

SEQ ID NO: 1221 - DNA, 19 bp, Artificial Sequence
  Synthetic construct
cgccgttgaactgagtctg

SEQ ID NO: 1222 - DNA, 19 bp, Artificial Sequence
  Synthetic construct
ggtctgcgtatcaacagcg

SEQ ID NO: 1223 - DNA, 42 bp, Artificial Sequence
  Synthetic construct
atcaacaacaacctgcagcgtgtggagtcgaggtcagactgg

SEQ ID NO: 1224 - DNA, 45 bp, Artificial Sequence
  Synthetic construct
ccttcagtggtctgcgcaatggcgaacatcaaaggtctgactcag

SEQ ID NO: 1225 - DNA, 19 bp, Artificial Sequence
  Synthetic construct
ggcggttcagtctgcgaat

SEQ ID NO: 1226 - DNA, 22 bp, Artificial Sequence
  Synthetic construct
gataccgtcgttagcgttacgg

SEQ ID NO: 1227 - DNA, 19 bp, Artificial Sequence
  Synthetic construct
ggagtcgaggtcagactgg

SEQ ID NO: 1228 - DNA, 23 bp, Artificial Sequence
  Synthetic construct
atcaacaacaacctgcagcgtgt

SEQ ID NO: 1229 - DNA, 23 bp, Artificial Sequence
  Synthetic construct
cgaacatcaaaggtctgactcag

SEQ ID NO: 1230 - DNA, 22 bp, Artificial Sequence
  Synthetic construct
ccttcagtggtctgcgcaatgg

SEQ ID NO: 1231 - DNA, 17 bp, Artificial Sequence
  Synthetic construct
cgccaggactttcacgc

SEQ ID NO: 1232 - DNA, 18 bp, Artificial Sequence
  Synthetic construct
tcagcctggatggagtcg

SEQ ID NO: 1233 - DNA, 20 bp, Artificial Sequence
  Synthetic construct
ctgcagcgtgtgcgtgaact

SEQ ID NO: 1234 - DNA, 21 bp, Artificial Sequence
  Synthetic construct
tcacgccgttgaactgagtct

SEQ ID NO: 1235 - DNA, 18 bp, Artificial Sequence
  Synthetic construct
agacgatgcggcaggaca

SEQ ID NO: 1236 - DNA, 44 bp, Artificial Sequence
  Synthetic construct
aacaacctgcagcgtgtgcgtgctggatggagtcgaggtcagac

SEQ ID NO: 1237 - DNA, 46 bp, Artificial Sequence
  Synthetic construct
gccttcagtggtctgcgcaatggtaccgcgaacatcaaaggtctga

SEQ ID NO: 1238 - DNA, 22 bp, Artificial Sequence
  Synthetic construct
ctggatggagtcgaggtcagac

SEQ ID NO: 1239 - DNA, 22 bp, Artificial Sequence
  Synthetic construct
aacaacctgcagcgtgtgcgtg

SEQ ID NO: 1240 - DNA, 23 bp, Artificial Sequence
  Synthetic construct
taccgcgaacatcaaaggtctga

SEQ ID NO: 1241 - DNA, 23 bp, Artificial Sequence
  Synthetic construct
gccttcagtggtctgcgcaatgg

SEQ ID NO: 1242 - DNA, 19 bp, Artificial Sequence
  Synthetic construct
tcctgcgccaggactttca

SEQ ID NO: 1243 - DNA, 42 bp, Artificial Sequence
  Synthetic construct
ctgcagcgtgtgcgtgaactggcagcctggatggagtcgagg

SEQ ID NO: 1244 - DNA, 45 bp, Artificial Sequence
  Synthetic construct
gccttcagtggtctgcgcaatggccgcgaacatcaaaggtctgac

SEQ ID NO: 1245 - DNA, 20 bp, Artificial Sequence
  Synthetic construct
cggttcagtctgcgaatggt

SEQ ID NO: 1246 - DNA, 20 bp, Artificial Sequence
  Synthetic construct
cagcctggatggagtcgagg

SEQ ID NO: 1247 - DNA, 22 bp, Artificial Sequence
  Synthetic construct
ctgcagcgtgtgcgtgaactgg

SEQ ID NO: 1248 - DNA, 22 bp, Artificial Sequence
  Synthetic construct
ccgcgaacatcaaaggtctgac

SEQ ID NO: 1249 - DNA, 23 bp, Artificial Sequence
  Synthetic construct
gccttcagtggtctgcgcaatgg

SEQ ID NO: 1250 - DNA, 19 bp, Artificial Sequence
  Synthetic construct
ctgcgccaggactttcacg

SEQ ID NO: 1251 - DNA, 42 bp, Artificial Sequence
  Synthetic construct
ctgcagcgtgtgcgtgaactgggcctggatggagtcgaggtc

SEQ ID NO: 1252 - DNA, 46 bp, Artificial Sequence; identical to SEQ ID NO: 1237
  Synthetic construct

SEQ ID NO: 1253 - DNA, 20 bp, Artificial Sequence
  Synthetic construct
gcctggatggagtcgaggtc

SEQ ID NO: 1254 - DNA, 23 bp, Artificial Sequence
  Synthetic construct
taccgcgaacatcaaaggtctga

SEQ ID NO: 1255 - DNA, 21 bp, Artificial Sequence
  Synthetic construct
tcacgccgttgaactgagtct

SEQ ID NO: 1256 - DNA, 21 bp, Artificial Sequence
  Synthetic construct
gcaggacaggcgattgctaac

SEQ ID NO: 1257 - DNA, 44 bp, Artificial Sequence
  Synthetic construct
caacaacctgcagcgtgtgcgtctggatggagtcgaggtcagac

SEQ ID NO: 1258 - DNA, 46 bp, Artificial Sequence; identical to SEQ ID NO: 1237
  Synthetic construct

SEQ ID NO: 1259 - DNA, 21 bp, Artificial Sequence
  Synthetic construct
gaactggcggttcagtctgcg

SEQ ID NO: 1260 - DNA, 22 bp, Artificial Sequence
  Synthetic construct
tcgttagcgttacgggaagcct

SEQ ID NO: 1261 - DNA, 22 bp, Artificial Sequence
  Synthetic construct
ctggatggagtcgaggtcagac

SEQ ID NO: 1262 - DNA, 22 bp, Artificial Sequence
  Synthetic construct
caacaacctgcagcgtgtgcgt

SEQ ID NO: 1263 - DNA, 23 bp, Artificial Sequence
  Synthetic construct
taccgcgaacatcaaaggtctga

SEQ ID NO: 1264 - DNA, 23 bp, Artificial Sequence
  Synthetic construct
gccttcagtggtctgcgcaatgg

SEQ ID NO: 1265 - DNA, 19 bp, Artificial Sequence
  Synthetic construct
tcctgcgccaggactttca

SEQ ID NO: 1266 - DNA, 42 bp, Artificial Sequence
  Synthetic construct
cctgcagcgtgtgcgtgaactgcagcctggatggagtcgagg

SEQ ID NO: 1267 - DNA, 45 bp, Artificial Sequence; identical to SEQ ID NO: 1244
  Synthetic construct

SEQ ID NO: 1268 - DNA, 23 bp, Artificial Sequence
  Synthetic construct
gcggttcagtctgcgaatggtac

SEQ ID NO: 1269 - DNA, 22 bp, Artificial Sequence
  Synthetic construct
tcgttagcgttacgggaagcct

SEQ ID NO: 1270 - DNA, 20 bp, Artificial Sequence
  Synthetic construct
cagcctggatggagtcgagg

SEQ ID NO: 1271 - DNA, 22 bp, Artificial Sequence
  Synthetic construct
cctgcagcgtgtgcgtgaactg

SEQ ID NO: 1272 - DNA, 22 bp, Artificial Sequence
  Synthetic construct
ccgcgaacatcaaaggtctgac

SEQ ID NO: 1273 - DNA, 19 bp, Artificial Sequence
  Synthetic construct
ctgcgccaggactttcacg

SEQ ID NO: 1274 - DNA, 42 bp, Artificial Sequence
  Synthetic construct
cctgcagcgtgtgcgtgaactggcctggatggagtcgaggtc

SEQ ID NO: 1275 - DNA, 46 bp, Artificial Sequence; identical to SEQ ID NO: 1237
  Synthetic construct

SEQ ID NO: 1276 - DNA, 20 bp, Artificial Sequence
  Synthetic construct
gcctggatggagtcgaggtc

SEQ ID NO: 1277 - DNA, 23 bp, Artificial Sequence
  Synthetic construct
taccgcgaacatcaaaggtctga

SEQ ID NO: 1278 - DNA, 20 bp, Artificial Sequence
  Synthetic construct
gacaggcgattgctaaccgt

SEQ ID NO: 1279 - DNA, 19 bp, Artificial Sequence
  Synthetic construct
cgttcaggcgctgggtgat

SEQ ID NO: 1280 - DNA, 45 bp, Artificial Sequence
  Synthetic construct
tcagcgcgccttcagtggtctttttgtctgactcaggcttcccgt

SEQ ID NO: 1281 - DNA, 46 bp, Artificial Sequence
  Synthetic construct
cctgcagcgtgtgcgtgaactgttttagcctggatggagtcgaggt

SEQ ID NO: 1282 - DNA, 22 bp, Artificial Sequence
  Synthetic construct
gcgcaatggagataccgtcgtt

SEQ ID NO: 1283 - DNA, 26 bp, Artificial Sequence
  Synthetic construct
tgcgaatggtactaactcccagtctg

SEQ ID NO: 1284 - DNA, 20 bp, Artificial Sequence
  Synthetic construct
gtctgactcaggcttcccgt

SEQ ID NO: 1285 - DNA, 21 bp, Artificial Sequence
  Synthetic construct
tcagcgcgccttcagtggtct

SEQ ID NO: 1286 - DNA, 20 bp, Artificial Sequence
  Synthetic construct
agcctggatggagtcgaggt

SEQ ID NO: 1287 - DNA, 22 bp, Artificial Sequence
  Synthetic construct
cctgcagcgtgtgcgtgaactg

SEQ ID NO: 1288 - DNA, 18 bp, Artificial Sequence
  Synthetic construct
gatggcaggccatgttcc

SEQ ID NO: 1289 - DNA, 20 bp, Artificial Sequence
  Synthetic construct
acaggatccattgcaagagg

SEQ ID NO: 1290 - DNA, 44 bp, Artificial Sequence
  Synthetic construct
acgaattcgggcagaagatcgcttttctggatgcgcttccatga

SEQ ID NO: 1291 - DNA, 44 bp, Artificial Sequence
  Synthetic construct
tgatgatggcgtctaaggacgctttttccggtaccaactgacca

SEQ ID NO: 1292 - DNA, 19 bp, Artificial Sequence
  Synthetic construct
tcctgtccacaatccgagg

SEQ ID NO: 1293 - DNA, 19 bp, Artificial Sequence
  Synthetic construct
caagcgtggatggcgctag

SEQ ID NO: 1294 - DNA, 18 bp, Artificial Sequence
  Synthetic construct
ctggatgcgcttccatga

SEQ ID NO: 1295 - DNA, 22 bp, Artificial Sequence
  Synthetic construct
acgaattcgggcagaagatcgc

SEQ ID NO: 1296 - DNA, 18 bp, Artificial Sequence
  Synthetic construct
tccggtaccaactgacca

SEQ ID NO: 1297 - DNA, 22 bp, Artificial Sequence
  Synthetic construct
tgatgatggcgtctaaggacgc

SEQ ID NO: 1298 - DNA, 18 bp, Artificial Sequence
  Synthetic construct
tttacgtgcccagacaag

SEQ ID NO: 1299 - DNA, 18 bp, Artificial Sequence
  Synthetic construct
aatagcggcaccaacaac

SEQ ID NO: 1300 - DNA, 40 bp, Artificial Sequence
  Synthetic construct
caaagctgggagccagattgagccaatgttcagatggatg

SEQ ID NO: 1301 - DNA, 42 bp, Artificial Sequence
  Synthetic construct
gtcgaatgacgccaacccatgccataacctcattattgacct

SEQ ID NO: 1302 - DNA, 21 bp, Artificial Sequence
  Synthetic construct
cccacgtgctcagatctgaga

SEQ ID NO: 1303 - DNA, 17 bp, Artificial Sequence
  Synthetic construct
tccgcagccaacctcgt

SEQ ID NO: 1304 - DNA, 20 bp, Artificial Sequence
  Synthetic construct
agccaatgttcagatggatg

SEQ ID NO: 1305 - DNA, 20 bp, Artificial Sequence
  Synthetic construct
caaagctgggagccagattg

SEQ ID NO: 1306 - DNA, 22 bp, Artificial Sequence
  Synthetic construct
gccataacctcattattgacct

SEQ ID NO: 1307 - DNA, 20 bp, Artificial Sequence
  Synthetic construct
gtcgaatgacgccaacccat

SEQ ID NO: 1308 - DNA, 18 bp, Artificial Sequence
  Synthetic construct
gacaagagccaatgttca

SEQ ID NO: 1309 - DNA, 19 bp, Artificial Sequence
  Synthetic construct
tctaatccaggggtcaatt

SEQ ID NO: 1310 - DNA, 40 bp, Artificial Sequence
  Synthetic construct
tcgacgccatcttcattcactggatgagattctcagatct

SEQ ID NO: 1311 - DNA, 36 bp, Artificial Sequence
  Synthetic construct
catctgatgggtccgcagccagagccataacctcat

SEQ ID NO: 1312 - DNA, 18 bp, Artificial Sequence
  Synthetic construct
aaagctgggagccagatt

SEQ ID NO: 1313 - DNA, 18 bp, Artificial Sequence
  Synthetic construct
tcgtcccagaggtcaata

SEQ ID NO: 1314 - DNA, 20 bp, Artificial Sequence
  Synthetic construct
tggatgagattctcagatct

SEQ ID NO: 1315 - DNA, 20 bp, Artificial Sequence
  Synthetic construct
tcgacgccatcttcattcac

SEQ ID NO: 1316 - DNA, 18 bp, Artificial Sequence
  Synthetic construct
ccagagccataacctcat

SEQ ID NO: 1317 - DNA, 18 bp, Artificial Sequence
  Synthetic construct
catctgatgggtccgcag

SEQ ID NO: 1318 - DNA, 19 bp, Artificial Sequence
  Synthetic construct
caagagccaatgttcagat

SEQ ID NO: 1319 - DNA, 41 bp, Artificial Sequence
  Synthetic construct
attcgacgccatcttcattcaggatgagattctcagatctg

SEQ ID NO: 1320 - DNA, 20 bp, Artificial Sequence
  Synthetic construct
ggatgagattctcagatctg

SEQ ID NO: 1321 - DNA, 21 bp, Artificial Sequence
  Synthetic construct
attcgacgccatcttcattca

SEQ ID NO: 1322 - DNA, 18 bp, Artificial Sequence
  Synthetic construct
gatttttacgtgcccaga

SEQ ID NO: 1323 - DNA, 38 bp, Artificial Sequence
  Synthetic construct
ttcacaaagctgggagcccaatgttcagatggatgaga

SEQ ID NO: 1324 - DNA, 18 bp, Artificial Sequence
  Synthetic construct
cacgtgctcagatctgag

SEQ ID NO: 1325 - DNA, 20 bp, Artificial Sequence
  Synthetic construct
caatgttcagatggatgaga

SEQ ID NO: 1326 - DNA, 18 bp, Artificial Sequence
  Synthetic construct
ttcacaaagctgggagcc

SEQ ID NO: 1327 - DNA, 18 bp, Artificial Sequence
  Synthetic construct
acaagagccaatgttcag

SEQ ID NO: 1328 - DNA, 40 bp, Artificial Sequence; identical to SEQ ID NO: 1310
  Synthetic construct

SEQ ID NO: 1329 - DNA, 20 bp, Artificial Sequence
  Synthetic construct
ctgggagccagattgcgatc

SEQ ID NO: 1330 - DNA, 21 bp, Artificial Sequence
  Synthetic construct
aacctcgtcccagaggtcaat

SEQ ID NO: 1331 - DNA, 20 bp, Artificial Sequence
  Synthetic construct
tggatgagattctcagatct

SEQ ID NO: 1332 - DNA, 11 bp, Artificial Sequence
  Synthetic construct
tcgacgccatc

SEQ ID NO: 1333 - DNA, 19 bp, Artificial Sequence
  Synthetic construct
caagagccaatgttcagat

SEQ ID NO: 1334 - DNA, 41 bp, Artificial Sequence; identical to SEQ ID NO: 1319
  Synthetic construct

SEQ ID NO: 1335 - DNA, 20 bp, Artificial Sequence
  Synthetic construct
ggatgagattctcagatctg

SEQ ID NO: 1336 - DNA, 21 bp, Artificial Sequence
  Synthetic construct
attcgacgccatcttcattca

SEQ ID NO: 1337 - DNA, 18 bp, Artificial Sequence
  Synthetic construct
gacaagagccaatgttca

SEQ ID NO: 1338 - DNA, 43 bp, Artificial Sequence
  Synthetic construc
gacgccatcttcattcacaaaggatggatgagattctcagatc

SEQ ID NO: 1339 - DNA, 20 bp, Artificial Sequence
  Synthetic construct
gatggatgagattctcagat

SEQ ID NO: 1340 - DNA, 22 bp, Artificial Sequence
  Synthetic construct
gacgccatcttcattcacaaag

SEQ ID NO: 1341 - DNA, 20 bp, Artificial Sequence
  Synthetic construct
cagacaagagccaatgttca

SEQ ID NO: 1342 - DNA, 18 bp, Artificial Sequence
  Synthetic construct
caatagcggcaccaacaa

SEQ ID NO: 1343 - DNA, 41 bp, Artificial Sequence; identical to SEQ ID NO: 70
  Synthetic construct

SEQ ID NO: 1344 - DNA, 40 bp, Artificial Sequence; identical to SEQ ID NO: 71
  Synthetic construct

SEQ ID NO: 1345 - DNA, 18 bp, Artificial Sequence
  Synthetic construct
ttcacaaagctgggagcc

SEQ ID NO: 1346 - DNA, 18 bp, Artificial Sequence
  Synthetic construct
cctcgtcccagaggtcaa

SEQ ID NO: 1347 - DNA, 18 bp, Artificial Sequence
  Synthetic construct
ttacgtgcccagacaaga

SEQ ID NO: 1348 - DNA, 19 bp, Artificial Sequence
  Synthetic construct
ctccagagccataacctca

SEQ ID NO: 1349 - DNA, 40 bp, Artificial Sequence
  Synthetic construct
ctgggagccagattgcgatcgccaatgttcagatggatga

SEQ ID NO: 1350 - DNA, 39 bp, Artificial Sequence
  Synthetic construct
tgaagatggcgtcgaatgacgattgacctctgggacgag

SEQ ID NO: 1351 - DNA, 21 bp, Artificial Sequence
  Synthetic construct
acgtgctcagatctgagaatc

SEQ ID NO: 1352 - DNA, 18 bp, Artificial Sequence
  Synthetic construct
catctgatgggtccgcag

SEQ ID NO: 1353 - DNA, 20 bp, Artificial Sequence
  Synthetic construct
gccaatgttcagatggatga

SEQ ID NO: 1354 - DNA, 18 bp, Artificial Sequence
  Synthetic construct
attgacctctgggacgag

SEQ ID NO: 1355 - DNA, 21 bp, Artificial Sequence
  Synthetic construct
tgaagatggcgtcgaatgacg

SEQ ID NO: 1356 - DNA, 18 bp, Artificial Sequence
  Synthetic construct
tacgtgcccagacaagag

SEQ ID NO: 1357 - DNA, 42 bp, Artificial Sequence
  Synthetic construct
ctgggagccagattgcgatcccaatgttcagatggatgagat

SEQ ID NO: 1358 - DNA, 18 bp, Artificial Sequence
  Synthetic construct
tcccacgtgctcagatct

SEQ ID NO: 1359 - DNA, 22 bp, Artificial Sequence
  Synthetic construct
ccaatgttcagatggatgagat

SEQ ID NO: 1360 - DNA, 20 bp, Artificial Sequence
  Synthetic construct
cctcccacgtgctcagatct

SEQ ID NO: 1361 - DNA, 20 bp, Artificial Sequence
  Synthetic construct
gccaatgttcagatggatga

SEQ ID NO: 1362 - DNA, 18 bp, Artificial Sequence
  Synthetic construct
tacgtgcccagacaagag

SEQ ID NO: 1363 - DNA, 22 bp, Artificial Sequence
  Synthetic construct
ccaatgttcagatggatgagat

SEQ ID NO: 1364 - DNA, 19 bp, Artificial Sequence
  Synthetic construct
gcccagacaagagccaatg

SEQ ID NO: 1365 - DNA, 22 bp, Artificial Sequence
  Synthetic construct
ccaggggtcaattacgttttgt

SEQ ID NO: 1366 - DNA, 39 bp, Artificial Sequence
  Synthetic construct
cccatcagatgggttggcgtccgatcgcaatctggctcc

SEQ ID NO: 1367 - DNA, 38 bp, Artificial Sequence
  Synthetic construct
agccaacctcgtcccagaggccacaggtgccgcaatag

SEQ ID NO: 1368 - DNA, 22 bp, Artificial Sequence
  Synthetic construct
tcgacgccatcttcattcacaa

SEQ ID NO: 1369 - DNA, 19 bp, Artificial Sequence
  Synthetic construct
ctctggagcccgttgttgg

SEQ ID NO: 1370 - DNA, 18 bp, Artificial Sequence
  Synthetic construct
cgatcgcaatctggctcc

SEQ ID NO: 1371 - DNA, 21 bp, Artificial Sequence
  Synthetic construct
cccatcagatgggttggcgtc

SEQ ID NO: 1372 - DNA, 18 bp, Artificial Sequence
  Synthetic construct
ccacaggtgccgcaatag

SEQ ID NO: 1373 - DNA, 20 bp, Artificial Sequence
  Synthetic construct
agccaacctcgtcccagagg

SEQ ID NO: 1374 - DNA, 22 bp, Artificial Sequence
  Synthetic construct
tccaggggtcaattacgttttg

SEQ ID NO: 1375 - DNA, 39 bp, Artificial Sequence; identical to SEQ ID NO: 1366
  Synthetic construct

SEQ ID NO: 1376 - DNA, 40 bp, Artificial Sequence
  Synthetic construct
cccatcagatgggttggcgtcacgatcgcaatctggctcc

SEQ ID NO: 1377 - DNA, 22 bp, Artificial Sequence
  Synthetic construct
cccatcagatgggttggcgtca

SEQ ID NO: 1378 - DNA, 22 bp, Artificial Sequence
  Synthetic construct
gcccagacaagagccaatgttc

SEQ ID NO: 1379 - DNA, 18 bp, Artificial Sequence
  Synthetic construc
tttgttggcccgccacag

SEQ ID NO: 1380 - DNA, 44 bp, Artificial Sequence
  Synthetic construct
gcggacccatcagatgggttggcaatctggctcccagctttgtg

SEQ ID NO: 1381 - DNA, 41 bp, Artificial Sequence
  Synthetic construct
gccaacctcgtcccagaggtcacgcaatagcggcaccaaca

SEQ ID NO: 1382 - DNA, 22 bp, Artificial Sequence
  Synthetic construct
aatgaggttatggctctggagc

SEQ ID NO: 1383 - DNA, 22 bp, Artificial Sequence
  Synthetic construct
caatctggctcccagctttgtg

SEQ ID NO: 1384 - DNA, 22 bp, Artificial Sequence
  Synthetic construct
gcggacccatcagatgggttgg

SEQ ID NO: 1385 - DNA, 19 bp, Artificial Sequence
  Synthetic construct
cgcaatagcggcaccaaca

SEQ ID NO: 1386 - DNA, 22 bp, Artificial Sequence
  Synthetic construct
gccaacctcgtcccagaggtca

SEQ ID NO: 1387 - DNA, 19 bp, Artificial Sequence
  Synthetic construct
gttttgttggcccgccaca

SEQ ID NO: 1388 - DNA, 25 bp, Artificial Sequence
  Synthetic construct
tggatgagattctcagatctgagca

SEQ ID NO: 1389 - DNA, 25 bp, Artificial Sequence
  Synthetic construct
tccaggggtcaattacgttttgttg

SEQ ID NO: 1390 - DNA, 44 bp, Artificial Sequence
  Synthetic construct
gacccatcagatgggttggcgtcattgggagggcgatcgcaatc

SEQ ID NO: 1391 - DNA, 41 bp, Artificial Sequence
  Synthetic construct
cagccaacctcgtcccagaggtcacaggtgccgcaatagcg

SEQ ID NO: 1392 - DNA, 24 bp, Artificial Sequence
  Synthetic construct
tcttcattcacaaagctgggagcc

SEQ ID NO: 1393 - DNA, 19 bp, Artificial Sequence
  Synthetic construct
tggagcccgttgttggtgc

SEQ ID NO: 1394 - DNA, 19 bp, Artificial Sequence
  Synthetic construct
tgggagggcgatcgcaatc

SEQ ID NO: 1395 - DNA, 25 bp, Artificial Sequence
  Synthetic construct
gacccatcagatgggttggcgtcat

SEQ ID NO: 1396 - DNA, 19 bp, Artificial Sequence
  Synthetic construct
cacaggtgccgcaatagcg

SEQ ID NO: 1397 - DNA, 22 bp, Artificial Sequence
  Synthetic construct
cagccaacctcgtcccagaggt

SEQ ID NO: 1398 - DNA, 18 bp, Artificial Sequence
  Synthetic construct
acgtgcccagacaagagc

SEQ ID NO: 1399 - DNA, 21 bp, Artificial Sequence
  Synthetic construct
gctccagagccataacctcat

SEQ ID NO: 1400 - DNA, 49 bp, Artificial Sequence
  Synthetic construct
gctgggagccagattgcgatttttatgttcagatggatgagattctcag

SEQ ID NO: 1401 - DNA, 43 bp, Artificial Sequence
  Synthetic construct
gatggcgtcgaatgacgccattttattgacctctgggacgagg

SEQ ID NO: 1402 - DNA, 17 bp, Artificial Sequence
  Synthetic construct
cctcccacgtgctcaga

SEQ ID NO: 1403 - DNA, 20 bp, Artificial Sequence
  Synthetic construct
acccatctgatgggtccgca

SEQ ID NO: 1404 - DNA, 25 bp, Artificial Sequence
  Synthetic construct
atgttcagatggatgagattctcag

SEQ ID NO: 1405 - DNA, 20 bp, Artificial Sequence
  Synthetic construct
gctgggagccagattgcgat

SEQ ID NO: 1406 - DNA, 19 bp, Artificial Sequence
  Synthetic construct
attgacctctgggacgagg

SEQ ID NO: 1407 - DNA, 20 bp, Artificial Sequence
  Synthetic construct
gatggcgtcgaatgacgcca